(19) United States
(12) Patent Application Publication
(10) Pub. No.: US 2023/0159569 A1
(43) Pub. Date: May 25, 2023

(54) DUAL HDAC6/PROTEASOME INHIBITORS, AND METHODS OF USE THEREOF

(71) Applicant: UNIVERSITY OF MARYLAND, BALTIMORE, Baltimore, MD (US)

(72) Inventors: Steven FLETCHER, Baltimore, MD (US); Alexandria CHAN, Baltimore, MD (US)

(73) Assignee: UNIVERSITY OF MARYLAND, BALTIMORE, Baltimore, MD (US)

(21) Appl. No.: 18/048,731

(22) Filed: Oct. 21, 2022

Related U.S. Application Data
(60) Provisional application No. 63/270,437, filed on Oct. 21, 2021.

(51) Int. Cl. C07F 5/02; C07D 209/90; C07C 237/44; C07C 237/40

(52) U.S. Cl. CPC C07F 5/027; C07D 209/90; C07C 237/44; C07C 237/40

(57) ABSTRACT

Dual HDAC6/proteasome inhibitors, and methods of using the same, are provided for treating disease.

### CROSS-REFERENCE TO RELATED APPLICATIONS

This application claims the benefit of U.S. Provisional Patent Application No. 63/270,437, filed Oct. 21, 2021, incorporated by reference herein in its entirety.

### FIELD

The disclosure relates generally to dual HDAC6/proteasome inhibitors and methods of using the same for treating conditions characterized by the overexpression or unregulated activity of the HDAC6 protein and/or the proteasome.

### BACKGROUND

Multiple myeloma (MM) is a cancer of the plasma cells, which overproduce abnormal proteins that need to be removed from the cell in order to promote malignant cell survival. However, there is considerable resistance to many treatments for MM, forcing patients into the relapsed/refractory category.

There is a need in the art for novel compounds that are useful in the treatment of multiple myeloma. The present disclosure addresses this need in the art.

### SUMMARY

In an embodiment, the disclosure includes a compound of formula (I), or comprising a substructure of formula (I), or a pharmaceutically acceptable salt, solvate, hydrate, cocrystal, or prodrug thereof:

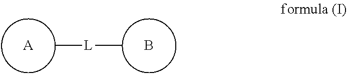

wherein in formula (I):
A comprises a zinc binding moiety;
L is a linking group; and
B comprises a proteasome inhibitor moiety, wherein the zinc binding moiety thereof, and the proteasome inhibitor moiety thereof are each connected to L at any chemically feasible site.

In some embodiments, B comprises a proteosome inhibitor moiety selected from bortezomib, ixazomib, carfilzomib, oprozomib, marizomib, CEP-18770, disulfiram, epigallocatechin-3-gallate, epoxomicin, lactacystin, MG132, MLN9708, ONX 0912, PR-924, PR-957, KZR-504, LMP7-IN-1, salinosporamide A, epoxomycine, eponemycine, aclacinomycine A, and any substructure thereof.

In some embodiments, B comprises a moiety selected from:

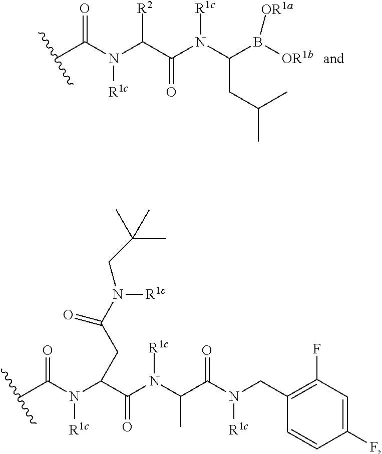

wherein R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups;
each R^{1c} is independently at each occurrence selected from hydrogen and —C₁₋₁₀ alkyl-; and
R² is selected from hydrogen, alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

In some embodiments, the compound of formula (I) is a compound of formula (10) or formula (12), or a pharmaceutically acceptable salt, solvate, hydrate, cocrystal, or prodrug thereof:

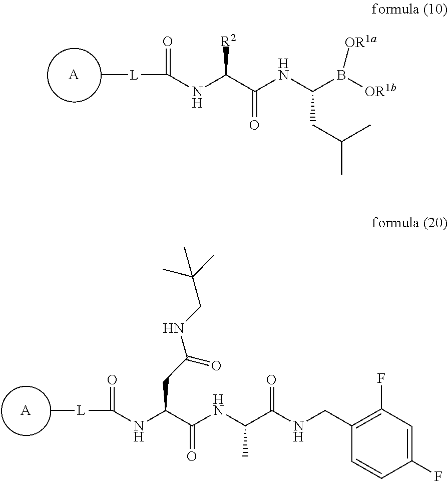

wherein in formula (10) and formula (20):
A comprises a zinc binding moiety;
L is a linking group;
R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups; and
R² is selected from hydrogen and optionally substituted —C₁₋₁₀ alkyl,
wherein the zinc binding moiety thereof is connected to L at any chemically feasible site.

In some embodiments, R² is selected from hydrogen and

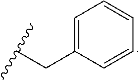 [SMILES: *Cc1ccccc1]

In some embodiments, the compound of formula (10) is a compound of formula (11) or formula (12), or a pharmaceutically acceptable salt, solvate, hydrate, cocrystal, or prodrug thereof:

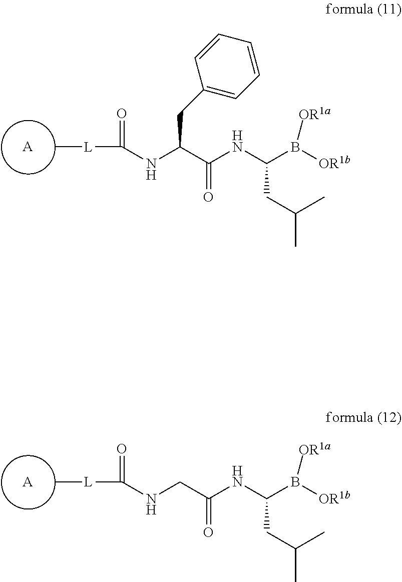

wherein in formula (11) and formula (12):
A comprises a zinc binding moiety;
L is a linking group; and
R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups.

In some embodiments, B comprises

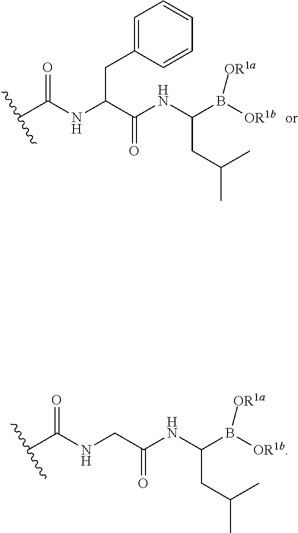

In some embodiments, R^{1a} and R^{1b} are each hydrogen or R^{1a} and R^{1b} are joined together to form a dioxaborolane, a dioxaborinane, or

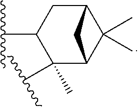 [SMILES: *C1C[C@H]2C[C@H](C2(C)C)[C@]1(*)C]

In some embodiments, the compound of formula (11) is a compound of formula (110) or formula (111), or a pharmaceutically acceptable salt, solvate, hydrate, cocrystal, or prodrug thereof:

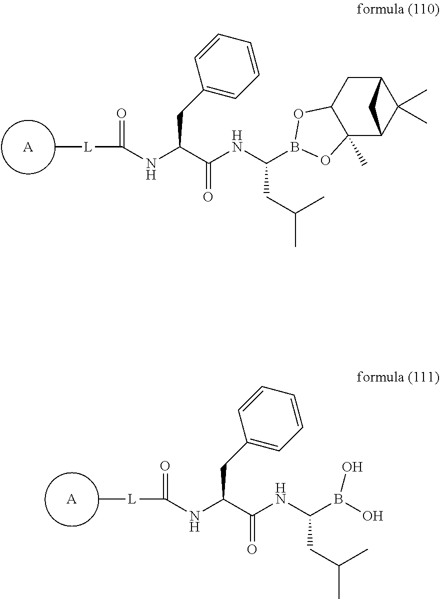

wherein in formula (110) and formula (111):
A comprises a zinc binding moiety; and
L is a linking group.

In some embodiments, the compound of formula (12) is a compound of formula (120) or formula (121), or a pharmaceutically acceptable salt, solvate, hydrate, cocrystal, or prodrug thereof:

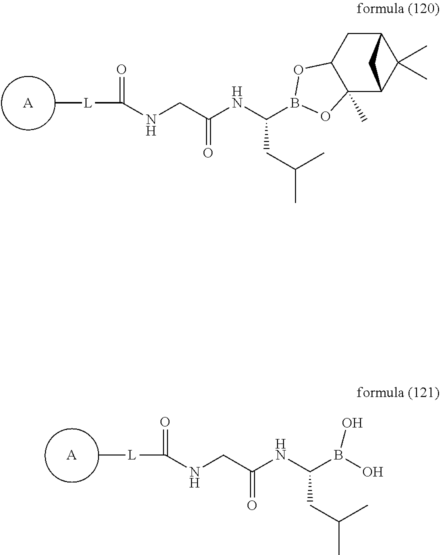

wherein in formula (120) and formula (121):
A comprises a zinc binding moiety; and
L is a linking group.

In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R)C(NR)N(R^{b})—, —N(Rh)S(O)_{w}—, —S(O)_{w}N(R^{b})—, —S(O)_{w}O—, —OS(O)_{w}—, —OS(O)_{w}O—, —O(O)P(OR)O—, (O)P(O—)₃, —O(S)P(OR)O—, and (S)P(O—)₃, wherein w is 1 or 2, and each R^{b} is independently hydrogen, optionally substituted alkyl, or optionally substituted aryl.

In some embodiments, L comprises one or more linking groups selected from optionally substituted —C₁₋₁₀ alkyl-, —C₁₋₁₀ alkenyl-, -aryl-, —C(O)—,

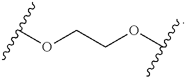 [SMILES: *OCCO*]

In some embodiments, L comprises one or more linking group selected from

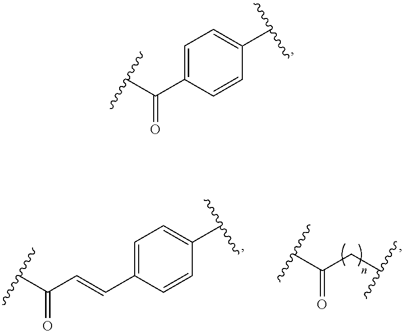

wherein n is an integer from 0 to 7,

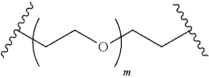

wherein m is an integer from 0 to 5,

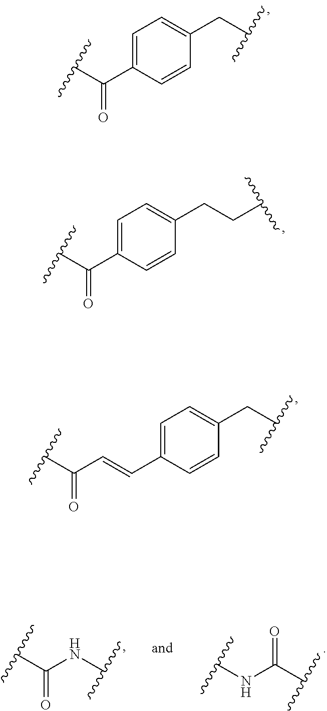 [SMILES: *Cc1ccc(C(*)=O)cc1 ; *CCc1ccc(C(*)=O)cc1 ; *Cc1ccc(/C=C/C(*)=O)cc1 ; *NC(*)=O ; *NC(*)=O]

In some embodiments, A comprises a moiety selected from:

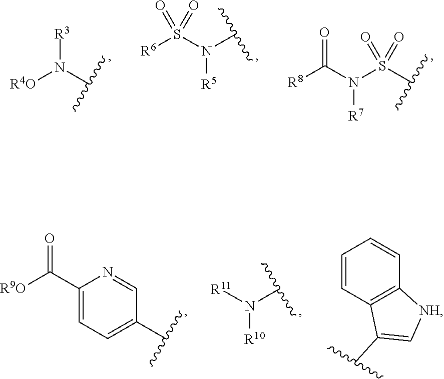

and R¹²O—,

wherein:
R³ is selected from hydrogen and optionally substituted —C₁₋₁₀ alkyl;
R⁴ is selected from hydrogen, N(R^{b})₂C(O)—, and R^{b}OC(O)—;
R⁵ is selected from hydrogen and optionally substituted —C₁₋₁₀ alkyl;
R⁶ is selected from optionally substituted —C₁₋₁₀ alkyl- and optionally substituted aryl;
R⁷ is selected from hydrogen and optionally substituted —C₁₋₁₀ alkyl;
R⁸ is optionally substituted —C₁₋₁₀ alkyl;
R⁹ is selected from hydrogen and optionally substituted —C₁₋₁₀ alkyl;
R¹⁰ is selected from hydrogen and optionally substituted —C₁₋₁₀ alkyl;
R¹¹ is optionally substituted aryl;
R¹² is optionally substituted —C₁₋₁₀ alkyl; and
each R^{b} is independently hydrogen, optionally substituted alkyl, optionally substituted heterocyclyl, or optionally substituted aryl.

In some embodiments, R⁷ is

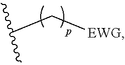

wherein EWG comprises an electron withdrawing group selected from —CN, —NO₂, —C(O)R¹³, —C(O)N(R¹⁴), and —N(R¹⁴)C(O)C(O)R¹⁴, wherein p is an integer between 1 and 5, R¹³ is selected from —C₁₋₁₀ alkyl, —C₁₋₁₀ alkoxy, and each R¹⁴ is independently selected from hydrogen and —C₁₋₁₀ alkyl.

In some embodiments,
R³ is hydrogen;
R⁴ is selected from hydrogen and N(R^{b})₂C(O)—;
R⁵ is selected from hydrogen, methyl, and

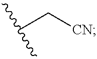 [SMILES: *CC#N]

R⁶ is selected from methyl, —CF₃, —CCl₃, and phenyl;
R⁷ is selected from hydrogen and

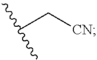 [SMILES: *CC#N]

R⁸ is methyl or —CF₃;
R⁹ is hydrogen;
R¹⁰ is hydrogen;
R¹¹ is amino substituted phenyl;
R¹² is methyl; and
each R^{b} is independently selected from hydrogen, ethyl, isopropyl, phenyl,

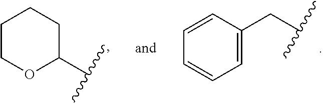 [SMILES: *C1CCCCO1 ; *Cc1ccccc1]

In some embodiments, A is selected from

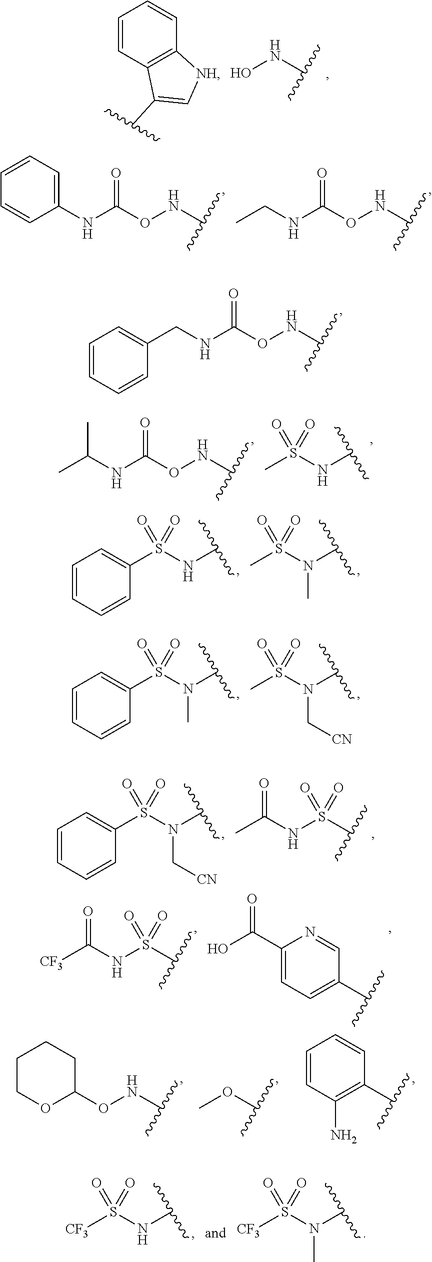

In some embodiments, the compound of formula (I) is a compound of any one of formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt, solvate, hydrate, cocrystal, or prodrug thereof.

In one aspect, the disclosure provides a pharmaceutical composition comprising one or more of compounds of any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium.

In one aspect, the disclosure provides a pharmaceutical composition for treating or preventing a disease or disorder alleviated by inhibiting HDAC6 protein activity, the pharmaceutical composition comprising one or more compounds according to any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium. In one aspect, the disclosure provides a pharmaceutical composition for treating or preventing a disease or disorder alleviated by inhibiting proteasome activity, the pharmaceutical composition comprising one or more compounds according to any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium. In one aspect, the disclosure provides a pharmaceutical composition for treating or preventing a disease or disorder alleviated by both inhibiting HDAC6 protein activity and inhibiting proteasome activity, the pharmaceutical composition comprising one or more compounds according to any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium. In some embodiments, the disease or disorder is cancer. In some embodiments, the cancer is selected from multiple myeloma (MM), mantle cell lymphoma, acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is a blood cancer. In some embodiments, the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma. In some embodiments, the cancer is multiple myeloma (MM). In some embodiments, the disease or disorder is an inflammatory disease or disorder. In some embodiments, the inflammatory disease or disorder is selected from autoimmune diseases, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis. In some embodiments, the disease or disorder is an autoimmune disease or disorder. In some embodiments, the autoimmune disease or disorder is selected from Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

In one aspect, the disclosure provides a pharmaceutical composition for treating or preventing from multiple myeloma (MM), the pharmaceutical composition comprising one or more compounds according any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium.

In one aspect, the disclosure provides a method of treating or preventing a disease or disorder alleviated by inhibiting HDAC6 protein activity in a patient in need of said treatment or prevention, the method comprising administering a therapeutically effective amount of one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof. In one aspect, the disclosure provides a method of treating or preventing a disease or disorder alleviated by inhibiting proteasome activity in a patient in need of said treatment or prevention, the method comprising administering a therapeutically effective amount of one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof. In one aspect, the disclosure provides a method of treating or preventing a disease or disorder alleviated by both inhibiting HDAC6 protein activity and inhibiting proteasome activity in a patient in need of said treatment or prevention, the method comprising administering a therapeutically effective amount of one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, a pharmaceutically acceptable salt thereof. In some embodiments, the disease or disorder is cancer. In some embodiments, the cancer is selected from multiple myeloma (MM), mantle cell lymphoma, acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is a blood cancer. In some embodiments, the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma. In some embodiments, the cancer is multiple myeloma (MM). In some embodiments, the disease or disorder is an inflammatory disease or disorder. In some embodiments, the inflammatory disease or disorder is selected from autoimmune diseases, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis. In some embodiments, the disease or disorder is an autoimmune disease or disorder. In some embodiments, the autoimmune disease or disorder is selected from Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

In one aspect, the disclosure provides a method of treating or preventing acute multiple myeloma (MM) in a patient in need of said treatment or prevention, the method comprising administering a therapeutically effective amount of one or more compounds of any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, a pharmaceutically acceptable salt thereof.

### BRIEF DESCRIPTION OF THE DRAWINGS

The foregoing summary, as well as the following detailed description of embodiments of the disclosure, will be better understood when read in conjunction with the appended drawings and figures.

In the drawings:

FIG. 1A and FIG. 1B illustrate non-limiting general strategies to prepare dual HDAC6/proteasome inhibitor compounds of the disclosure.

FIG. 2 illustrates a scheme of the Ubiquitin-Proteasome System (UPS).

FIG. 3 illustrates a scheme of the aggresome pathway.

FIG. 4 is an image illustrating the overlay of crystal structures for HDACs 1, 2, 3, and 6.

FIG. 5 is an image illustrating Site Identification by Ligand Competitive Saturation (SILCS) modeling, which determines areas on the protein that certain small molecules are likely to bind, termed “FragMaps.” The image shows an overlay of two covalent dual inhibitors of the disclosure containing phenyl and isopropyl carbamates. The carbamates lay in the green FragMap, denoting areas of potential hydrophobic interactions, which can be useful for carbamates to further contribute to HDAC6 binding.

FIG. 6 illustrates non-limiting examples of dual HDAC6/proteasome inhibitor compounds of the disclosure.

FIG. 7 illustrates non-limiting embodiments of compounds of the disclosure.

FIG. 8 illustrates a non-limiting example of the synthesis of a linker for dual HDAC6/proteasome inhibitor compounds of the disclosure.

FIG. 9 illustrates a non-limiting example of the synthesis of prodrug of dual HDAC6/proteasome inhibitor compounds of the disclosure.

FIG. 10 illustrates a non-limiting example of the synthesis of a dual HDAC6/proteasome inhibitor compound of the disclosure comprising a hydroxamic acid moiety as a zinc binding group (ZBG).

FIG. 11 illustrates experimental data demonstrating the potency and selectivity for HDAC6 of compounds of the disclosure.

FIG. 12A illustrates experimental data demonstrating the percent viability (MTT) of compound AMC-2-052. FIG. 12B illustrates experimental data demonstrating the percent viability (MTT) of compound AMC-2-067.

FIGS. 13A-13C illustrate the structures of non-limiting embodiments of dual HDAC6/covalent proteasome inhibitors of the disclosure.

FIGS. 14A-14B illustrate the structures of non-limiting embodiments of dual HDAC6/non-covalent proteasome inhibitors of the disclosure.

FIGS. 15A-15B illustrate the structures of non-limiting embodiments of HDAC6 inhibitors of the disclosure that include novel zinc binding groups (acyl sulfonamides).

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include the compounds described herein and, more specifically, the one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225. The terms “active pharmaceutical ingredient” and “drug” may also include those compounds described herein that inhibit the HDAC6 protein and thereby modulate HDAC6 protein activity and/or inhibit the proteasome and thereby modulate proteasome activity.

The term “isostere” refers to a group or molecule whose chemical and/or physical properties are similar to those of another group or molecule. A “bioisostere” is a type of isostere and refers to a group or molecule whose biological properties are similar to those of another group or molecule. For example, for the compounds described herein, a carboxylic acid may be replaced by one of the following bioisosteres for carboxylic acids, including, without limitation, alkyl esters (COOR), acylsulfonamides (CONR—SO₂R), hydroxamic acids (CONR—OH), hydroxamates (CONR—OR), tetrazoles, hydroxyisoxazoles, isoxazol-3-ones, and sulfonamides (SO₂NR), where each R may independently represent hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “treat,” “treatment,” and/or “treating” may refer to the management of a disease, disorder, or pathological condition (e.g., pain, a neurological disorder, diarrhea, coughing, muscular tension, and glaucoma), or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. In some embodiments, the biological molecules modulated by the methods and compounds of the disclosure to effect treatment may include one or both of the δ-opioid receptor and the p-opioid receptor.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” or “physiologically compatible” carrier or carrier medium is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

A “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Acylsulfonamide” refers to the group —C(═O)NR^{a}—S(═O)R^{a}, where each R^{a} is independently hydrogen, alkyl, fluoroalkyl, carbocyclyl, carbocyclylalkyl, aryl, aralkyl, heterocycloalkyl, heterocycloalkylalkyl, heteroaryl, or heteroarylalkyl.

“Carboxaldehyde” refers to a —(C═O)H radical.

“Carbonyl” refers to the group —C(═O)—. Carbonyl groups may be substituted with the following exemplary substituents: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —NR^{a}—OR^{a}—, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, acylsulfonamido, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, acylsulfonamido, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}—, (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, amino, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, acylsulfonamido, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, isoxazol-3-one, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” or “heterocyclyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“hydroxamate” refers to the —C(O)NR^{a}OR^{a} moiety, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, 3rd Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxamate, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)— (optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### DETAILED DESCRIPTION

Polypharmacology (i.e., a single drug hitting multiple targets) is touted to be superior than the corresponding combination therapy. HDAC6 upregulation is a known resistance mechanism in proteasome inhibitor therapy for the treatment of multiple myeloma. In part, the disclosure provides novel compounds that include both HDAC6 inhibitor and proteasome inhibitor pharmacophores, and can be useful for treating diseases and disorders such as multiple myeloma. In some embodiments, the compounds of the disclosure are useful to overcome resistance to proteasome inhibitors in multiple myeloma.

Polypharmacologic agents have increased in popularity, especially in the treatment of multifactorial diseases such as cancer. Polypharmacologic drug compounds contain pharmacophores for two or more targets of interest and may result in several advantages over traditional combination treatments, such as a potential synergistic therapeutic affect due to the simultaneous presence of both pharmacophores in the sites of need. The ability of these agents to incapacitate multiple targets can be applied to the treatment of cancer therapeutic resistance, which is often due to a compensatory upregulation of related proteins. The advantages of a polypharmacological approach include, but are not limited to, increased synergistic effect when compared to combination treatment, simultaneous presence of both pharmacophores in an active site, suggesting reduced doses may be required for full therapeutic effect, reducing side effects and increasing patient compliance, and more predictable pharmacokinetic profile and minimization of drug-drug interactions (DDIs).

Multiple myeloma (MM) is a cancer of the plasma cells, which overproduce abnormal proteins that need to be removed from the cell in order to promote malignant cell survival. For this reason, proteasome inhibitors have become a first-line treatment for MM by inhibiting the main method of protein degradation; however, there is considerable resistance to these treatments, forcing patients into the relapsed/refractory category. One major cause of resistance is the upregulation of histone deacetylase-6 (HDAC6). This enzyme plays a key role in the aggresome pathway, a compensatory protein degradation method. Recently, a phase I/II clinical trial of the combination treatment of bortezomib (proteasome inhibitor) and ricolinostat (HDAC inhibitor) showed therapeutic efficacy in resistant MM.

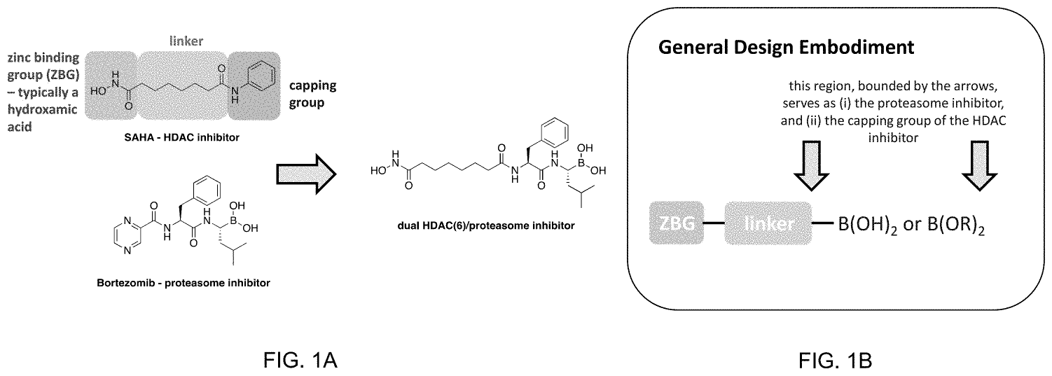

In one aspect, the disclosure provides novel dual HDAC6/proteasome inhibitors that include pharmacophores from HDAC6 inhibitors and proteasome inhibitors, since both are involved in pathways that promote multiple myeloma. In some embodiments, the HDAC(6) pharmacophore is composed of three subunits (FIG. 1A). In a non-limiting example, the capping group of HDAC inhibitors (that preferentially target HDAC6) is replaced with the crucial part of proteasome inhibitor pharmacophores. In some embodiments, the critical part of an HDAC inhibitor may include the hydroxamic acid, which is a zinc binding group (ZBG). In some embodiments, the compounds of the disclosure comprise a hydroxamic acid or other novel ZBGs, such as the N-acyl-N-alkyl sulfonamide (NASA) electrophilic warhead. In some embodiments, the critical part of a proteasome inhibitor includes the boronic acid (or boronate ester prodrug (FIG. 1), which can form a reversible covalent bond with a threonine in the proteasome through an empty p orbital on the boron atom. In some embodiments, the dual HDAC6/proteasome inhibitors of the disclosure have increased therapeutic efficacy when compared to traditionally used combination treatments.

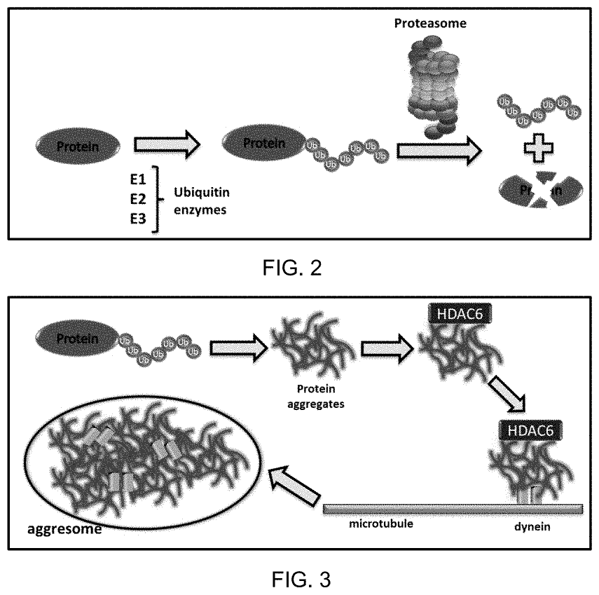

The Ubiquitin-Proteasome System (UPS) (FIG. 2) is a main method of protein degradation in cells, and works in tandem with E1, E2, and E3 enzymes to ubiquitinate target proteins, tagging them for degradation. Ubiquitin tagged proteins are then degraded by the proteasome and broken down into peptides, and the ubiquitin is recycled for future use. When ubiquitinated proteins are not degraded by the proteasome, they form protein aggregates (FIG. 3). HDAC6 aids in moving protein aggregates to aggresomes. Intact microtubules are required to transport protein aggregates through the cytoplasm to the aggresome through the use of motor dyneins, and proteins are stored in aggresomes until they are degraded through autophagy.

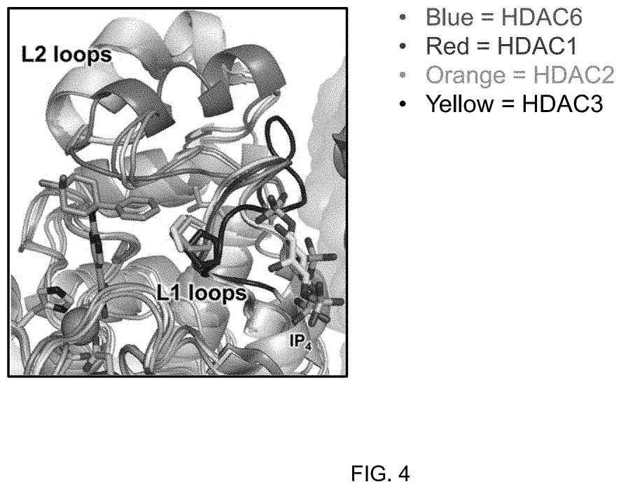

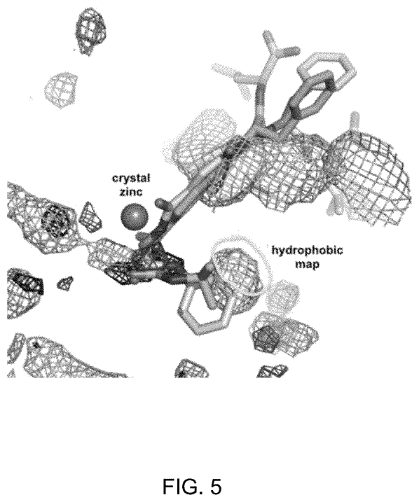

In some embodiments, the compounds of the disclosure are selective to HDAC6 over other HDACs, such as HDAC1, HDAC2, or HDAC3. In some embodiments, HDAC6 selectivity can be achieved through modification of the general formula for HDAC inhibitors. Non-limiting examples of modifications that can provide HDAC6 selectivity include increasing steric bulk of the linking and/or capping groups and including a wider binding pocket of HDAC6 compared to other isozymes readily accommodates these larger inhibitors (FIG. 4).

In one aspect, the present disclosure provides novel compounds that function as inhibitors of HDAC6 and/or proteasome inhibitors and are useful in treating diseases or disorders associated with dysregulation of HDAC6 and/or the proteasome. In some embodiments, the compounds of the disclosure are useful as treatments for a range of cancers, anti-inflammatory diseases or disorders, and/or autoimmune diseases or disorders.

### Dual HDAC6/Proteasome Inhibitors

In one aspect, the disclosure provides compounds that are inhibitors of HDAC6 protein activity and/or inhibitors of proteasome activity.

In some embodiments, the compounds described above may be delivered as listed or as a pharmaceutically acceptable salt, solvate, hydrate, cocrystal, tautomer, or prodrug thereof.

In one aspect, the disclosure provides a compound of formula (I), or comprising a substructure of formula (I), or a pharmaceutically acceptable salt, solvate, hydrate, cocrystal, or prodrug thereof:

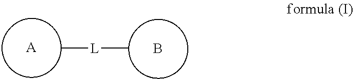

wherein in formula (I):
A comprises a zinc binding moiety;
L is a linking group; and
B comprises a proteasome inhibitor moiety, wherein the zinc binding moiety thereof, and the proteasome inhibitor moiety thereof are each connected to L at any chemically feasible site.

Any proteasome inhibitor, or substructure thereof, is contemplated by the present disclosure. In some embodiments, B comprises a proteosome inhibitor moiety selected from bortezomib, ixazomib, carfilzomib, oprozomib, marizomib, CEP-18770, disulfiram, epigallocatechin-3-gallate, epoxomicin, lactacystin, MG132, MLN9708, ONX 0912, PR-924, PR-957, KZR-504, LMP7-IN-1, salinosporamide A, epoxomycine, eponemycine, aclacinomycine A, and any substructure thereof.

In some embodiments, B comprises a moiety selected from:

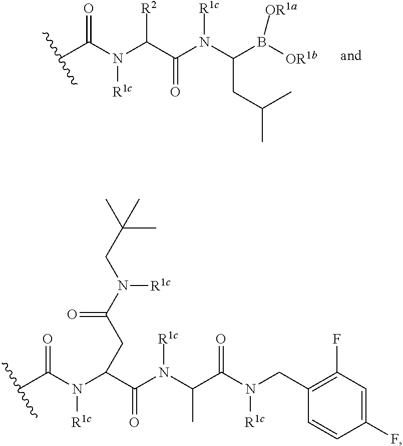

wherein R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups;
each R^{1c} is independently at each occurrence selected from hydrogen and —C₁₋₁₀ alkyl-; and
R² is selected from hydrogen, alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

In some embodiments, each R^{1c} is hydrogen.

In some embodiments, B comprises a moiety selected from:

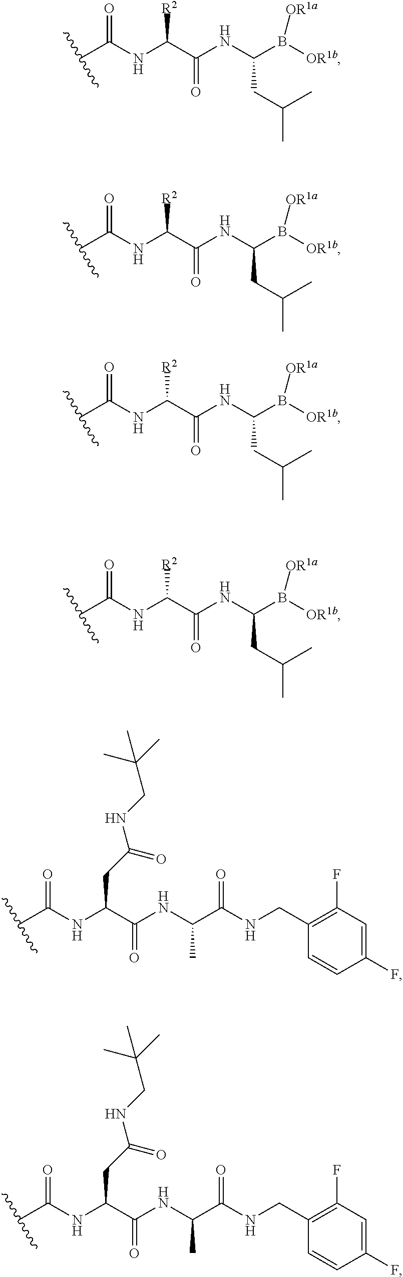

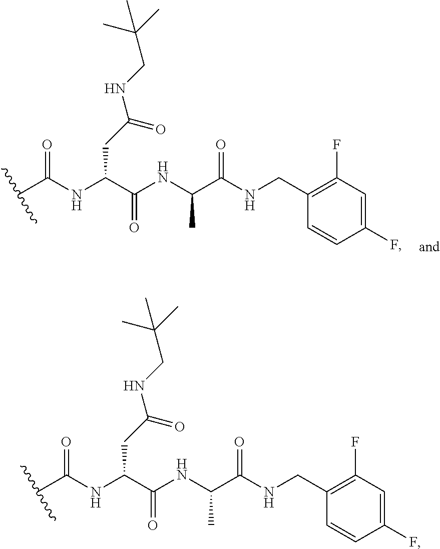 [SMILES: *C(=O)N[C@H](CC(=O)NCC(C)(C)C)C(=O)N[C@H](C)C(=O)NCc1ccc(F)cc1F ; *C(=O)N[C@H](CC(=O)NCC(C)(C)C)C(=O)N[C@@H](C)C(=O)NCc1ccc(F)cc1F]

In some embodiments, R² is selected from hydrogen and optionally substituted —C₁₋₁₀ alkyl.

In some embodiments, R² is selected from hydrogen and optionally substituted —C₁₋₁₀ alkyl, wherein —C₁₋₁₀ alkyl is optionally substituted with one or more substituents independently selected from the group consisting of heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

In some embodiments, B comprises a moiety selected from:

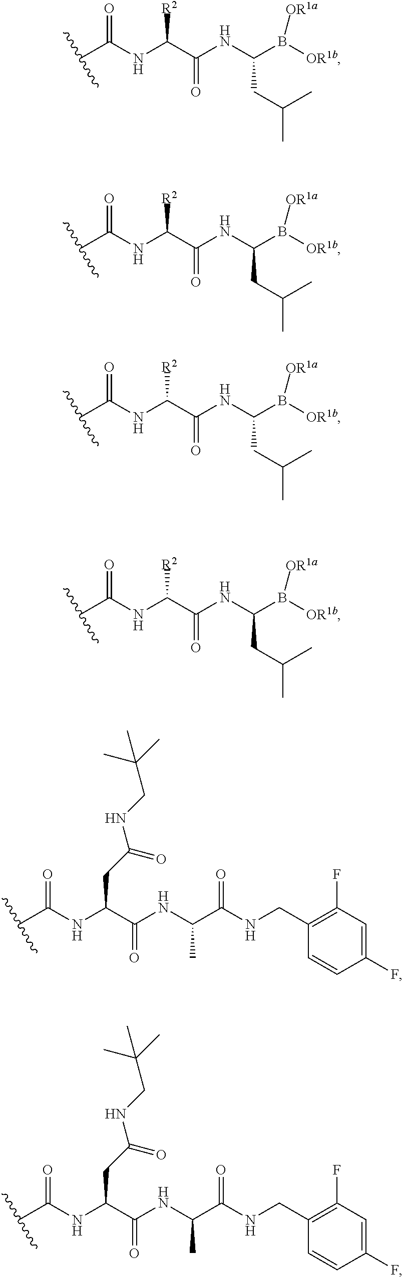

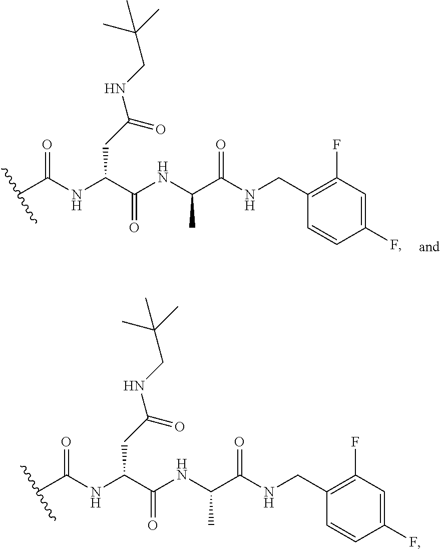

wherein R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups; and
R² is selected from hydrogen and optionally substituted —C₁₋₁₀ alkyl.

In some embodiments, B comprises a moiety selected from:

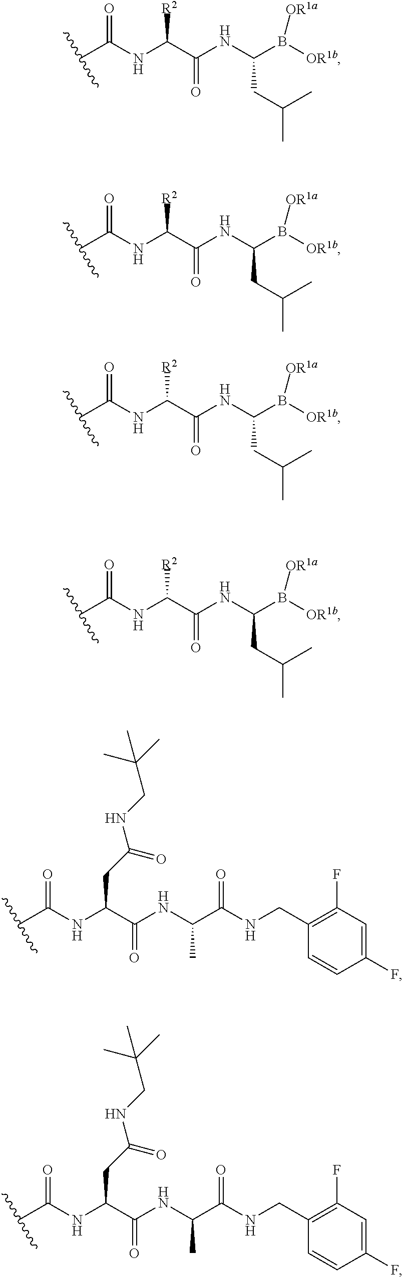

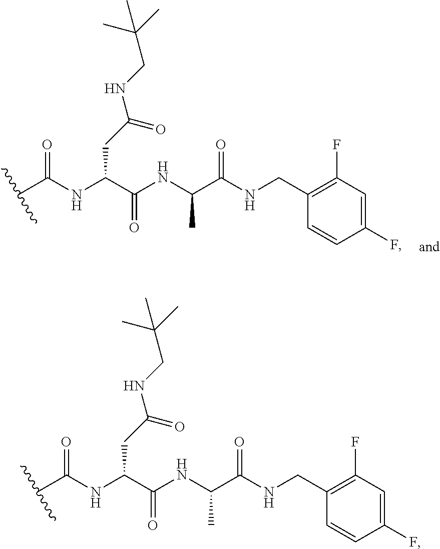

wherein R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups; and
R² is selected from hydrogen and optionally substituted —C₁₋₁₀ alkyl, wherein —C₁₋₁₀ alkyl is optionally substituted with one or more substituents independently selected from the group consisting of heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

In some embodiments, the compound of formula (I) is a compound of formula (10) or formula (12), or a pharmaceutically acceptable salt, solvate, hydrate, cocrystal, or prodrug thereof:

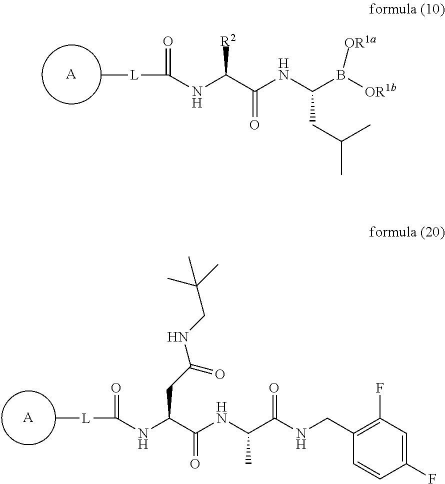

wherein in formula (10) and formula (20):
A comprises a zinc binding moiety;
L is a linking group;
R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups; and
R² is selected from hydrogen and optionally substituted —C₁₋₁₀ alkyl,
wherein the zinc binding moiety thereof is connected to L at any chemically feasible site.

In some embodiments, R² is selected from hydrogen and

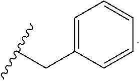 [SMILES: *Cc1ccccc1]

In some embodiments, the compound of formula (10) is a compound of formula (11) or formula (12), or a pharmaceutically acceptable salt, solvate, hydrate, cocrystal, or prodrug thereof:

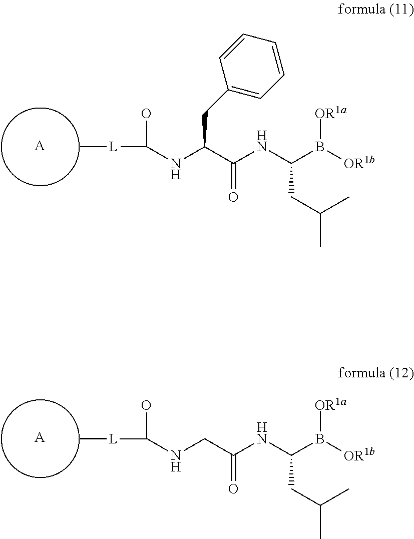

wherein in formula (11) and formula (12):
A comprises a zinc binding moiety;
L is a linking group; and
R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups.

In some embodiments, B comprises:

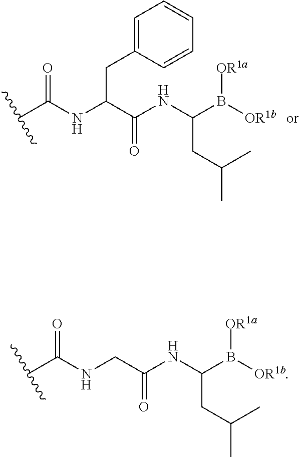

In some embodiments, R^{1a} and R^{1b} form an acyclic boronate ester. In some embodiments, R^{1a} and R^{1b} form a cyclic boronate ester. In some embodiments, R^{1a} and R^{1b} are each hydrogen, or R^{1a} and R^{1b} are joined together to form a dioxaborolane, a dioxaborinane, or

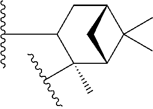 [SMILES: *C1C[C@H]2C[C@H](C2(C)C)[C@]1(*)C]

In some embodiments, the compound of formula (10) is a compound of formula (11) or formula (12), or a pharmaceutically acceptable salt, solvate, hydrate, cocrystal, or prodrug thereof:

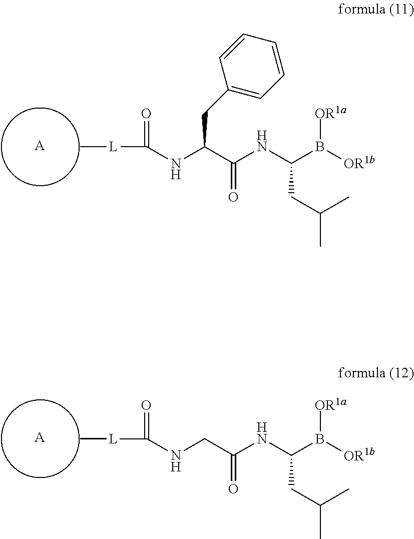

wherein in formula (11) and formula (12):
A comprises a zinc binding moiety;
L is a linking group; and
R^{1a} and R^{1b} are each hydrogen, or R^{1a} and R^{1b} are joined together to form a dioxaborolane, a dioxaborinane, or

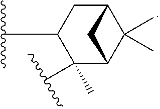 [SMILES: *C1C[C@H]2C[C@H](C2(C)C)[C@]1(*)C]

In some embodiments, the compound of formula (11) is a compound of formula (110) or formula (111), or a pharmaceutically acceptable salt, solvate, hydrate, cocrystal, or prodrug thereof:

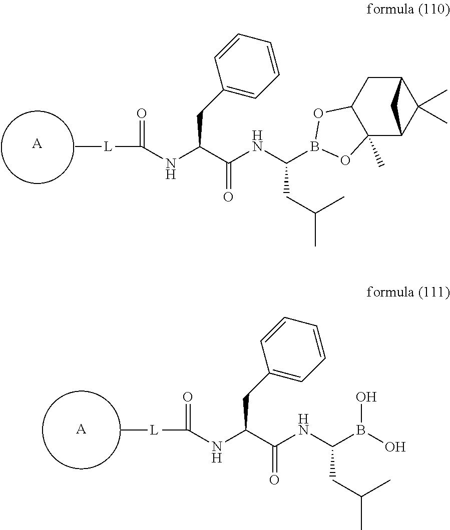

wherein in formula (110) and formula (111):
A comprises a zinc binding moiety; and
L is a linking group.

In some embodiments, the compound of formula (12) is a compound of formula (120) or formula (121), or a pharmaceutically acceptable salt, solvate, hydrate, cocrystal, or prodrug thereof:

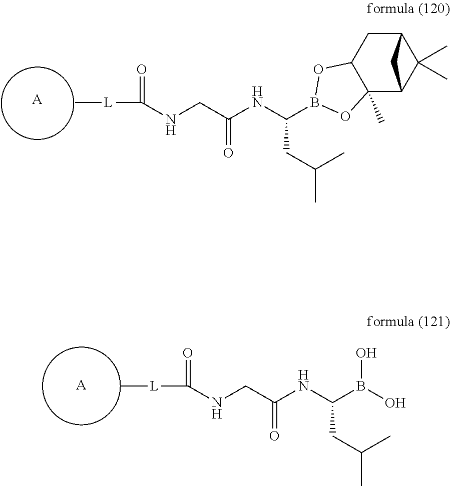

wherein in formula (120) and formula (121):
A comprises a zinc binding moiety; and
L is a linking group.

Any organic linker L is contemplated by the present disclosure. In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and each R^{b} is independently hydrogen, optionally substituted alkyl, or optionally substituted aryl.

In some embodiments, wherein L comprises one or more linking groups selected from optionally substituted —C₁₋₁₀ alkyl-, —C₁₋₁₀ alkenyl-, -aryl-, —C(O)—,

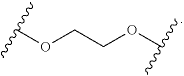 [SMILES: *OCCO*]

In some embodiments, L comprises one or more linking groups selected from

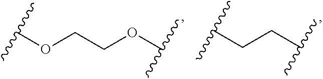

### —NHC(O)—, and —C(O)NH—.

In some embodiments, L comprises

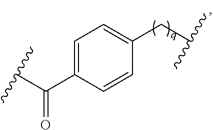

wherein q is an integer from 1 to 5 (e.g., 1, 2, 3, 4, or 5).

In some embodiments L comprises one or more linking groups selected from

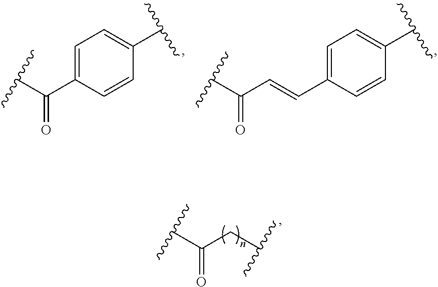

wherein n is an integer from 0 to 7 (e.g., 0, 1, 2, 3, 4, 5, 6, or 7; from 3 to 7; from 5 to 7; etc.),

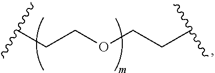

wherein m is an integer from 0 to 5 (e.g., 0, 1, 2, 3, 4, or 5; from 1 to 5; etc.),

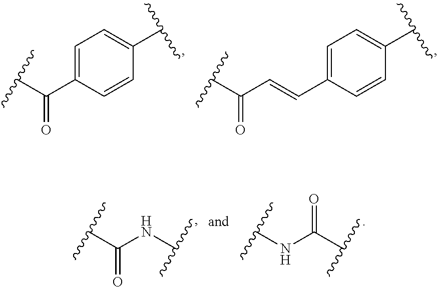 [SMILES: *C(=O)c1ccc(*)cc1 ; *C(=O)/C=C/c1ccc(*)cc1 ; *NC(*)=O ; *NC(*)=O]

Any zinc binding moiety is contemplated by the present disclosure. In some embodiments, A comprises an acyl sulfonamide moiety. In a non-limiting embodiment, the acyl sulfonamide moiety comprises a moiety selected from:

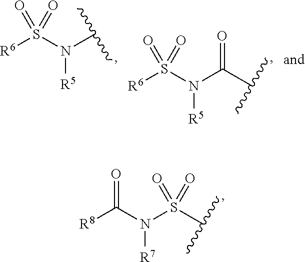

wherein:
R⁵ is selected from hydrogen and optionally substituted —C₁₋₁₀ alkyl;
R⁶ is selected from optionally substituted —C₁₋₁₀ alkyl- and optionally substituted aryl;
R⁷ is selected from hydrogen and optionally substituted —C₁₋₁₀ alkyl; and
R⁸ is optionally substituted —C₁₋₁₀ alkyl.

In some embodiments, A comprises a moiety selected from:

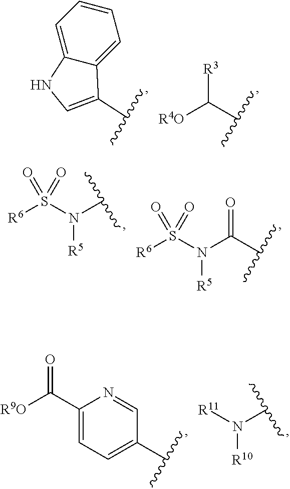

### and R¹²O—,

wherein:
R³ is selected from hydrogen and optionally substituted —C₁₋₁₀ alkyl;
R⁴ is selected from hydrogen, N(R^{b})₂C(O)—, and R^{b}OC(O)—;
R⁵ is selected from hydrogen and optionally substituted —C₁₋₁₀ alkyl;
R⁶ is selected from optionally substituted —C₁₋₁₀ alkyl- and optionally substituted aryl;
R⁷ is selected from hydrogen and optionally substituted —C₁₋₁₀ alkyl;
R⁸ is optionally substituted —C₁₋₁₀ alkyl;
R⁹ is selected from hydrogen and optionally substituted —C₁₋₁₀ alkyl;
R¹⁰ is selected from hydrogen and optionally substituted —C₁₋₁₀ alkyl;
R¹¹ is optionally substituted aryl;
R¹² is optionally substituted —C₁₋₁₀ alkyl; and
each R^{b} is independently hydrogen, optionally substituted alkyl, optionally substituted heterocyclyl, or optionally substituted aryl.

In some embodiments, R⁷ is

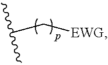

wherein EWG comprises an electron withdrawing group. Non-limiting examples of electron withdrawing groups include carbonyls, ketones, esters, amides, —SO₂—, —SO—, —CO—CO—, —CO—COOR, sulfonamides, trihalomethyl (such as trifluoromethyl), sulfonamides, halides, maleimides, maleates, or combinations thereof. In some embodiments, the EWG comprises an electron withdrawing group selected from —CN, —NO₂, —C(O)R¹³, —C(O)N(R¹⁴), and —N(R¹⁴)C(O)C(O)R¹⁴, wherein p is an integer between 1 and 5, R¹³ is selected from —C₁₋₁₀ alkyl, —C₁₋₁₀ alkoxy, and each R¹⁴ is independently selected from hydrogen and —C₁₋₁₀ alkyl.

In some embodiments, A comprises a moiety selected from:

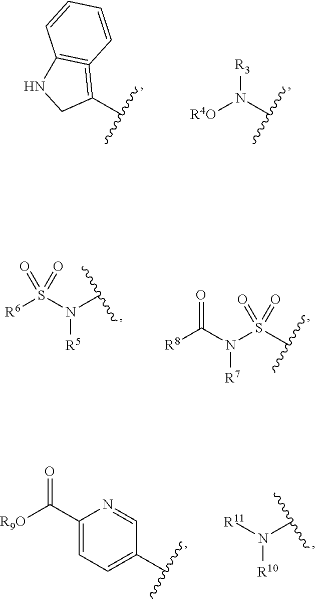

### and R¹²O—,

wherein:
R³ is selected from hydrogen and optionally substituted —C₁₋₁₀ alkyl-;
R⁴ is selected from hydrogen, N(R^{b})₂C(O)—, and —R^{b}OC(O)—;
R⁵ is selected from hydrogen and optionally substituted —C₁₋₁₀ alkyl-;
R⁶ is selected from optionally substituted —C₁₋₁₀ alkyl- and optionally substituted aryl;
R⁷ is selected from hydrogen and

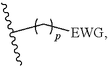

wherein EWG comprises an electron withdrawing group selected from —CN, —NO₂, —C(O)R¹³, —C(O)N(R¹⁴), and —N(R¹⁴)C(O)C(O)R¹⁴, wherein p is an integer between 1 and 5, R¹³ is selected from —C₁₋₁₀ alkyl, —C₁₋₁₀ alkoxy, and each R¹⁴ is independently selected from hydrogen and —C₁₋₁₀ alkyl;
R⁸ is optionally substituted —C₁₋₁₀ alkyl-;
R⁹ is selected from hydrogen and optionally substituted —C₁₋₁₀ alkyl-;
R¹⁰ is selected from hydrogen and optionally substituted —C₁₋₁₀ alkyl-;
R¹¹ is optionally substituted aryl;
R¹² is optionally substituted —C₁₋₁₀ alkyl; and
each R^{b} is independently hydrogen, optionally substituted alkyl, optionally substituted heterocyclyl, or optionally substituted aryl.

In some embodiments,
R³ is hydrogen;
R⁴ is selected from hydrogen and N(R^{b})₂C(O)—;
R⁵ is selected from hydrogen, methyl, and

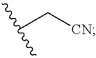 [SMILES: *CC#N]

R⁶ is selected from methyl, —CF₃, —CCl₃, and phenyl;
R⁷ is selected from hydrogen and

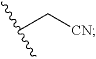 [SMILES: *CC#N]

R⁸ is methyl or —CF₃;
R⁹ is hydrogen;
R¹⁰ is hydrogen;
R¹¹ is amino substituted phenyl;
R¹² is methyl; and
each R^{b} is independently selected from hydrogen, ethyl, isopropyl, phenyl,

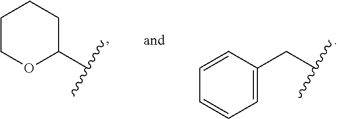 [SMILES: *C1CCCCO1 ; *Cc1ccccc1]

In some embodiments, A is selected from

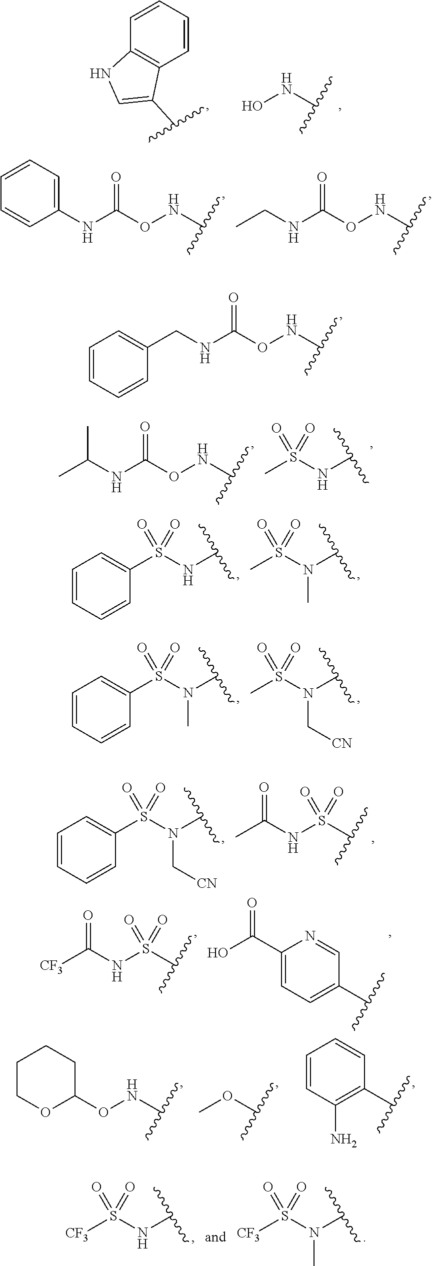

In some embodiments, the compound of formula (I) is a compound of any one of formula 1001-1114, or a pharmaceutically acceptable salt, solvate, hydrate, cocrystal, or prodrug thereof, wherein in formula 1001-1114:
B is:

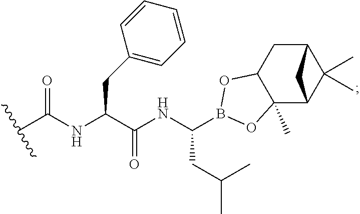 [SMILES: *C(=O)N[C@@H](Cc1ccccc1)C(=O)N[C@@H](CC(C)C)B1OC2C[C@H]3C[C@H](C3(C)C)[C@@]2(C)O1]

and:

| Formula No. | A | L | n |
|---|---|---|---|
| 1001 | 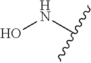 [SMILES: *NO] | 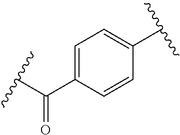 [SMILES: *C(=O)c1ccc(*)cc1] | — |
| 1002 | 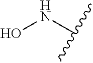 [SMILES: *NO] | 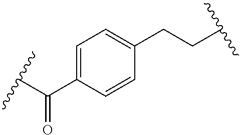 [SMILES: *CCc1ccc(C(*)=O)cc1] | |
| 1003 | 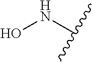 [SMILES: *NO] | 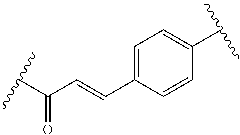 [SMILES: *C(=O)/C=C/c1ccc(*)cc1] | — |
| 1004 | 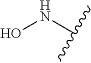 [SMILES: *NO] | 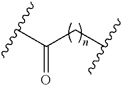 | 5 |
| 1005 | 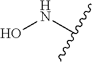 [SMILES: *NO] | 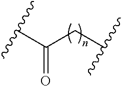 | 6 |
| 1006 | 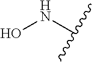 [SMILES: *NO] | 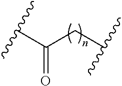 | 7 |
| 1007 | 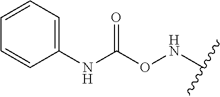 [SMILES: *NOC(=O)Nc1ccccc1] | 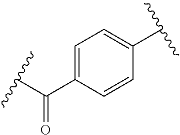 [SMILES: *C(=O)c1ccc(*)cc1] | — |
| 1008 | 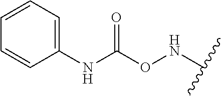 [SMILES: *NOC(=O)Nc1ccccc1] | 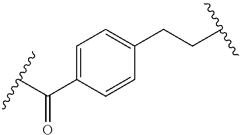 [SMILES: *CCc1ccc(C(*)=O)cc1] | |
| 1009 | 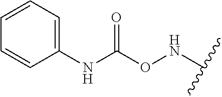 [SMILES: *NOC(=O)Nc1ccccc1] | 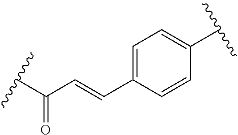 [SMILES: *C(=O)/C=C/c1ccc(*)cc1] | — |
| 1010 | 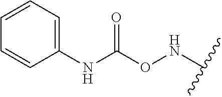 [SMILES: *NOC(=O)Nc1ccccc1] | 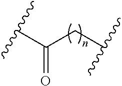 | 5 |
| 1011 | 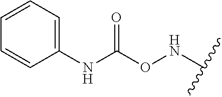 [SMILES: *NOC(=O)Nc1ccccc1] | 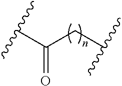 | 6 |
| 1012 | 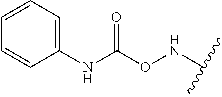 [SMILES: *NOC(=O)Nc1ccccc1] | 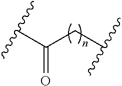 | 7 |
| 1013 | 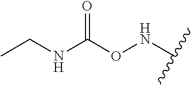 [SMILES: *NOC(=O)NCC] | 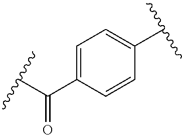 [SMILES: *C(=O)c1ccc(*)cc1] | — |
| 1014 | 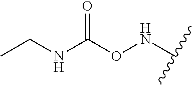 [SMILES: *NOC(=O)NCC] | 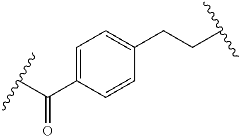 [SMILES: *CCc1ccc(C(*)=O)cc1] | |
| 1015 | 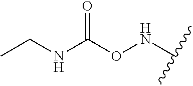 [SMILES: *NOC(=O)NCC] | 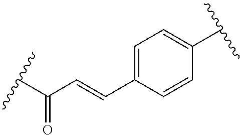 [SMILES: *C(=O)/C=C/c1ccc(*)cc1] | — |
| 1016 | 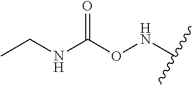 [SMILES: *NOC(=O)NCC] | 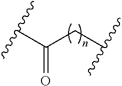 | 5 |
| 1017 | 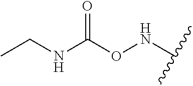 [SMILES: *NOC(=O)NCC] | 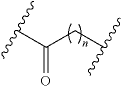 | 6 |
| 1018 | 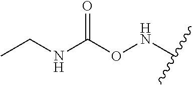 [SMILES: *NOC(=O)NCC] | 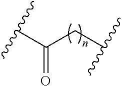 | 7 |
| 1019 | 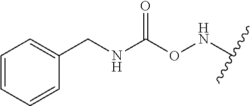 [SMILES: *NOC(=O)NCc1ccccc1] | 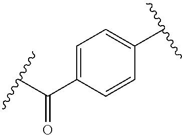 [SMILES: *C(=O)c1ccc(*)cc1] | — |
| 1020 | 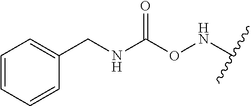 [SMILES: *NOC(=O)NCc1ccccc1] | 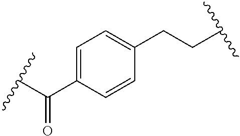 [SMILES: *CCc1ccc(C(*)=O)cc1] | |
| 1021 | 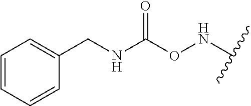 [SMILES: *NOC(=O)NCc1ccccc1] | 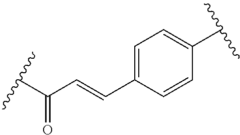 [SMILES: *C(=O)/C=C/c1ccc(*)cc1] | — |
| 1022 | 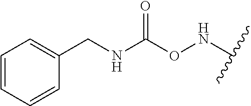 [SMILES: *NOC(=O)NCc1ccccc1] | 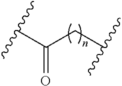 | 5 |
| 1023 | 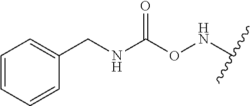 [SMILES: *NOC(=O)NCc1ccccc1] | 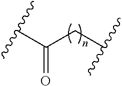 | 6 |
| 1024 | 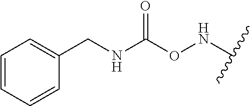 [SMILES: *NOC(=O)NCc1ccccc1] | 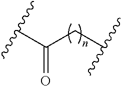 | 7 |
| 1025 | 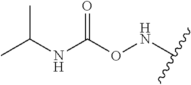 [SMILES: *NOC(=O)NC(C)C] | 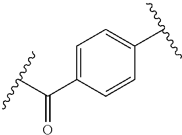 [SMILES: *C(=O)c1ccc(*)cc1] | — |
| 1026 | 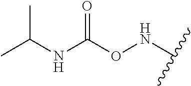 [SMILES: *NOC(=O)NC(C)C] | 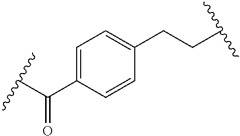 [SMILES: *CCc1ccc(C(*)=O)cc1] | |
| 1027 | 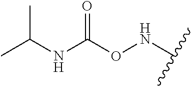 [SMILES: *NOC(=O)NC(C)C] | 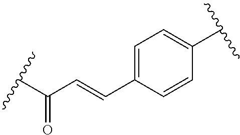 [SMILES: *C(=O)/C=C/c1ccc(*)cc1] | — |
| 1028 | 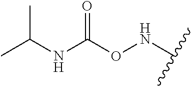 [SMILES: *NOC(=O)NC(C)C] | 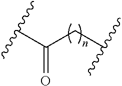 | 5 |
| 1029 | 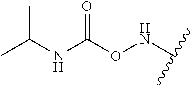 [SMILES: *NOC(=O)NC(C)C] | 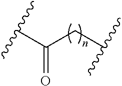 | 6 |
| 1030 | 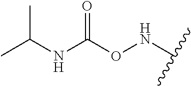 [SMILES: *NOC(=O)NC(C)C] | 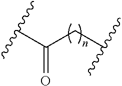 | 7 |
| 1031 | 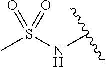 [SMILES: *NS(C)(=O)=O] | 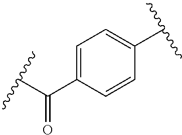 [SMILES: *C(=O)c1ccc(*)cc1] | — |
| 1032 | 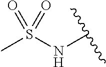 [SMILES: *NS(C)(=O)=O] | 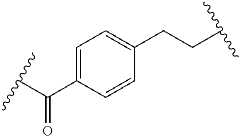 [SMILES: *CCc1ccc(C(*)=O)cc1] | |
| 1033 | 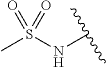 [SMILES: *NS(C)(=O)=O] | 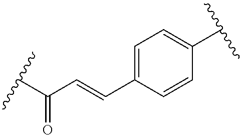 [SMILES: *C(=O)/C=C/c1ccc(*)cc1] | — |
| 1034 | 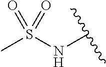 [SMILES: *NS(C)(=O)=O] | 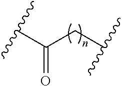 | 5 |
| 1035 | 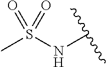 [SMILES: *NS(C)(=O)=O] | 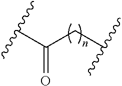 | 6 |
| 1036 | 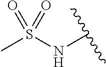 [SMILES: *NS(C)(=O)=O] | 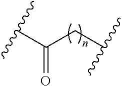 | 7 |
| 1037 | 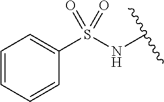 [SMILES: *NS(=O)(=O)c1ccccc1] | 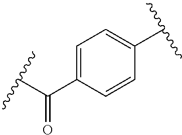 [SMILES: *C(=O)c1ccc(*)cc1] | — |
| 1038 | 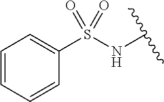 [SMILES: *NS(=O)(=O)c1ccccc1] | 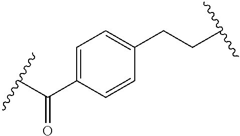 [SMILES: *CCc1ccc(C(*)=O)cc1] | |
| 1039 | 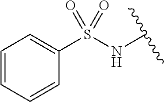 [SMILES: *NS(=O)(=O)c1ccccc1] | 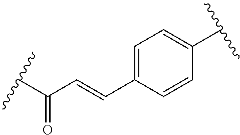 [SMILES: *C(=O)/C=C/c1ccc(*)cc1] | — |
| 1040 | 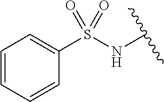 [SMILES: *NS(=O)(=O)c1ccccc1] | 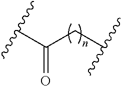 | 5 |
| 1041 | 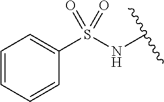 [SMILES: *NS(=O)(=O)c1ccccc1] | 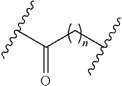 | 6 |
| 1042 | 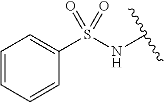 [SMILES: *NS(=O)(=O)c1ccccc1] | 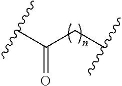 | 7 |
| 1043 | 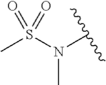 [SMILES: *N(C)S(C)(=O)=O] | 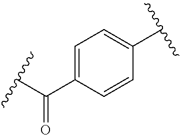 [SMILES: *C(=O)c1ccc(*)cc1] | — |
| 1044 | 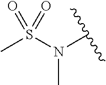 [SMILES: *N(C)S(C)(=O)=O] | 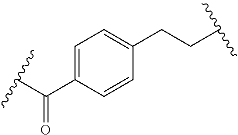 [SMILES: *CCc1ccc(C(*)=O)cc1] | |
| 1045 | 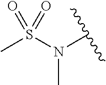 [SMILES: *N(C)S(C)(=O)=O] | 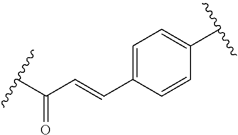 [SMILES: *C(=O)/C=C/c1ccc(*)cc1] | — |
| 1046 | 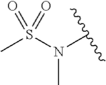 [SMILES: *N(C)S(C)(=O)=O] | 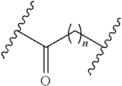 | 5 |
| 1047 | 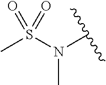 [SMILES: *N(C)S(C)(=O)=O] | 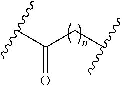 | 6 |
| 1048 | 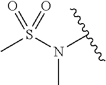 [SMILES: *N(C)S(C)(=O)=O] | 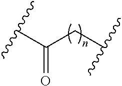 | 7 |
| 1049 | 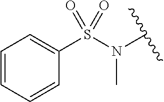 [SMILES: *N(C)S(=O)(=O)c1ccccc1] | 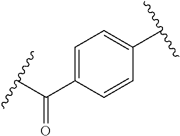 [SMILES: *C(=O)c1ccc(*)cc1] | — |
| 1050 | 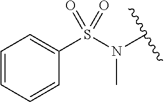 [SMILES: *N(C)S(=O)(=O)c1ccccc1] | 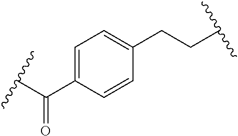 [SMILES: *CCc1ccc(C(*)=O)cc1] | |
| 1051 | 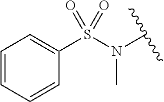 [SMILES: *N(C)S(=O)(=O)c1ccccc1] | 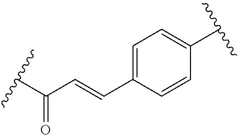 [SMILES: *C(=O)/C=C/c1ccc(*)cc1] | — |
| 1052 | 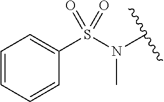 [SMILES: *N(C)S(=O)(=O)c1ccccc1] | 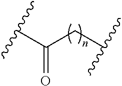 | 5 |
| 1053 | 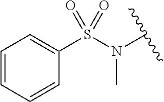 [SMILES: *N(C)S(=O)(=O)c1ccccc1] | 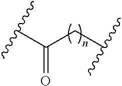 | 6 |
| 1054 | 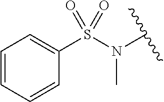 [SMILES: *N(C)S(=O)(=O)c1ccccc1] | 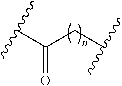 | 7 |
| 1055 | 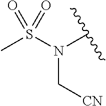 [SMILES: *N(CC#N)S(C)(=O)=O] | 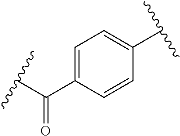 [SMILES: *C(=O)c1ccc(*)cc1] | — |
| 1056 | 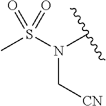 [SMILES: *N(CC#N)S(C)(=O)=O] | 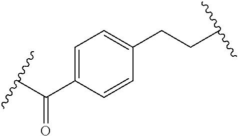 [SMILES: *CCc1ccc(C(*)=O)cc1] | |
| 1057 | 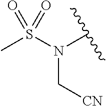 [SMILES: *N(CC#N)S(C)(=O)=O] | 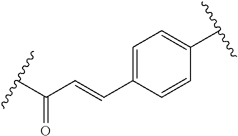 [SMILES: *C(=O)/C=C/c1ccc(*)cc1] | — |
| 1058 | 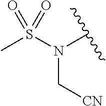 [SMILES: *N(CC#N)S(C)(=O)=O] | 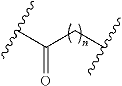 | 5 |
| 1059 | 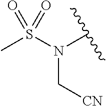 [SMILES: *N(CC#N)S(C)(=O)=O] | 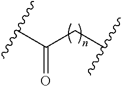 | 6 |
| 1060 | 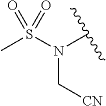 [SMILES: *N(CC#N)S(C)(=O)=O] | 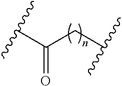 | 7 |
| 1061 | 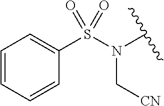 [SMILES: *N(CC#N)S(=O)(=O)c1ccccc1] | 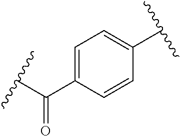 [SMILES: *C(=O)c1ccc(*)cc1] | — |
| 1062 | 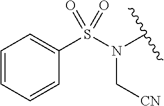 [SMILES: *N(CC#N)S(=O)(=O)c1ccccc1] | 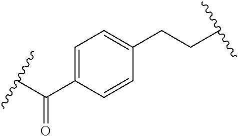 [SMILES: *CCc1ccc(C(*)=O)cc1] | |
| 1063 | 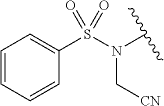 [SMILES: *N(CC#N)S(=O)(=O)c1ccccc1] | 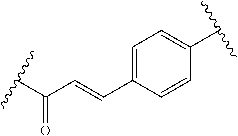 [SMILES: *C(=O)/C=C/c1ccc(*)cc1] | — |
| 1064 | 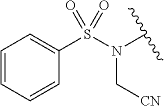 [SMILES: *N(CC#N)S(=O)(=O)c1ccccc1] | 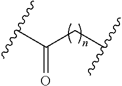 | 5 |
| 1065 | 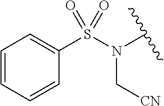 [SMILES: *N(CC#N)S(=O)(=O)c1ccccc1] | 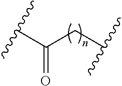 | 6 |
| 1066 | 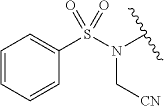 [SMILES: *N(CC#N)S(=O)(=O)c1ccccc1] | 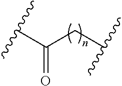 | 7 |
| 1067 | 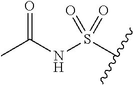 [SMILES: *S(=O)(=O)NC(C)=O] | 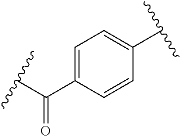 [SMILES: *C(=O)c1ccc(*)cc1] | — |
| 1068 | 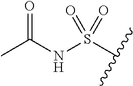 [SMILES: *S(=O)(=O)NC(C)=O] | 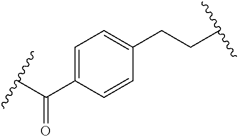 [SMILES: *CCc1ccc(C(*)=O)cc1] | |
| 1069 | 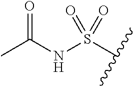 [SMILES: *S(=O)(=O)NC(C)=O] | 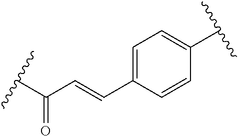 [SMILES: *C(=O)/C=C/c1ccc(*)cc1] | — |
| 1070 | 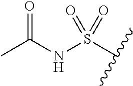 [SMILES: *S(=O)(=O)NC(C)=O] | 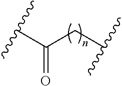 | 5 |
| 1071 | 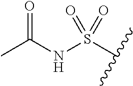 [SMILES: *S(=O)(=O)NC(C)=O] | 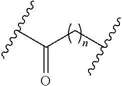 | 6 |
| 1072 | 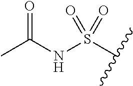 [SMILES: *S(=O)(=O)NC(C)=O] | 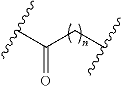 | 7 |
| 1073 | 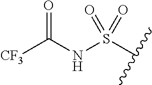 [SMILES: *S(=O)(=O)NC(=O)C(F)(F)F] | 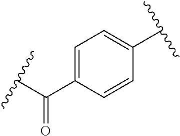 [SMILES: *C(=O)c1ccc(*)cc1] | — |
| 1074 | 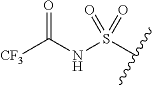 [SMILES: *S(=O)(=O)NC(=O)C(F)(F)F] | 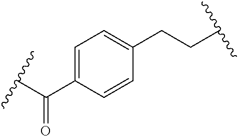 [SMILES: *CCc1ccc(C(*)=O)cc1] | |
| 1075 | 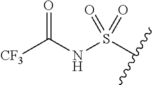 [SMILES: *S(=O)(=O)NC(=O)C(F)(F)F] | 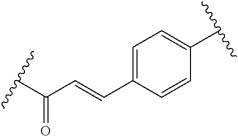 [SMILES: *C(=O)/C=C/c1ccc(*)cc1] | — |
| 1076 | 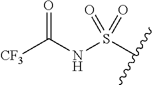 [SMILES: *S(=O)(=O)NC(=O)C(F)(F)F] | 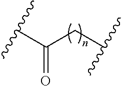 | 5 |
| 1077 | 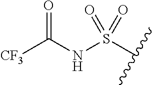 [SMILES: *S(=O)(=O)NC(=O)C(F)(F)F] | 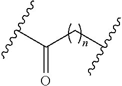 | 6 |
| 1078 | 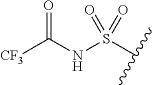 [SMILES: *S(=O)(=O)NC(=O)C(F)(F)F] | 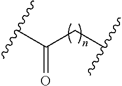 | 7 |
| 1079 | 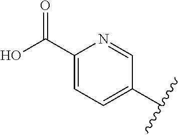 [SMILES: *c1ccc(C(=O)O)nc1] | 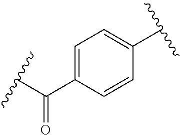 [SMILES: *C(=O)c1ccc(*)cc1] | — |
| 1080 | 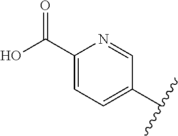 [SMILES: *c1ccc(C(=O)O)nc1] | 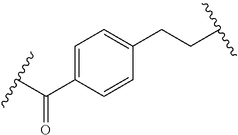 [SMILES: *CCc1ccc(C(*)=O)cc1] | |
| 1081 | 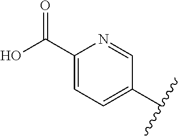 [SMILES: *c1ccc(C(=O)O)nc1] | 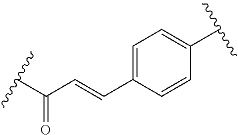 [SMILES: *C(=O)/C=C/c1ccc(*)cc1] | — |
| 1082 | 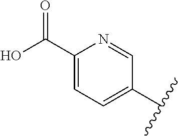 [SMILES: *c1ccc(C(=O)O)nc1] | 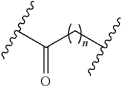 | 5 |
| 1083 | 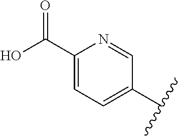 [SMILES: *c1ccc(C(=O)O)nc1] | 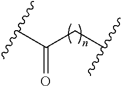 | 6 |
| 1084 | 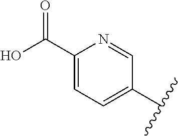 [SMILES: *c1ccc(C(=O)O)nc1] | 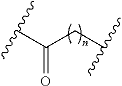 | 7 |
| 1085 | 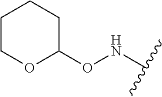 [SMILES: *NOC1CCCCO1] | 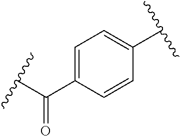 [SMILES: *C(=O)c1ccc(*)cc1] | — |
| 1086 | 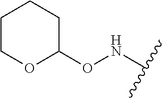 [SMILES: *NOC1CCCCO1] | 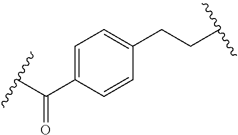 [SMILES: *CCc1ccc(C(*)=O)cc1] | |
| 1087 | 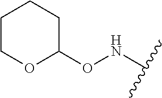 [SMILES: *NOC1CCCCO1] | 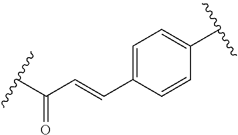 [SMILES: *C(=O)/C=C/c1ccc(*)cc1] | — |
| 1088 | 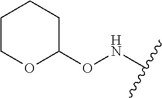 [SMILES: *NOC1CCCCO1] | 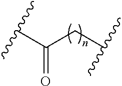 | 5 |
| 1089 | 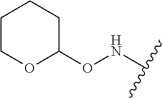 [SMILES: *NOC1CCCCO1] | 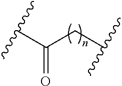 | 6 |
| 1090 | 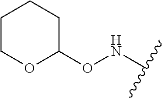 [SMILES: *NOC1CCCCO1] | 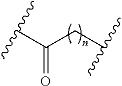 | 7 |
| 1091 | 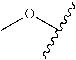 [SMILES: *OC] | 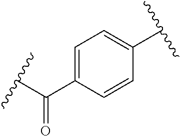 [SMILES: *C(=O)c1ccc(*)cc1] | — |
| 1092 | 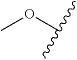 [SMILES: *OC] | 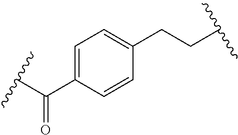 [SMILES: *CCc1ccc(C(*)=O)cc1] | |
| 1093 | 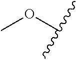 [SMILES: *OC] | 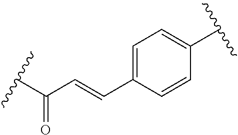 [SMILES: *C(=O)/C=C/c1ccc(*)cc1] | — |
| 1094 | 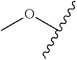 [SMILES: *OC] | 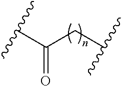 | 5 |
| 1095 | 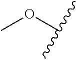 [SMILES: *OC] | 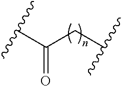 | 6 |
| 1096 | 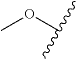 [SMILES: *OC] | 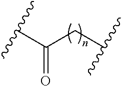 | 7 |
| 1097 | 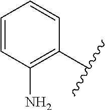 [SMILES: *c1ccccc1N] | 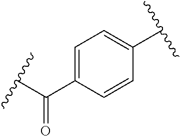 [SMILES: *C(=O)c1ccc(*)cc1] | — |
| 1098 | 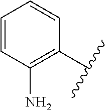 [SMILES: *c1ccccc1N] | 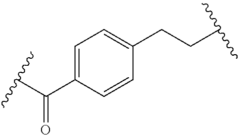 [SMILES: *CCc1ccc(C(*)=O)cc1] | |
| 1099 | 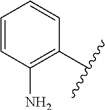 [SMILES: *c1ccccc1N] | 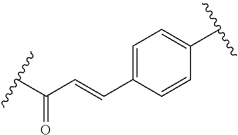 [SMILES: *C(=O)/C=C/c1ccc(*)cc1] | — |
| 1100 | 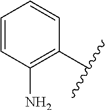 [SMILES: *c1ccccc1N] | 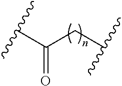 | 5 |
| 1101 | 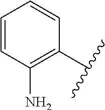 [SMILES: *c1ccccc1N] | 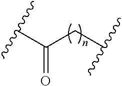 | 6 |
| 1102 | 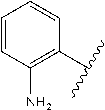 [SMILES: *c1ccccc1N] | 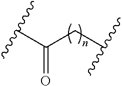 | 7 |
| 1103 | 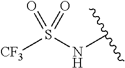 [SMILES: *NS(=O)(=O)C(F)(F)F] | 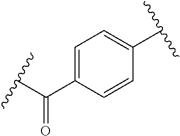 [SMILES: *C(=O)c1ccc(*)cc1] | — |
| 1104 | 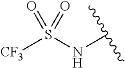 [SMILES: *NS(=O)(=O)C(F)(F)F] | 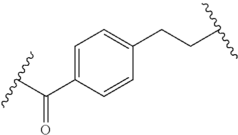 [SMILES: *CCc1ccc(C(*)=O)cc1] | |
| 1105 | 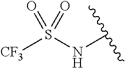 [SMILES: *NS(=O)(=O)C(F)(F)F] | 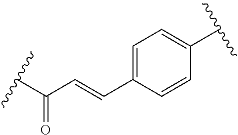 [SMILES: *C(=O)/C=C/c1ccc(*)cc1] | — |
| 1106 | 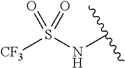 [SMILES: *NS(=O)(=O)C(F)(F)F] | 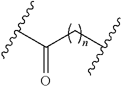 | 5 |
| 1107 | 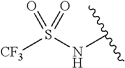 [SMILES: *NS(=O)(=O)C(F)(F)F] | 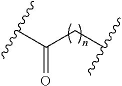 | 6 |
| 1108 | 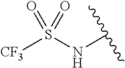 [SMILES: *NS(=O)(=O)C(F)(F)F] | 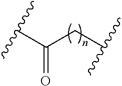 | 7 |
| 1109 | 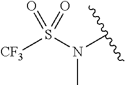 [SMILES: *N(C)S(=O)(=O)C(F)(F)F] | 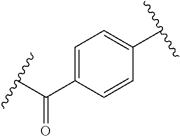 [SMILES: *C(=O)c1ccc(*)cc1] | — |
| 1110 | 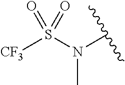 [SMILES: *N(C)S(=O)(=O)C(F)(F)F] | 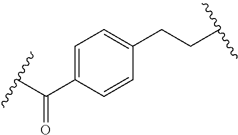 [SMILES: *CCc1ccc(C(*)=O)cc1] | |
| 1111 | 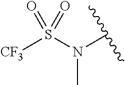 [SMILES: *N(C)S(=O)(=O)C(F)(F)F] | 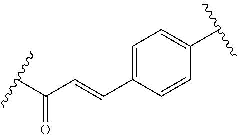 [SMILES: *C(=O)/C=C/c1ccc(*)cc1] | — |
| 1112 | 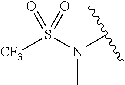 [SMILES: *N(C)S(=O)(=O)C(F)(F)F] | 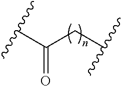 | 5 |
| 1113 | 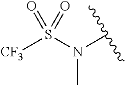 [SMILES: *N(C)S(=O)(=O)C(F)(F)F] | 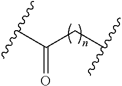 | 6 |
| 1114 | 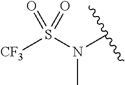 [SMILES: *N(C)S(=O)(=O)C(F)(F)F] | 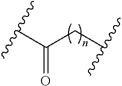 | 7 |

In some embodiments, the compound of formula (I) is a compound of any one of formula 1115-1228, or a pharmaceutically acceptable salt, solvate, hydrate, cocrystal, or prodrug thereof, wherein in formula 1115-1228:
B is:

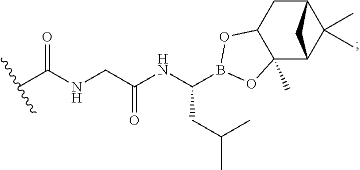 [SMILES: *C(=O)NCC(=O)N[C@@H](CC(C)C)B1OC2C[C@H]3C[C@H](C3(C)C)[C@@]2(C)O1]

and:

| Formula No. | A | L | n |
|---|---|---|---|
| 1115 | 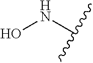 [SMILES: *NO] | 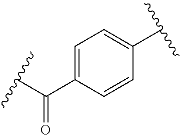 [SMILES: *C(=O)c1ccc(*)cc1] | — |
| 1116 | 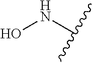 [SMILES: *NO] | 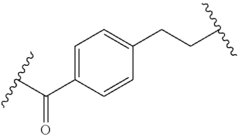 [SMILES: *CCc1ccc(C(*)=O)cc1] | |
| 1117 | 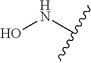 [SMILES: *NO] | 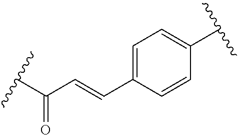 [SMILES: *C(=O)/C=C/c1ccc(*)cc1] | — |
| 1118 | 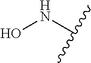 [SMILES: *NO] | 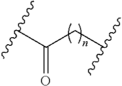 | 5 |
| 1119 | 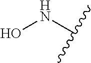 [SMILES: *NO] | 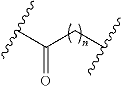 | 6 |
| 1120 | 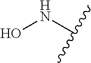 [SMILES: *NO] | 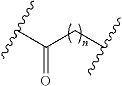 | 7 |
| 1121 | 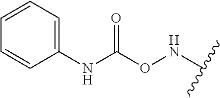 [SMILES: *NOC(=O)Nc1ccccc1] | 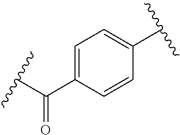 [SMILES: *C(=O)c1ccc(*)cc1] | — |
| 1122 | 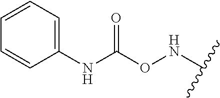 [SMILES: *NOC(=O)Nc1ccccc1] | 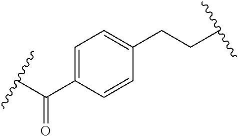 [SMILES: *CCc1ccc(C(*)=O)cc1] | |
| 1123 | 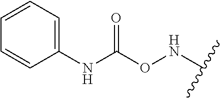 [SMILES: *NOC(=O)Nc1ccccc1] | 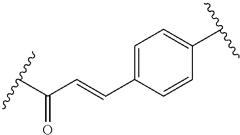 [SMILES: *C(=O)/C=C/c1ccc(*)cc1] | — |
| 1124 | 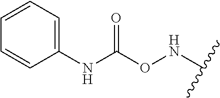 [SMILES: *NOC(=O)Nc1ccccc1] | 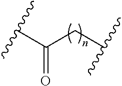 | 5 |
| 1125 | 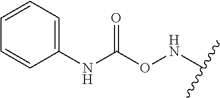 [SMILES: *NOC(=O)Nc1ccccc1] | 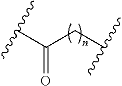 | 6 |
| 1126 | 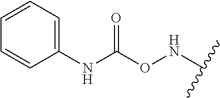 [SMILES: *NOC(=O)Nc1ccccc1] | 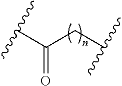 | 7 |
| 1127 | 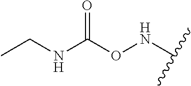 [SMILES: *NOC(=O)NCC] | 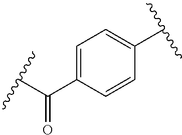 [SMILES: *C(=O)c1ccc(*)cc1] | — |
| 1128 | 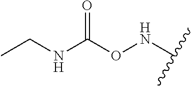 [SMILES: *NOC(=O)NCC] | 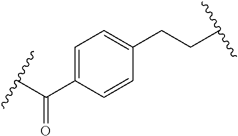 [SMILES: *CCc1ccc(C(*)=O)cc1] | |
| 1129 | 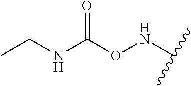 [SMILES: *NOC(=O)NCC] | 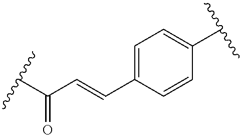 [SMILES: *C(=O)/C=C/c1ccc(*)cc1] | — |
| 1130 | 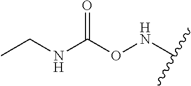 [SMILES: *NOC(=O)NCC] | 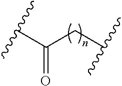 | 5 |
| 1131 | 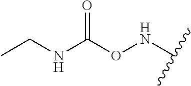 [SMILES: *NOC(=O)NCC] | 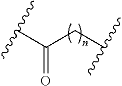 | 6 |
| 1132 | 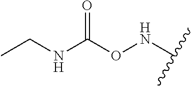 [SMILES: *NOC(=O)NCC] | 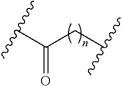 | 7 |
| 1133 | 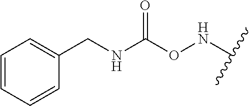 [SMILES: *NOC(=O)NCc1ccccc1] | 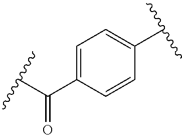 [SMILES: *C(=O)c1ccc(*)cc1] | — |
| 1134 | 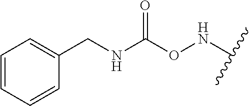 [SMILES: *NOC(=O)NCc1ccccc1] | 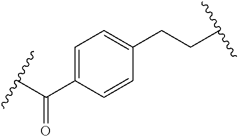 [SMILES: *CCc1ccc(C(*)=O)cc1] | |
| 1135 | 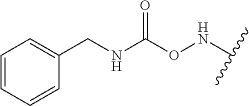 [SMILES: *NOC(=O)NCc1ccccc1] | 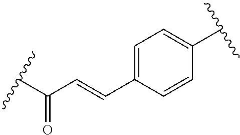 [SMILES: *C(=O)/C=C/c1ccc(*)cc1] | — |
| 1136 | 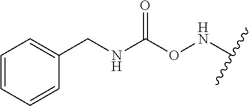 [SMILES: *NOC(=O)NCc1ccccc1] | 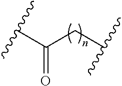 | 5 |
| 1137 | 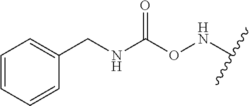 [SMILES: *NOC(=O)NCc1ccccc1] | 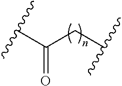 | 6 |
| 1138 | 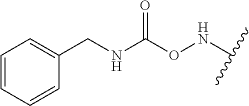 [SMILES: *NOC(=O)NCc1ccccc1] | 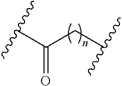 | 7 |
| 1139 | 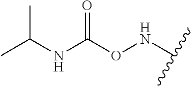 [SMILES: *NOC(=O)NC(C)C] | 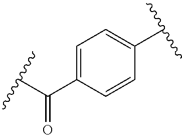 [SMILES: *C(=O)c1ccc(*)cc1] | — |
| 1140 | 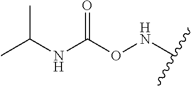 [SMILES: *NOC(=O)NC(C)C] | 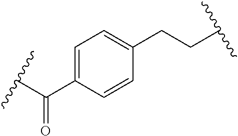 [SMILES: *CCc1ccc(C(*)=O)cc1] | |
| 1141 | 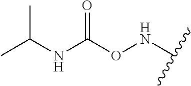 [SMILES: *NOC(=O)NC(C)C] | 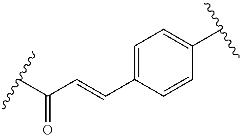 [SMILES: *C(=O)/C=C/c1ccc(*)cc1] | — |
| 1142 | 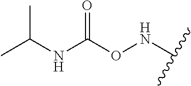 [SMILES: *NOC(=O)NC(C)C] | 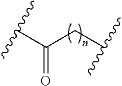 | 5 |
| 1143 | 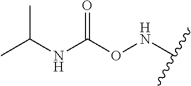 [SMILES: *NOC(=O)NC(C)C] | 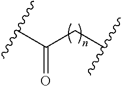 | 6 |
| 1144 | 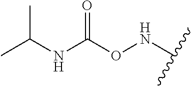 [SMILES: *NOC(=O)NC(C)C] | 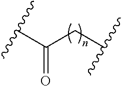 | 7 |
| 1145 | 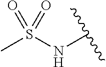 [SMILES: *NS(C)(=O)=O] | 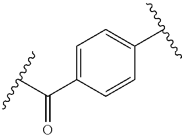 [SMILES: *C(=O)c1ccc(*)cc1] | — |
| 1146 | 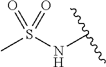 [SMILES: *NS(C)(=O)=O] | 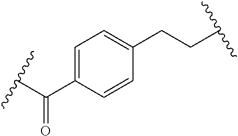 [SMILES: *CCc1ccc(C(*)=O)cc1] | |
| 1147 | 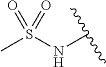 [SMILES: *NS(C)(=O)=O] | 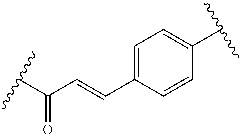 [SMILES: *C(=O)/C=C/c1ccc(*)cc1] | — |
| 1148 | 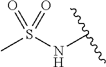 [SMILES: *NS(C)(=O)=O] | 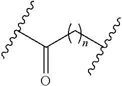 | 5 |
| 1149 | 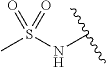 [SMILES: *NS(C)(=O)=O] | 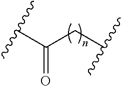 | 6 |
| 1150 | 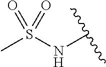 [SMILES: *NS(C)(=O)=O] | 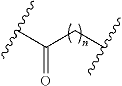 | 7 |
| 1151 | 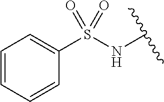 [SMILES: *NS(=O)(=O)c1ccccc1] | 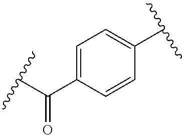 [SMILES: *C(=O)c1ccc(*)cc1] | — |
| 1152 | 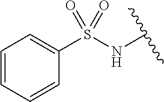 [SMILES: *NS(=O)(=O)c1ccccc1] | 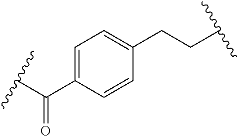 [SMILES: *CCc1ccc(C(*)=O)cc1] | |
| 1153 | 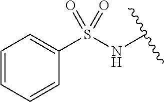 [SMILES: *NS(=O)(=O)c1ccccc1] | 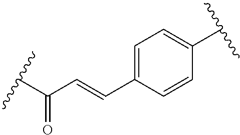 [SMILES: *C(=O)/C=C/c1ccc(*)cc1] | — |
| 1154 | 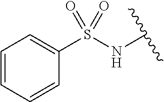 [SMILES: *NS(=O)(=O)c1ccccc1] | 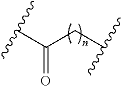 | 5 |
| 1155 | 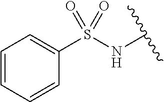 [SMILES: *NS(=O)(=O)c1ccccc1] | 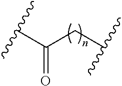 | 6 |
| 1156 | 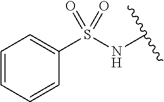 [SMILES: *NS(=O)(=O)c1ccccc1] | 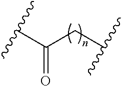 | 7 |
| 1157 | 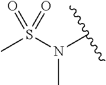 [SMILES: *N(C)S(C)(=O)=O] | 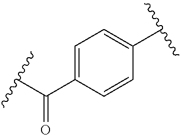 [SMILES: *C(=O)c1ccc(*)cc1] | — |
| 1158 | 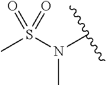 [SMILES: *N(C)S(C)(=O)=O] | 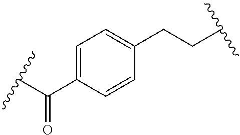 [SMILES: *CCc1ccc(C(*)=O)cc1] | |
| 1159 | 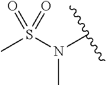 [SMILES: *N(C)S(C)(=O)=O] | 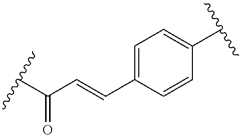 [SMILES: *C(=O)/C=C/c1ccc(*)cc1] | — |
| 1160 | 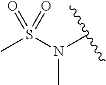 [SMILES: *N(C)S(C)(=O)=O] | 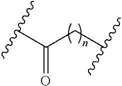 | 5 |
| 1161 | 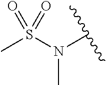 [SMILES: *N(C)S(C)(=O)=O] | 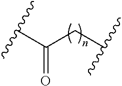 | 6 |
| 1162 | 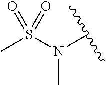 [SMILES: *N(C)S(C)(=O)=O] | 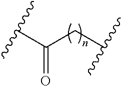 | 7 |
| 1163 | 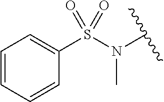 [SMILES: *N(C)S(=O)(=O)c1ccccc1] | 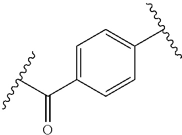 [SMILES: *C(=O)c1ccc(*)cc1] | — |
| 1164 | 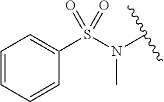 [SMILES: *N(C)S(=O)(=O)c1ccccc1] | 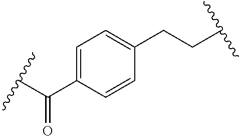 [SMILES: *CCc1ccc(C(*)=O)cc1] | |
| 1165 | 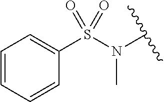 [SMILES: *N(C)S(=O)(=O)c1ccccc1] | 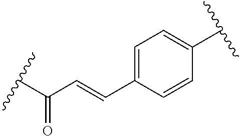 [SMILES: *C(=O)/C=C/c1ccc(*)cc1] | — |
| 1166 | 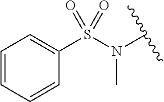 [SMILES: *N(C)S(=O)(=O)c1ccccc1] | 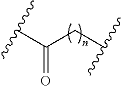 | 5 |
| 1167 | 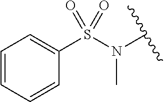 [SMILES: *N(C)S(=O)(=O)c1ccccc1] | 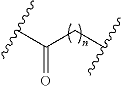 | 6 |
| 1168 | 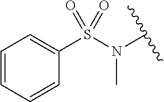 [SMILES: *N(C)S(=O)(=O)c1ccccc1] | 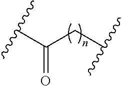 | 7 |
| 1169 | 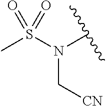 [SMILES: *N(CC#N)S(C)(=O)=O] | 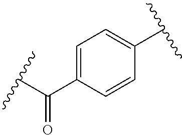 [SMILES: *C(=O)c1ccc(*)cc1] | — |
| 1170 | 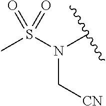 [SMILES: *N(CC#N)S(C)(=O)=O] | 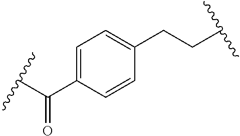 [SMILES: *CCc1ccc(C(*)=O)cc1] | |
| 1171 | 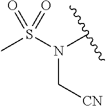 [SMILES: *N(CC#N)S(C)(=O)=O] | 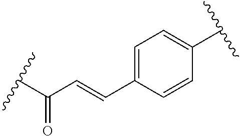 [SMILES: *C(=O)/C=C/c1ccc(*)cc1] | — |
| 1172 | 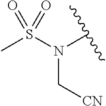 [SMILES: *N(CC#N)S(C)(=O)=O] | 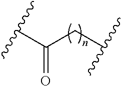 | 5 |
| 1173 | 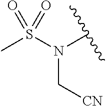 [SMILES: *N(CC#N)S(C)(=O)=O] | 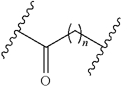 | 6 |
| 1174 | 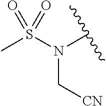 [SMILES: *N(CC#N)S(C)(=O)=O] | 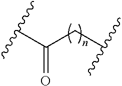 | 7 |
| 1175 | 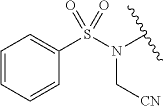 [SMILES: *N(CC#N)S(=O)(=O)c1ccccc1] | 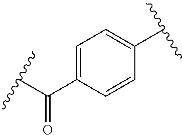 [SMILES: *C(=O)c1ccc(*)cc1] | — |
| 1176 | 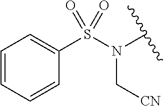 [SMILES: *N(CC#N)S(=O)(=O)c1ccccc1] | 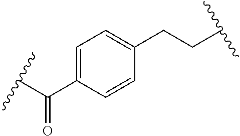 [SMILES: *CCc1ccc(C(*)=O)cc1] | |
| 1177 | 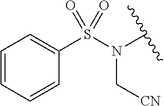 [SMILES: *N(CC#N)S(=O)(=O)c1ccccc1] | 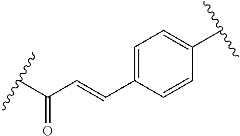 [SMILES: *C(=O)/C=C/c1ccc(*)cc1] | — |
| 1178 | 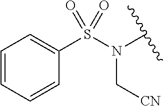 [SMILES: *N(CC#N)S(=O)(=O)c1ccccc1] | 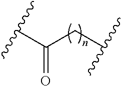 | 5 |
| 1179 | 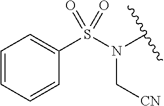 [SMILES: *N(CC#N)S(=O)(=O)c1ccccc1] | 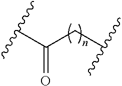 | 6 |
| 1180 | 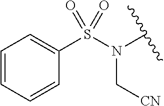 [SMILES: *N(CC#N)S(=O)(=O)c1ccccc1] | 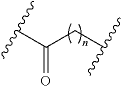 | 7 |
| 1181 | 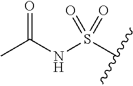 [SMILES: *S(=O)(=O)NC(C)=O] | 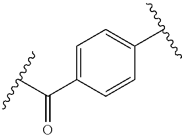 [SMILES: *C(=O)c1ccc(*)cc1] | — |
| 1182 | 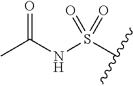 [SMILES: *S(=O)(=O)NC(C)=O] | 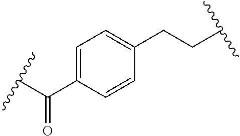 [SMILES: *CCc1ccc(C(*)=O)cc1] | |
| 1183 | 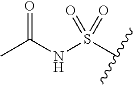 [SMILES: *S(=O)(=O)NC(C)=O] | 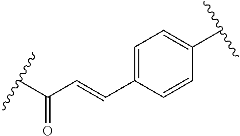 [SMILES: *C(=O)/C=C/c1ccc(*)cc1] | — |
| 1184 | 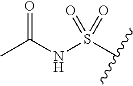 [SMILES: *S(=O)(=O)NC(C)=O] | 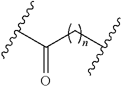 | 5 |
| 1185 | 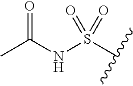 [SMILES: *S(=O)(=O)NC(C)=O] | 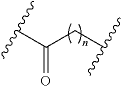 | 6 |
| 1186 | 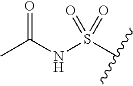 [SMILES: *S(=O)(=O)NC(C)=O] | 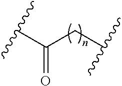 | 7 |
| 1187 | 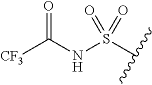 [SMILES: *S(=O)(=O)NC(=O)C(F)(F)F] | 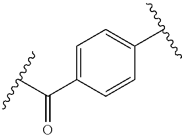 [SMILES: *C(=O)c1ccc(*)cc1] | — |
| 1188 | 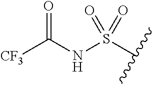 [SMILES: *S(=O)(=O)NC(=O)C(F)(F)F] | 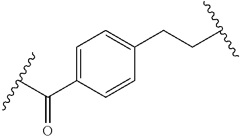 [SMILES: *CCc1ccc(C(*)=O)cc1] | |
| 1189 | 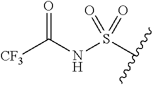 [SMILES: *S(=O)(=O)NC(=O)C(F)(F)F] | 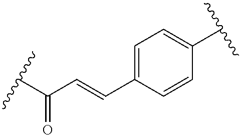 [SMILES: *C(=O)/C=C/c1ccc(*)cc1] | — |
| 1190 | 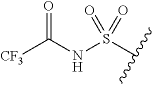 [SMILES: *S(=O)(=O)NC(=O)C(F)(F)F] | 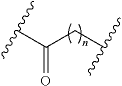 | 5 |
| 1191 | 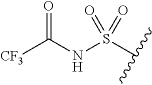 [SMILES: *S(=O)(=O)NC(=O)C(F)(F)F] | 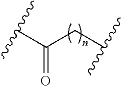 | 6 |
| 1192 | 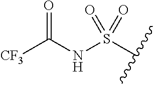 [SMILES: *S(=O)(=O)NC(=O)C(F)(F)F] | 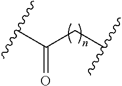 | 7 |
| 1193 | 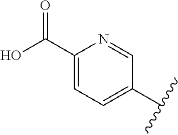 [SMILES: *c1ccc(C(=O)O)nc1] | 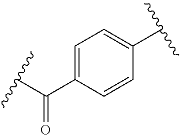 [SMILES: *C(=O)c1ccc(*)cc1] | — |
| 1194 | 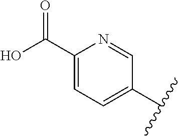 [SMILES: *c1ccc(C(=O)O)nc1] | 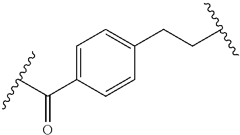 [SMILES: *CCc1ccc(C(*)=O)cc1] | |
| 1195 | 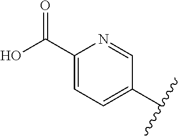 [SMILES: *c1ccc(C(=O)O)nc1] | 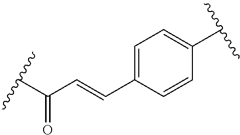 [SMILES: *C(=O)/C=C/c1ccc(*)cc1] | — |
| 1196 | 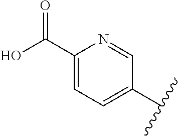 [SMILES: *c1ccc(C(=O)O)nc1] | 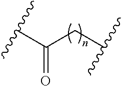 | 5 |
| 1197 | 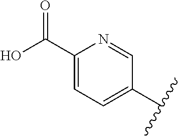 [SMILES: *c1ccc(C(=O)O)nc1] | 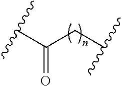 | 6 |
| 1198 | 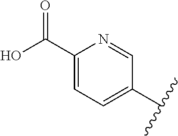 [SMILES: *c1ccc(C(=O)O)nc1] | 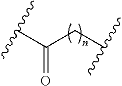 | 7 |
| 1199 | 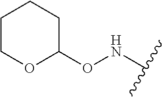 [SMILES: *NOC1CCCCO1] | 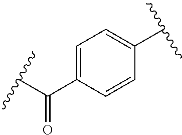 [SMILES: *C(=O)c1ccc(*)cc1] | — |
| 1200 | 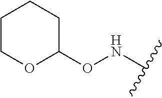 [SMILES: *NOC1CCCCO1] | 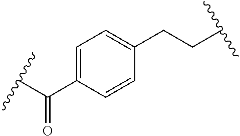 [SMILES: *CCc1ccc(C(*)=O)cc1] | |
| 1201 | 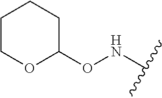 [SMILES: *NOC1CCCCO1] | 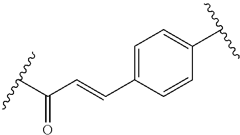 [SMILES: *C(=O)/C=C/c1ccc(*)cc1] | — |
| 1202 | 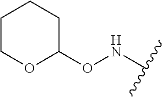 [SMILES: *NOC1CCCCO1] | 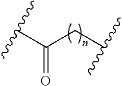 | 5 |
| 1203 | 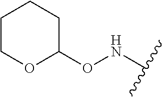 [SMILES: *NOC1CCCCO1] | 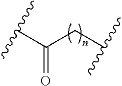 | 6 |
| 1204 | 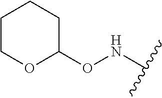 [SMILES: *NOC1CCCCO1] | 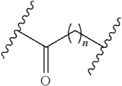 | 7 |
| 1205 | 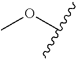 [SMILES: *OC] | 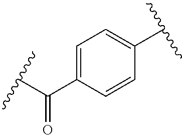 [SMILES: *C(=O)c1ccc(*)cc1] | — |
| 1206 | 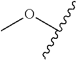 [SMILES: *OC] | 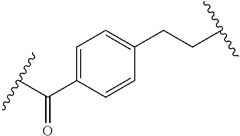 [SMILES: *CCc1ccc(C(*)=O)cc1] | |
| 1207 | 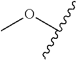 [SMILES: *OC] | 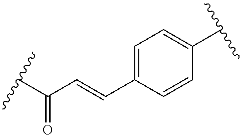 [SMILES: *C(=O)/C=C/c1ccc(*)cc1] | — |
| 1208 | 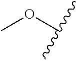 [SMILES: *OC] | 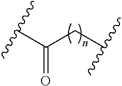 | 5 |
| 1209 | 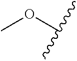 [SMILES: *OC] | 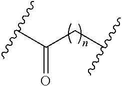 | 6 |
| 1210 | 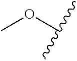 [SMILES: *OC] | 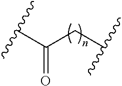 | 7 |
| 1211 | 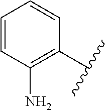 [SMILES: *c1ccccc1N] | 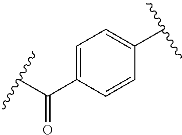 [SMILES: *C(=O)c1ccc(*)cc1] | — |
| 1212 | 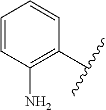 [SMILES: *c1ccccc1N] | 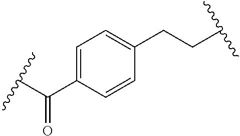 [SMILES: *CCc1ccc(C(*)=O)cc1] | |
| 1213 | 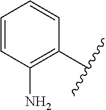 [SMILES: *c1ccccc1N] | 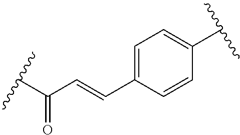 [SMILES: *C(=O)/C=C/c1ccc(*)cc1] | — |
| 1214 | 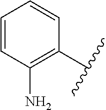 [SMILES: *c1ccccc1N] | 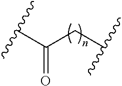 | 5 |
| 1215 | 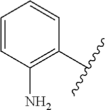 [SMILES: *c1ccccc1N] | 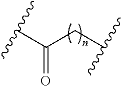 | 6 |
| 1216 | 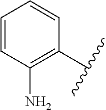 [SMILES: *c1ccccc1N] | 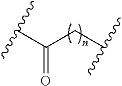 | 7 |
| 1217 | 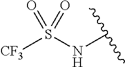 [SMILES: *NS(=O)(=O)C(F)(F)F] | 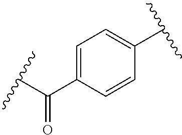 [SMILES: *C(=O)c1ccc(*)cc1] | — |
| 1218 | 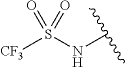 [SMILES: *NS(=O)(=O)C(F)(F)F] | 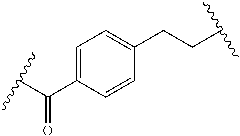 [SMILES: *CCc1ccc(C(*)=O)cc1] | |
| 1219 | 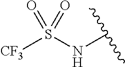 [SMILES: *NS(=O)(=O)C(F)(F)F] | 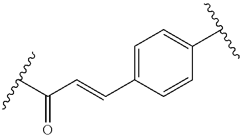 [SMILES: *C(=O)/C=C/c1ccc(*)cc1] | — |
| 1220 | 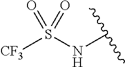 [SMILES: *NS(=O)(=O)C(F)(F)F] | 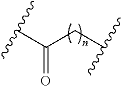 | 5 |
| 1221 | 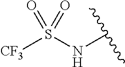 [SMILES: *NS(=O)(=O)C(F)(F)F] | 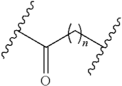 | 6 |
| 1222 | 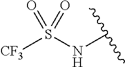 [SMILES: *NS(=O)(=O)C(F)(F)F] | 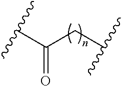 | 7 |
| 1223 | 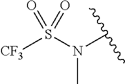 [SMILES: *N(C)S(=O)(=O)C(F)(F)F] | 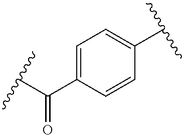 [SMILES: *C(=O)c1ccc(*)cc1] | — |
| 1224 | 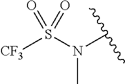 [SMILES: *N(C)S(=O)(=O)C(F)(F)F] | 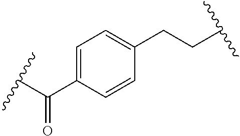 [SMILES: *CCc1ccc(C(*)=O)cc1] | |
| 1225 | 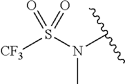 [SMILES: *N(C)S(=O)(=O)C(F)(F)F] | 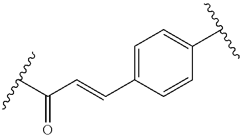 [SMILES: *C(=O)/C=C/c1ccc(*)cc1] | — |
| 1226 | 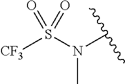 [SMILES: *N(C)S(=O)(=O)C(F)(F)F] | 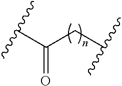 | 5 |
| 1227 | 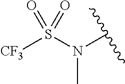 [SMILES: *N(C)S(=O)(=O)C(F)(F)F] | 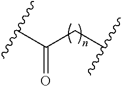 | 6 |
| 1228 | 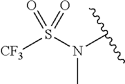 [SMILES: *N(C)S(=O)(=O)C(F)(F)F] | 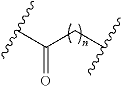 | 7 |

In some embodiments, the compound of formula (I) is a compound ofany one of formula 1229-1342, or a pharmaceutically acceptable salt, solvate, hydrate, cocrystal, or prodrug thereof, wherein in formula 1229-1342:
B is:

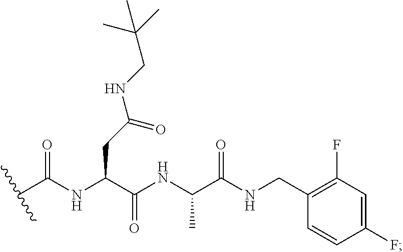 [SMILES: *C(=O)N[C@@H](CC(=O)NCC(C)(C)C)C(=O)N[C@@H](C)C(=O)NCc1ccc(F)cc1F]

and:

| Formula No. | A | L | n |
|---|---|---|---|
| 1229 | 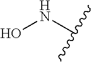 [SMILES: *NO] | 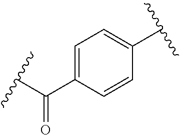 [SMILES: *C(=O)c1ccc(*)cc1] | — |
| 1230 | 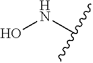 [SMILES: *NO] | 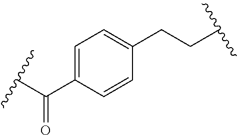 [SMILES: *CCc1ccc(C(*)=O)cc1] | |
| 1231 | 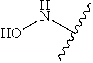 [SMILES: *NO] | 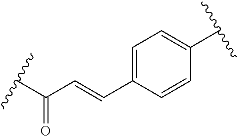 [SMILES: *C(=O)/C=C/c1ccc(*)cc1] | — |
| 1232 | 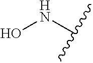 [SMILES: *NO] | 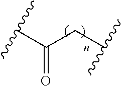 | 5 |
| 1233 | 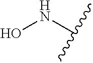 [SMILES: *NO] | 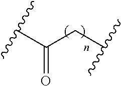 | 6 |
| 1234 | 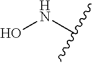 [SMILES: *NO] | 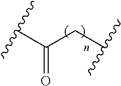 | 7 |
| 1235 | 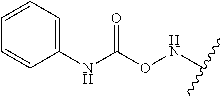 [SMILES: *NOC(=O)Nc1ccccc1] | 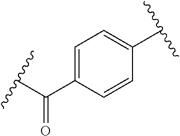 [SMILES: *C(=O)c1ccc(*)cc1] | — |
| 1236 | 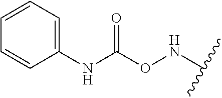 [SMILES: *NOC(=O)Nc1ccccc1] | 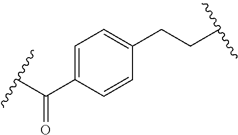 [SMILES: *CCc1ccc(C(*)=O)cc1] | |
| 1237 | 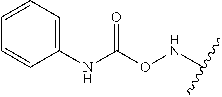 [SMILES: *NOC(=O)Nc1ccccc1] | 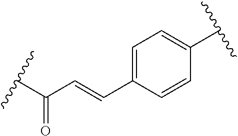 [SMILES: *C(=O)/C=C/c1ccc(*)cc1] | — |
| 1238 | 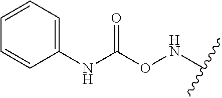 [SMILES: *NOC(=O)Nc1ccccc1] | 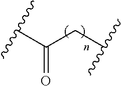 | 5 |
| 1239 | 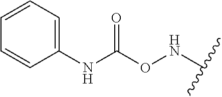 [SMILES: *NOC(=O)Nc1ccccc1] | 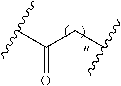 | 6 |
| 1240 | 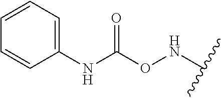 [SMILES: *NOC(=O)Nc1ccccc1] | 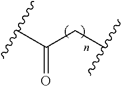 | 7 |
| 1241 | 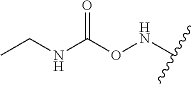 [SMILES: *NOC(=O)NCC] | 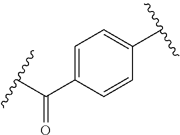 [SMILES: *C(=O)c1ccc(*)cc1] | — |
| 1242 | 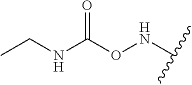 [SMILES: *NOC(=O)NCC] | 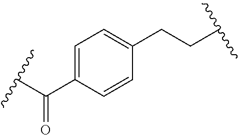 [SMILES: *CCc1ccc(C(*)=O)cc1] | |
| 1243 | 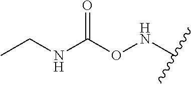 [SMILES: *NOC(=O)NCC] | 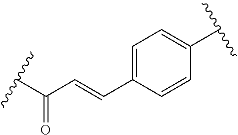 [SMILES: *C(=O)/C=C/c1ccc(*)cc1] | — |
| 1244 | 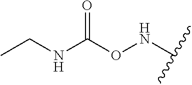 [SMILES: *NOC(=O)NCC] | 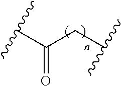 | 5 |
| 1245 | 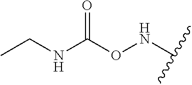 [SMILES: *NOC(=O)NCC] | 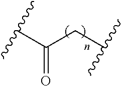 | 6 |
| 1246 | 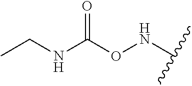 [SMILES: *NOC(=O)NCC] | 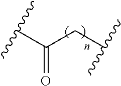 | 7 |
| 1247 | 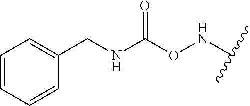 [SMILES: *NOC(=O)NCc1ccccc1] | 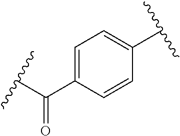 [SMILES: *C(=O)c1ccc(*)cc1] | — |
| 1248 | 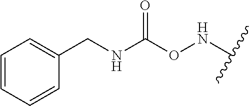 [SMILES: *NOC(=O)NCc1ccccc1] | 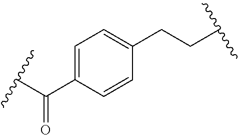 [SMILES: *CCc1ccc(C(*)=O)cc1] | |
| 1249 | 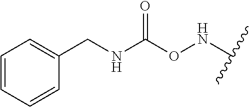 [SMILES: *NOC(=O)NCc1ccccc1] | 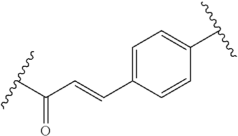 [SMILES: *C(=O)/C=C/c1ccc(*)cc1] | — |
| 1250 | 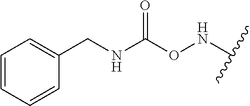 [SMILES: *NOC(=O)NCc1ccccc1] | 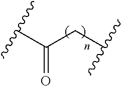 | 5 |
| 1251 | 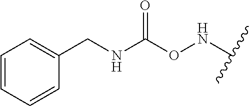 [SMILES: *NOC(=O)NCc1ccccc1] | 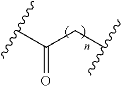 | 6 |
| 1252 | 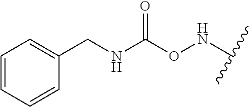 [SMILES: *NOC(=O)NCc1ccccc1] | 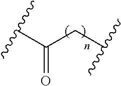 | 7 |
| 1253 | 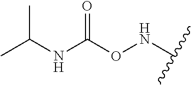 [SMILES: *NOC(=O)NC(C)C] | 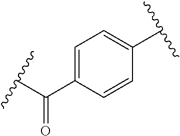 [SMILES: *C(=O)c1ccc(*)cc1] | — |
| 1254 | 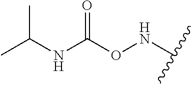 [SMILES: *NOC(=O)NC(C)C] | 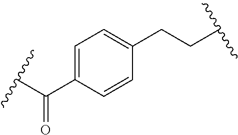 [SMILES: *CCc1ccc(C(*)=O)cc1] | |
| 1255 | 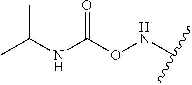 [SMILES: *NOC(=O)NC(C)C] | 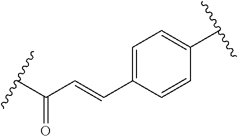 [SMILES: *C(=O)/C=C/c1ccc(*)cc1] | — |
| 1256 | 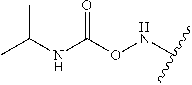 [SMILES: *NOC(=O)NC(C)C] | 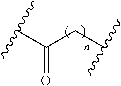 | 5 |
| 1257 | 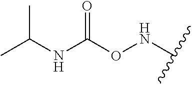 [SMILES: *NOC(=O)NC(C)C] | 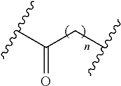 | 6 |
| 1258 | 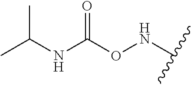 [SMILES: *NOC(=O)NC(C)C] | 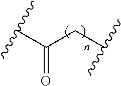 | 7 |
| 1259 | 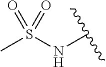 [SMILES: *NS(C)(=O)=O] | 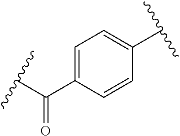 [SMILES: *C(=O)c1ccc(*)cc1] | — |
| 1260 | 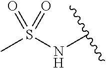 [SMILES: *NS(C)(=O)=O] | 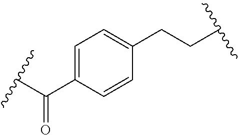 [SMILES: *CCc1ccc(C(*)=O)cc1] | |
| 1261 | 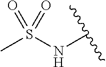 [SMILES: *NS(C)(=O)=O] | 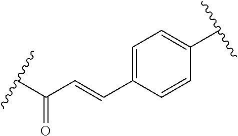 [SMILES: *C(=O)/C=C/c1ccc(*)cc1] | — |
| 1262 | 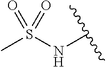 [SMILES: *NS(C)(=O)=O] | 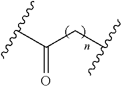 | 5 |
| 1263 | 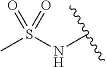 [SMILES: *NS(C)(=O)=O] | 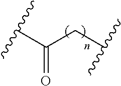 | 6 |
| 1264 | 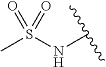 [SMILES: *NS(C)(=O)=O] | 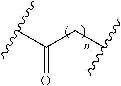 | 7 |
| 1265 | 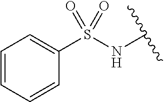 [SMILES: *NS(=O)(=O)c1ccccc1] | 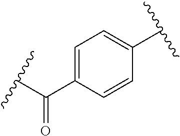 [SMILES: *C(=O)c1ccc(*)cc1] | — |
| 1266 | 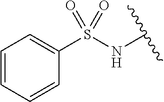 [SMILES: *NS(=O)(=O)c1ccccc1] | 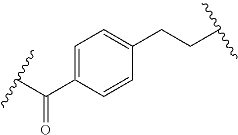 [SMILES: *CCc1ccc(C(*)=O)cc1] | |
| 1267 | 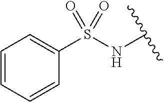 [SMILES: *NS(=O)(=O)c1ccccc1] | 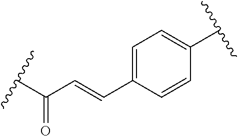 [SMILES: *C(=O)/C=C/c1ccc(*)cc1] | — |
| 1268 | 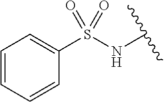 [SMILES: *NS(=O)(=O)c1ccccc1] | 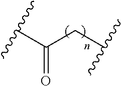 | 5 |
| 1269 | 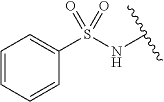 [SMILES: *NS(=O)(=O)c1ccccc1] | 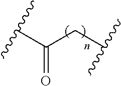 | 6 |
| 1270 | 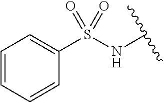 [SMILES: *NS(=O)(=O)c1ccccc1] | 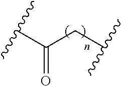 | 7 |
| 1271 | 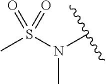 [SMILES: *N(C)S(C)(=O)=O] | 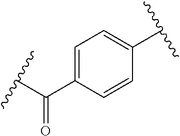 [SMILES: *C(=O)c1ccc(*)cc1] | — |
| 1272 | 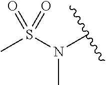 [SMILES: *N(C)S(C)(=O)=O] | 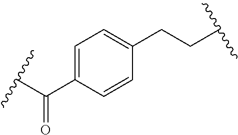 [SMILES: *CCc1ccc(C(*)=O)cc1] | |
| 1273 | 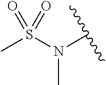 [SMILES: *N(C)S(C)(=O)=O] | 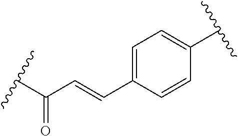 [SMILES: *C(=O)/C=C/c1ccc(*)cc1] | — |
| 1274 | 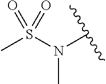 [SMILES: *N(C)S(C)(=O)=O] | 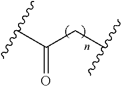 | 5 |
| 1275 | 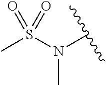 [SMILES: *N(C)S(C)(=O)=O] | 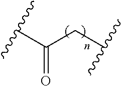 | 6 |
| 1276 | 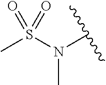 [SMILES: *N(C)S(C)(=O)=O] | 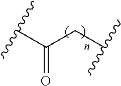 | 7 |
| 1277 | 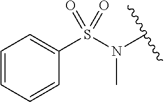 [SMILES: *N(C)S(=O)(=O)c1ccccc1] | 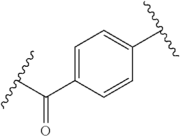 [SMILES: *C(=O)c1ccc(*)cc1] | — |
| 1278 | 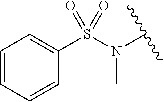 [SMILES: *N(C)S(=O)(=O)c1ccccc1] | 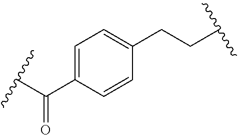 [SMILES: *CCc1ccc(C(*)=O)cc1] | |
| 1279 | 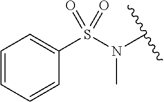 [SMILES: *N(C)S(=O)(=O)c1ccccc1] | 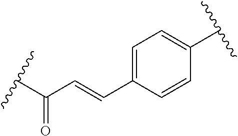 [SMILES: *C(=O)/C=C/c1ccc(*)cc1] | — |
| 1280 | 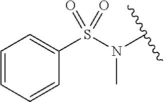 [SMILES: *N(C)S(=O)(=O)c1ccccc1] | 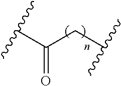 | 5 |
| 1281 | 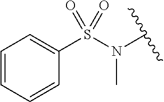 [SMILES: *N(C)S(=O)(=O)c1ccccc1] | 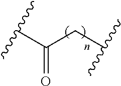 | 6 |
| 1282 | 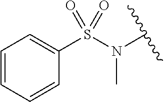 [SMILES: *N(C)S(=O)(=O)c1ccccc1] | 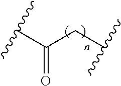 | 7 |
| 1283 | 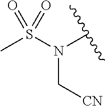 [SMILES: *N(CC#N)S(C)(=O)=O] | 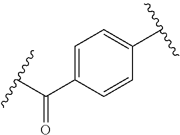 [SMILES: *C(=O)c1ccc(*)cc1] | — |
| 1284 | 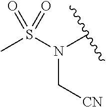 [SMILES: *N(CC#N)S(C)(=O)=O] | 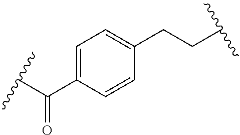 [SMILES: *CCc1ccc(C(*)=O)cc1] | |
| 1285 | 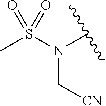 [SMILES: *N(CC#N)S(C)(=O)=O] | 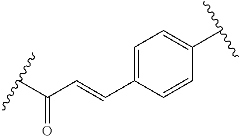 [SMILES: *C(=O)/C=C/c1ccc(*)cc1] | — |
| 1286 | 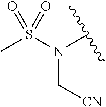 [SMILES: *N(CC#N)S(C)(=O)=O] | 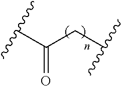 | 5 |
| 1287 | 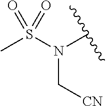 [SMILES: *N(CC#N)S(C)(=O)=O] | 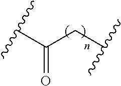 | 6 |
| 1288 | 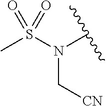 [SMILES: *N(CC#N)S(C)(=O)=O] | 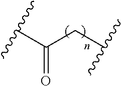 | 7 |
| 1289 | 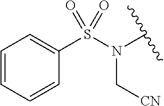 [SMILES: *N(CC#N)S(=O)(=O)c1ccccc1] | 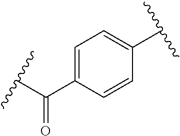 [SMILES: *C(=O)c1ccc(*)cc1] | — |
| 1290 | 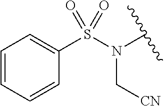 [SMILES: *N(CC#N)S(=O)(=O)c1ccccc1] | 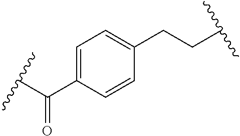 [SMILES: *CCc1ccc(C(*)=O)cc1] | |
| 1291 | 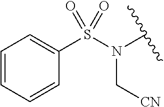 [SMILES: *N(CC#N)S(=O)(=O)c1ccccc1] | 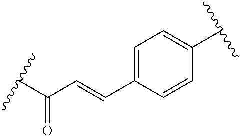 [SMILES: *C(=O)/C=C/c1ccc(*)cc1] | — |
| 1292 | 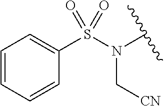 [SMILES: *N(CC#N)S(=O)(=O)c1ccccc1] | 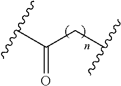 | 5 |
| 1293 | 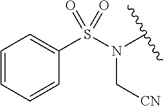 [SMILES: *N(CC#N)S(=O)(=O)c1ccccc1] | 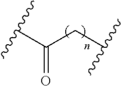 | 6 |
| 1294 | 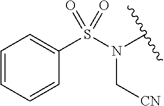 [SMILES: *N(CC#N)S(=O)(=O)c1ccccc1] | 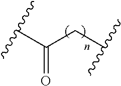 | 7 |
| 1295 | 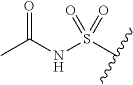 [SMILES: *S(=O)(=O)NC(C)=O] | 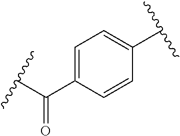 [SMILES: *C(=O)c1ccc(*)cc1] | — |
| 1296 | 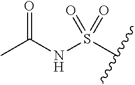 [SMILES: *S(=O)(=O)NC(C)=O] | 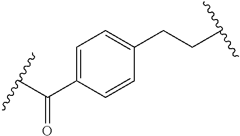 [SMILES: *CCc1ccc(C(*)=O)cc1] | |
| 1297 | 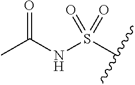 [SMILES: *S(=O)(=O)NC(C)=O] | 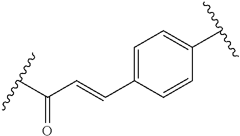 [SMILES: *C(=O)/C=C/c1ccc(*)cc1] | — |
| 1298 | 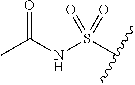 [SMILES: *S(=O)(=O)NC(C)=O] | 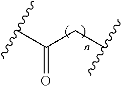 | 5 |
| 1299 | 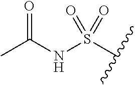 [SMILES: *S(=O)(=O)NC(C)=O] | 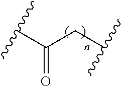 | 6 |
| 1300 | 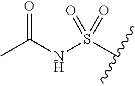 [SMILES: *S(=O)(=O)NC(C)=O] | 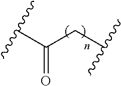 | 7 |
| 1301 | 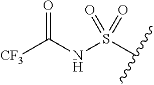 [SMILES: *S(=O)(=O)NC(=O)C(F)(F)F] | 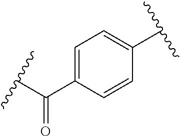 [SMILES: *C(=O)c1ccc(*)cc1] | — |
| 1302 | 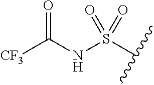 [SMILES: *S(=O)(=O)NC(=O)C(F)(F)F] | 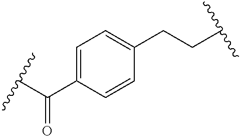 [SMILES: *CCc1ccc(C(*)=O)cc1] | |
| 1303 | 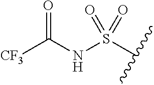 [SMILES: *S(=O)(=O)NC(=O)C(F)(F)F] | 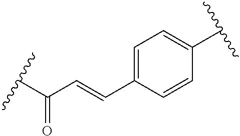 [SMILES: *C(=O)/C=C/c1ccc(*)cc1] | — |
| 1304 | 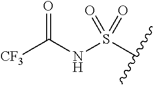 [SMILES: *S(=O)(=O)NC(=O)C(F)(F)F] | 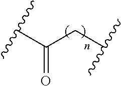 | 5 |
| 1305 | 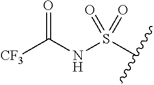 [SMILES: *S(=O)(=O)NC(=O)C(F)(F)F] | 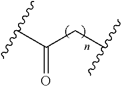 | 6 |
| 1306 | 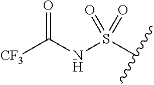 [SMILES: *S(=O)(=O)NC(=O)C(F)(F)F] | 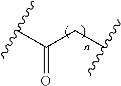 | 7 |
| 1307 | 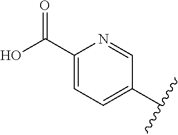 [SMILES: *c1ccc(C(=O)O)nc1] | 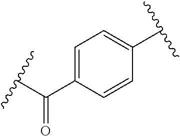 [SMILES: *C(=O)c1ccc(*)cc1] | — |
| 1308 | 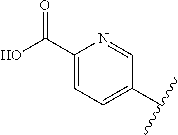 [SMILES: *c1ccc(C(=O)O)nc1] | 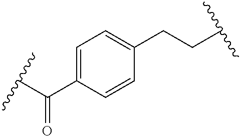 [SMILES: *CCc1ccc(C(*)=O)cc1] | |
| 1309 | 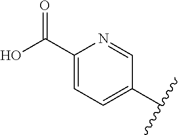 [SMILES: *c1ccc(C(=O)O)nc1] | 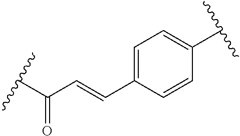 [SMILES: *C(=O)/C=C/c1ccc(*)cc1] | — |
| 1310 | 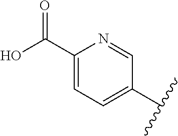 [SMILES: *c1ccc(C(=O)O)nc1] | 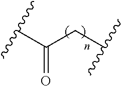 | 5 |
| 1311 | 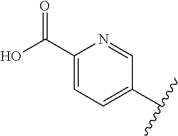 [SMILES: *c1ccc(C(=O)O)nc1] | 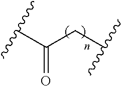 | 6 |
| 1312 | 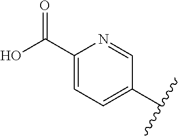 [SMILES: *c1ccc(C(=O)O)nc1] | 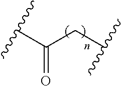 | 7 |
| 1313 | 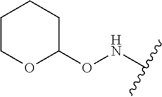 [SMILES: *NOC1CCCCO1] | 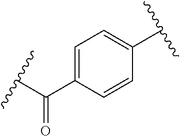 [SMILES: *C(=O)c1ccc(*)cc1] | — |
| 1314 | 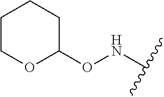 [SMILES: *NOC1CCCCO1] | 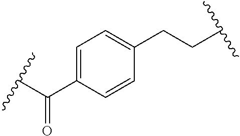 [SMILES: *CCc1ccc(C(*)=O)cc1] | |
| 1315 | 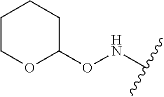 [SMILES: *NOC1CCCCO1] | 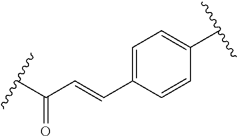 [SMILES: *C(=O)/C=C/c1ccc(*)cc1] | — |
| 1316 | 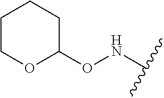 [SMILES: *NOC1CCCCO1] | 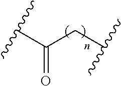 | 5 |
| 1317 | 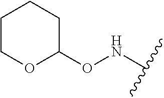 [SMILES: *NOC1CCCCO1] | 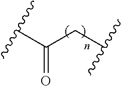 | 6 |
| 1318 | 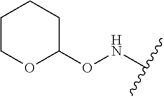 [SMILES: *NOC1CCCCO1] | 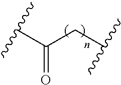 | 7 |
| 1319 | 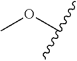 [SMILES: *OC] | 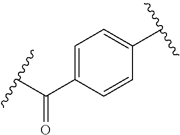 [SMILES: *C(=O)c1ccc(*)cc1] | — |
| 1320 | 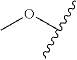 [SMILES: *OC] | 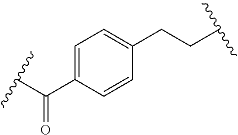 [SMILES: *CCc1ccc(C(*)=O)cc1] | |
| 1321 | 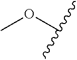 [SMILES: *OC] | 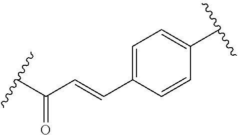 [SMILES: *C(=O)/C=C/c1ccc(*)cc1] | — |
| 1322 | 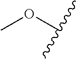 [SMILES: *OC] | 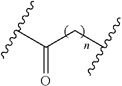 | 5 |
| 1323 | 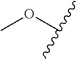 [SMILES: *OC] | 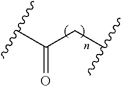 | 6 |
| 1324 | 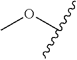 [SMILES: *OC] | 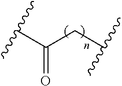 | 7 |
| 1325 | 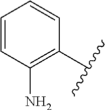 [SMILES: *c1ccccc1N] | 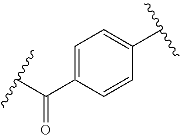 [SMILES: *C(=O)c1ccc(*)cc1] | — |
| 1326 | 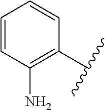 [SMILES: *c1ccccc1N] | 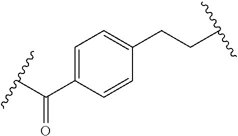 [SMILES: *CCc1ccc(C(*)=O)cc1] | |
| 1327 | 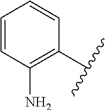 [SMILES: *c1ccccc1N] | 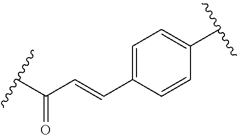 [SMILES: *C(=O)/C=C/c1ccc(*)cc1] | — |
| 1328 | 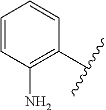 [SMILES: *c1ccccc1N] | 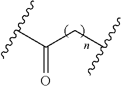 | 5 |
| 1329 | 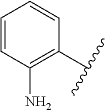 [SMILES: *c1ccccc1N] | 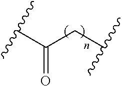 | 6 |
| 1330 | 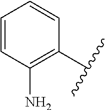 [SMILES: *c1ccccc1N] | 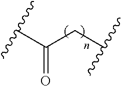 | 7 |
| 1331 | 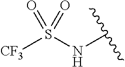 [SMILES: *NS(=O)(=O)C(F)(F)F] | 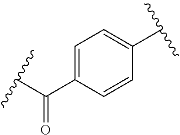 [SMILES: *C(=O)c1ccc(*)cc1] | — |
| 1332 | 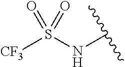 [SMILES: *NS(=O)(=O)C(F)(F)F] | 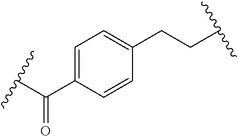 [SMILES: *CCc1ccc(C(*)=O)cc1] | |
| 1333 | 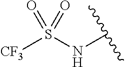 [SMILES: *NS(=O)(=O)C(F)(F)F] | 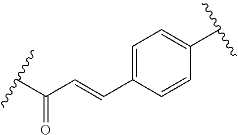 [SMILES: *C(=O)/C=C/c1ccc(*)cc1] | — |
| 1334 | 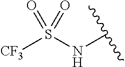 [SMILES: *NS(=O)(=O)C(F)(F)F] | 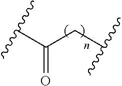 | 5 |
| 1335 | 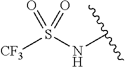 [SMILES: *NS(=O)(=O)C(F)(F)F] | 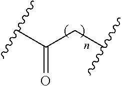 | 6 |
| 1336 | 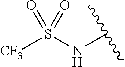 [SMILES: *NS(=O)(=O)C(F)(F)F] | 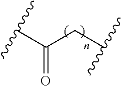 | 7 |
| 1337 | 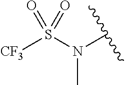 [SMILES: *N(C)S(=O)(=O)C(F)(F)F] | 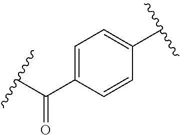 [SMILES: *C(=O)c1ccc(*)cc1] | — |
| 1338 | 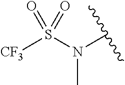 [SMILES: *N(C)S(=O)(=O)C(F)(F)F] | 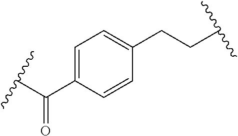 [SMILES: *CCc1ccc(C(*)=O)cc1] | |
| 1339 | 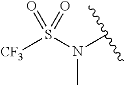 [SMILES: *N(C)S(=O)(=O)C(F)(F)F] | 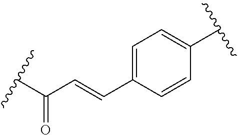 [SMILES: *C(=O)/C=C/c1ccc(*)cc1] | — |
| 1340 | 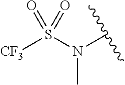 [SMILES: *N(C)S(=O)(=O)C(F)(F)F] | 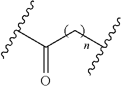 | 5 |
| 1341 | 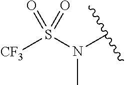 [SMILES: *N(C)S(=O)(=O)C(F)(F)F] | 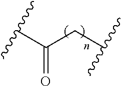 | 6 |
| 1342 | 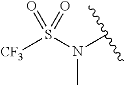 [SMILES: *N(C)S(=O)(=O)C(F)(F)F] | 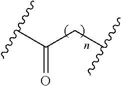 | 7 |

In some embodiments, the compound of formula (I) is a compound ofany one of formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt, solvate, hydrate, cocrystal, or prodrug thereof:

| Formula No. | Structure |
|---|---|
| AMC-3-017 | 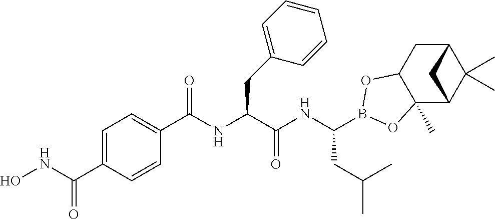 [SMILES: CC(C)C[C@H](NC(=O)[C@H](Cc1ccccc1)NC(=O)c1ccc(C(=O)NO)cc1)B1OC2C[C@H]3C[C@H](C3(C)C)[C@@]2(C)O1] |
| AMC-3-041 | 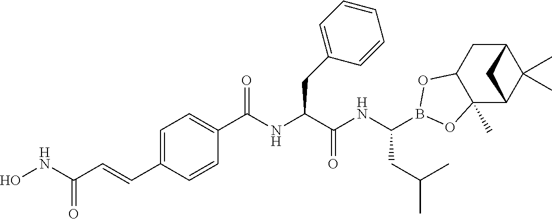 [SMILES: CC(C)C[C@H](NC(=O)[C@H](Cc1ccccc1)NC(=O)c1ccc(/C=C/C(=O)NO)cc1)B1OC2C[C@H]3C[C@H](C3(C)C)[C@@]2(C)O1] |
| AMC-2-248 | 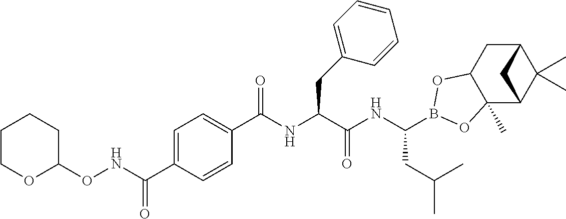 [SMILES: CC(C)C[C@H](NC(=O)[C@H](Cc1ccccc1)NC(=O)c1ccc(C(=O)NOC2CCCCO2)cc1)B1OC2C[C@H]3C[C@H](C3(C)C)[C@@]2(C)O1] |
| AMC-3-033 | 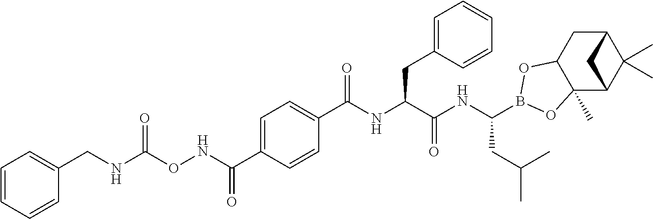 [SMILES: CC(C)C[C@H](NC(=O)[C@H](Cc1ccccc1)NC(=O)c1ccc(C(=O)NOC(=O)NCc2ccccc2)cc1)B1OC2C[C@H]3C[C@H](C3(C)C)[C@@]2(C)O1] |
| AMC-3-019 | 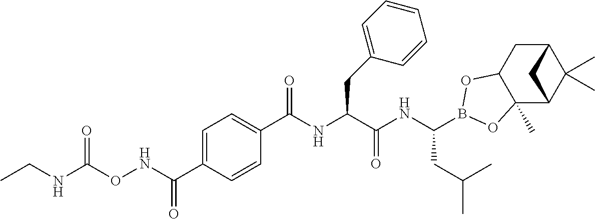 [SMILES: CCNC(=O)ONC(=O)c1ccc(C(=O)N[C@@H](Cc2ccccc2)C(=O)N[C@@H](CC(C)C)B2OC3C[C@H]4C[C@H](C4(C)C)[C@@]3(C)O2)cc1] |
| AMC-3-031 | 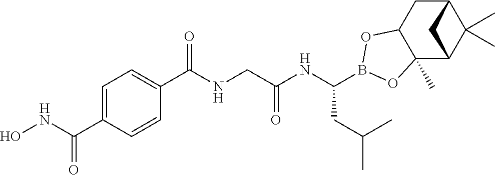 [SMILES: CC(C)C[C@H](NC(=O)CNC(=O)c1ccc(C(=O)NO)cc1)B1OC2C[C@H]3C[C@H](C3(C)C)[C@@]2(C)O1] |
| AMC-3-047 | 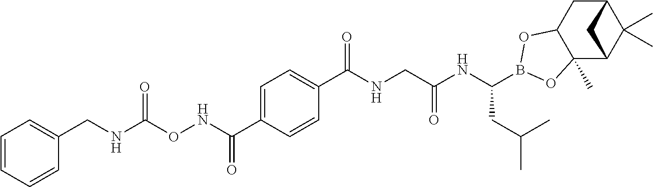 [SMILES: CC(C)C[C@H](NC(=O)CNC(=O)c1ccc(C(=O)NOC(=O)NCc2ccccc2)cc1)B1OC2C[C@H]3C[C@H](C3(C)C)[C@@]2(C)O1] |
| AMC-3-030 | 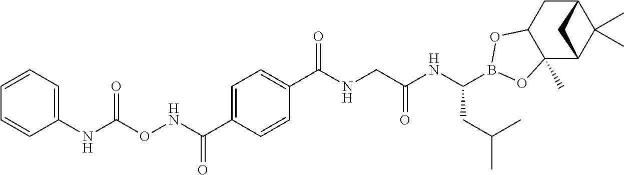 [SMILES: CC(C)C[C@H](NC(=O)CNC(=O)c1ccc(C(=O)NOC(=O)Nc2ccccc2)cc1)B1OC2C[C@H]3C[C@H](C3(C)C)[C@@]2(C)O1] |
| AMC-3-048 | 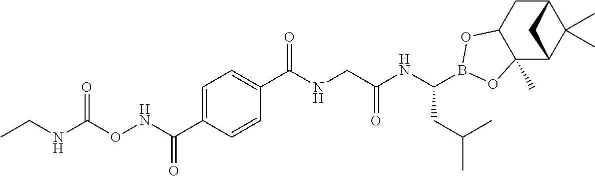 [SMILES: CCNC(=O)ONC(=O)c1ccc(C(=O)NCC(=O)N[C@@H](CC(C)C)B2OC3C[C@H]4C[C@H](C4(C)C)[C@@]3(C)O2)cc1] |
| AMC-3-055 | 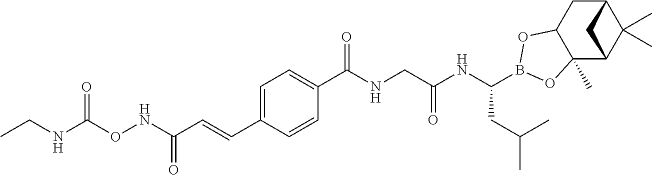 [SMILES: CCNC(=O)ONC(=O)/C=C/c1ccc(C(=O)NCC(=O)N[C@@H](CC(C)C)B2OC3C[C@H]4C[C@H](C4(C)C)[C@@]3(C)O2)cc1] |
| AMC-2-052 | 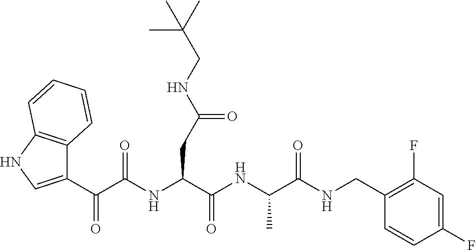 [SMILES: C[C@H](NC(=O)[C@H](CC(=O)NCC(C)(C)C)NC(=O)C(=O)c1c[nH]c2ccccc12)C(=O)NCc1ccc(F)cc1F] |
| AMC-2-036 | 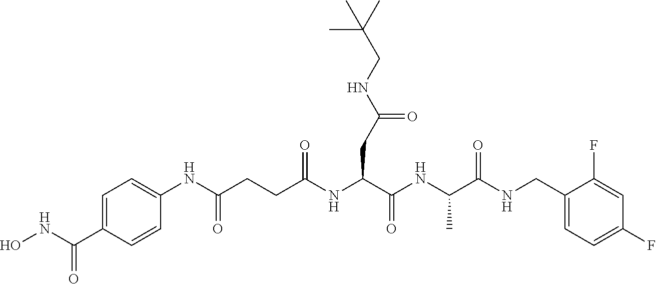 [SMILES: C[C@H](NC(=O)[C@H](CC(=O)NCC(C)(C)C)NC(=O)CCC(=O)Nc1ccc(C(=O)NO)cc1)C(=O)NCc1ccc(F)cc1F] |
| AMC-2-072 | 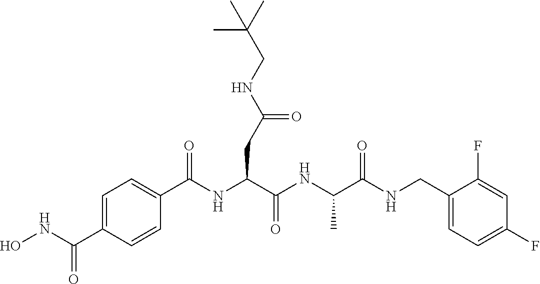 [SMILES: C[C@H](NC(=O)[C@H](CC(=O)NCC(C)(C)C)NC(=O)c1ccc(C(=O)NO)cc1)C(=O)NCc1ccc(F)cc1F] |
| AMC-2-067 | 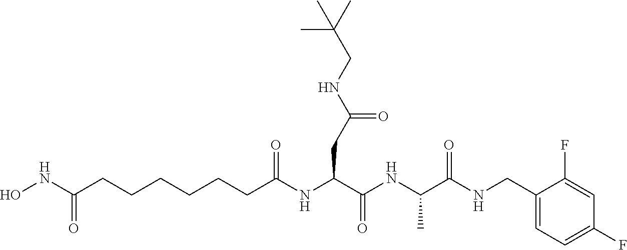 [SMILES: C[C@H](NC(=O)[C@H](CC(=O)NCC(C)(C)C)NC(=O)CCCCCCC(=O)NO)C(=O)NCc1ccc(F)cc1F] |
| AMC-2-215 | 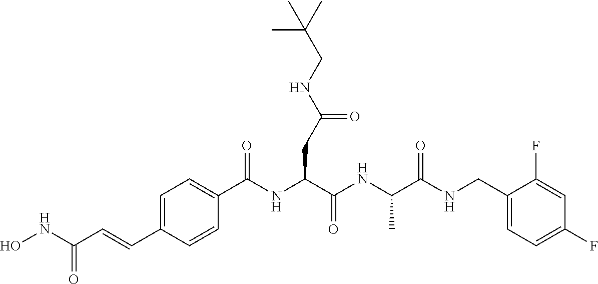 [SMILES: C[C@H](NC(=O)[C@H](CC(=O)NCC(C)(C)C)NC(=O)c1ccc(/C=C/C(=O)NO)cc1)C(=O)NCc1ccc(F)cc1F] |
| AMC-2-091 | 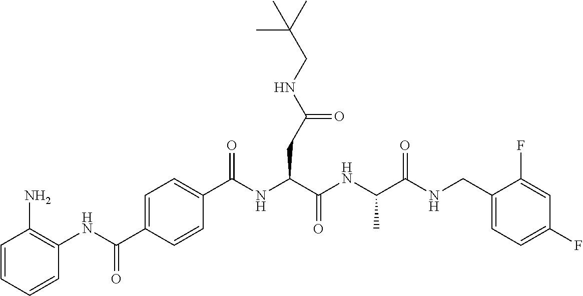 [SMILES: C[C@H](NC(=O)[C@H](CC(=O)NCC(C)(C)C)NC(=O)c1ccc(C(=O)Nc2ccccc2N)cc1)C(=O)NCc1ccc(F)cc1F] |
| AMC-2-152 | 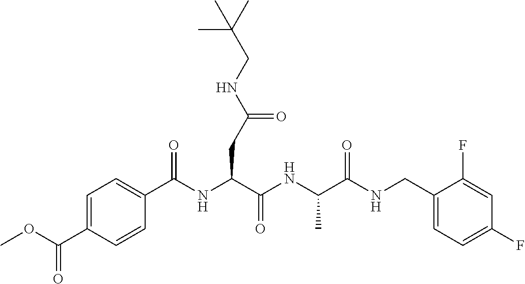 [SMILES: COC(=O)c1ccc(C(=O)N[C@@H](CC(=O)NCC(C)(C)C)C(=O)N[C@@H](C)C(=O)NCc2ccc(F)cc2F)cc1] |
| AMC-2-198 | 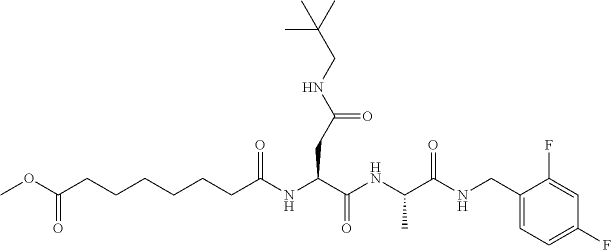 [SMILES: COC(=O)CCCCCCC(=O)N[C@@H](CC(=O)NCC(C)(C)C)C(=O)N[C@@H](C)C(=O)NCc1ccc(F)cc1F] |
| AMC-2-204 | 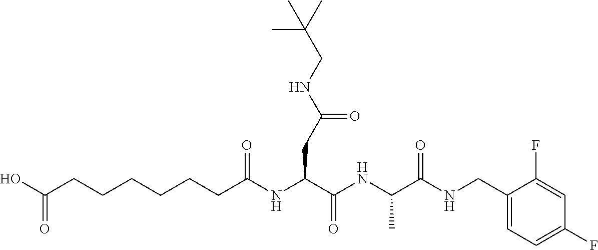 [SMILES: C[C@H](NC(=O)[C@H](CC(=O)NCC(C)(C)C)NC(=O)CCCCCCC(=O)O)C(=O)NCc1ccc(F)cc1F] |
| AMC-2-225 | 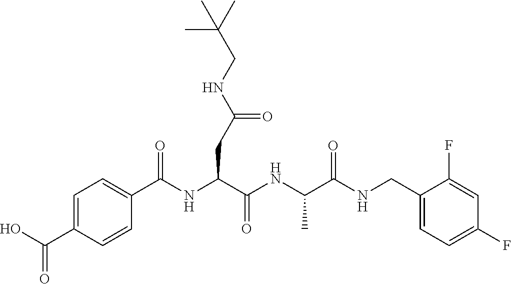 [SMILES: C[C@H](NC(=O)[C@H](CC(=O)NCC(C)(C)C)NC(=O)c1ccc(C(=O)O)cc1)C(=O)NCc1ccc(F)cc1F] |

In some embodiments, the compound of formula (I) is a compound of any one of formula AMC-3-017, formula AMC-3-041, formula AMC-2-248, formula AMC-3-033, or formula AMC-3-019, or a pharmaceutically acceptable salt, solvate, hydrate, cocrystal, or prodrug thereof:

| Formula No. | Structure |
|---|---|
| AMC-3-017 | 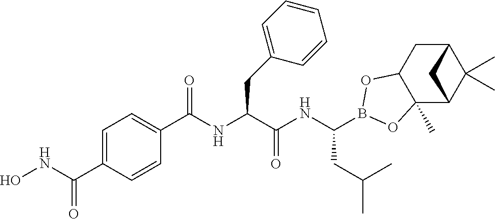 [SMILES: CC(C)C[C@H](NC(=O)[C@H](Cc1ccccc1)NC(=O)c1ccc(C(=O)NO)cc1)B1OC2C[C@H]3C[C@H](C3(C)C)[C@@]2(C)O1] |
| AMC-3-041 | 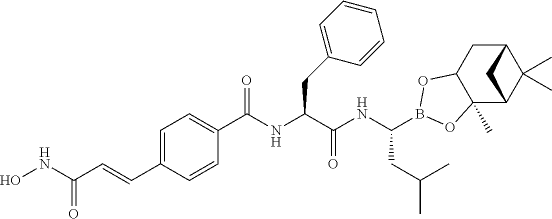 [SMILES: CC(C)C[C@H](NC(=O)[C@H](Cc1ccccc1)NC(=O)c1ccc(/C=C/C(=O)NO)cc1)B1OC2C[C@H]3C[C@H](C3(C)C)[C@@]2(C)O1] |
| AMC-2-248 | 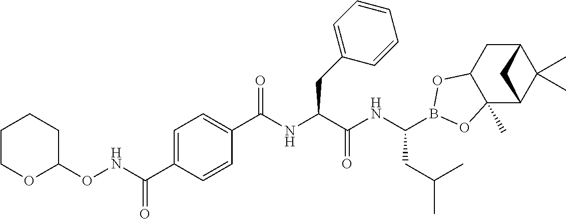 [SMILES: CC(C)C[C@H](NC(=O)[C@H](Cc1ccccc1)NC(=O)c1ccc(C(=O)NOC2CCCCO2)cc1)B1OC2C[C@H]3C[C@H](C3(C)C)[C@@]2(C)O1] |
| AMC-3-033 | 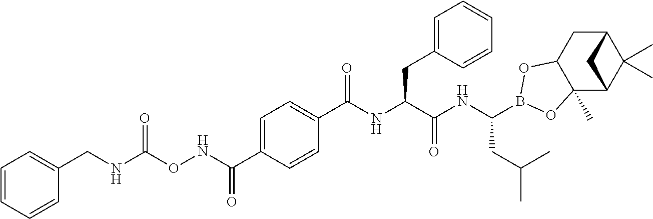 [SMILES: CC(C)C[C@H](NC(=O)[C@H](Cc1ccccc1)NC(=O)c1ccc(C(=O)NOC(=O)NCc2ccccc2)cc1)B1OC2C[C@H]3C[C@H](C3(C)C)[C@@]2(C)O1] |
| AMC-3-019 | 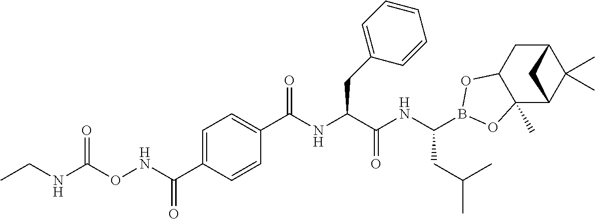 [SMILES: CCNC(=O)ONC(=O)c1ccc(C(=O)N[C@@H](Cc2ccccc2)C(=O)N[C@@H](CC(C)C)B2OC3C[C@H]4C[C@H](C4(C)C)[C@@]3(C)O2)cc1] |

In some embodiments, the compound of formula (I) is a compound of any one of formula AMC-3-031, formula AMC-3-047, formula AMC-3-030, formula AMC-3-048, or formula AMC-3-055, or a pharmaceutically acceptable salt, solvate, hydrate, cocrystal, or prodrug thereof:

| Formula No. | Structure |
|---|---|
| AMC-3-031 | 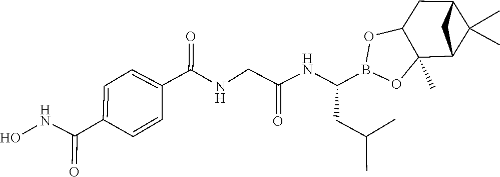 [SMILES: CC(C)C[C@H](NC(=O)CNC(=O)c1ccc(C(=O)NO)cc1)B1OC2C[C@H]3C[C@H](C3(C)C)[C@@]2(C)O1] |
| AMC-3-047 | 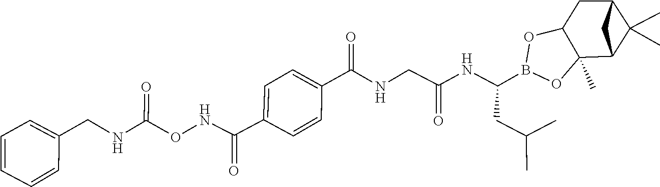 [SMILES: CC(C)C[C@H](NC(=O)CNC(=O)c1ccc(C(=O)NOC(=O)NCc2ccccc2)cc1)B1OC2C[C@H]3C[C@H](C3(C)C)[C@@]2(C)O1] |
| AMC-3-030 | 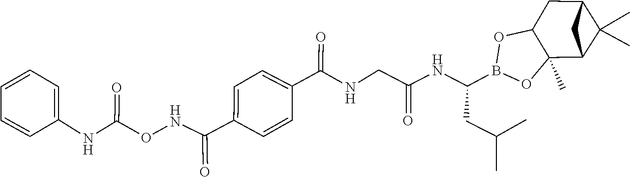 [SMILES: CC(C)C[C@H](NC(=O)CNC(=O)c1ccc(C(=O)NOC(=O)Nc2ccccc2)cc1)B1OC2C[C@H]3C[C@H](C3(C)C)[C@@]2(C)O1] |
| AMC-3-048 | 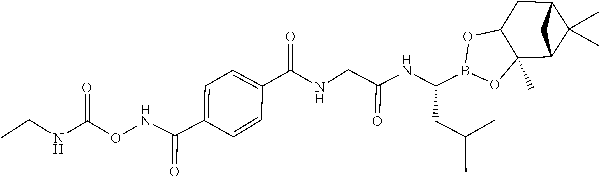 [SMILES: CCNC(=O)ONC(=O)c1ccc(C(=O)NCC(=O)N[C@@H](CC(C)C)B2OC3C[C@H]4C[C@H](C4(C)C)[C@@]3(C)O2)cc1] |
| AMC-3-055 | 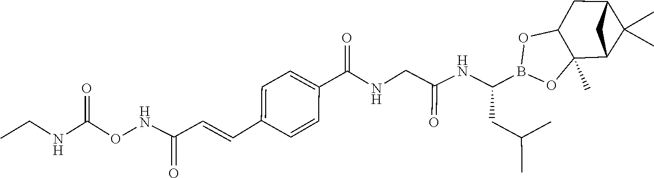 [SMILES: CCNC(=O)ONC(=O)/C=C/c1ccc(C(=O)NCC(=O)N[C@@H](CC(C)C)B2OC3C[C@H]4C[C@H](C4(C)C)[C@@]3(C)O2)cc1] |

In some embodiments, the compound of formula (I) is a compound of any one of formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt, solvate, hydrate, cocrystal, or prodrug thereof:

| Formula No. | Structure |
|---|---|
| AMC-2-052 | 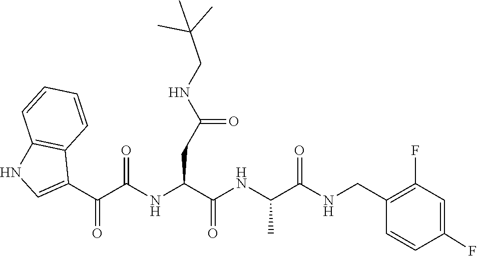 [SMILES: C[C@H](NC(=O)[C@H](CC(=O)NCC(C)(C)C)NC(=O)C(=O)c1c[nH]c2ccccc12)C(=O)NCc1ccc(F)cc1F] |
| AMC-2-036 | 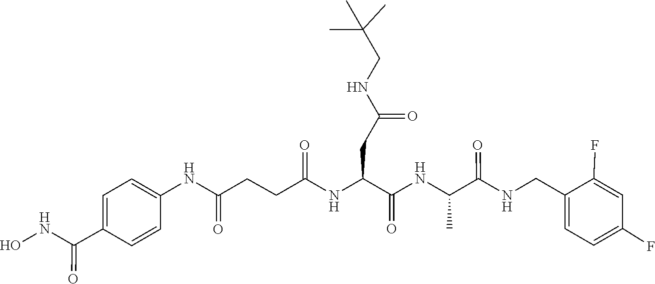 [SMILES: C[C@H](NC(=O)[C@H](CC(=O)NCC(C)(C)C)NC(=O)CCC(=O)Nc1ccc(C(=O)NO)cc1)C(=O)NCc1ccc(F)cc1F] |
| AMC-2-072 | 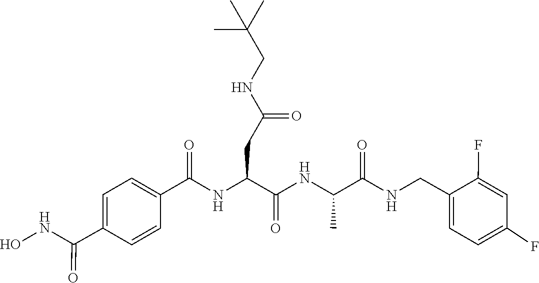 [SMILES: C[C@H](NC(=O)[C@H](CC(=O)NCC(C)(C)C)NC(=O)c1ccc(C(=O)NO)cc1)C(=O)NCc1ccc(F)cc1F] |
| AMC-2-067 | 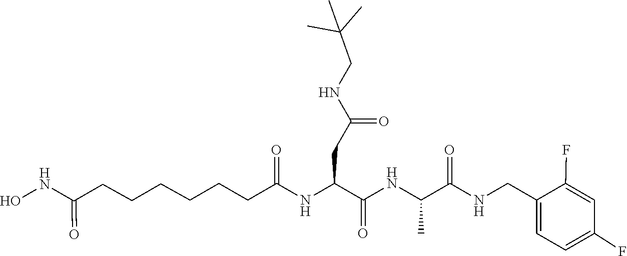 [SMILES: C[C@H](NC(=O)[C@H](CC(=O)NCC(C)(C)C)NC(=O)CCCCCCC(=O)NO)C(=O)NCc1ccc(F)cc1F] |
| AMC-2-215 | 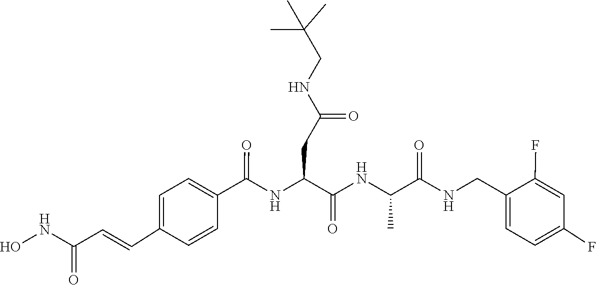 [SMILES: C[C@H](NC(=O)[C@H](CC(=O)NCC(C)(C)C)NC(=O)c1ccc(/C=C/C(=O)NO)cc1)C(=O)NCc1ccc(F)cc1F] |
| AMC-2-091 | 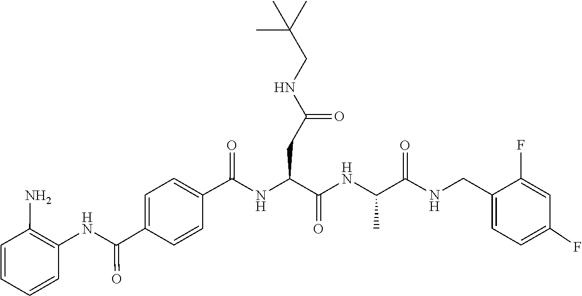 [SMILES: C[C@H](NC(=O)[C@H](CC(=O)NCC(C)(C)C)NC(=O)c1ccc(C(=O)Nc2ccccc2N)cc1)C(=O)NCc1ccc(F)cc1F] |
| AMC-2-152 | 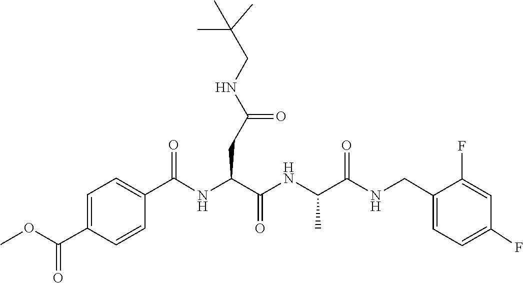 [SMILES: COC(=O)c1ccc(C(=O)N[C@@H](CC(=O)NCC(C)(C)C)C(=O)N[C@@H](C)C(=O)NCc2ccc(F)cc2F)cc1] |
| AMC-2-198 | 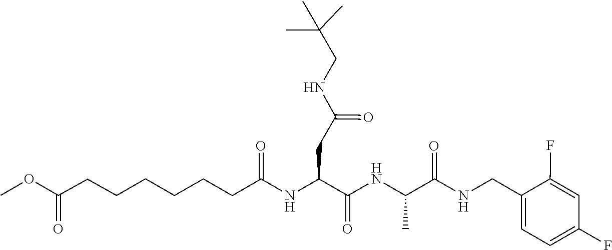 [SMILES: COC(=O)CCCCCCC(=O)N[C@@H](CC(=O)NCC(C)(C)C)C(=O)N[C@@H](C)C(=O)NCc1ccc(F)cc1F] |
| AMC-2-204 | 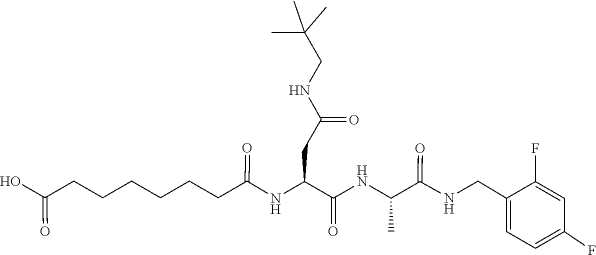 [SMILES: C[C@H](NC(=O)[C@H](CC(=O)NCC(C)(C)C)NC(=O)CCCCCCC(=O)O)C(=O)NCc1ccc(F)cc1F] |
| AMC-2-225 | 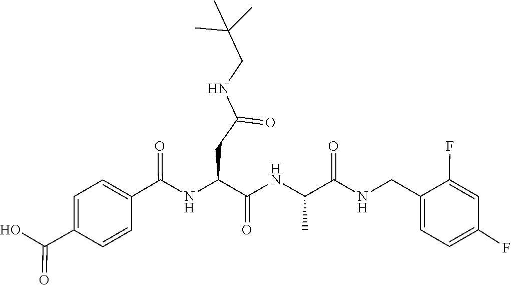 [SMILES: C[C@H](NC(=O)[C@H](CC(=O)NCC(C)(C)C)NC(=O)c1ccc(C(=O)O)cc1)C(=O)NCc1ccc(F)cc1F] |

In some embodiments, the compound of formula (I) is a compound of formula 1230, or a pharmaceutically acceptable salt, solvate, hydrate, cocrystal, or prodrug thereof.

In some embodiments, the compound of formula (I) is a compound of formula 1231, or a pharmaceutically acceptable salt, solvate, hydrate, cocrystal, or prodrug thereof.

### Methods of Treatment

The compounds and compositions described herein can be used in methods for treating diseases and disorders. In some embodiments, the compounds and compositions described herein can be used in methods for treating diseases associated with the upregulation of the HDAC6 protein. In some embodiments, the compounds and compositions described herein can be used in methods for treating diseases associated with the upregulation of the proteasome. In some embodiments, the compounds and compositions described herein can be used for the treatment of cancer. In some embodiments, the compounds and compositions described herein can be used for the treatment of inflammatory diseases and disorders. In some embodiments, the compounds and compositions described herein can be used for the treatment of autoimmune diseases and disorders. The compounds and compositions described herein may also be used in treating other disorders as described herein and in the following paragraphs.

In one aspect, the disclosure provides a method of treating or preventing a disease or disorder in a patient in need thereof, the method comprising administering a therapeutically effective amount of one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof. In some embodiments, the disease or disorder is alleviated by inhibiting HDAC6 protein activity in the patient. In some embodiments, the disease or disorder is alleviated by inhibiting proteasome activity in the patient. In some embodiments, the disease or disorder is alleviated by inhibiting both HDAC6 protein activity and inhibiting proteasome activity in the patient.

In one aspect, the disclosure provides a method of treating or preventing a disease or disorder alleviated by inhibiting HDAC6 protein activity in a patient in need of said treatment or prevention. In some embodiments, the method comprises administering a therapeutically effective amount of one or more compounds of the disclosure, or a pharmaceutically acceptable salt thereof.

In one aspect, the disclosure provides a method of treating or preventing a disease or disorder alleviated by inhibiting proteasome activity in a patient in need of said treatment or prevention. In some embodiments, the method comprises administering a therapeutically effective amount of one or more compounds of the disclosure, or a pharmaceutically acceptable salt thereof.

In one aspect, the disclosure provides a method of treating or preventing a disease or disorder alleviated by inhibiting HDAC6 protein activity and/or inhibiting proteasome activity in a patient in need of said treatment or prevention. In some embodiments, the method comprises administering a therapeutically effective amount of one or more compounds of the disclosure, or a pharmaceutically acceptable salt thereof.

In some embodiments, the disease or disorder is cancer, an inflammatory disease or disorder, or an autoimmune disease or disorder.

In some embodiments, the disease or disorder is cancer. In some embodiments, the cancer is selected from the cancer is selected from multiple myeloma (MM), mantle cell lymphoma, acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like. In some embodiments, the cancer is multiple myeloma (MM).

In some embodiments, the cancer is a blood cancer. In some embodiments, the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

In one aspect, the disclosure provides a method of treating or preventing multiple myeloma (MM) in a patient in need of said treatment or prevention. In some embodiments, the method comprises administering a therapeutically effective amount of one or more compounds of the disclosure, or a pharmaceutically acceptable salt thereof.

In some embodiments, the disease or disorder is an inflammatory disease or disorder. In some embodiments, the inflammatory disease or disorder is selected from autoimmune diseases, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

In some embodiments, the disease or disorder is an autoimmune disease or disorder. In some embodiments, the autoimmune disease or disorder is selected from Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

Efficacy of the compounds and combinations of compounds described herein treating the indicated diseases or disorders can be tested using various models known in the art, and described herein, which provide guidance for treatment of human disease.

### Pharmaceutical Compositions

In an embodiment, an active pharmaceutical ingredient or combination of active pharmaceutical ingredients, such as any of the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is provided as a pharmaceutically acceptable composition.

In some embodiments, the concentration of each of the active pharmaceutical ingredients provided in the pharmaceutical compositions of the disclosure, such as any of the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of each of the active pharmaceutical ingredients provided in the pharmaceutical compositions of the disclosure, such as any of the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of each of the active pharmaceutical ingredients provided in the pharmaceutical compositions of the disclosure, such as any of the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of each of the active pharmaceutical ingredients provided in the pharmaceutical compositions of the disclosure, such as any of the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the disclosure, such as any of the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the disclosure is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, may also be used if appropriate.

In an embodiment, the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In one aspect, the disclosure provides a pharmaceutical composition comprising one or more of compounds of any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium.

In one aspect, the disclosure provides a pharmaceutical composition for treating or preventing a disease or disorder alleviated by inhibiting HDAC6 protein activity, the pharmaceutical composition comprising one or more compounds according to any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium. In some embodiments, the disease or disorder is cancer. In some embodiments, the disease or disorder is an inflammatory disease or disorder. In some embodiments, the disease or disorder is an autoimmune disease or disorder.

In one aspect, the disclosure provides a pharmaceutical composition for treating or preventing a disease or disorder alleviated by inhibiting proteasome activity, the pharmaceutical composition comprising one or more compounds according to any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium. In some embodiments, the disease or disorder is cancer. In some embodiments, the disease or disorder is an inflammatory disease or disorder. In some embodiments, the disease or disorder is an autoimmune disease or disorder.

In one aspect, the disclosure provides a pharmaceutical composition for treating or preventing a disease or disorder alleviated by both inhibiting HDAC6 protein activity and inhibiting proteasome activity, the pharmaceutical composition comprising one or more compounds according to any one of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium. In some embodiments, the disease or disorder is cancer. In some embodiments, the disease or disorder is an inflammatory disease or disorder. In some embodiments, the disease or disorder is an autoimmune disease or disorder.

In some embodiments, the cancer is selected from multiple myeloma (MM), mantle cell lymphoma, acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is acute myeloid leukemia (AML). In some embodiments, the cancer is chronic lymphocytic leukemia (CLL).

In some embodiments, the cancer is a blood cancer. In some embodiments, the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

In some embodiments, the inflammatory disease or disorder is selected from autoimmune diseases, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

In some embodiments, the autoimmune disease or disorder is selected from Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

Furthermore, the described methods of treatment may normally include medical follow-up to determine the therapeutic or prophylactic effect brought about in the subject undergoing treatment with the compound(s) and/or composition(s) described herein.

In one aspect, the disclosure provides a pharmaceutical composition for treating or preventing from multiple myeloma (MM) the pharmaceutical composition comprising one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a physiologically compatible carrier medium.

Described below are non-limiting pharmaceutical compositions and methods for preparing the same.

### Pharmaceutical Compositions for Oral Administration

In an embodiment, the disclosure provides a pharmaceutical composition for oral administration containing the active pharmaceutical ingredient or combination of active pharmaceutical ingredients, such as one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a pharmaceutical excipient suitable for oral administration.

In some embodiments, the disclosure provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient and optionally (iv) an effective amount of a fourth active pharmaceutical ingredient.

In some embodiments, the pharmaceutical composition may be a liquid pharmaceutical composition suitable for oral consumption. Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogs thereof; polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, a pharmaceutical composition is provided for injection containing an active pharmaceutical ingredient or combination of active pharmaceutical ingredients, such as one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt thereof, and a pharmaceutical excipient suitable for injection.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, a pharmaceutical composition is provided for transdermal delivery containing an active pharmaceutical ingredient or combination of active pharmaceutical ingredients, such as compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of an active pharmaceutical ingredient or combination of active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139, the entirety of which are incorporated herein by reference. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra and the compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, described herein. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions comprising one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The active pharmaceutical ingredient or combination of active pharmaceutical ingredients can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

The disclosure also provides kits. The kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the disclosure provides a kit comprising a composition comprising a therapeutically effective amount of an active pharmaceutical ingredient or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the disclosure provides a kit comprising (1) a composition comprising a therapeutically effective amount of an active pharmaceutical ingredient or combination of active pharmaceutical ingredients or a pharmaceutically acceptable salt, solvate, hydrate, cocrystal, or prodrug thereof, and (2) a diagnostic test for determining whether a patient's cancer is a particular subtype of a cancer. Any of the foregoing diagnostic methods may be utilized in the kit.

The kits described above are preferably for use in the treatment of the diseases and conditions described herein. In a particular embodiment, the kits are for use in the treatment of hyperproliferative disorders.

In a particular embodiment, the kits described herein are for use in the treatment of cancer. In some embodiments, the kits described herein are for use in the treatment of a cancer selected from acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In particular embodiments, the kits described herein are for use in the treatment of acute myeloid leukemia (AML). In particular embodiments, the kits described herein are for use in the treatment of chronic lymphocytic leukemia (CLL).

### Dosages and Dosing Regimens

The amounts of the pharmaceutical compositions administered using the methods herein, such as the dosages of compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, the pharmaceutical composition comprising one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is administered in combination with one or more anti-cancer agents. Non-limiting examples of anti-cancer agents include abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dateparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gem tuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, pegasparagase, pegfilgrastim, peginterferon alpha-2b, pemetrexed, pentostatin, pipobrman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, and analogues thereof. In some embodiments, the pharmaceutical composition comprising one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is administered prior to, concurrently with, and/or after the administration of one or more anti-cancer agents.

In some embodiments, the pharmaceutical composition comprising one or more compounds of formula (I), formula (10), formula (20), formula (11), formula (12), formula (110), formula (111), formula (120), formula (121), formula 1001-1342, formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, is administered in combination with one or more agents useful for the treatment of autoimmune or inflammatory diseases or disorders. The agents can be, for example, a pain reliever such as aspirin or Tylenol® (acetaminophen), a nonsteroidal anti-inflammatory drug (NSAID) such as ibuprofen, a corticosteroid such as cortisone or prednisone, a therapeutic antibody such as etanercept (Enbrel™), infliximab (Remicade™) or anakinra (Kineret™), an immunomodulatory agent such as methotrexate, cyclophosphamide, or cyclosporine, an antibiotic such as Flagyl™ (Metronidazole) or Cipro™ (Ciprofloxacin), or small molecule compounds such as sulfasalazine (Azulfidine™) or hydroxychloroquine (Plaquenil™).

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the preferred oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients may continue as long as necessary. In selected embodiments, a pharmaceutical composition is administered for more than 1, 2, 3, 4, 5, 6, 7, 14, or 28 days. In some embodiments, a pharmaceutical composition is administered for less than 28, 14, 7, 6, 5, 4, 3, 2, or 1 day. In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an active pharmaceutical ingredient disclosed herein is in the range of about 0.01 mg/kg to about 200 mg/kg, or about 0.1 to 100 mg/kg, or about 1 to 50 mg/kg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect—e.g., by dividing such larger doses into several small doses for administration throughout the day. Of course, as those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the compositions described herein further include controlled-release, sustained release, or extended-release therapeutic dosage forms for administration of the compounds described herein, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

The following examples describe the disclosure in further detail. These examples are provided for illustrative purposes only, and should in no way be considered as limiting the disclosure.

### EXAMPLES

### Example 1: Polypharmacologic Approach to Relapsed/Refractory Multiple Myeloma: Dual Inhibition of the HDAC6 and Proteasome Pathways

This example describes the development of dual HDAC6/proteasome inhibitors, including compounds with high HDAC6 selectivity. While not wishing to be bound by any particular theory; the increased HDAC selectivity is hypothesized to decrease the cytotoxic effects of pan-HDAC inhibitors while retaining therapeutic efficacy through inhibition of the aggresome pathway. Furthermore, novel covalent HDAC6 inhibitors which do not use the traditional hydroxamic acid moiety as a zinc binding group (ZBG) are developed. Hydroxamic acids are known to be highly promiscuous binders, contributing to the toxicities seen by HDAC inhibitors. Once developed as an HDAC6 inhibitor, the novel covalent moiety is applied to the dual inhibitors. It is hypothesized that dual HDAC6/proteasome inhibitors, especially those containing the novel HDAC inhibiting moiety, will result in higher therapeutic effect compared to the traditional combination treatments.

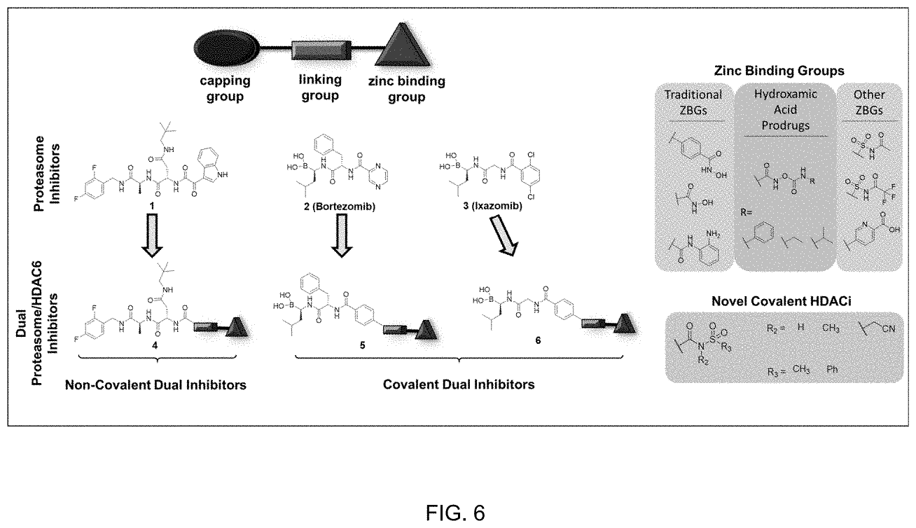

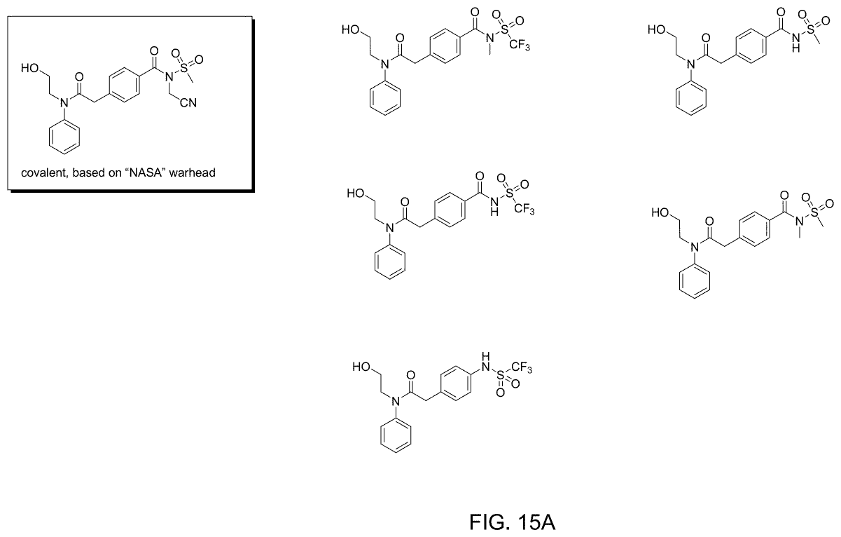

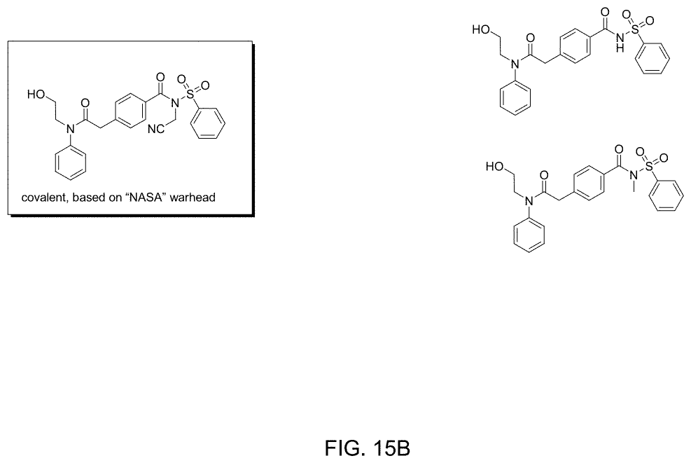

In a non-limiting example, proteasome inhibitors act as the capping group to the dual inhibitors (FIG. 6). A variety of different linkers and zinc binding groups (ZBGs) are examined, such as carbamates. For example, carbamates of hydroxamic acids have been shown to be prodrugs for the hydroxamic acid moiety as well as chelate zinc in its original structure. In some embodiments, novel covalent zinc binding groups based on the N-acyl-N-alkyl sulfonamide (NASA warhead) are incorporated into the dual inhibitors. Such compounds target a tyrosine residue in the HDAC binding pocket while also chelating the zinc ion, and are developed as solely HDAC6 inhibitors (see FIGS. 15A-15B for non-limiting examples of structures.

PROTEASOME ACTIVITY ASSAY: Testing for proteasome activity consists of a fluorescence-based assay; a fluorescently tagged peptide is used as a substrate, which is cleaved by the proteolytic activity of the proteasome. This results in the release of the fluorescent tag that is quantified to measure remaining proteasome activity after treatment. A non-limiting example of a Proteasome Activity Assay Kit includes product ab107921 from Abcam.

HDAC ACTIVITY ASSAYS: HDAC activity is measured using a microfluidics mobility shift platform. A fluorescently labeled substrate is reacted with the enzyme along with addition of test compounds followed by reading through microfluidic chips. Enzyme activity is determined through quantitative comparison of the more positive substrate and more negative product that results from enzymatic activity.

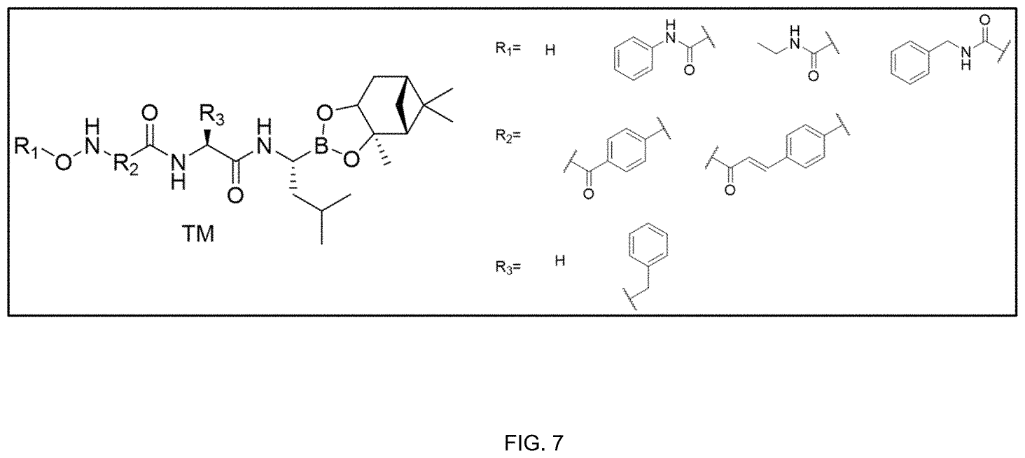

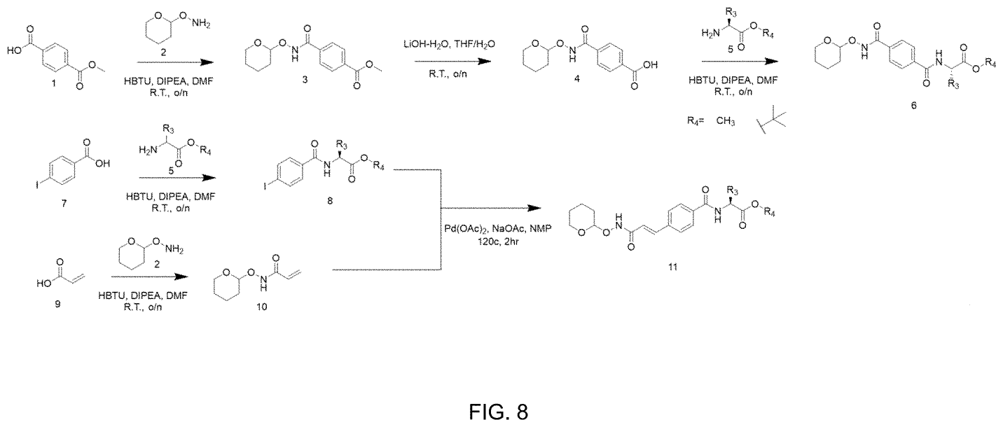

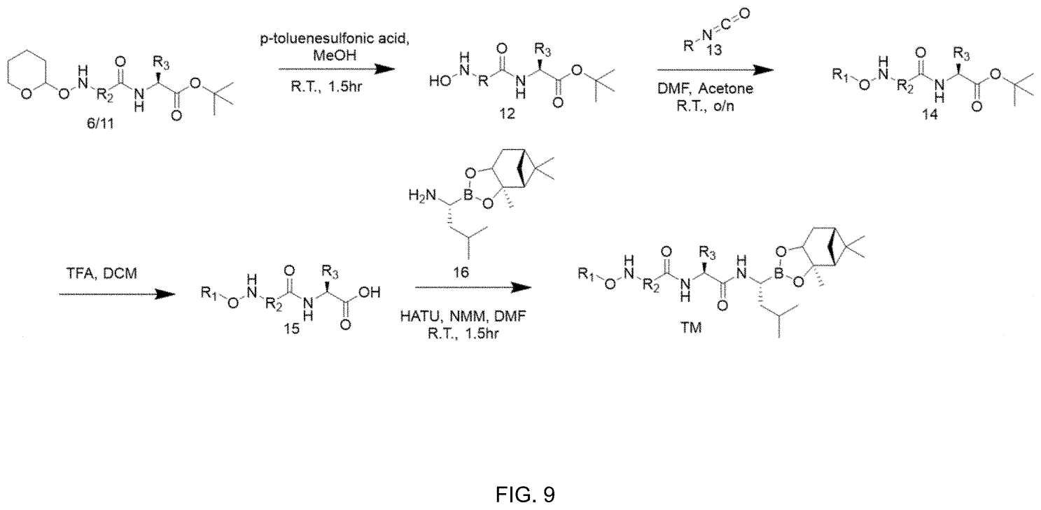

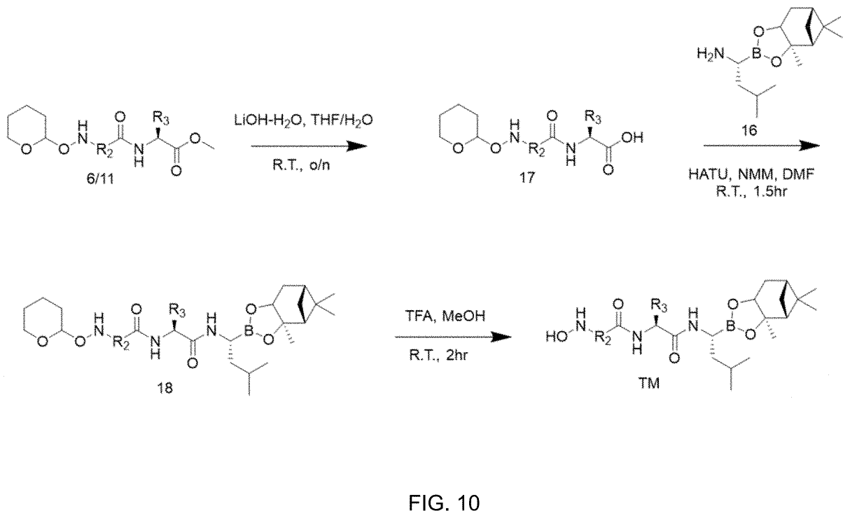

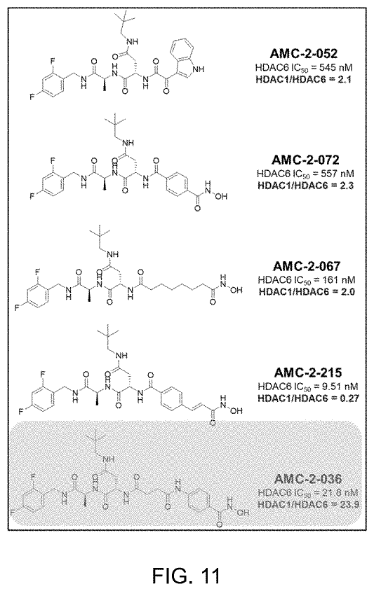

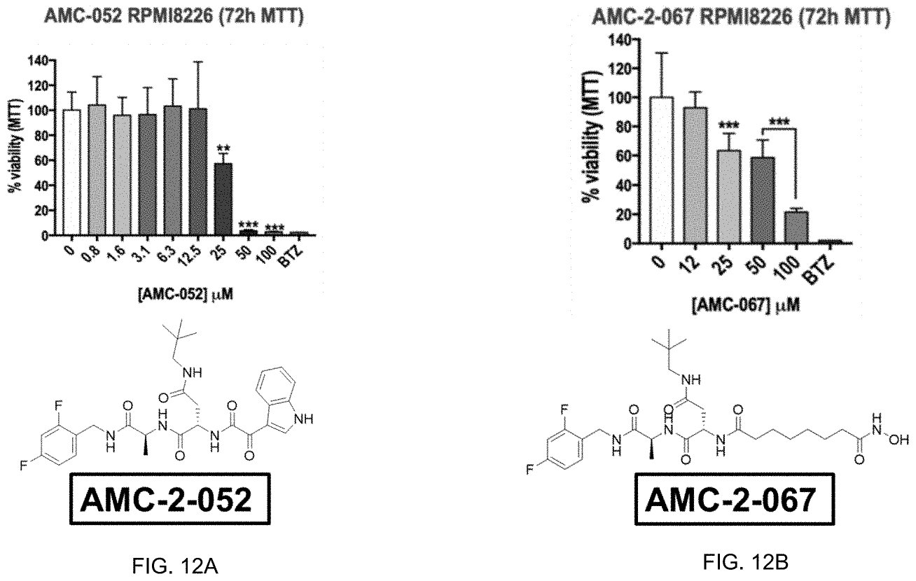

Non-limiting examples of the synthesis of covalent dual HDAC6/proteasome inhibitors are shown in FIGS. 7-10. FIG. 11 shows experimental data for dual HDAC6/Non-covalent proteasome inhibitors of the disclosure, as well as-fold improvement over HDAC1 inhibition. Of the compounds tested, compound AMC-2-215 showed the best potency for HDAC6. Compound AMC-2-036 (highlighted) was identified as a highly potent compound based on both potency and selectivity for HDAC6. FIG. 12A illustrates experimental data demonstrating the percent viability (MTT) of compound AMC-2-052. FIG. 12B illustrates experimental data demonstrating the percent viability (MTT) of compound AMC-2-067.

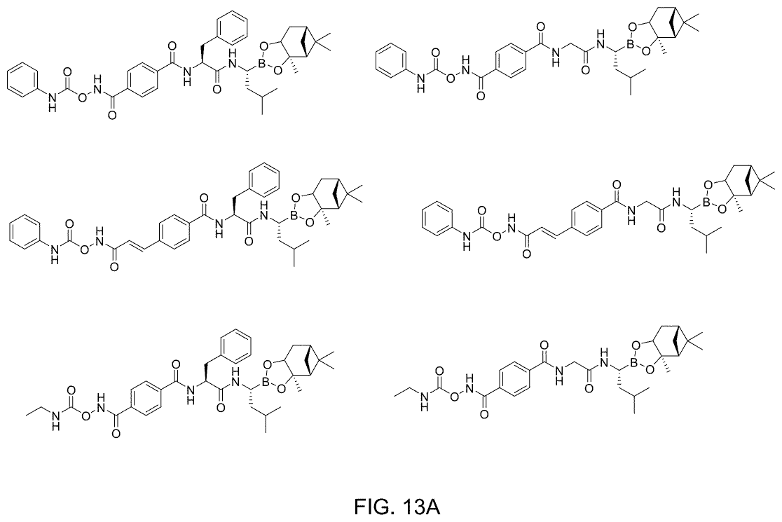

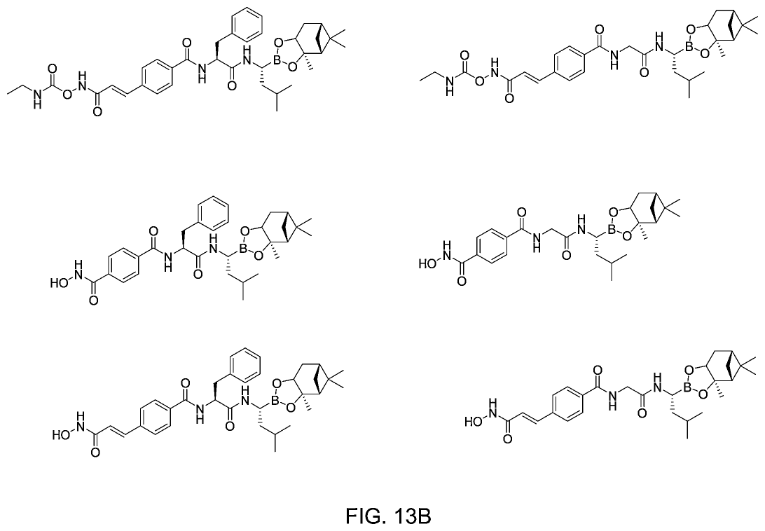

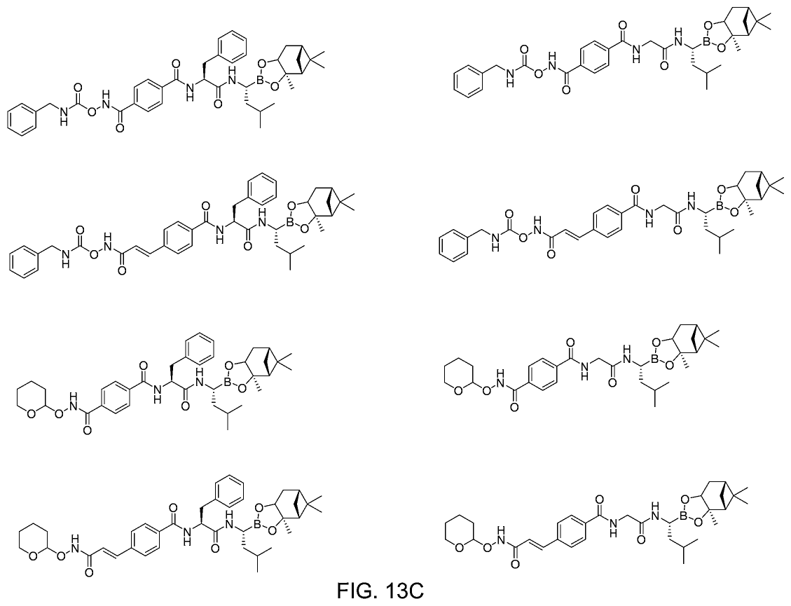

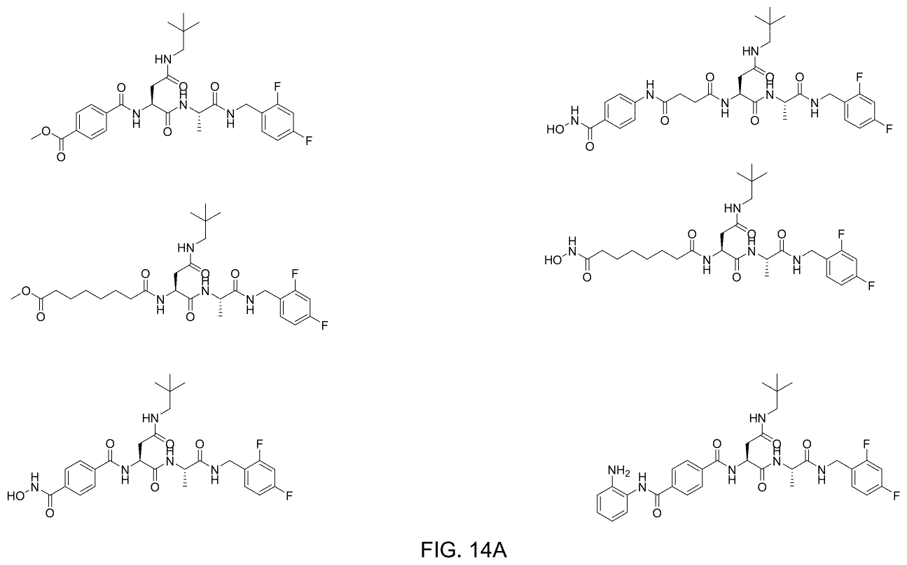

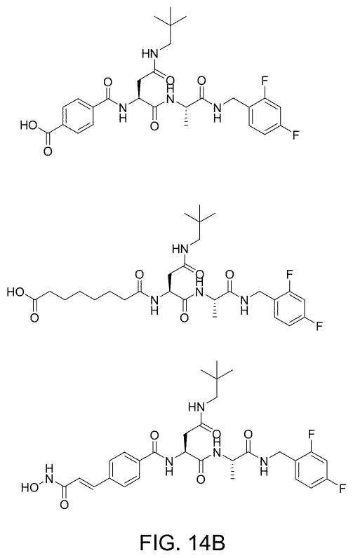

FIGS. 13A-13C show the structures of non-limiting examples of dual HDAC6/covalent proteasome inhibitors of the disclosure. FIGS. 14A-14B show the structures of non-limiting examples of dual HDAC6/non-covalent proteasome inhibitors of the disclosure.

### Example 2: HDAC6 Inhibition and HDAC6 Selectivity Studies Using Non-Covalent Dual Inhibitors and Covalent Dual Inhibitors of the Disclosure

The percent inhibition of both HDAC1 and HDAC6 was determined for several dual HDAC6/non-covalent proteasome inhibitors of the disclosure, as set forth below in Table 1. The concentration of each non-covalent inhibitor compound tested in this study was 1 μM.

TABLE 1
Percent Inhibition of HDAC1 and HDAC6 for Non-Covalent Dual Inhibitors

| | HDAC1 % | HDAC6 % |
|---|---|---|
| Compound | Inhibition | Inhibition |
| AMC-2-052 | 41 | 59 |
| 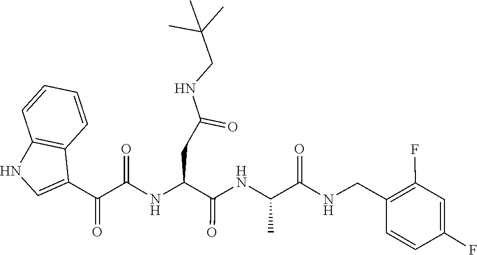 [SMILES: C[C@H](NC(=O)[C@H](CC(=O)NCC(C)(C)C)NC(=O)C(=O)c1c[nH]c2ccccc12)C(=O)NCc1ccc(F)cc1F] | | |
| AMC-2-036 | 61 | 95 |
| 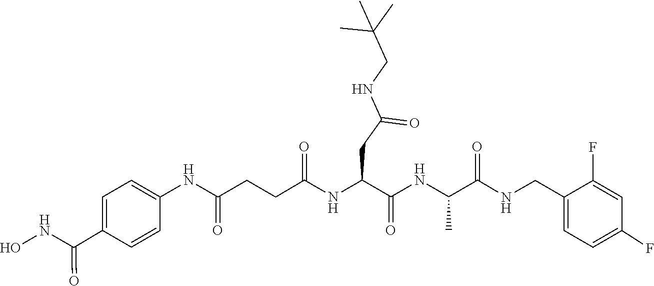 [SMILES: C[C@H](NC(=O)[C@H](CC(=O)NCC(C)(C)C)NC(=O)CCC(=O)Nc1ccc(C(=O)NO)cc1)C(=O)NCc1ccc(F)cc1F] | | |
| AMC-2-072 | 36 | 60 |
| 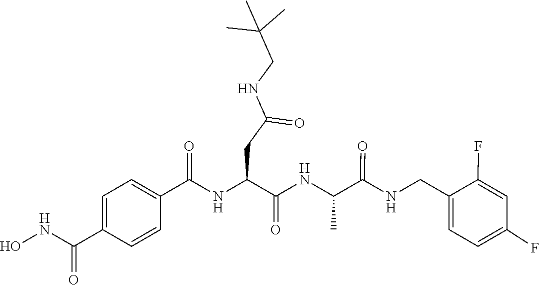 [SMILES: C[C@H](NC(=O)[C@H](CC(=O)NCC(C)(C)C)NC(=O)c1ccc(C(=O)NO)cc1)C(=O)NCc1ccc(F)cc1F] | | |
| AMC-2-067 | 74 | 86 |
| 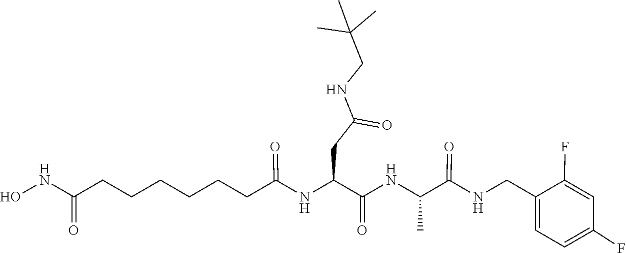 [SMILES: C[C@H](NC(=O)[C@H](CC(=O)NCC(C)(C)C)NC(=O)CCCCCCC(=O)NO)C(=O)NCc1ccc(F)cc1F] | | |
| AMC-2-215 | 99 | 98 |
| 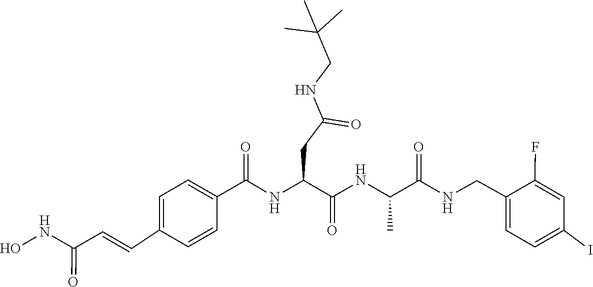 [SMILES: C[C@H](NC(=O)[C@H](CC(=O)NCC(C)(C)C)NC(=O)c1ccc(/C=C/C(=O)NO)cc1)C(=O)NCc1ccc(I)cc1F] | | |
| AMC-2-091 | 65 | 13 |
| 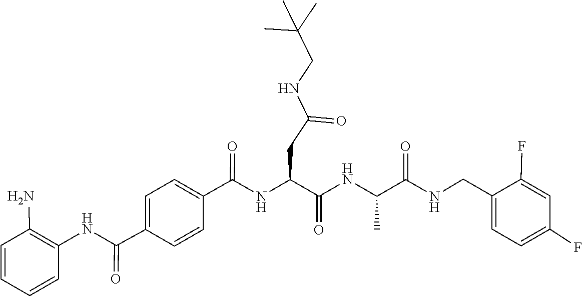 [SMILES: C[C@H](NC(=O)[C@H](CC(=O)NCC(C)(C)C)NC(=O)c1ccc(C(=O)Nc2ccccc2N)cc1)C(=O)NCc1ccc(F)cc1F] | | |
| AMC-2-152 | 3 | 18 |
| 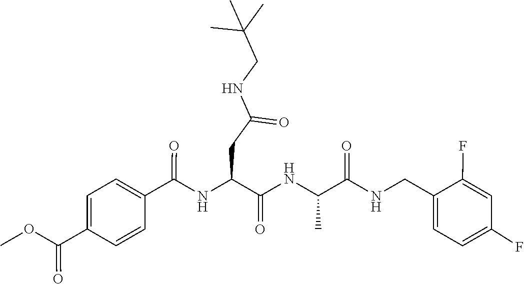 [SMILES: COC(=O)c1ccc(C(=O)N[C@@H](CC(=O)NCC(C)(C)C)C(=O)N[C@@H](C)C(=O)NCc2ccc(F)cc2F)cc1] | | |
| AMC-2-198 | 1 | 8 |
| 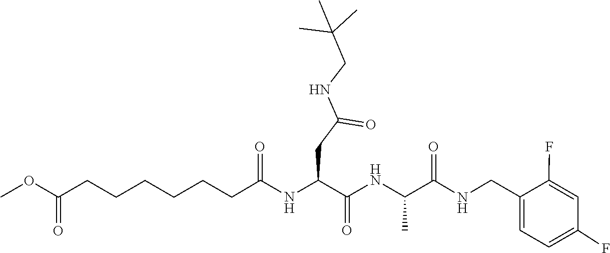 [SMILES: COC(=O)CCCCCCC(=O)N[C@@H](CC(=O)NCC(C)(C)C)C(=O)N[C@@H](C)C(=O)NCc1ccc(F)cc1F] | | |
| AMC-2-204 | 3 | 8 |
| 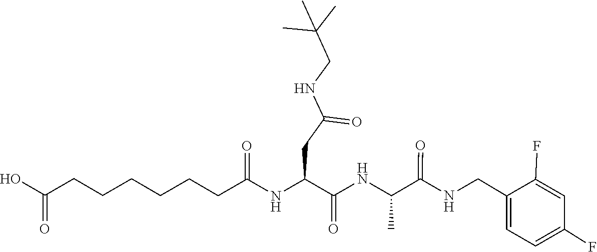 [SMILES: C[C@H](NC(=O)[C@H](CC(=O)NCC(C)(C)C)NC(=O)CCCCCCC(=O)O)C(=O)NCc1ccc(F)cc1F] | | |
| AMC-2-225 | −3 | 4 |
| 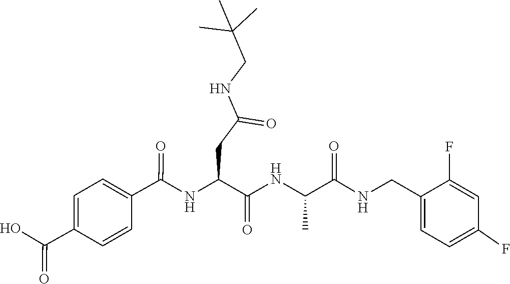 [SMILES: C[C@H](NC(=O)[C@H](CC(=O)NCC(C)(C)C)NC(=O)c1ccc(C(=O)O)cc1)C(=O)NCc1ccc(F)cc1F] | | |

The potency for HDAC6 inhibition (i.e., HDAC6 IC₅₀) and the selectivity for HDAC6 inhibition (i.e., HDAC1/HDAC6) were determined for several dual HDAC6/covalent proteasome inhibitors of the disclosure, as set forth below in Table 2. Selective HDAC6 inhibition was based on the value of HDAC1 IC₅₀/HDAC6 IC₅₀ for each tested compound.

TABLE 2
Potency and Selectivity for HDAC6 of Covalent Dual Inhibitors

| Compound | HDAC6 IC₅₀ | HDAC1/HDAC6 |
|---|---|---|
| AMC-3-017 | 0.198 μM | 14.3 |
| 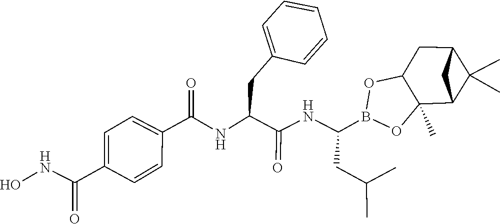 [SMILES: CC(C)C[C@H](NC(=O)[C@H](Cc1ccccc1)NC(=O)c1ccc(C(=O)NO)cc1)B1OC2C[C@H]3C[C@H](C3(C)C)[C@@]2(C)O1] | | |
| AMC-3-041 | 0.061 μM | 0.93 |
| 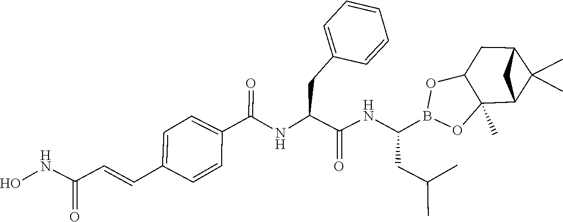 [SMILES: CC(C)C[C@H](NC(=O)[C@H](Cc1ccccc1)NC(=O)c1ccc(/C=C/C(=O)NO)cc1)B1OC2C[C@H]3C[C@H](C3(C)C)[C@@]2(C)O1] | | |
| AMC-2-248 | >30 μM | — |
| 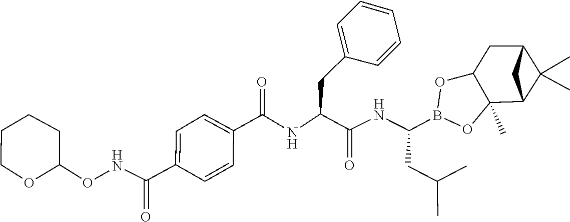 [SMILES: CC(C)C[C@H](NC(=O)[C@H](Cc1ccccc1)NC(=O)c1ccc(C(=O)NOC2CCCCO2)cc1)B1OC2C[C@H]3C[C@H](C3(C)C)[C@@]2(C)O1] | | |
| AMC-3-033 | 6.32 uM | 4.75 |
| 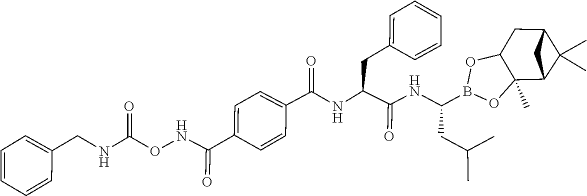 [SMILES: CC(C)C[C@H](NC(=O)[C@H](Cc1ccccc1)NC(=O)c1ccc(C(=O)NOC(=O)NCc2ccccc2)cc1)B1OC2C[C@H]3C[C@H](C3(C)C)[C@@]2(C)O1] | | |
| AMC-3-019 | 8.12 μM | >3.69 |
| 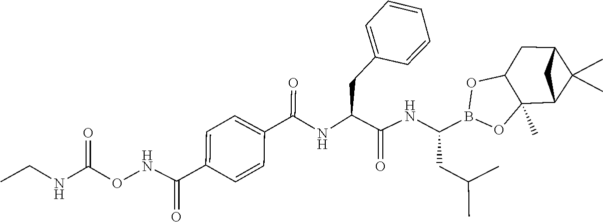 [SMILES: CCNC(=O)ONC(=O)c1ccc(C(=O)N[C@@H](Cc2ccccc2)C(=O)N[C@@H](CC(C)C)B2OC3C[C@H]4C[C@H](C4(C)C)[C@@]3(C)O2)cc1] | | |
| AMC-3-031 | 0.258 uM | 20.4 |
| 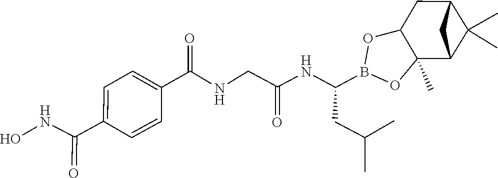 [SMILES: CC(C)C[C@H](NC(=O)CNC(=O)c1ccc(C(=O)NO)cc1)B1OC2C[C@H]3C[C@H](C3(C)C)[C@@]2(C)O1] | | |
| AMC-3-047 | 3.42 μM | 8.77 |
| 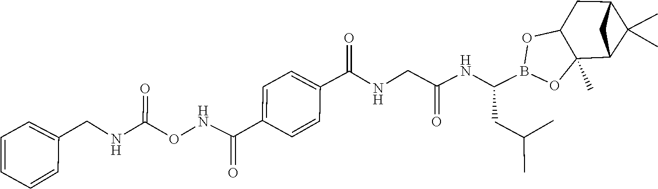 [SMILES: CC(C)C[C@H](NC(=O)CNC(=O)c1ccc(C(=O)NOC(=O)NCc2ccccc2)cc1)B1OC2C[C@H]3C[C@H](C3(C)C)[C@@]2(C)O1] | | |
| AMC-3-030 | 0.884 μM | >33.9 |
| 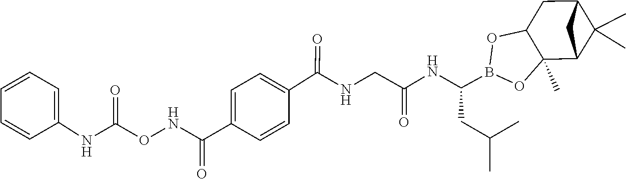 | | |
| AMC-3-048 | 5.38 μM | >5.58 |
| 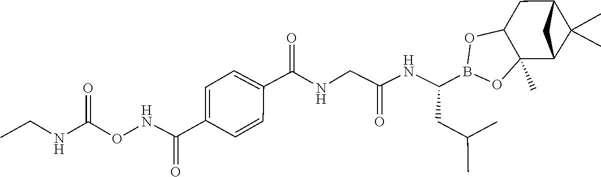 [SMILES: CCNC(=O)ONC(=O)c1ccc(C(=O)NCC(=O)N[C@@H](CC(C)C)B2OC3C[C@H]4C[C@H](C4(C)C)[C@@]3(C)O2)cc1] | | |
| AMC-3-055 | 1.15 μM | 2.30 |
| 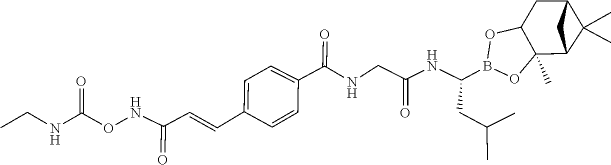 [SMILES: CCNC(=O)ONC(=O)/C=C/c1ccc(C(=O)NCC(=O)N[C@@H](CC(C)C)B2OC3C[C@H]4C[C@H](C4(C)C)[C@@]3(C)O2)cc1] | | |

The instant disclosure also provides certain embodiments as follows:

Embodiment 1: A compound of formula (I), or comprising a substructure of formula (I), or a pharmaceutically acceptable salt, solvate, hydrate, cocrystal, or prodrug thereof:

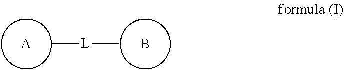

wherein in formula (I):
A comprises a zinc binding moiety;
L is a linking group; and
B comprises a proteasome inhibitor moiety, wherein the zinc binding moiety thereof, and the proteasome inhibitor moiety thereof are each connected to L at any chemically feasible site.

Embodiment 2: The compound according to Embodiment 1, wherein B comprises a proteosome inhibitor moiety selected from bortezomib, ixazomib, carfilzomib, oprozomib, marizomib, CEP-18770, disulfiram, epigallocatechin-3-gallate, epoxomicin, lactacystin, MG132, MLN9708, ONX 0912, PR-924, PR-957, KZR-504, LMP7-IN-1, salinosporamide A, epoxomycine, eponemycine, aclacinomycine A, and any substructure thereof.

Embodiment 3: The compound according to Embodiment 1 or Embodiment 2, wherein B comprises a moiety selected from:

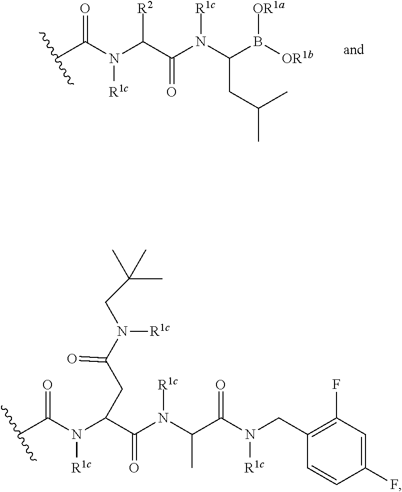

wherein R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups;
each R^{1c} is independently at each occurrence selected from hydrogen and —C₁₋₁₀ alkyl-; and
R² is selected from hydrogen, alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Embodiment 4: The compound according to any one of Embodiments 1 to 3, wherein the compound of formula (I) is a compound of formula (10) or formula (12), or a pharmaceutically acceptable salt, solvate, hydrate, cocrystal, or prodrug thereof:

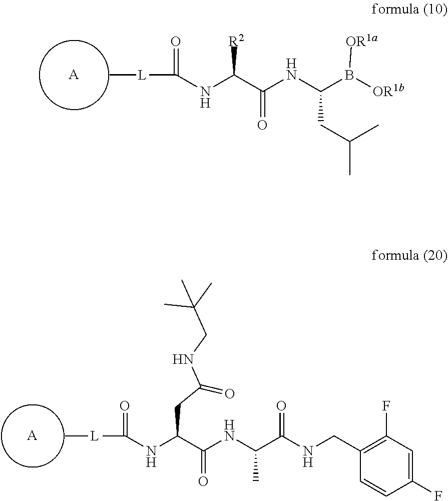

wherein in formula (10) and formula (20):
A comprises a zinc binding moiety;
L is a linking group;
R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups; and
R² is selected from hydrogen and optionally substituted —C₁₋₁₀ alkyl,
wherein the zinc binding moiety thereof is connected to L at any chemically feasible site.

Embodiment 5: The compound according to Embodiment 3 or Embodiment 4, wherein R² is selected from hydrogen and

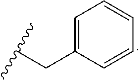 [SMILES: *Cc1ccccc1]

Embodiment 6: The compound according to Embodiment 4 or Embodiment 5, wherein the compound of formula (10) is a compound of formula (11) or formula (12), or a pharmaceutically acceptable salt, solvate, hydrate, cocrystal, or prodrug thereof:

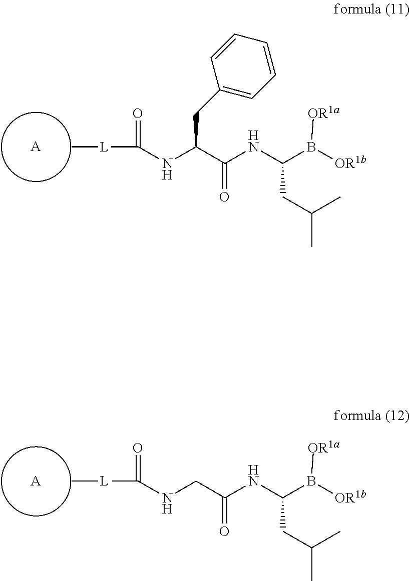

wherein in formula (11) and formula (12):
A comprises a zinc binding moiety;
L is a linking group; and
R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups.

Embodiment 7: The compound according to any one of Embodiments 1 to 3, wherein B comprises:

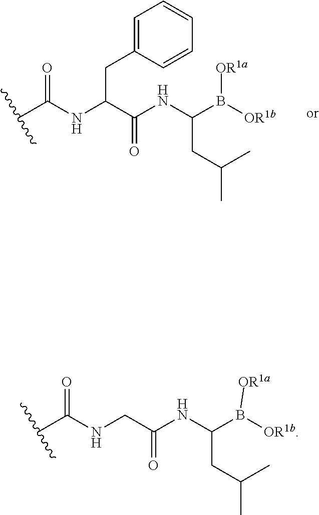

Embodiment 8: The compound according to any one of Embodiments 3 to 7, wherein R^{1a} and R^{1b} are each hydrogen or R^{1a} and R^{1b} are joined together to form a dioxaborolane, a dioxaborinane, or

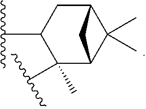 [SMILES: *C1C[C@H]2C[C@H](C2(C)C)[C@]1(*)C]

Embodiment 9: The compound according to Embodiment 6 or Embodiment 8, wherein the compound of formula (11) is a compound of formula (110) or formula (111), or a pharmaceutically acceptable salt, solvate, hydrate, cocrystal, or prodrug thereof:

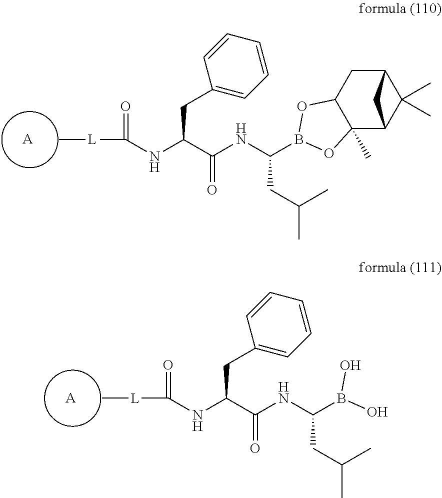

wherein in formula (110) and formula (111):
A comprises a zinc binding moiety; and
L is a linking group.

Embodiment 10: The compound according to Embodiment 6 or Embodiment 8, wherein the compound of formula (12) is a compound of formula (120) or formula (121), or a pharmaceutically acceptable salt, solvate, hydrate, cocrystal, or prodrug thereof:

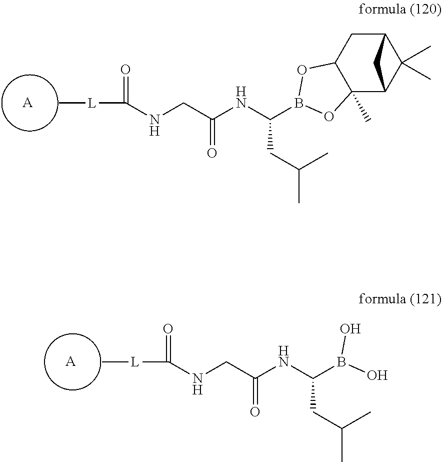

wherein in formula (120) and formula (121):
A comprises a zinc binding moiety; and
L is a linking group.

Embodiment 11: The compound according to any one of Embodiments 1 to 10, wherein L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R)—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and each R^{b} is independently hydrogen, optionally substituted alkyl, or optionally substituted aryl.

Embodiment 12: The compound according to any one of Embodiments 1 to 11, wherein L comprises one or more linking groups selected from optionally substituted —C₁₋₁₀ alkyl-, —C₁₋₁₀ alkenyl-, -aryl-, —C(O)—,

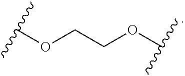 [SMILES: *OCCO*]

Embodiment 13: The compound according to any one of Embodiments 1 to 12, wherein L comprises one or more linking group selected from

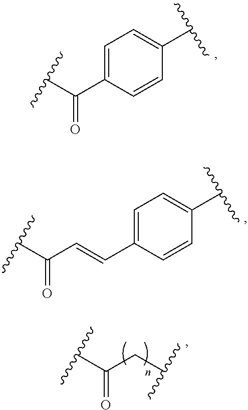

wherein n is an integer from 0 to 7,

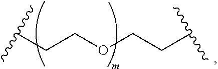

wherein m is an integer from 0 to 5,

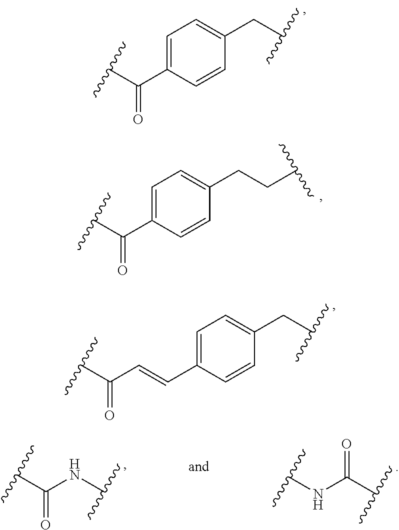 [SMILES: *Cc1ccc(C(*)=O)cc1 ; *CCc1ccc(C(*)=O)cc1 ; *Cc1ccc(/C=C/C(*)=O)cc1 ; *NC(*)=O ; *NC(*)=O]

Embodiment 14: The compound according to any one of Embodiments 1 to 13, wherein A comprises a moiety selected from:

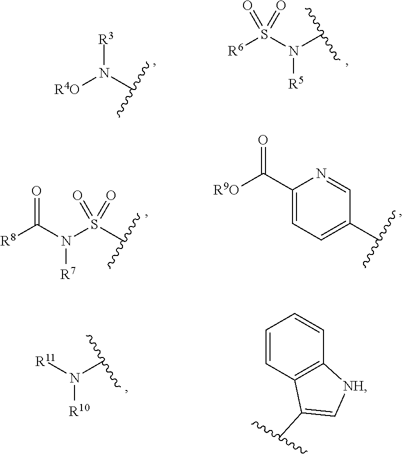

wherein:
R³ is selected from hydrogen and optionally substituted —C₁₋₁₀ alkyl-;
R⁴ is selected from hydrogen, N(R^{b})₂C(O)—, and —R^{b}OC(O)—;
R⁵ is selected from hydrogen and optionally substituted —C₁₋₁₀ alkyl-;
R⁶ is selected from optionally substituted —C₁₋₁₀ alkyl- and optionally substituted aryl;
R⁷ is selected from hydrogen and optionally substituted —C₁₋₁₀ alkyl-;
R⁸ is optionally substituted —C₁₋₁₀ alkyl-;
R⁹ is selected from hydrogen and optionally substituted —C₁₋₁₀ alkyl-;
R¹⁰ is selected from hydrogen and optionally substituted —C₁₋₁₀ alkyl-;
R¹¹ is optionally substituted aryl;
R¹² is optionally substituted —C₁₋₁₀ alkyl; and
each R^{b} is independently hydrogen, optionally substituted alkyl, optionally substituted heterocyclyl, or optionally substituted aryl.

Embodiment 15: The compound according to Embodiment 14, wherein R⁷ is

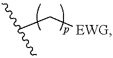

wherein EWG comprises an electron withdrawing group selected from —CN, —NO₂, —C(O)R¹³, —C(O)N(R¹⁴), and —N(R¹⁴)C(O)C(O)R¹⁴, wherein p is an integer between 1 and 5, R¹³ is selected from —C₁₋₁₀ alkyl, —C₁₋₁₀ alkoxy, and each R¹⁴ is independently selected from hydrogen and —C₁₋₁₀ alkyl.

Embodiment 16: The compound according to Embodiment 14 or Embodiment 15, wherein:
R³ is hydrogen;
R⁴ is selected from hydrogen and N(R^{b})₂C(O)—;
R⁵ is selected from hydrogen, methyl, and

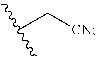 [SMILES: *CC#N]

R⁶ is selected from methyl, —CF₃, —CCl₃, and phenyl;
R⁷ is selected from hydrogen and

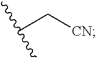 [SMILES: *CC#N]

R⁸ is methyl or —CF₃;
R⁹ is hydrogen;
R¹⁰ is hydrogen;
R¹¹ is amino substituted phenyl;
R¹² is methyl; and
each R^{b} is independently selected from hydrogen, ethyl, isopropyl, phenyl,

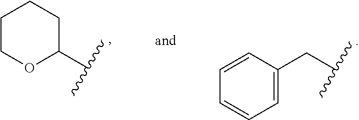 [SMILES: *C1CCCCO1 ; *Cc1ccccc1]

Embodiment 17: The compound according to any one of Embodiments 1 to 16, wherein A is selected from:

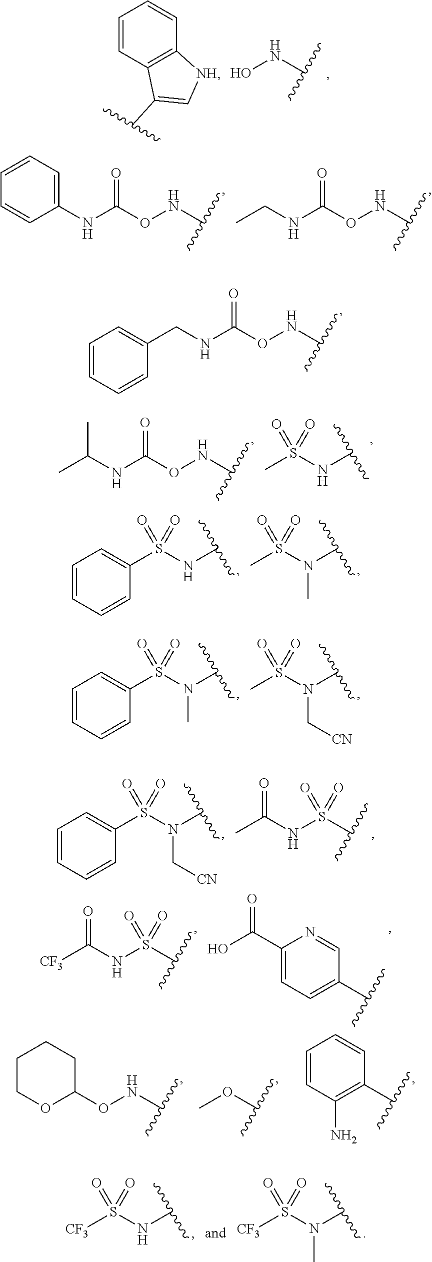

Embodiment 18: The compound according to any one of Embodiments 1 to 17, wherein the compound of formula (I) is a compound of any one of formula 1001-1114, or a pharmaceutically acceptable salt, solvate, hydrate, cocrystal, or prodrug thereof, wherein in formula 1001-1114:
B is:

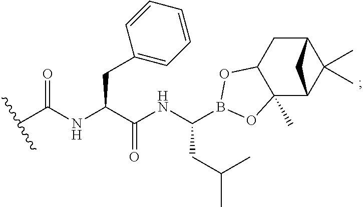 [SMILES: *C(=O)N[C@@H](Cc1ccccc1)C(=O)N[C@@H](CC(C)C)B1OC2C[C@H]3C[C@H](C3(C)C)[C@@]2(C)O1]

and:

| Formula No. | A | L | n |
|---|---|---|---|
| 1001 | 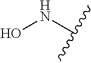 [SMILES: *NO] | 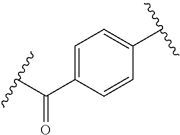 [SMILES: *C(=O)c1ccc(*)cc1] | — |
| 1002 | 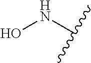 [SMILES: *NO] | 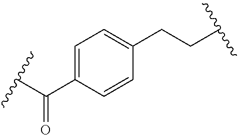 [SMILES: *CCc1ccc(C(*)=O)cc1] | |
| 1003 | 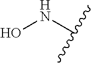 [SMILES: *NO] | 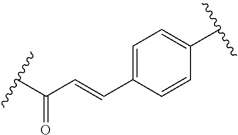 [SMILES: *C(=O)/C=C/c1ccc(*)cc1] | — |
| 1004 | 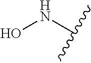 [SMILES: *NO] | 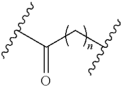 | 5 |
| 1005 | 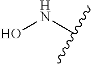 [SMILES: *NO] | 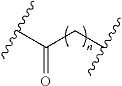 | 6 |
| 1006 | 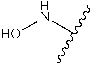 [SMILES: *NO] | 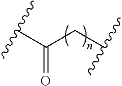 | 7 |
| 1007 | 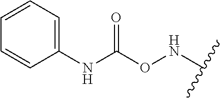 [SMILES: *NOC(=O)Nc1ccccc1] | 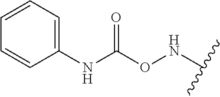 [SMILES: *NOC(=O)Nc1ccccc1] | — |
| 1008 | 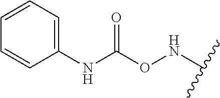 [SMILES: *NOC(=O)Nc1ccccc1] | 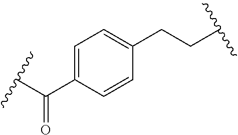 [SMILES: *CCc1ccc(C(*)=O)cc1] | |
| 1009 | 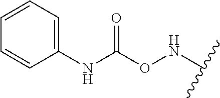 [SMILES: *NOC(=O)Nc1ccccc1] | 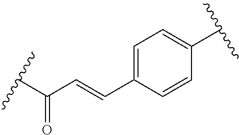 [SMILES: *C(=O)/C=C/c1ccc(*)cc1] | — |
| 1010 | 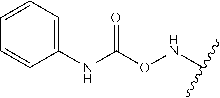 [SMILES: *NOC(=O)Nc1ccccc1] | 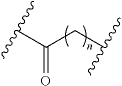 | 5 |
| 1011 | 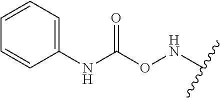 [SMILES: *NOC(=O)Nc1ccccc1] | 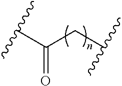 | 6 |
| 1012 | 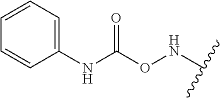 [SMILES: *NOC(=O)Nc1ccccc1] | 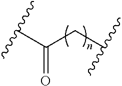 | 7 |
| 1013 | 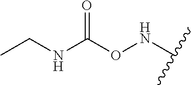 [SMILES: *NOC(=O)NCC] | 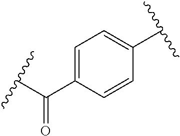 [SMILES: *C(=O)c1ccc(*)cc1] | — |
| 1014 | 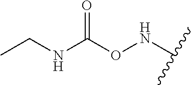 [SMILES: *NOC(=O)NCC] | 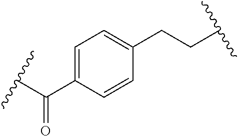 [SMILES: *CCc1ccc(C(*)=O)cc1] | |
| 1015 | 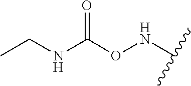 [SMILES: *NOC(=O)NCC] | 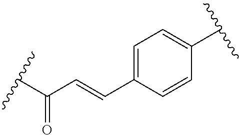 [SMILES: *C(=O)/C=C/c1ccc(*)cc1] | — |
| 1016 | 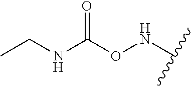 [SMILES: *NOC(=O)NCC] | 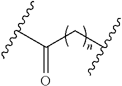 | 5 |
| 1017 | 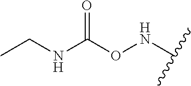 [SMILES: *NOC(=O)NCC] | 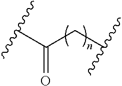 | 6 |
| 1018 | 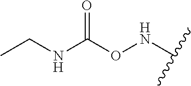 [SMILES: *NOC(=O)NCC] | 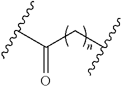 | 7 |
| 1019 | 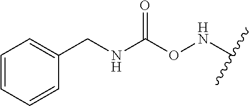 [SMILES: *NOC(=O)NCc1ccccc1] | 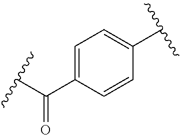 [SMILES: *C(=O)c1ccc(*)cc1] | — |
| 1020 | 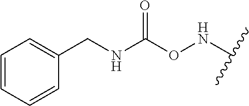 [SMILES: *NOC(=O)NCc1ccccc1] | 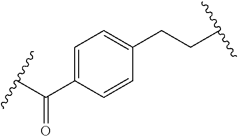 [SMILES: *CCc1ccc(C(*)=O)cc1] | |
| 1021 | 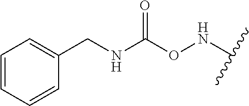 [SMILES: *NOC(=O)NCc1ccccc1] | 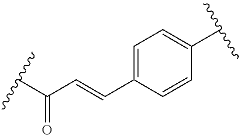 [SMILES: *C(=O)/C=C/c1ccc(*)cc1] | — |
| 1022 | 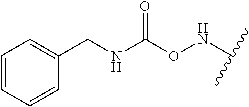 [SMILES: *NOC(=O)NCc1ccccc1] | 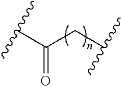 | 5 |
| 1023 | 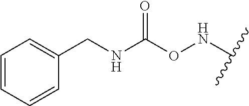 [SMILES: *NOC(=O)NCc1ccccc1] | 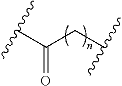 | 6 |
| 1024 | 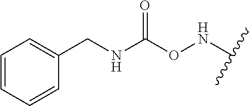 [SMILES: *NOC(=O)NCc1ccccc1] | 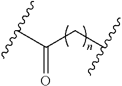 | 7 |
| 1025 | 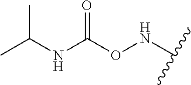 [SMILES: *NOC(=O)NC(C)C] | 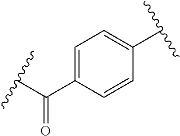 [SMILES: *C(=O)c1ccc(*)cc1] | — |
| 1026 | 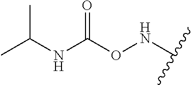 [SMILES: *NOC(=O)NC(C)C] | 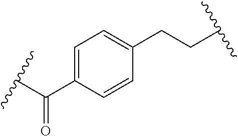 [SMILES: *CCc1ccc(C(*)=O)cc1] | |
| 1027 | 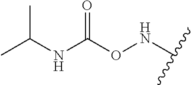 [SMILES: *NOC(=O)NC(C)C] | 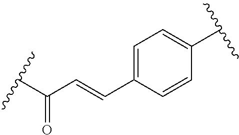 [SMILES: *C(=O)/C=C/c1ccc(*)cc1] | — |
| 1028 | 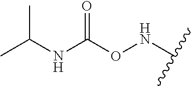 [SMILES: *NOC(=O)NC(C)C] | 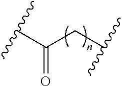 | 5 |
| 1029 | 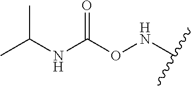 [SMILES: *NOC(=O)NC(C)C] | 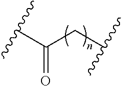 | 6 |
| 1030 | 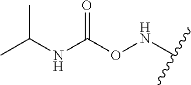 [SMILES: *NOC(=O)NC(C)C] | 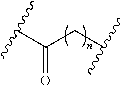 | 7 |
| 1031 | 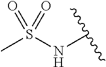 [SMILES: *NS(C)(=O)=O] | 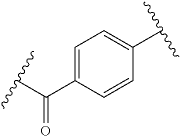 [SMILES: *C(=O)c1ccc(*)cc1] | — |
| 1032 | 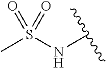 [SMILES: *NS(C)(=O)=O] | 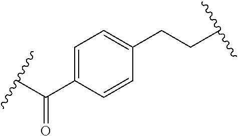 [SMILES: *CCc1ccc(C(*)=O)cc1] | |
| 1033 | 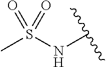 [SMILES: *NS(C)(=O)=O] | 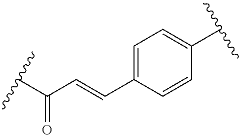 [SMILES: *C(=O)/C=C/c1ccc(*)cc1] | — |
| 1034 | 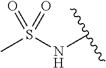 [SMILES: *NS(C)(=O)=O] | 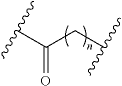 | 5 |
| 1035 | 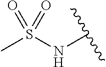 [SMILES: *NS(C)(=O)=O] | 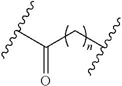 | 6 |
| 1036 | 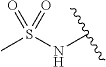 [SMILES: *NS(C)(=O)=O] | 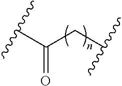 | 7 |
| 1037 | 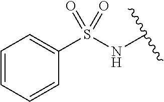 [SMILES: *NS(=O)(=O)c1ccccc1] | 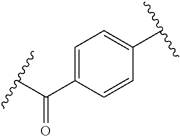 [SMILES: *C(=O)c1ccc(*)cc1] | — |
| 1038 | 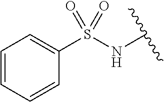 [SMILES: *NS(=O)(=O)c1ccccc1] | 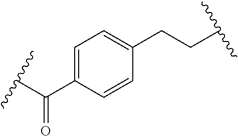 [SMILES: *CCc1ccc(C(*)=O)cc1] | |
| 1039 | 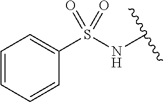 [SMILES: *NS(=O)(=O)c1ccccc1] | 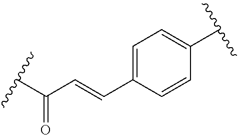 [SMILES: *C(=O)/C=C/c1ccc(*)cc1] | — |
| 1040 | 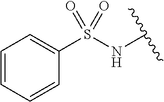 [SMILES: *NS(=O)(=O)c1ccccc1] | 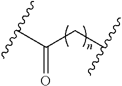 | 5 |
| 1041 | 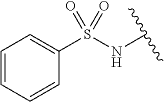 [SMILES: *NS(=O)(=O)c1ccccc1] | 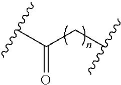 | 6 |
| 1042 | 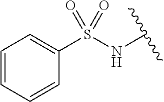 [SMILES: *NS(=O)(=O)c1ccccc1] | 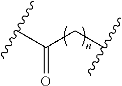 | 7 |
| 1043 | 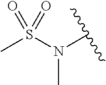 [SMILES: *N(C)S(C)(=O)=O] | 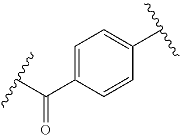 [SMILES: *C(=O)c1ccc(*)cc1] | — |
| 1044 | 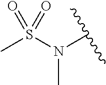 [SMILES: *N(C)S(C)(=O)=O] | 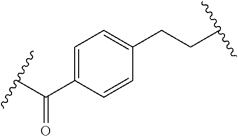 [SMILES: *CCc1ccc(C(*)=O)cc1] | |
| 1045 | 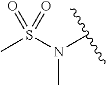 [SMILES: *N(C)S(C)(=O)=O] | 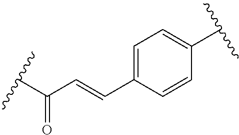 [SMILES: *C(=O)/C=C/c1ccc(*)cc1] | — |
| 1046 | 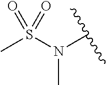 [SMILES: *N(C)S(C)(=O)=O] | 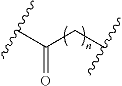 | 5 |
| 1047 | 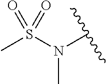 [SMILES: *N(C)S(C)(=O)=O] | 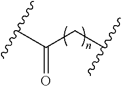 | 6 |
| 1048 | 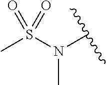 [SMILES: *N(C)S(C)(=O)=O] | 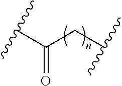 | 7 |
| 1049 | 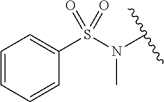 [SMILES: *N(C)S(=O)(=O)c1ccccc1] | 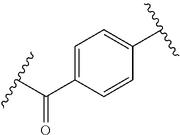 [SMILES: *C(=O)c1ccc(*)cc1] | — |
| 1050 | 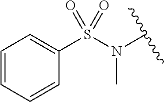 [SMILES: *N(C)S(=O)(=O)c1ccccc1] | 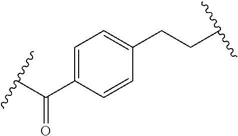 [SMILES: *CCc1ccc(C(*)=O)cc1] | |
| 1051 | 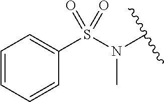 [SMILES: *N(C)S(=O)(=O)c1ccccc1] | 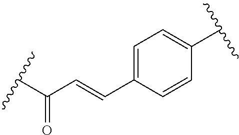 [SMILES: *C(=O)/C=C/c1ccc(*)cc1] | — |
| 1052 | 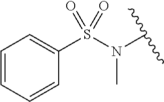 [SMILES: *N(C)S(=O)(=O)c1ccccc1] | 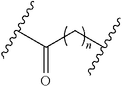 | 5 |
| 1053 | 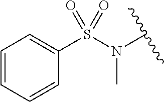 [SMILES: *N(C)S(=O)(=O)c1ccccc1] | 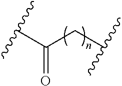 | 6 |
| 1054 | 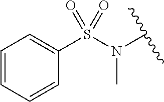 [SMILES: *N(C)S(=O)(=O)c1ccccc1] | 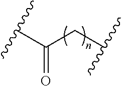 | 7 |
| 1055 | 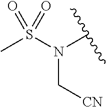 [SMILES: *N(CC#N)S(C)(=O)=O] | 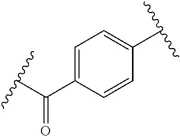 [SMILES: *C(=O)c1ccc(*)cc1] | — |
| 1056 | 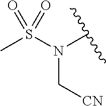 [SMILES: *N(CC#N)S(C)(=O)=O] | 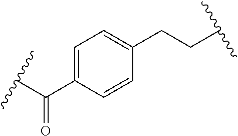 [SMILES: *CCc1ccc(C(*)=O)cc1] | |
| 1057 | 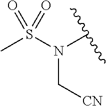 [SMILES: *N(CC#N)S(C)(=O)=O] | 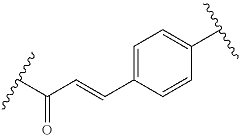 [SMILES: *C(=O)/C=C/c1ccc(*)cc1] | — |
| 1058 | 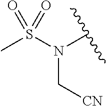 [SMILES: *N(CC#N)S(C)(=O)=O] | 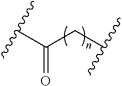 | 5 |
| 1059 | 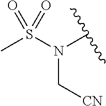 [SMILES: *N(CC#N)S(C)(=O)=O] | 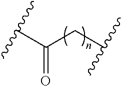 | 6 |
| 1060 | 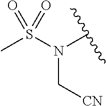 [SMILES: *N(CC#N)S(C)(=O)=O] | 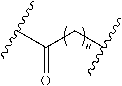 | 7 |
| 1061 | 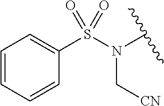 [SMILES: *N(CC#N)S(=O)(=O)c1ccccc1] | 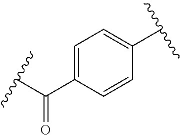 [SMILES: *C(=O)c1ccc(*)cc1] | — |
| 1062 | 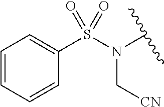 [SMILES: *N(CC#N)S(=O)(=O)c1ccccc1] | 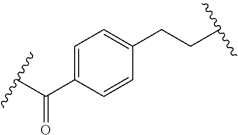 [SMILES: *CCc1ccc(C(*)=O)cc1] | |
| 1063 | 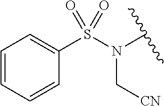 [SMILES: *N(CC#N)S(=O)(=O)c1ccccc1] | 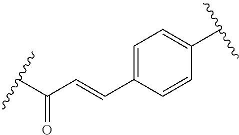 [SMILES: *C(=O)/C=C/c1ccc(*)cc1] | — |
| 1064 | 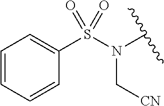 [SMILES: *N(CC#N)S(=O)(=O)c1ccccc1] | 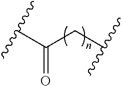 | 5 |
| 1065 | 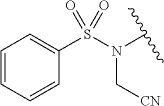 [SMILES: *N(CC#N)S(=O)(=O)c1ccccc1] | 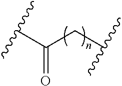 | 6 |
| 1066 | 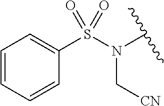 [SMILES: *N(CC#N)S(=O)(=O)c1ccccc1] | 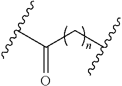 | 7 |
| 1067 | 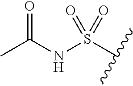 [SMILES: *S(=O)(=O)NC(C)=O] | 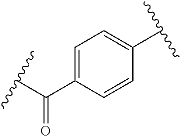 [SMILES: *C(=O)c1ccc(*)cc1] | — |
| 1068 | 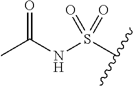 [SMILES: *S(=O)(=O)NC(C)=O] | 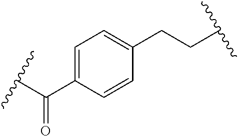 [SMILES: *CCc1ccc(C(*)=O)cc1] | |
| 1069 | 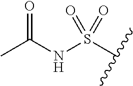 [SMILES: *S(=O)(=O)NC(C)=O] | 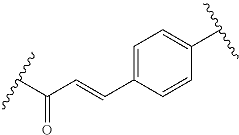 [SMILES: *C(=O)/C=C/c1ccc(*)cc1] | — |
| 1070 | 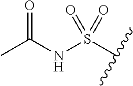 [SMILES: *S(=O)(=O)NC(C)=O] | 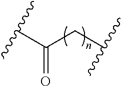 | 5 |
| 1071 | 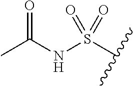 [SMILES: *S(=O)(=O)NC(C)=O] | 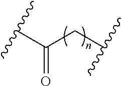 | 6 |
| 1072 | 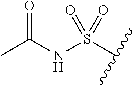 [SMILES: *S(=O)(=O)NC(C)=O] | 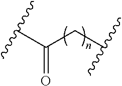 | 7 |
| 1073 | 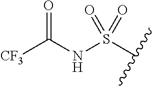 [SMILES: *S(=O)(=O)NC(=O)C(F)(F)F] | 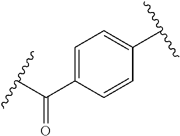 [SMILES: *C(=O)c1ccc(*)cc1] | — |
| 1074 | 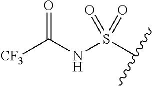 [SMILES: *S(=O)(=O)NC(=O)C(F)(F)F] | 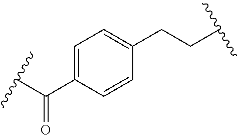 [SMILES: *CCc1ccc(C(*)=O)cc1] | |
| 1075 | 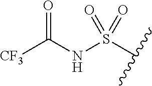 [SMILES: *S(=O)(=O)NC(=O)C(F)(F)F] | 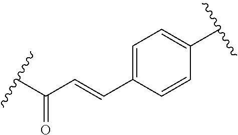 [SMILES: *C(=O)/C=C/c1ccc(*)cc1] | — |
| 1076 | 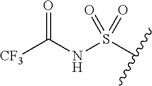 [SMILES: *S(=O)(=O)NC(=O)C(F)(F)F] | 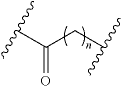 | 5 |
| 1077 | 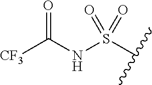 [SMILES: *S(=O)(=O)NC(=O)C(F)(F)F] | 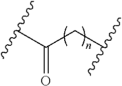 | 6 |
| 1078 | 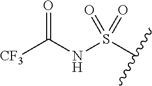 [SMILES: *S(=O)(=O)NC(=O)C(F)(F)F] | 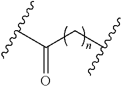 | 7 |
| 1079 | 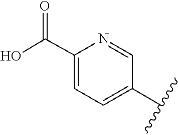 [SMILES: *c1ccc(C(=O)O)nc1] | 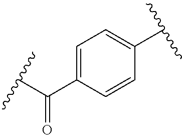 [SMILES: *C(=O)c1ccc(*)cc1] | — |
| 1080 | 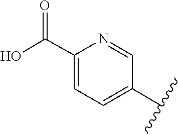 [SMILES: *c1ccc(C(=O)O)nc1] | 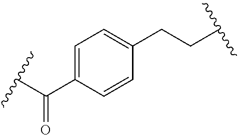 [SMILES: *CCc1ccc(C(*)=O)cc1] | |
| 1081 | 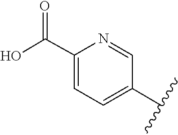 [SMILES: *c1ccc(C(=O)O)nc1] | 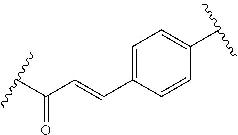 [SMILES: *C(=O)/C=C/c1ccc(*)cc1] | — |
| 1082 | 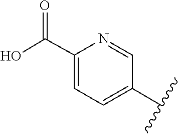 [SMILES: *c1ccc(C(=O)O)nc1] | 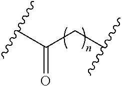 | 5 |
| 1083 | 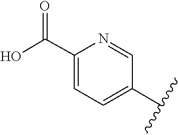 [SMILES: *c1ccc(C(=O)O)nc1] | 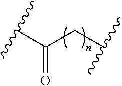 | 6 |
| 1084 | 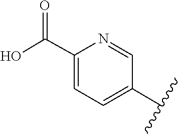 [SMILES: *c1ccc(C(=O)O)nc1] | 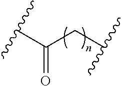 | 7 |
| 1085 | 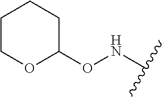 [SMILES: *NOC1CCCCO1] | 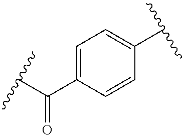 [SMILES: *C(=O)c1ccc(*)cc1] | — |
| 1086 | 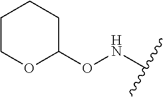 [SMILES: *NOC1CCCCO1] | 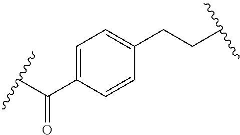 [SMILES: *CCc1ccc(C(*)=O)cc1] | |
| 1087 | 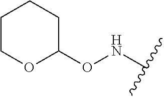 [SMILES: *NOC1CCCCO1] | 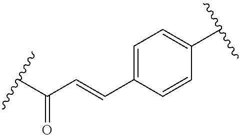 [SMILES: *C(=O)/C=C/c1ccc(*)cc1] | — |
| 1088 | 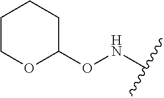 [SMILES: *NOC1CCCCO1] | 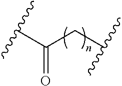 | 5 |
| 1089 | 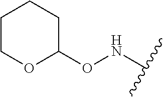 [SMILES: *NOC1CCCCO1] | 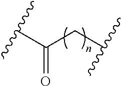 | 6 |
| 1090 | 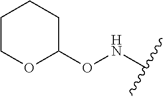 [SMILES: *NOC1CCCCO1] | 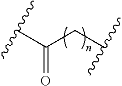 | 7 |
| 1091 | 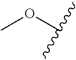 [SMILES: *OC] | 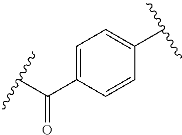 [SMILES: *C(=O)c1ccc(*)cc1] | — |
| 1092 | 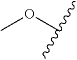 [SMILES: *OC] | 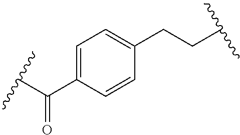 [SMILES: *CCc1ccc(C(*)=O)cc1] | |
| 1093 | 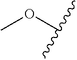 [SMILES: *OC] | 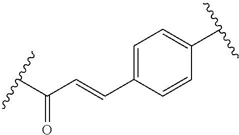 [SMILES: *C(=O)/C=C/c1ccc(*)cc1] | — |
| 1094 | 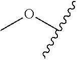 [SMILES: *OC] | 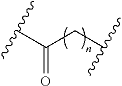 | 5 |
| 1095 | 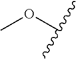 [SMILES: *OC] | 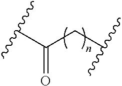 | 6 |
| 1096 | 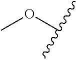 [SMILES: *OC] | 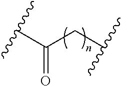 | 7 |
| 1097 | 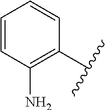 [SMILES: *c1ccccc1N] | 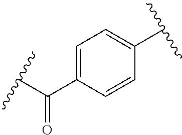 [SMILES: *C(=O)c1ccc(*)cc1] | — |
| 1098 | 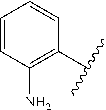 [SMILES: *c1ccccc1N] | 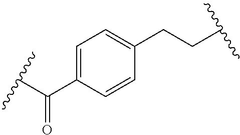 [SMILES: *CCc1ccc(C(*)=O)cc1] | |
| 1099 | 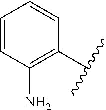 [SMILES: *c1ccccc1N] | 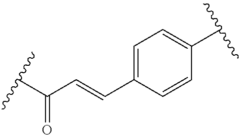 [SMILES: *C(=O)/C=C/c1ccc(*)cc1] | — |
| 1100 | 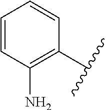 [SMILES: *c1ccccc1N] | 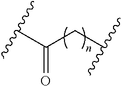 | 5 |
| 1101 | 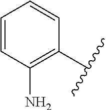 [SMILES: *c1ccccc1N] | 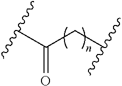 | 6 |
| 1102 | 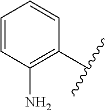 [SMILES: *c1ccccc1N] | 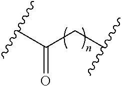 | 7 |
| 1103 | 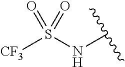 [SMILES: *NS(=O)(=O)C(F)(F)F] | 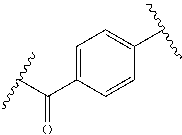 [SMILES: *C(=O)c1ccc(*)cc1] | — |
| 1104 | 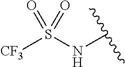 [SMILES: *NS(=O)(=O)C(F)(F)F] | 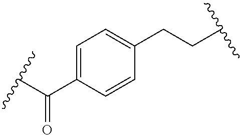 [SMILES: *CCc1ccc(C(*)=O)cc1] | |
| 1105 | 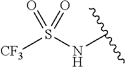 [SMILES: *NS(=O)(=O)C(F)(F)F] | 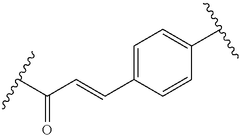 [SMILES: *C(=O)/C=C/c1ccc(*)cc1] | — |
| 1106 | 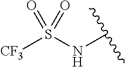 [SMILES: *NS(=O)(=O)C(F)(F)F] | 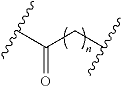 | 5 |
| 1107 | 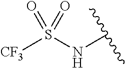 [SMILES: *NS(=O)(=O)C(F)(F)F] | 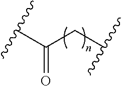 | 6 |
| 1108 | 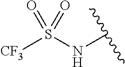 [SMILES: *NS(=O)(=O)C(F)(F)F] | 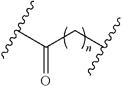 | 7 |
| 1109 | 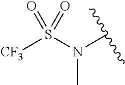 [SMILES: *N(C)S(=O)(=O)C(F)(F)F] | 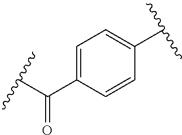 [SMILES: *C(=O)c1ccc(*)cc1] | — |
| 1110 | 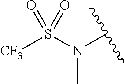 [SMILES: *N(C)S(=O)(=O)C(F)(F)F] | 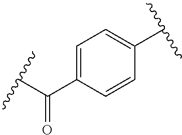 [SMILES: *C(=O)c1ccc(*)cc1] | |
| 1111 | 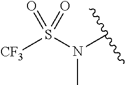 [SMILES: *N(C)S(=O)(=O)C(F)(F)F] | 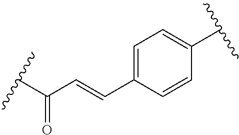 [SMILES: *C(=O)/C=C/c1ccc(*)cc1] | — |
| 1112 | 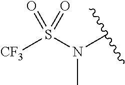 [SMILES: *N(C)S(=O)(=O)C(F)(F)F] | 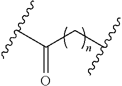 | 5 |
| 1113 | 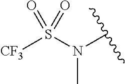 [SMILES: *N(C)S(=O)(=O)C(F)(F)F] | 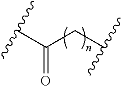 | 6 |
| 1114 | 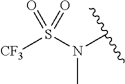 [SMILES: *N(C)S(=O)(=O)C(F)(F)F] | 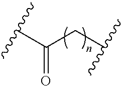 | 7 |

Embodiment 19: The compound according to any one of Embodiments 1 to 17, wherein the compound of formula (I) is a compound of any one of formula 1115-1228, or a pharmaceutically acceptable salt, solvate, hydrate, cocrystal, or prodrug thereof, wherein in formula 1115-1228:
B is:

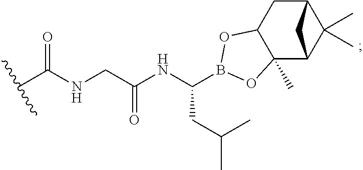 [SMILES: *C(=O)NCC(=O)N[C@@H](CC(C)C)B1OC2C[C@H]3C[C@H](C3(C)C)[C@@]2(C)O1]

and:

| Formula No. | A | L | n |
|---|---|---|---|
| 1115 | 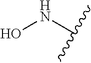 [SMILES: *NO] | 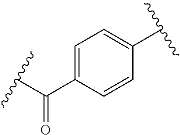 [SMILES: *C(=O)c1ccc(*)cc1] | — |
| 1116 | 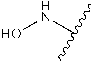 [SMILES: *NO] | 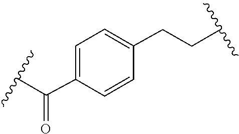 [SMILES: *CCc1ccc(C(*)=O)cc1] | |
| 1117 | 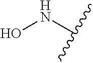 [SMILES: *NO] | 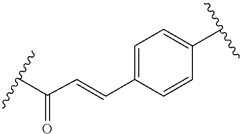 [SMILES: *C(=O)/C=C/c1ccc(*)cc1] | — |
| 1118 | 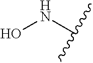 [SMILES: *NO] | 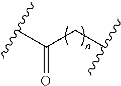 | 5 |
| 1119 | 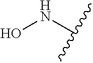 [SMILES: *NO] | 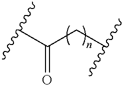 | 6 |
| 1120 | 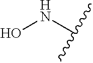 [SMILES: *NO] | 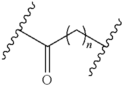 | 7 |
| 1121 | 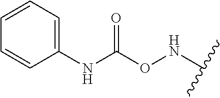 [SMILES: *NOC(=O)Nc1ccccc1] | 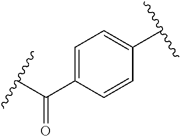 [SMILES: *C(=O)c1ccc(*)cc1] | — |
| 1122 | 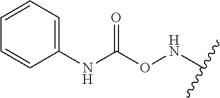 [SMILES: *NOC(=O)Nc1ccccc1] | 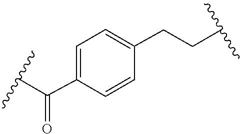 [SMILES: *CCc1ccc(C(*)=O)cc1] | |
| 1123 | 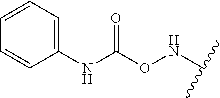 [SMILES: *NOC(=O)Nc1ccccc1] | 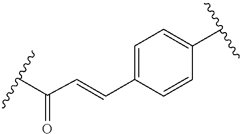 [SMILES: *C(=O)/C=C/c1ccc(*)cc1] | — |
| 1124 | 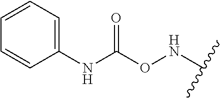 [SMILES: *NOC(=O)Nc1ccccc1] | 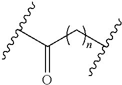 | 5 |
| 1125 | 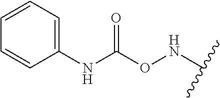 [SMILES: *NOC(=O)Nc1ccccc1] | 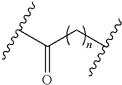 | 6 |
| 1126 | 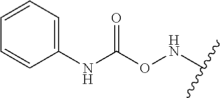 [SMILES: *NOC(=O)Nc1ccccc1] | 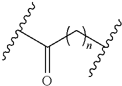 | 7 |
| 1127 | 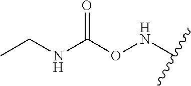 [SMILES: *NOC(=O)NCC] | 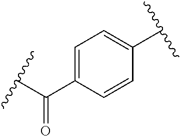 [SMILES: *C(=O)c1ccc(*)cc1] | — |
| 1128 | 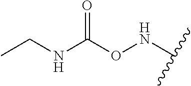 [SMILES: *NOC(=O)NCC] | 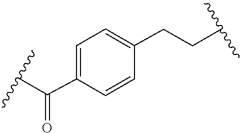 [SMILES: *CCc1ccc(C(*)=O)cc1] | |
| 1129 | 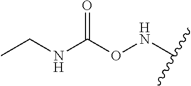 [SMILES: *NOC(=O)NCC] | 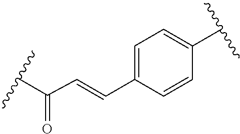 [SMILES: *C(=O)/C=C/c1ccc(*)cc1] | — |
| 1130 | 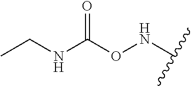 [SMILES: *NOC(=O)NCC] | 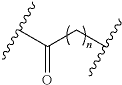 | 5 |
| 1131 | 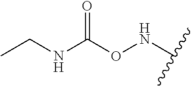 [SMILES: *NOC(=O)NCC] | 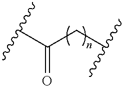 | 6 |
| 1132 | 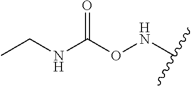 [SMILES: *NOC(=O)NCC] | 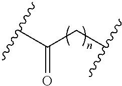 | 7 |
| 1133 | 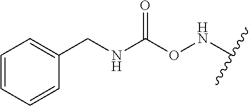 [SMILES: *NOC(=O)NCc1ccccc1] | 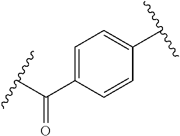 [SMILES: *C(=O)c1ccc(*)cc1] | — |
| 1134 | 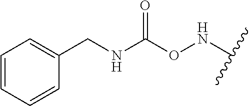 [SMILES: *NOC(=O)NCc1ccccc1] | 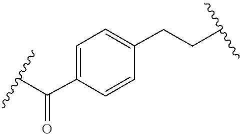 [SMILES: *CCc1ccc(C(*)=O)cc1] | |
| 1135 | 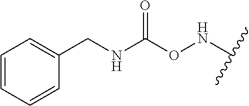 [SMILES: *NOC(=O)NCc1ccccc1] | 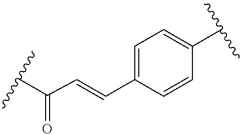 [SMILES: *C(=O)/C=C/c1ccc(*)cc1] | — |
| 1136 | 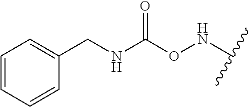 [SMILES: *NOC(=O)NCc1ccccc1] | 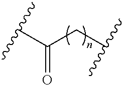 | 5 |
| 1137 | 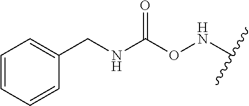 [SMILES: *NOC(=O)NCc1ccccc1] | 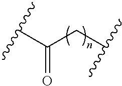 | 6 |
| 1138 | 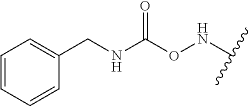 [SMILES: *NOC(=O)NCc1ccccc1] | 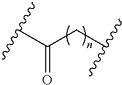 | 7 |
| 1139 | 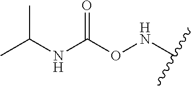 [SMILES: *NOC(=O)NC(C)C] | 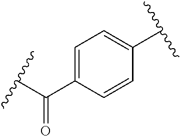 [SMILES: *C(=O)c1ccc(*)cc1] | — |
| 1140 | 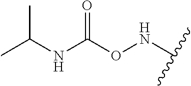 [SMILES: *NOC(=O)NC(C)C] | 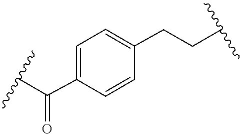 [SMILES: *CCc1ccc(C(*)=O)cc1] | |
| 1141 | 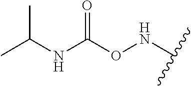 [SMILES: *NOC(=O)NC(C)C] | 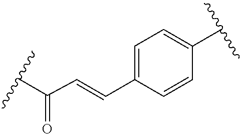 [SMILES: *C(=O)/C=C/c1ccc(*)cc1] | — |
| 1142 | 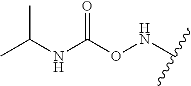 [SMILES: *NOC(=O)NC(C)C] | 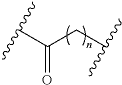 | 5 |
| 1143 | 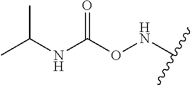 [SMILES: *NOC(=O)NC(C)C] | 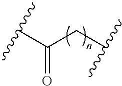 | 6 |
| 1144 | 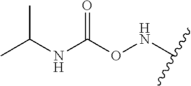 [SMILES: *NOC(=O)NC(C)C] | 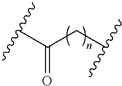 | 7 |
| 1145 | 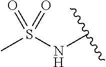 [SMILES: *NS(C)(=O)=O] | 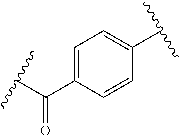 [SMILES: *C(=O)c1ccc(*)cc1] | — |
| 1146 | 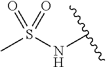 [SMILES: *NS(C)(=O)=O] | 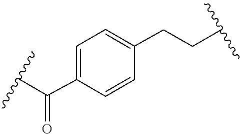 [SMILES: *CCc1ccc(C(*)=O)cc1] | |
| 1147 | 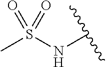 [SMILES: *NS(C)(=O)=O] | 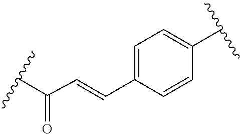 [SMILES: *C(=O)/C=C/c1ccc(*)cc1] | — |
| 1148 | 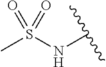 [SMILES: *NS(C)(=O)=O] | 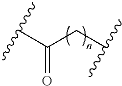 | 5 |
| 1149 | 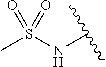 [SMILES: *NS(C)(=O)=O] | 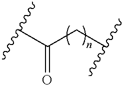 | 6 |
| 1150 | 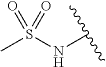 [SMILES: *NS(C)(=O)=O] | 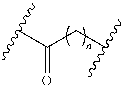 | 7 |
| 1151 | 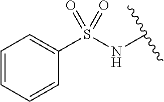 [SMILES: *NS(=O)(=O)c1ccccc1] | 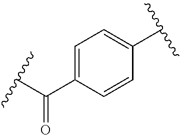 [SMILES: *C(=O)c1ccc(*)cc1] | — |
| 1152 | 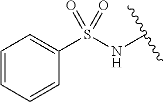 [SMILES: *NS(=O)(=O)c1ccccc1] | 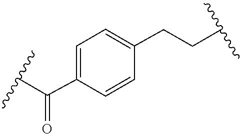 [SMILES: *CCc1ccc(C(*)=O)cc1] | |
| 1153 | 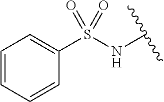 [SMILES: *NS(=O)(=O)c1ccccc1] | 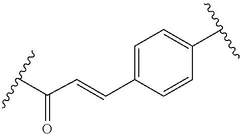 [SMILES: *C(=O)/C=C/c1ccc(*)cc1] | — |
| 1154 | 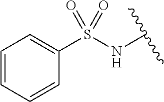 [SMILES: *NS(=O)(=O)c1ccccc1] | 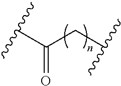 | 5 |
| 1155 | 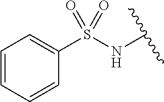 [SMILES: *NS(=O)(=O)c1ccccc1] | 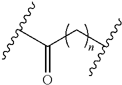 | 6 |
| 1156 | 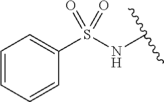 [SMILES: *NS(=O)(=O)c1ccccc1] | 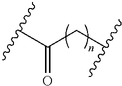 | 7 |
| 1157 | 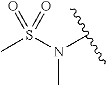 [SMILES: *N(C)S(C)(=O)=O] | 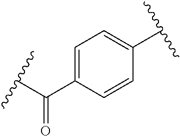 [SMILES: *C(=O)c1ccc(*)cc1] | — |
| 1158 | 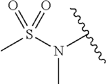 [SMILES: *N(C)S(C)(=O)=O] | 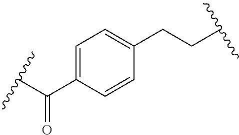 [SMILES: *CCc1ccc(C(*)=O)cc1] | |
| 1159 | 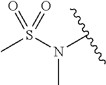 [SMILES: *N(C)S(C)(=O)=O] | 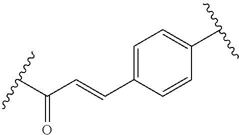 [SMILES: *C(=O)/C=C/c1ccc(*)cc1] | — |
| 1160 | 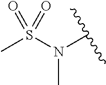 [SMILES: *N(C)S(C)(=O)=O] | 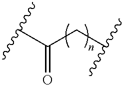 | 5 |
| 1161 | 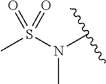 [SMILES: *N(C)S(C)(=O)=O] | 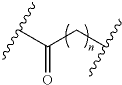 | 6 |
| 1162 | 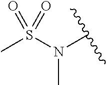 [SMILES: *N(C)S(C)(=O)=O] | 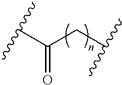 | 7 |
| 1163 | 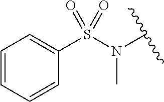 [SMILES: *N(C)S(=O)(=O)c1ccccc1] | 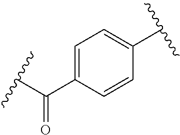 [SMILES: *C(=O)c1ccc(*)cc1] | — |
| 1164 | 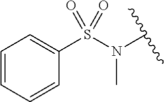 [SMILES: *N(C)S(=O)(=O)c1ccccc1] | 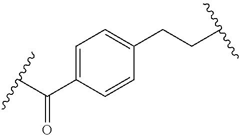 [SMILES: *CCc1ccc(C(*)=O)cc1] | |
| 1165 | 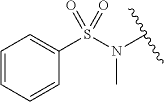 [SMILES: *N(C)S(=O)(=O)c1ccccc1] | 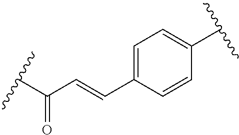 [SMILES: *C(=O)/C=C/c1ccc(*)cc1] | — |
| 1166 | 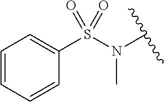 [SMILES: *N(C)S(=O)(=O)c1ccccc1] | 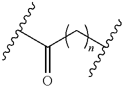 | 5 |
| 1167 | 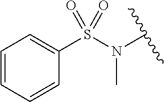 [SMILES: *N(C)S(=O)(=O)c1ccccc1] | 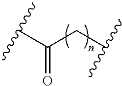 | 6 |
| 1168 | 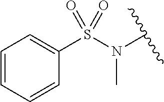 [SMILES: *N(C)S(=O)(=O)c1ccccc1] | 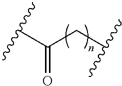 | 7 |
| 1169 | 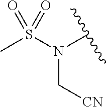 [SMILES: *N(CC#N)S(C)(=O)=O] | 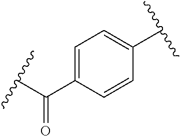 [SMILES: *C(=O)c1ccc(*)cc1] | — |
| 1170 | 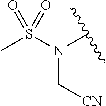 [SMILES: *N(CC#N)S(C)(=O)=O] | 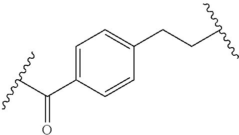 [SMILES: *CCc1ccc(C(*)=O)cc1] | |
| 1171 | 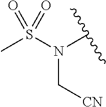 [SMILES: *N(CC#N)S(C)(=O)=O] | 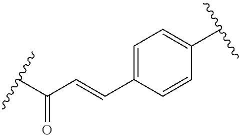 [SMILES: *C(=O)/C=C/c1ccc(*)cc1] | — |
| 1172 | 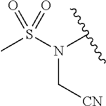 [SMILES: *N(CC#N)S(C)(=O)=O] | 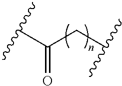 | 5 |
| 1173 | 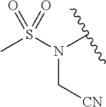 [SMILES: *N(CC#N)S(C)(=O)=O] | 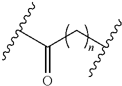 | 6 |
| 1174 | 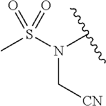 [SMILES: *N(CC#N)S(C)(=O)=O] | 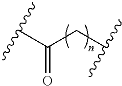 | 7 |
| 1175 | 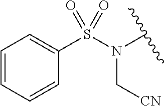 [SMILES: *N(CC#N)S(=O)(=O)c1ccccc1] | 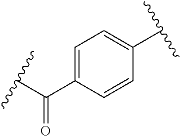 [SMILES: *C(=O)c1ccc(*)cc1] | — |
| 1176 | 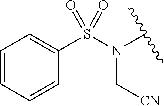 [SMILES: *N(CC#N)S(=O)(=O)c1ccccc1] | 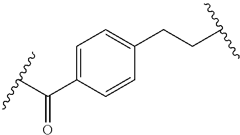 [SMILES: *CCc1ccc(C(*)=O)cc1] | |
| 1177 | 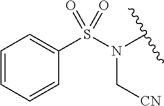 [SMILES: *N(CC#N)S(=O)(=O)c1ccccc1] | 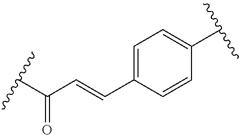 [SMILES: *C(=O)/C=C/c1ccc(*)cc1] | — |
| 1178 | 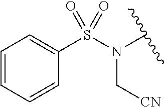 [SMILES: *N(CC#N)S(=O)(=O)c1ccccc1] | 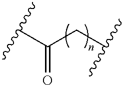 | 5 |
| 1179 | 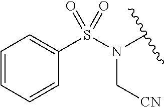 [SMILES: *N(CC#N)S(=O)(=O)c1ccccc1] | 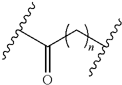 | 6 |
| 1180 | 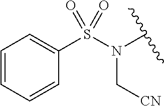 [SMILES: *N(CC#N)S(=O)(=O)c1ccccc1] | 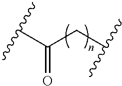 | 7 |
| 1181 | 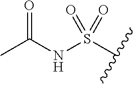 [SMILES: *S(=O)(=O)NC(C)=O] | 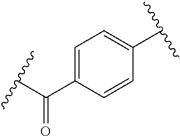 [SMILES: *C(=O)c1ccc(*)cc1] | — |
| 1182 | 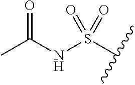 [SMILES: *S(=O)(=O)NC(C)=O] | 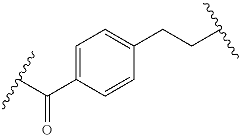 [SMILES: *CCc1ccc(C(*)=O)cc1] | |
| 1183 | 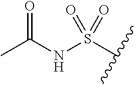 [SMILES: *S(=O)(=O)NC(C)=O] | 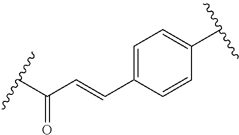 [SMILES: *C(=O)/C=C/c1ccc(*)cc1] | — |
| 1184 | 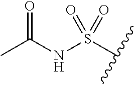 [SMILES: *S(=O)(=O)NC(C)=O] | 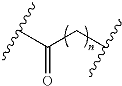 | 5 |
| 1185 | 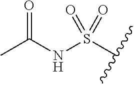 [SMILES: *S(=O)(=O)NC(C)=O] | 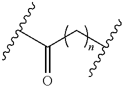 | 6 |
| 1186 | 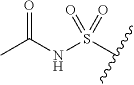 [SMILES: *S(=O)(=O)NC(C)=O] | 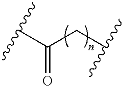 | 7 |
| 1187 | 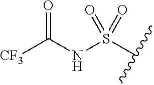 [SMILES: *S(=O)(=O)NC(=O)C(F)(F)F] | 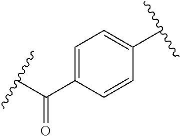 [SMILES: *C(=O)c1ccc(*)cc1] | — |
| 1188 | 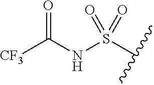 [SMILES: *S(=O)(=O)NC(=O)C(F)(F)F] | 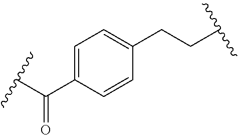 [SMILES: *CCc1ccc(C(*)=O)cc1] | |
| 1189 | 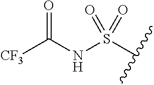 [SMILES: *S(=O)(=O)NC(=O)C(F)(F)F] | 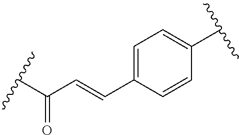 [SMILES: *C(=O)/C=C/c1ccc(*)cc1] | — |
| 1190 | 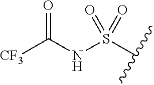 [SMILES: *S(=O)(=O)NC(=O)C(F)(F)F] | 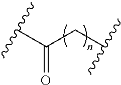 | 5 |
| 1191 | 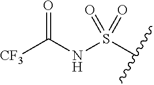 [SMILES: *S(=O)(=O)NC(=O)C(F)(F)F] | 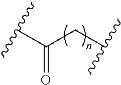 | 6 |
| 1192 | 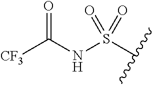 [SMILES: *S(=O)(=O)NC(=O)C(F)(F)F] | 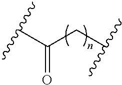 | 7 |
| 1193 | 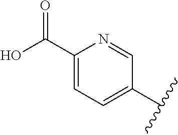 [SMILES: *c1ccc(C(=O)O)nc1] | 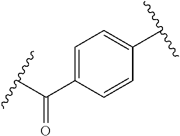 [SMILES: *C(=O)c1ccc(*)cc1] | — |
| 1194 | 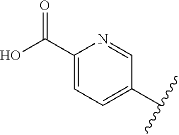 [SMILES: *c1ccc(C(=O)O)nc1] | 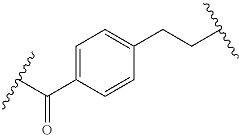 [SMILES: *CCc1ccc(C(*)=O)cc1] | |
| 1195 | 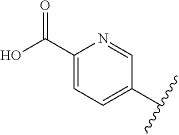 [SMILES: *c1ccc(C(=O)O)nc1] | 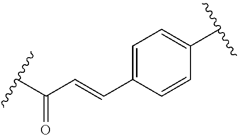 [SMILES: *C(=O)/C=C/c1ccc(*)cc1] | — |
| 1196 | 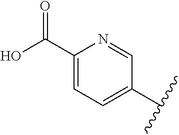 [SMILES: *c1ccc(C(=O)O)nc1] | 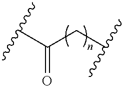 | 5 |
| 1197 | 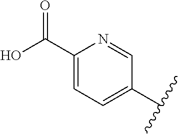 [SMILES: *c1ccc(C(=O)O)nc1] | 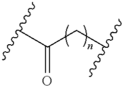 | 6 |
| 1198 | 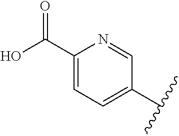 [SMILES: *c1ccc(C(=O)O)nc1] | 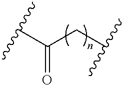 | 7 |
| 1199 | 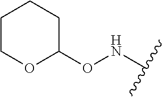 [SMILES: *NOC1CCCCO1] | 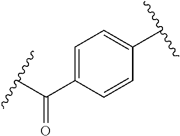 [SMILES: *C(=O)c1ccc(*)cc1] | — |
| 1200 | 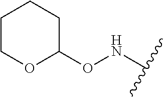 [SMILES: *NOC1CCCCO1] | 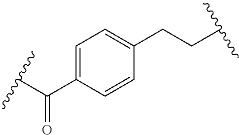 [SMILES: *CCc1ccc(C(*)=O)cc1] | |
| 1201 | 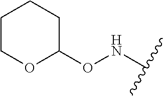 [SMILES: *NOC1CCCCO1] | 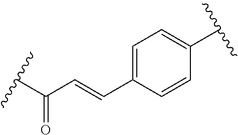 [SMILES: *C(=O)/C=C/c1ccc(*)cc1] | — |
| 1202 | 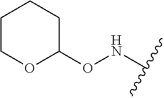 [SMILES: *NOC1CCCCO1] | 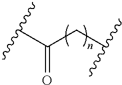 | 5 |
| 1203 | 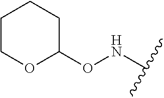 [SMILES: *NOC1CCCCO1] | 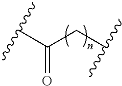 | 6 |
| 1204 | 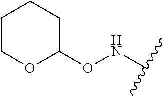 [SMILES: *NOC1CCCCO1] | 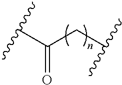 | 7 |
| 1205 | 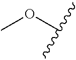 [SMILES: *OC] | 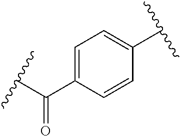 [SMILES: *C(=O)c1ccc(*)cc1] | — |
| 1206 | 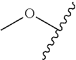 [SMILES: *OC] | 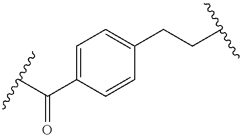 [SMILES: *CCc1ccc(C(*)=O)cc1] | |
| 1207 | 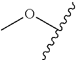 [SMILES: *OC] | 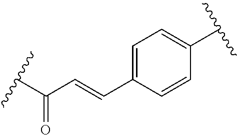 [SMILES: *C(=O)/C=C/c1ccc(*)cc1] | — |
| 1208 | 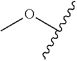 [SMILES: *OC] | 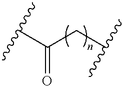 | 5 |
| 1209 | 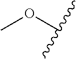 [SMILES: *OC] | 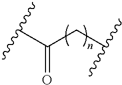 | 6 |
| 1210 | 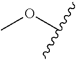 [SMILES: *OC] | 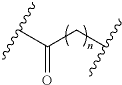 | 7 |
| 1211 | 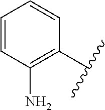 [SMILES: *c1ccccc1N] | 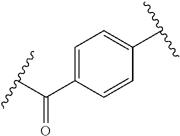 [SMILES: *C(=O)c1ccc(*)cc1] | — |
| 1212 | 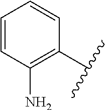 [SMILES: *c1ccccc1N] | 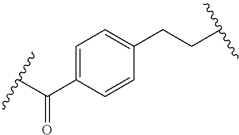 [SMILES: *CCc1ccc(C(*)=O)cc1] | |
| 1213 | 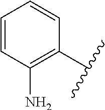 [SMILES: *c1ccccc1N] | 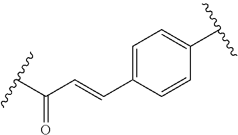 [SMILES: *C(=O)/C=C/c1ccc(*)cc1] | — |
| 1214 | 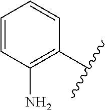 [SMILES: *c1ccccc1N] | 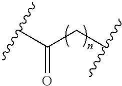 | 5 |
| 1215 | 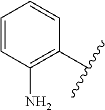 [SMILES: *c1ccccc1N] | 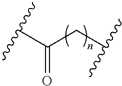 | 6 |
| 1216 | 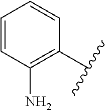 [SMILES: *c1ccccc1N] | 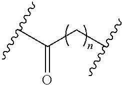 | 7 |
| 1217 | 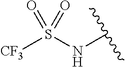 [SMILES: *NS(=O)(=O)C(F)(F)F] | 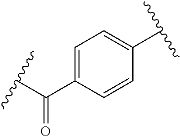 [SMILES: *C(=O)c1ccc(*)cc1] | — |
| 1218 | 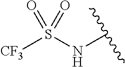 [SMILES: *NS(=O)(=O)C(F)(F)F] | 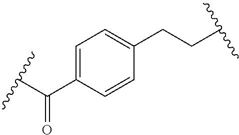 [SMILES: *CCc1ccc(C(*)=O)cc1] | |
| 1219 | 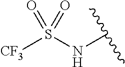 [SMILES: *NS(=O)(=O)C(F)(F)F] | 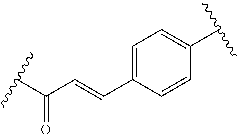 [SMILES: *C(=O)/C=C/c1ccc(*)cc1] | — |
| 1220 | 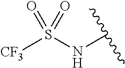 [SMILES: *NS(=O)(=O)C(F)(F)F] | 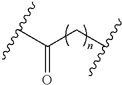 | 5 |
| 1221 | 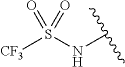 [SMILES: *NS(=O)(=O)C(F)(F)F] | 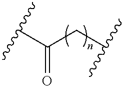 | 6 |
| 1222 | 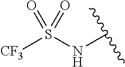 [SMILES: *NS(=O)(=O)C(F)(F)F] | 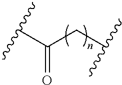 | 7 |
| 1223 | 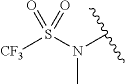 [SMILES: *N(C)S(=O)(=O)C(F)(F)F] | 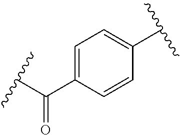 [SMILES: *C(=O)c1ccc(*)cc1] | — |
| 1224 | 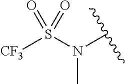 [SMILES: *N(C)S(=O)(=O)C(F)(F)F] | 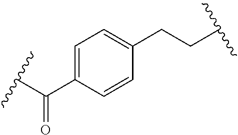 [SMILES: *CCc1ccc(C(*)=O)cc1] | |
| 1225 | 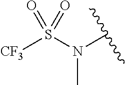 [SMILES: *N(C)S(=O)(=O)C(F)(F)F] | 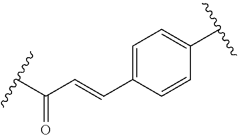 [SMILES: *C(=O)/C=C/c1ccc(*)cc1] | — |
| 1226 | 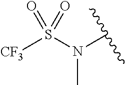 [SMILES: *N(C)S(=O)(=O)C(F)(F)F] | 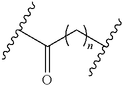 | 5 |
| 1227 | 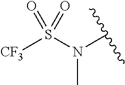 [SMILES: *N(C)S(=O)(=O)C(F)(F)F] | 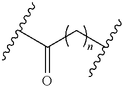 | 6 |
| 1228 | 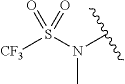 [SMILES: *N(C)S(=O)(=O)C(F)(F)F] | 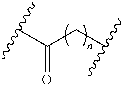 | 7 |

Embodiment 20: The compound according to any one of Embodiments 1 to 4 or Embodiments 11 to 17, wherein the compound of formula (I) is a compound of any one of formula 1229-1342, or a pharmaceutically acceptable salt, solvate, hydrate, cocrystal, or prodrug thereof, wherein in formula 1229-1342:
B is:

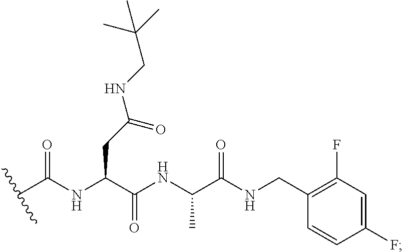 [SMILES: *C(=O)N[C@@H](CC(=O)NCC(C)(C)C)C(=O)N[C@@H](C)C(=O)NCc1ccc(F)cc1F]

and:

| Formula No. | A | L | n |
|---|---|---|---|
| 1229 | 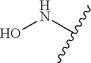 [SMILES: *NO] | 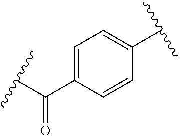 [SMILES: *C(=O)c1ccc(*)cc1] | — |
| 1230 | 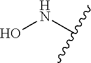 [SMILES: *NO] | 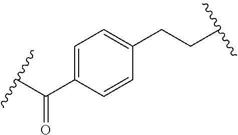 [SMILES: *CCc1ccc(C(*)=O)cc1] | |
| 1231 | 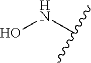 [SMILES: *NO] | 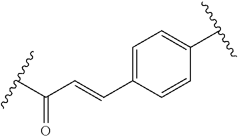 [SMILES: *C(=O)/C=C/c1ccc(*)cc1] | — |
| 1232 | 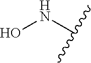 [SMILES: *NO] | 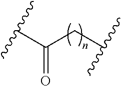 | 5 |
| 1233 | 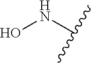 [SMILES: *NO] | 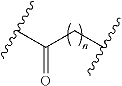 | 6 |
| 1234 | 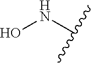 [SMILES: *NO] | 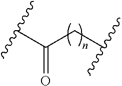 | 7 |
| 1235 | 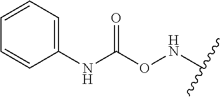 [SMILES: *NOC(=O)Nc1ccccc1] | 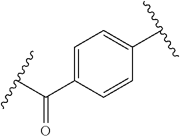 [SMILES: *C(=O)c1ccc(*)cc1] | — |
| 1236 | 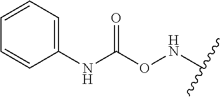 [SMILES: *NOC(=O)Nc1ccccc1] | 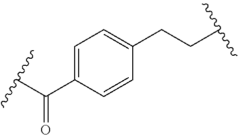 [SMILES: *CCc1ccc(C(*)=O)cc1] | |
| 1237 | 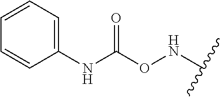 [SMILES: *NOC(=O)Nc1ccccc1] | 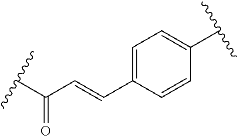 [SMILES: *C(=O)/C=C/c1ccc(*)cc1] | — |
| 1238 | 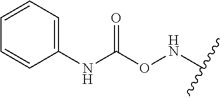 [SMILES: *NOC(=O)Nc1ccccc1] | 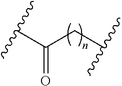 | 5 |
| 1239 | 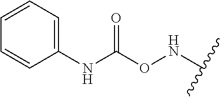 [SMILES: *NOC(=O)Nc1ccccc1] | 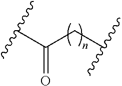 | 6 |
| 1240 | 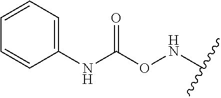 [SMILES: *NOC(=O)Nc1ccccc1] | 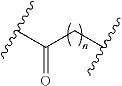 | 7 |
| 1241 | 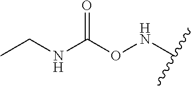 [SMILES: *NOC(=O)NCC] | 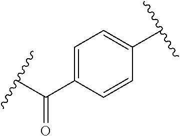 [SMILES: *C(=O)c1ccc(*)cc1] | — |
| 1242 | 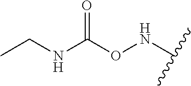 [SMILES: *NOC(=O)NCC] | 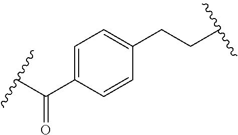 [SMILES: *CCc1ccc(C(*)=O)cc1] | |
| 1243 | 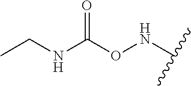 [SMILES: *NOC(=O)NCC] | 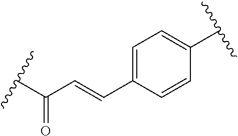 [SMILES: *C(=O)/C=C/c1ccc(*)cc1] | — |
| 1244 | 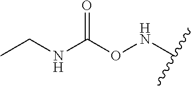 [SMILES: *NOC(=O)NCC] | 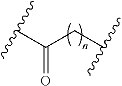 | 5 |
| 1245 | 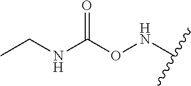 [SMILES: *NOC(=O)NCC] | 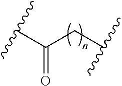 | 6 |
| 1246 | 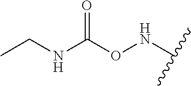 [SMILES: *NOC(=O)NCC] | 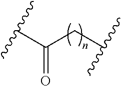 | 7 |
| 1247 | 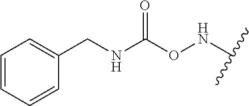 [SMILES: *NOC(=O)NCc1ccccc1] | 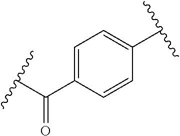 [SMILES: *C(=O)c1ccc(*)cc1] | — |
| 1248 | 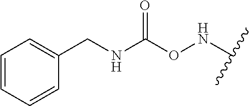 [SMILES: *NOC(=O)NCc1ccccc1] | 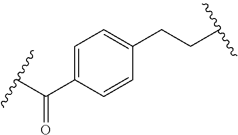 [SMILES: *CCc1ccc(C(*)=O)cc1] | |
| 1249 | 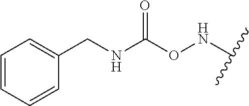 [SMILES: *NOC(=O)NCc1ccccc1] | 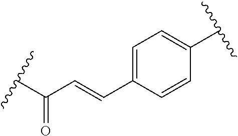 [SMILES: *C(=O)/C=C/c1ccc(*)cc1] | — |
| 1250 | 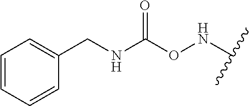 [SMILES: *NOC(=O)NCc1ccccc1] | 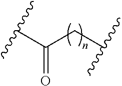 | 5 |
| 1251 | 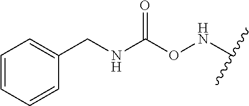 [SMILES: *NOC(=O)NCc1ccccc1] | 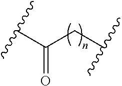 | 6 |
| 1252 | 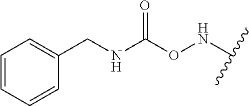 [SMILES: *NOC(=O)NCc1ccccc1] | 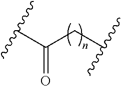 | 7 |
| 1253 | 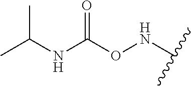 [SMILES: *NOC(=O)NC(C)C] | 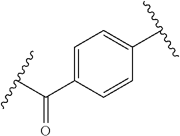 [SMILES: *C(=O)c1ccc(*)cc1] | — |
| 1254 | 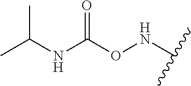 [SMILES: *NOC(=O)NC(C)C] | 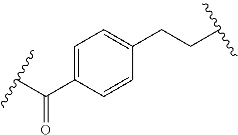 [SMILES: *CCc1ccc(C(*)=O)cc1] | |
| 1255 | 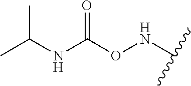 [SMILES: *NOC(=O)NC(C)C] | 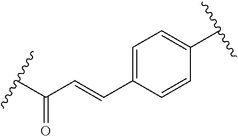 [SMILES: *C(=O)/C=C/c1ccc(*)cc1] | — |
| 1256 | 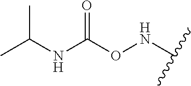 [SMILES: *NOC(=O)NC(C)C] | 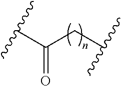 | 5 |
| 1257 | 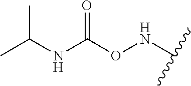 [SMILES: *NOC(=O)NC(C)C] | 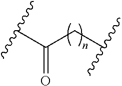 | 6 |
| 1258 | 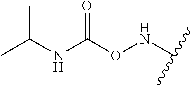 [SMILES: *NOC(=O)NC(C)C] | 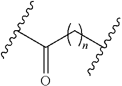 | 7 |
| 1259 | 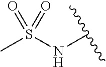 [SMILES: *NS(C)(=O)=O] | 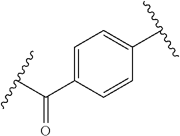 [SMILES: *C(=O)c1ccc(*)cc1] | — |
| 1260 | 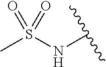 [SMILES: *NS(C)(=O)=O] | 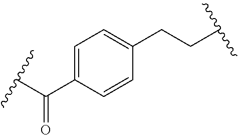 [SMILES: *CCc1ccc(C(*)=O)cc1] | |
| 1261 | 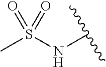 [SMILES: *NS(C)(=O)=O] | 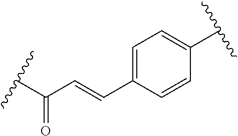 [SMILES: *C(=O)/C=C/c1ccc(*)cc1] | — |
| 1262 | 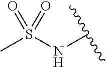 [SMILES: *NS(C)(=O)=O] | 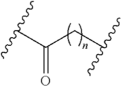 | 5 |
| 1263 | 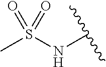 [SMILES: *NS(C)(=O)=O] | 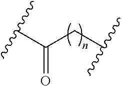 | 6 |
| 1264 | 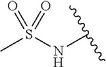 [SMILES: *NS(C)(=O)=O] | 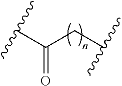 | 7 |
| 1265 | 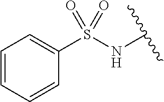 [SMILES: *NS(=O)(=O)c1ccccc1] | 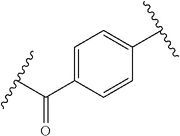 [SMILES: *C(=O)c1ccc(*)cc1] | — |
| 1266 | 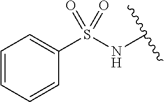 [SMILES: *NS(=O)(=O)c1ccccc1] | 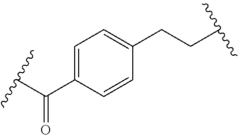 [SMILES: *CCc1ccc(C(*)=O)cc1] | |
| 1267 | 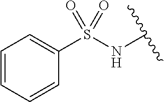 [SMILES: *NS(=O)(=O)c1ccccc1] | 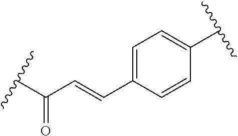 [SMILES: *C(=O)/C=C/c1ccc(*)cc1] | — |
| 1268 | 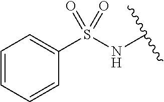 [SMILES: *NS(=O)(=O)c1ccccc1] | 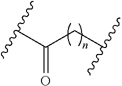 | 5 |
| 1269 | 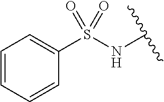 [SMILES: *NS(=O)(=O)c1ccccc1] | 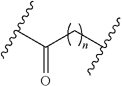 | 6 |
| 1270 | 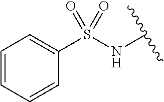 [SMILES: *NS(=O)(=O)c1ccccc1] | 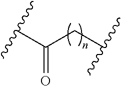 | 7 |
| 1271 | 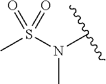 [SMILES: *N(C)S(C)(=O)=O] | 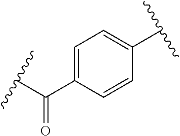 [SMILES: *C(=O)c1ccc(*)cc1] | — |
| 1272 | 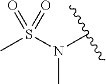 [SMILES: *N(C)S(C)(=O)=O] | 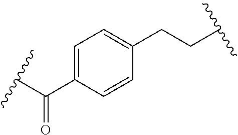 [SMILES: *CCc1ccc(C(*)=O)cc1] | |
| 1273 | 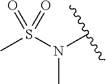 [SMILES: *N(C)S(C)(=O)=O] | 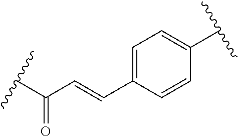 [SMILES: *C(=O)/C=C/c1ccc(*)cc1] | — |
| 1274 | 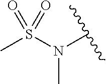 [SMILES: *N(C)S(C)(=O)=O] | 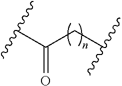 | 5 |
| 1275 | 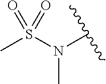 [SMILES: *N(C)S(C)(=O)=O] | 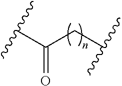 | 6 |
| 1276 | 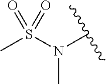 [SMILES: *N(C)S(C)(=O)=O] | 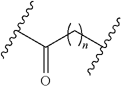 | 7 |
| 1277 | 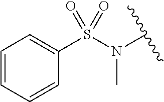 [SMILES: *N(C)S(=O)(=O)c1ccccc1] | 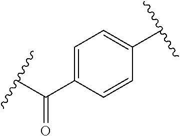 [SMILES: *C(=O)c1ccc(*)cc1] | — |
| 1278 | 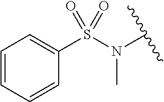 [SMILES: *N(C)S(=O)(=O)c1ccccc1] | 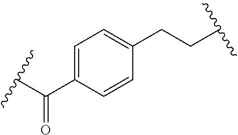 [SMILES: *CCc1ccc(C(*)=O)cc1] | |
| 1279 | 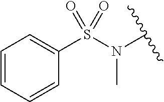 [SMILES: *N(C)S(=O)(=O)c1ccccc1] | 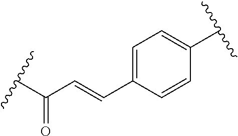 [SMILES: *C(=O)/C=C/c1ccc(*)cc1] | — |
| 1280 | 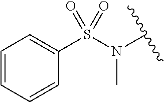 [SMILES: *N(C)S(=O)(=O)c1ccccc1] | 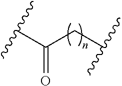 | 5 |
| 1281 | 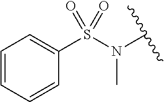 [SMILES: *N(C)S(=O)(=O)c1ccccc1] | 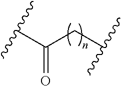 | 6 |
| 1282 | 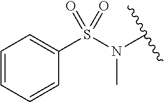 [SMILES: *N(C)S(=O)(=O)c1ccccc1] | 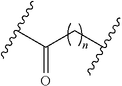 | 7 |
| 1283 | 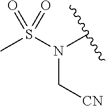 [SMILES: *N(CC#N)S(C)(=O)=O] | 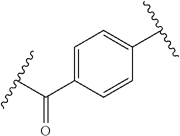 [SMILES: *C(=O)c1ccc(*)cc1] | — |
| 1284 | 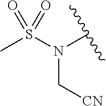 [SMILES: *N(CC#N)S(C)(=O)=O] | 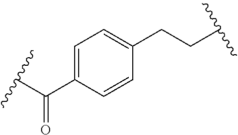 [SMILES: *CCc1ccc(C(*)=O)cc1] | |
| 1285 | 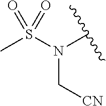 [SMILES: *N(CC#N)S(C)(=O)=O] | 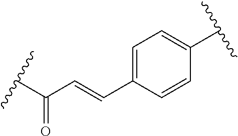 [SMILES: *C(=O)/C=C/c1ccc(*)cc1] | — |
| 1286 | 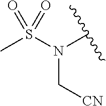 [SMILES: *N(CC#N)S(C)(=O)=O] | 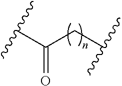 | 5 |
| 1287 | 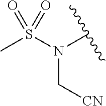 [SMILES: *N(CC#N)S(C)(=O)=O] | 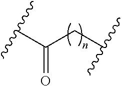 | 6 |
| 1288 | 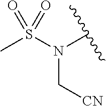 [SMILES: *N(CC#N)S(C)(=O)=O] | 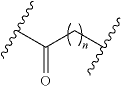 | 7 |
| 1289 | 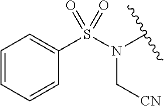 [SMILES: *N(CC#N)S(=O)(=O)c1ccccc1] | 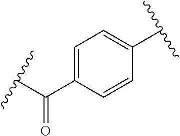 [SMILES: *C(=O)c1ccc(*)cc1] | — |
| 1290 | 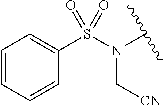 [SMILES: *N(CC#N)S(=O)(=O)c1ccccc1] | 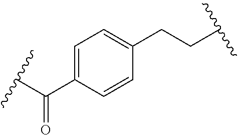 [SMILES: *CCc1ccc(C(*)=O)cc1] | |
| 1291 | 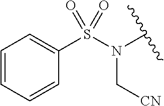 [SMILES: *N(CC#N)S(=O)(=O)c1ccccc1] | 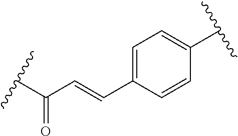 [SMILES: *C(=O)/C=C/c1ccc(*)cc1] | — |
| 1292 | 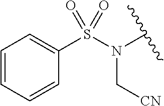 [SMILES: *N(CC#N)S(=O)(=O)c1ccccc1] | 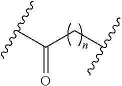 | 5 |
| 1293 | 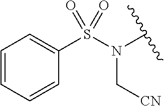 [SMILES: *N(CC#N)S(=O)(=O)c1ccccc1] | 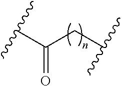 | 6 |
| 1294 | 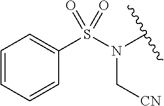 [SMILES: *N(CC#N)S(=O)(=O)c1ccccc1] | 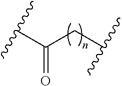 | 7 |
| 1295 | 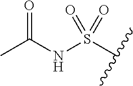 [SMILES: *S(=O)(=O)NC(C)=O] | 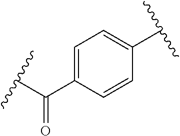 [SMILES: *C(=O)c1ccc(*)cc1] | — |
| 1296 | 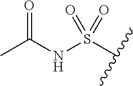 [SMILES: *S(=O)(=O)NC(C)=O] | 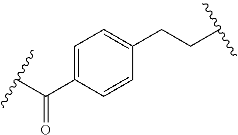 [SMILES: *CCc1ccc(C(*)=O)cc1] | |
| 1297 | 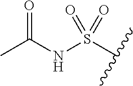 [SMILES: *S(=O)(=O)NC(C)=O] | 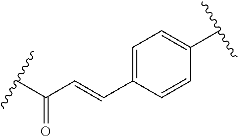 [SMILES: *C(=O)/C=C/c1ccc(*)cc1] | — |
| 1298 | 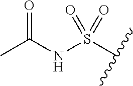 [SMILES: *S(=O)(=O)NC(C)=O] | 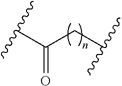 | 5 |
| 1299 | 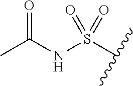 [SMILES: *S(=O)(=O)NC(C)=O] | 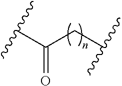 | 6 |
| 1300 | 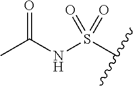 [SMILES: *S(=O)(=O)NC(C)=O] | 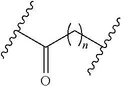 | 7 |
| 1301 | 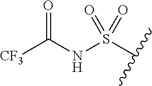 [SMILES: *S(=O)(=O)NC(=O)C(F)(F)F] | 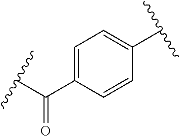 [SMILES: *C(=O)c1ccc(*)cc1] | — |
| 1302 | 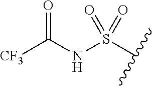 [SMILES: *S(=O)(=O)NC(=O)C(F)(F)F] | 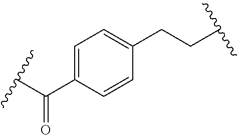 [SMILES: *CCc1ccc(C(*)=O)cc1] | |
| 1303 | 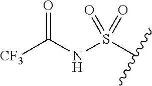 [SMILES: *S(=O)(=O)NC(=O)C(F)(F)F] | 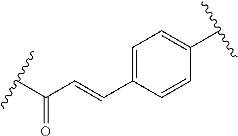 [SMILES: *C(=O)/C=C/c1ccc(*)cc1] | — |
| 1304 | 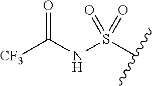 [SMILES: *S(=O)(=O)NC(=O)C(F)(F)F] | 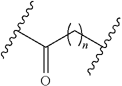 | 5 |
| 1305 | 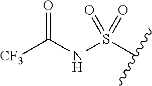 [SMILES: *S(=O)(=O)NC(=O)C(F)(F)F] | 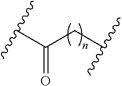 | 6 |
| 1306 | 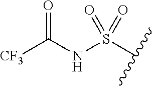 [SMILES: *S(=O)(=O)NC(=O)C(F)(F)F] | 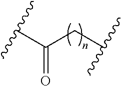 | 7 |
| 1307 | 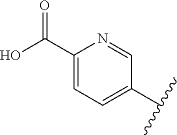 [SMILES: *c1ccc(C(=O)O)nc1] | 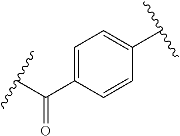 [SMILES: *C(=O)c1ccc(*)cc1] | — |
| 1308 | 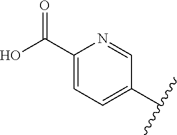 [SMILES: *c1ccc(C(=O)O)nc1] | 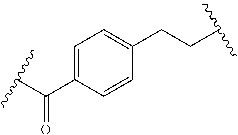 [SMILES: *CCc1ccc(C(*)=O)cc1] | |
| 1309 | 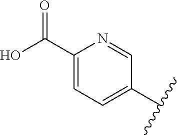 [SMILES: *c1ccc(C(=O)O)nc1] | 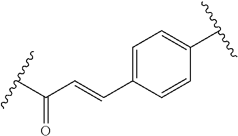 [SMILES: *C(=O)/C=C/c1ccc(*)cc1] | — |
| 1310 | 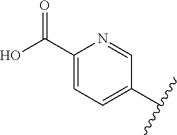 [SMILES: *c1ccc(C(=O)O)nc1] | 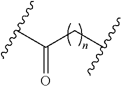 | 5 |
| 1311 | 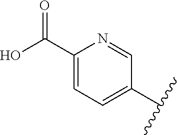 [SMILES: *c1ccc(C(=O)O)nc1] | 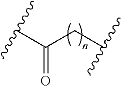 | 6 |
| 1312 | 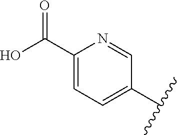 [SMILES: *c1ccc(C(=O)O)nc1] | 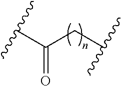 | 7 |
| 1313 | 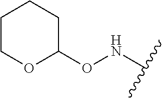 [SMILES: *NOC1CCCCO1] | 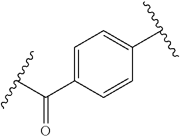 [SMILES: *C(=O)c1ccc(*)cc1] | — |
| 1314 | 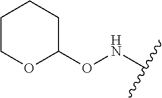 [SMILES: *NOC1CCCCO1] | 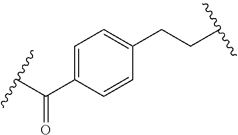 [SMILES: *CCc1ccc(C(*)=O)cc1] | |
| 1315 | 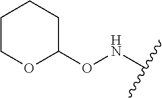 [SMILES: *NOC1CCCCO1] | 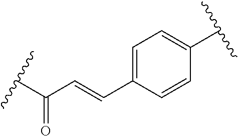 [SMILES: *C(=O)/C=C/c1ccc(*)cc1] | — |
| 1316 | 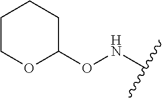 [SMILES: *NOC1CCCCO1] | 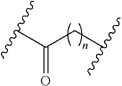 | 5 |
| 1317 | 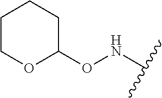 [SMILES: *NOC1CCCCO1] | 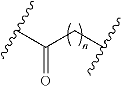 | 6 |
| 1318 | 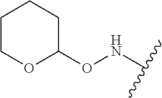 [SMILES: *NOC1CCCCO1] | 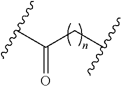 | 7 |
| 1319 | 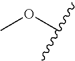 [SMILES: *OC] | 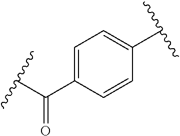 [SMILES: *C(=O)c1ccc(*)cc1] | — |
| 1320 | 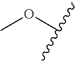 [SMILES: *OC] | 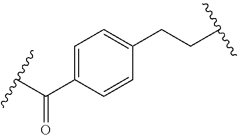 [SMILES: *CCc1ccc(C(*)=O)cc1] | |
| 1321 | 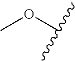 [SMILES: *OC] | 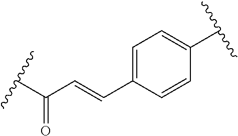 [SMILES: *C(=O)/C=C/c1ccc(*)cc1] | — |
| 1322 | 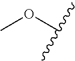 [SMILES: *OC] | 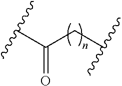 | 5 |
| 1323 | 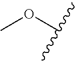 [SMILES: *OC] | 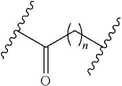 | 6 |
| 1324 | 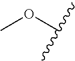 [SMILES: *OC] | 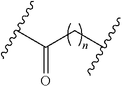 | 7 |
| 1325 | 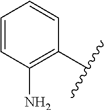 [SMILES: *c1ccccc1N] | 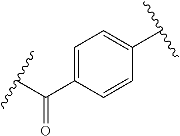 [SMILES: *C(=O)c1ccc(*)cc1] | — |
| 1326 | 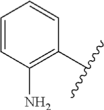 [SMILES: *c1ccccc1N] | 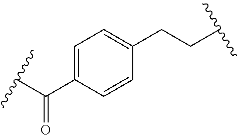 [SMILES: *CCc1ccc(C(*)=O)cc1] | |
| 1327 | 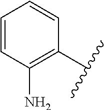 [SMILES: *c1ccccc1N] | 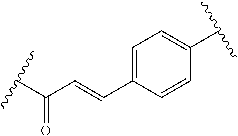 [SMILES: *C(=O)/C=C/c1ccc(*)cc1] | — |
| 1328 | 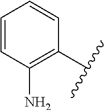 [SMILES: *c1ccccc1N] | 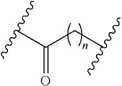 | 5 |
| 1329 | 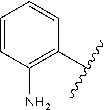 [SMILES: *c1ccccc1N] | 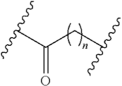 | 6 |
| 1330 | 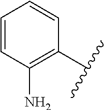 [SMILES: *c1ccccc1N] | 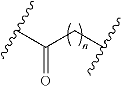 | 7 |
| 1331 | 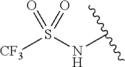 [SMILES: *NS(=O)(=O)C(F)(F)F] | 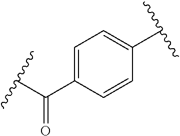 [SMILES: *C(=O)c1ccc(*)cc1] | — |
| 1332 | 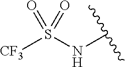 [SMILES: *NS(=O)(=O)C(F)(F)F] | 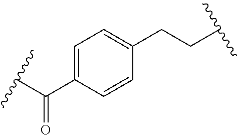 [SMILES: *CCc1ccc(C(*)=O)cc1] | |
| 1333 | 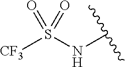 [SMILES: *NS(=O)(=O)C(F)(F)F] | 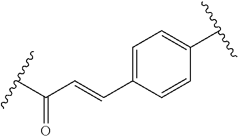 [SMILES: *C(=O)/C=C/c1ccc(*)cc1] | — |
| 1334 | 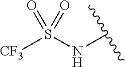 [SMILES: *NS(=O)(=O)C(F)(F)F] | 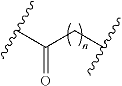 | 5 |
| 1335 | 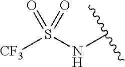 [SMILES: *NS(=O)(=O)C(F)(F)F] | 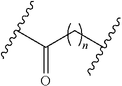 | 6 |
| 1336 | 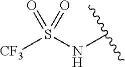 [SMILES: *NS(=O)(=O)C(F)(F)F] | 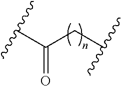 | 7 |
| 1337 | 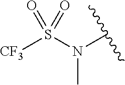 [SMILES: *N(C)S(=O)(=O)C(F)(F)F] | 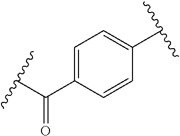 [SMILES: *C(=O)c1ccc(*)cc1] | — |
| 1338 | 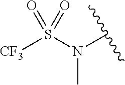 [SMILES: *N(C)S(=O)(=O)C(F)(F)F] | 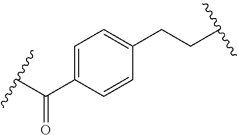 [SMILES: *CCc1ccc(C(*)=O)cc1] | |
| 1339 | 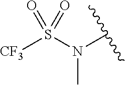 [SMILES: *N(C)S(=O)(=O)C(F)(F)F] | 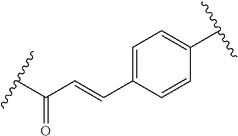 [SMILES: *C(=O)/C=C/c1ccc(*)cc1] | — |
| 1340 | 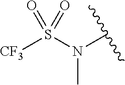 [SMILES: *N(C)S(=O)(=O)C(F)(F)F] | 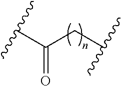 | 5 |
| 1341 | 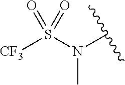 [SMILES: *N(C)S(=O)(=O)C(F)(F)F] | 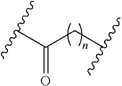 | 6 |
| 1342 | 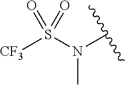 [SMILES: *N(C)S(=O)(=O)C(F)(F)F] | 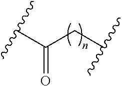 | 7 |

Embodiment 21: The compound according to any one of Embodiments 1 to 19, wherein the compound of formula (I) is a compound of any one of formula AMC-3-017, formula AMC-3-041, formula AMC-2-248, formula AMC-3-033, or formula AMC-3-019, or a pharmaceutically acceptable salt, solvate, hydrate, cocrystal, or prodrug thereof:

| Formula No. | Structure |
|---|---|
| AMC-3-017 | 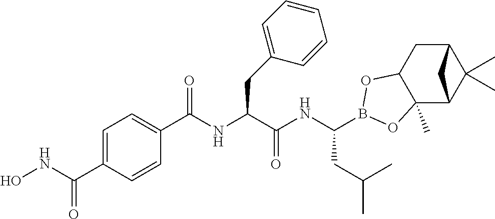 [SMILES: CC(C)C[C@H](NC(=O)[C@H](Cc1ccccc1)NC(=O)c1ccc(C(=O)NO)cc1)B1OC2C[C@H]3C[C@H](C3(C)C)[C@@]2(C)O1] |
| AMC-3-041 | 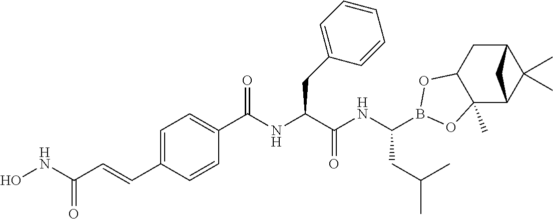 [SMILES: CC(C)C[C@H](NC(=O)[C@H](Cc1ccccc1)NC(=O)c1ccc(/C=C/C(=O)NO)cc1)B1OC2C[C@H]3C[C@H](C3(C)C)[C@@]2(C)O1] |
| AMC-2-248 | 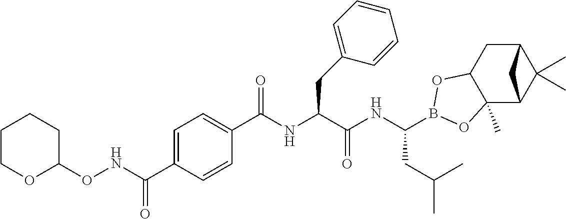 [SMILES: CC(C)C[C@H](NC(=O)[C@H](Cc1ccccc1)NC(=O)c1ccc(C(=O)NOC2CCCCO2)cc1)B1OC2C[C@H]3C[C@H](C3(C)C)[C@@]2(C)O1] |
| AMC-3-033 | 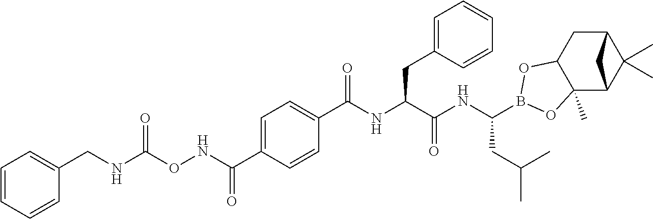 [SMILES: CC(C)C[C@H](NC(=O)[C@H](Cc1ccccc1)NC(=O)c1ccc(C(=O)NOC(=O)NCc2ccccc2)cc1)B1OC2C[C@H]3C[C@H](C3(C)C)[C@@]2(C)O1] |
| AMC-3-019 | 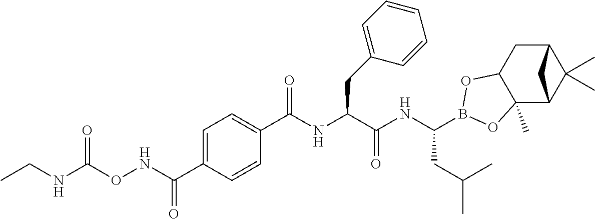 [SMILES: CCNC(=O)ONC(=O)c1ccc(C(=O)N[C@@H](Cc2ccccc2)C(=O)N[C@@H](CC(C)C)B2OC3C[C@H]4C[C@H](C4(C)C)[C@@]3(C)O2)cc1] |

Embodiment 23: The compound according to any one of Embodiments 1 to 4, Embodiments 11 to 17, or Embodiment 20, wherein the compound of formula (I) is a compound ofany one of formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt, solvate, hydrate, cocrystal, or prodrug thereof:

| Formula No. | Structure |
|---|---|
| AMC-3-031 | 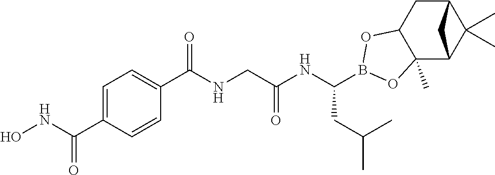 [SMILES: CC(C)C[C@H](NC(=O)CNC(=O)c1ccc(C(=O)NO)cc1)B1OC2C[C@H]3C[C@H](C3(C)C)[C@@]2(C)O1] |
| AMC-3-047 | 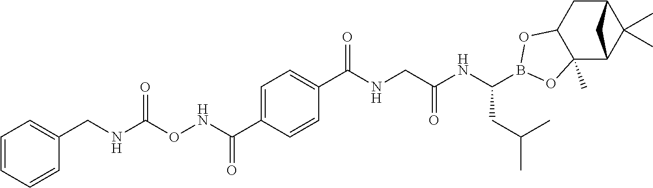 [SMILES: CC(C)C[C@H](NC(=O)CNC(=O)c1ccc(C(=O)NOC(=O)NCc2ccccc2)cc1)B1OC2C[C@H]3C[C@H](C3(C)C)[C@@]2(C)O1] |
| AMC-3-030 | 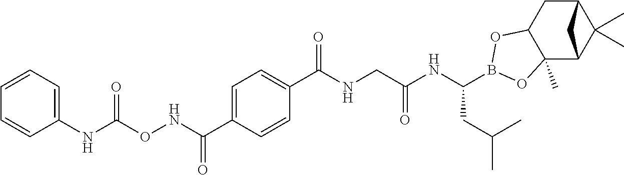 [SMILES: CC(C)C[C@H](NC(=O)CNC(=O)c1ccc(C(=O)NOC(=O)Nc2ccccc2)cc1)B1OC2C[C@H]3C[C@H](C3(C)C)[C@@]2(C)O1] |
| AMC-3-048 | 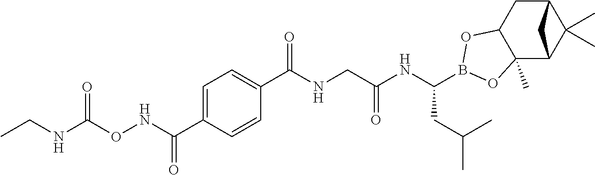 [SMILES: CCNC(=O)ONC(=O)c1ccc(C(=O)NCC(=O)N[C@@H](CC(C)C)B2OC3C[C@H]4C[C@H](C4(C)C)[C@@]3(C)O2)cc1] |
| AMC-3-055 | 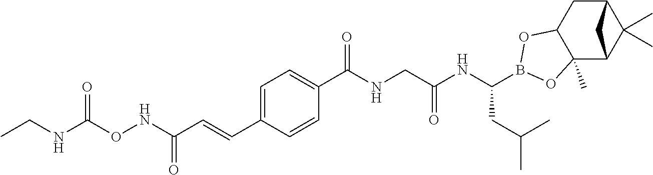 [SMILES: CCNC(=O)ONC(=O)/C=C/c1ccc(C(=O)NCC(=O)N[C@@H](CC(C)C)B2OC3C[C@H]4C[C@H](C4(C)C)[C@@]3(C)O2)cc1] |

Embodiment 23: The compound according to any one of Embodiments 1 to 4, Embodiments 11 to 17, or Embodiment 20, wherein the compound of formula (I) is a compound of any one of formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt, solvate, hydrate, cocrystal, or prodrug thereof:

| Formula No. | Structure |
|---|---|
| AMC-2-052 | 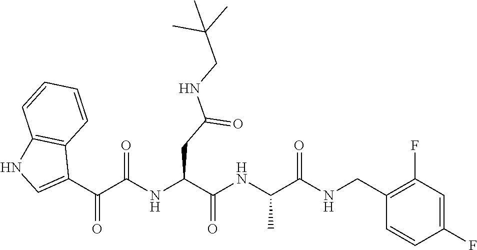 [SMILES: C[C@H](NC(=O)[C@H](CC(=O)NCC(C)(C)C)NC(=O)C(=O)c1c[nH]c2ccccc12)C(=O)NCc1ccc(F)cc1F] |
| AMC-2-036 | 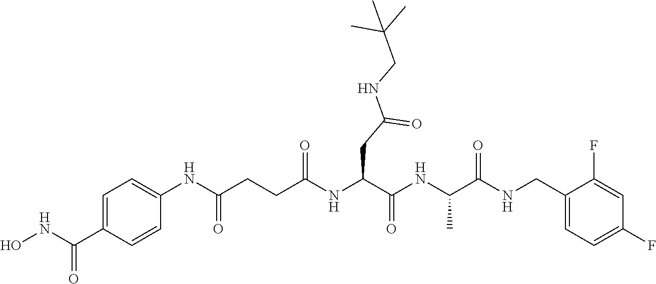 [SMILES: C[C@H](NC(=O)[C@H](CC(=O)NCC(C)(C)C)NC(=O)CCC(=O)Nc1ccc(C(=O)NO)cc1)C(=O)NCc1ccc(F)cc1F] |
| AMC-2-072 | 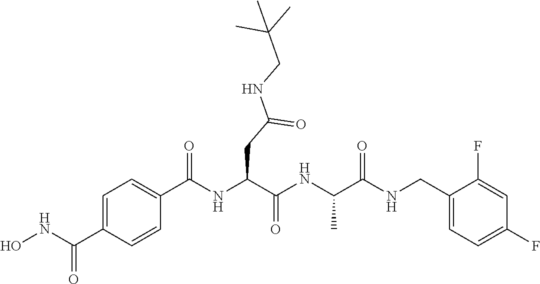 [SMILES: C[C@H](NC(=O)[C@H](CC(=O)NCC(C)(C)C)NC(=O)c1ccc(C(=O)NO)cc1)C(=O)NCc1ccc(F)cc1F] |
| AMC-2-067 | 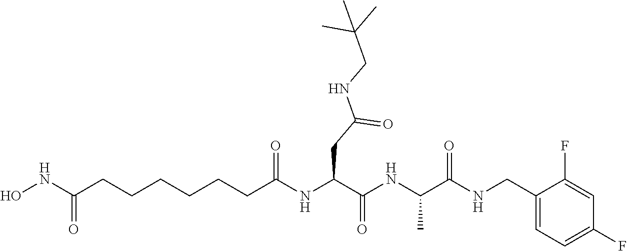 [SMILES: C[C@H](NC(=O)[C@H](CC(=O)NCC(C)(C)C)NC(=O)CCCCCCC(=O)NO)C(=O)NCc1ccc(F)cc1F] |
| AMC-2-215 | 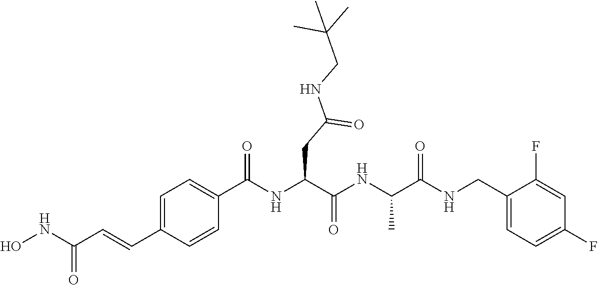 [SMILES: C[C@H](NC(=O)[C@H](CC(=O)NCC(C)(C)C)NC(=O)c1ccc(/C=C/C(=O)NO)cc1)C(=O)NCc1ccc(F)cc1F] |
| AMC-2-091 | 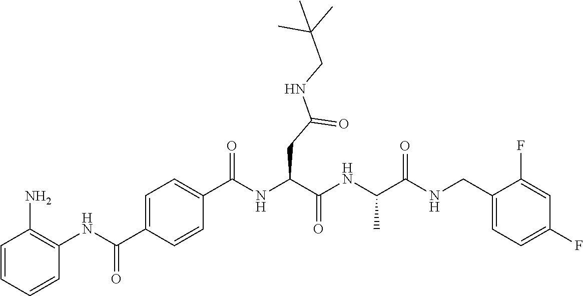 [SMILES: C[C@H](NC(=O)[C@H](CC(=O)NCC(C)(C)C)NC(=O)c1ccc(C(=O)Nc2ccccc2N)cc1)C(=O)NCc1ccc(F)cc1F] |
| AMC-2-152 | 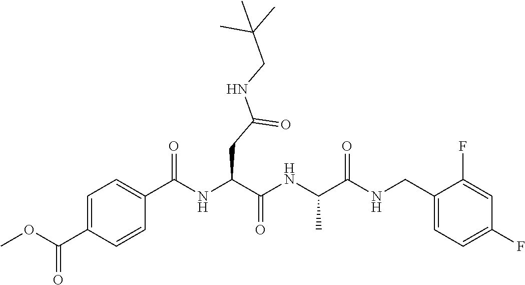 [SMILES: COC(=O)c1ccc(C(=O)N[C@@H](CC(=O)NCC(C)(C)C)C(=O)N[C@@H](C)C(=O)NCc2ccc(F)cc2F)cc1] |
| AMC-2-198 | 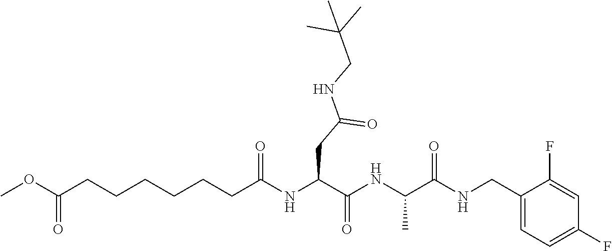 [SMILES: COC(=O)CCCCCCC(=O)N[C@@H](CC(=O)NCC(C)(C)C)C(=O)N[C@@H](C)C(=O)NCc1ccc(F)cc1F] |
| AMC-2-204 | 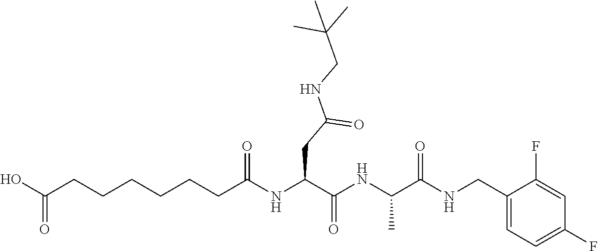 [SMILES: C[C@H](NC(=O)[C@H](CC(=O)NCC(C)(C)C)NC(=O)CCCCCCC(=O)O)C(=O)NCc1ccc(F)cc1F] |
| AMC-2-225 | 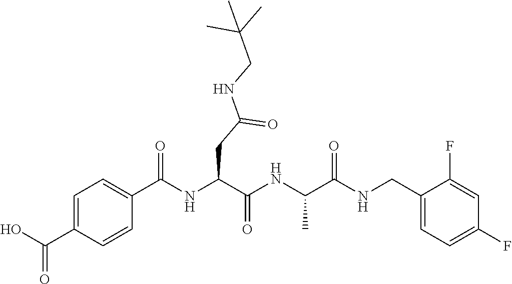 [SMILES: C[C@H](NC(=O)[C@H](CC(=O)NCC(C)(C)C)NC(=O)c1ccc(C(=O)O)cc1)C(=O)NCc1ccc(F)cc1F] |

Embodiment 24: A pharmaceutical composition comprising one or more of compounds according to any one of Embodiments 1 to 23 or a pharmaceutically acceptable salt thereof, and a physiologically compatible carrier medium.

Embodiment 25: A pharmaceutical composition for treating or preventing a disease or disorder alleviated by inhibiting HDAC6 protein activity, the pharmaceutical composition comprising one or more compounds according to any one of Embodiments 1 to 23, or a pharmaceutically acceptable salt thereof, and a physiologically compatible carrier medium.

Embodiment 26: A pharmaceutical composition for treating or preventing a disease or disorder alleviated by inhibiting proteasome activity, the pharmaceutical composition comprising one or more compounds according to any one of Embodiments 1 to 23, or a pharmaceutically acceptable salt thereof, and a physiologically compatible carrier medium.

Embodiment 27: A pharmaceutical composition for treating or preventing a disease or disorder alleviated by both inhibiting HDAC6 protein activity and inhibiting proteasome activity, the pharmaceutical composition comprising one or more compounds according to any one of Embodiments 1 to 23, or a pharmaceutically acceptable salt thereof, and a physiologically compatible carrier medium.

Embodiment 28: The pharmaceutical composition according to any one of Embodiments 25 to 27, wherein the disease or disorder is cancer.

Embodiment 29: The pharmaceutical composition according to Embodiment 28, wherein the cancer is selected from multiple myeloma (MM), mantle cell lymphoma, acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

Embodiment 30: The pharmaceutical composition according to Embodiment 28, wherein the cancer is a blood cancer.

Embodiment 31: The pharmaceutical composition according to Embodiment 29, wherein the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

Embodiment 32: The pharmaceutical composition according to Embodiment 28 or Embodiment 29, wherein the cancer is multiple myeloma (MM).

Embodiment 33: A pharmaceutical composition for treating or preventing multiple myeloma (MM), the pharmaceutical composition comprising one or more compounds according to any one of Embodiments 1-23, or a pharmaceutically acceptable salt thereof, and a physiologically compatible carrier medium.

Embodiment 34: A method of treating or preventing a disease or disorder alleviated by inhibiting HDAC6 protein activity in a patient in need of said treatment or prevention, the method comprising administering a therapeutically effective amount of one or more compounds according to any one of Embodiments 1-23, or a pharmaceutically acceptable salt thereof.

Embodiment 35: A method of treating or preventing a disease or disorder alleviated by inhibiting proteasome activity in a patient in need of said treatment or prevention, the method comprising administering a therapeutically effective amount of one or more compounds according to any one of Embodiments 1-23, or a pharmaceutically acceptable salt thereof.

Embodiment 36: A method of treating or preventing a disease or disorder alleviated by both inhibiting HDAC6 protein activity and inhibiting proteasome activity in a patient in need of said treatment or prevention, the method comprising administering a therapeutically effective amount of one or more compounds according to any one of Embodiments 1-23, or a pharmaceutically acceptable salt thereof.

Embodiment 37: The method according to any one of Embodiments 34 to 36, wherein the disease or disorder is cancer.

Embodiment 38: The method according to Embodiment 37, wherein the cancer is selected from multiple myeloma (MM), mantle cell lymphoma, acute myeloid leukemia (AML), chronic lymphocytic leukemia (CLL), chronic myeloid leukemia (CML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

Embodiment 39: The method according to Embodiment 38, wherein the cancer is a blood cancer.

Embodiment 40: The method according to Embodiment 39, wherein the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

Embodiment 41: The method according to Embodiment 37 or Embodiment 38, wherein the cancer is acute multiple myeloma (MN).

Embodiment 42: A method of treating or preventing acute multiple myeloma (MN) in a patient in need of said treatment or prevention, the method comprising administering a therapeutically effective amount of one or more compounds according to any one of Embodiments 1-23, or a pharmaceutically acceptable salt thereof.

Embodiment 43: The pharmaceutical composition according to any one of Embodiments 25-27, wherein the disease or disorder is an inflammatory disease or disorder.

Embodiment 44: The pharmaceutical composition according to Embodiment 43, wherein the inflammatory disease or disorder is selected from autoimmune diseases, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

Embodiment 45: The method according to any one of Embodiments 34-36, wherein the disease or disorder is an inflammatory disease or disorder.

Embodiment 46: The method according to Embodiment 45, wherein the inflammatory disease or disorder is selected from autoimmune diseases, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

Embodiment 47: The pharmaceutical composition according to any one of Embodiments 25-27, wherein the disease or disorder is an autoimmune disease or disorder.

Embodiment 48: The pharmaceutical composition according to Embodiment 47, wherein the autoimmune disease or disorder is selected from Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

Embodiment 49: The method according to any one of Embodiments 34-36, wherein the disease or disorder is an autoimmune disease or disorder.

Embodiment 50: The method according to Embodiment 49, wherein the autoimmune disease or disorder is selected from cute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claims. All compounds, compositions, formulations, and methods described herein that embody the present disclosure can, in alternate embodiments, be more specifically defined by any of the transitional terms “comprising,” “consisting essentially of,” and “consisting of”

### REFERENCES

1. Tanaka, K. Proc. Jpn. Acad. Ser. B Phys. Biol. Sci. 2009, 85 (1), 12-36.
2. Anighoro, A.; Bajorath, J.; Rastelli, G. J. Med. Chem. 2014, 57 (19), 7874-7887.
3. Rodriguez-Gonzalez, A.; et al. Cancer Res. 2008, 68 (8), 2557-2560.
4. Porter et al, Proc. Natl. Acad. Sci. U.S.A., 2017, 114 (51), 13459-13464.
5. Rodriguez-Gonzalez, A., et al. Cancer Res. 2008, 68 (8), 2557-2560.
6. King, K, et al. Molecules. 2018, 23 (2), 321.
7. Ueda, T. et al, J. Am. Chem. Soc., 2021, 143 (23), 4766-4774.
8. Perrin D., et al. Expert Opin. Drug Discov. 2010, 5, 51-63.
9. nanosyn.bio/technology/general-principle/ (accessed on Oct. 21, 2021).

1.-50. (canceled)

51. A compound of formula (I), or comprising a substructure of formula (I), or a pharmaceutically acceptable salt, solvate, hydrate, cocrystal, or prodrug thereof: 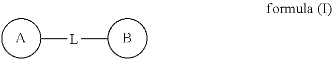
wherein in formula (I):
A comprises a zinc binding moiety;
L is a linking group; and
B comprises a proteasome inhibitor moiety, wherein the zinc binding moiety thereof, and the proteasome inhibitor moiety thereof are each connected to L at any chemically feasible site.

52. The compound of claim 51, wherein B comprises a proteosome inhibitor moiety selected from bortezomib, ixazomib, carfilzomib, oprozomib, marizomib, CEP-18770, disulfiram, epigallocatechin-3-gallate, epoxomicin, lactacystin, MG132, MLN9708, ONX 0912, PR-924, PR-957, KZR-504, LMP7-IN-1, salinosporamide A, epoxomycine, eponemycine, aclacinomycine A, and any substructure thereof.

53. The compound of claim 51, wherein B comprises a moiety selected from: 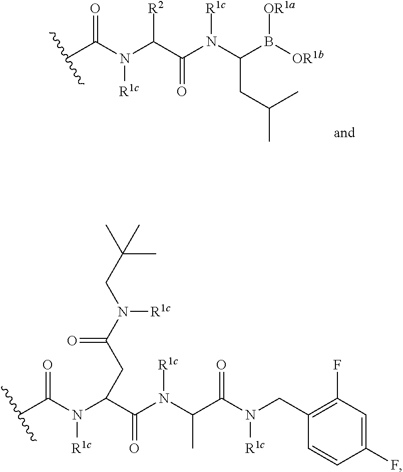
wherein R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups;
each R^{1c} is independently at each occurrence selected from hydrogen and —C₁₋₁₀ alkyl-; and
R² is selected from hydrogen, alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a}), —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

54. The compound of claim 51, wherein the compound of formula (I) is a compound of formula (10) or formula (12), or a pharmaceutically acceptable salt, solvate, hydrate, cocrystal, or prodrug thereof: 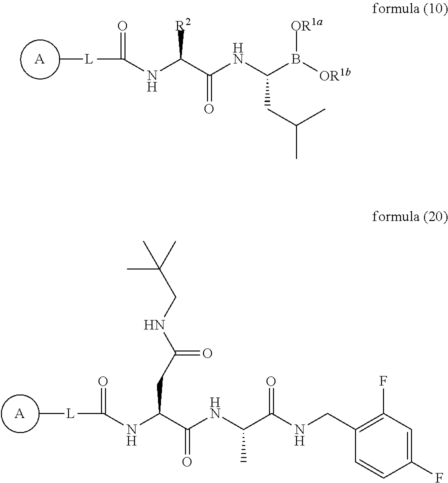
wherein in formula (10) and formula (20):
A comprises a zinc binding moiety;
L is a linking group;
R^{1a} and R^{1b} are independently at each occurrence selected from hydrogen and —C₁₋₁₀ alkyl-, or R^{1a} and R^{1b} are joined together to form a monocyclic or polycyclic cycloalkyl ring optionally substituted with one or more C₁₋₁₀ alkyl groups; and
R² is selected from hydrogen and optionally substituted —C₁₋₁₀ alkyl,
wherein the zinc binding moiety thereof is connected to L at any chemically feasible site.

55. The compound of claim 54, wherein the compound of formula (10) is a compound of formula (11) or formula (12), or a pharmaceutically acceptable salt, solvate, hydrate, cocrystal, or prodrug thereof: 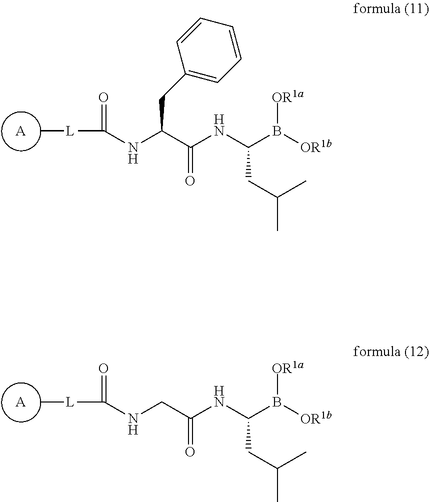
wherein in formula (11) and formula (12):
A comprises a zinc binding moiety;
L is a linking group; and
R^{1a} and R^{1b} are each hydrogen, or R^{1a} and R^{1b} are joined together to form a dioxaborolane, a dioxaborinane, or

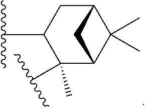 [SMILES: *C1C[C@H]2C[C@H](C2(C)C)[C@]1(*)C]

56. The compound of claim 51, wherein L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and each R^{b} is independently hydrogen, optionally substituted alkyl, or optionally substituted aryl.

57. The compound of claim 51, wherein L comprises one or more linking groups selected from 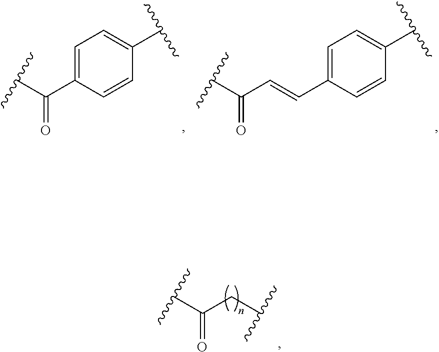
wherein n is an integer from 0 to 7, 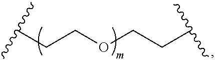
wherein m is an integer from 0 to 5,

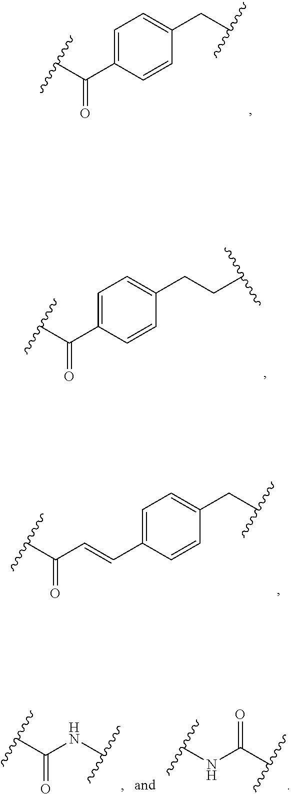 [SMILES: *Cc1ccc(C(*)=O)cc1 ; *CCc1ccc(C(*)=O)cc1 ; *Cc1ccc(/C=C/C(*)=O)cc1 ; *NC(*)=O ; *NC(*)=O]

58. The compound of claim 51, wherein A comprises a moiety selected from: 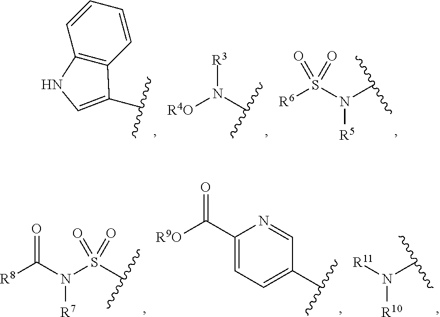
and R¹²O—,
wherein
R³ is selected from hydrogen and optionally substituted —C₁₋₁₀ alkyl-;
R⁴ is selected from hydrogen, N(R^{b})₂C(O)—, and —R^{b}OC(O)—;
R⁵ is selected from hydrogen and optionally substituted —C₁₋₁₀ alkyl-;
R⁶ is selected from optionally substituted —C₁₋₁₀ alkyl- and optionally substituted aryl;
R⁷ is selected from hydrogen and 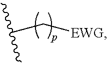
wherein EWG comprises an electron withdrawing group selected from —CN, —NO₂, —C(O)R¹³, —C(O)N(R¹⁴), and —N(R¹⁴)C(O)C(O)R¹⁴, wherein p is an integer between 1 and 5, R¹³ is selected from —C₁₋₁₀ alkyl, —C₁₋₁₀ alkoxy, and each R¹⁴ is independently selected from hydrogen and —C₁₋₁₀ alkyl;
R⁸ is optionally substituted —C₁₋₁₀ alkyl-;
R⁹ is selected from hydrogen and optionally substituted —C₁₋₁₀ alkyl-;
R¹⁰ is selected from hydrogen and optionally substituted —C₁₋₁₀ alkyl-;
R¹¹ is optionally substituted aryl;
R¹² is optionally substituted —C₁₋₁₀ alkyl; and
each R^{b} is independently hydrogen, optionally substituted alkyl, optionally substituted heterocyclyl, or optionally substituted aryl.

59. The compound of claim 51, wherein A is selected from:

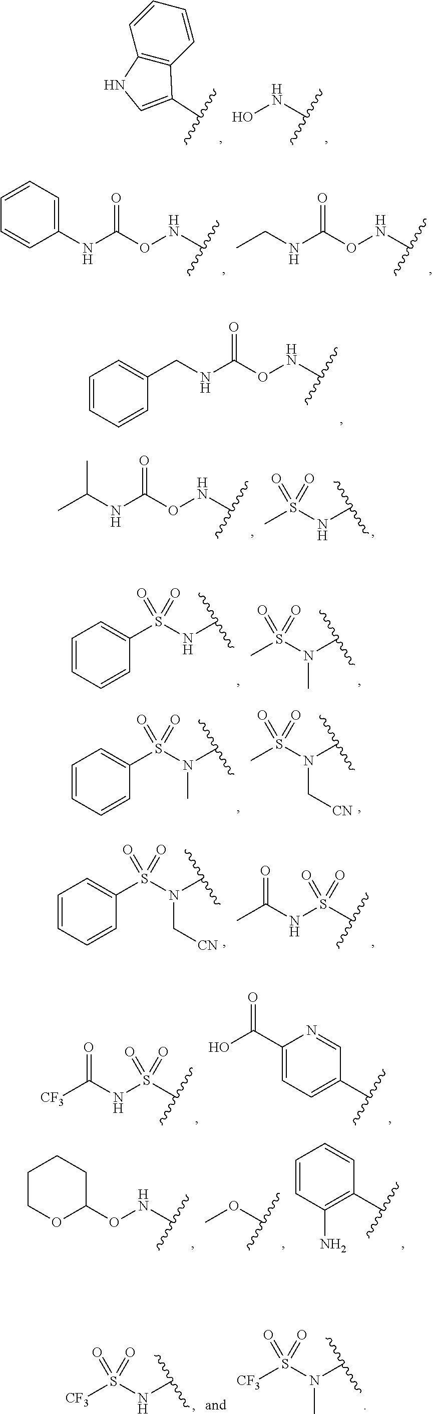

60. The compound of claim 51, wherein the compound of formula (I) is a compound of any one of formula 1001-1114, or a pharmaceutically acceptable salt, solvate, hydrate, cocrystal, or prodrug thereof, wherein in formula 1001-1114:
B is: 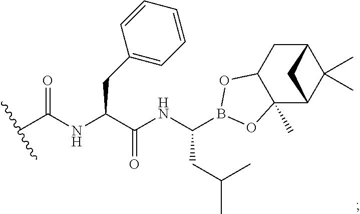 [SMILES: *C(=O)N[C@@H](Cc1ccccc1)C(=O)N[C@@H](CC(C)C)B1OC2C[C@H]3C[C@H](C3(C)C)[C@@]2(C)O1]
and:

| Formula No. | A | L | n |
|---|---|---|---|
| 1001 | 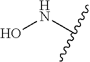 [SMILES: *NO] | 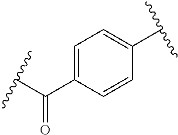 [SMILES: *C(=O)c1ccc(*)cc1] | — |
| 1002 | 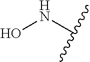 [SMILES: *NO] | 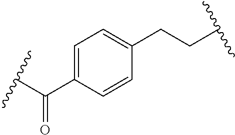 [SMILES: *CCc1ccc(C(*)=O)cc1] | |
| 1003 | 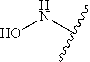 [SMILES: *NO] | 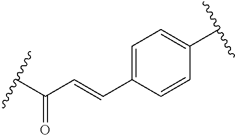 [SMILES: *C(=O)/C=C/c1ccc(*)cc1] | — |
| 1004 | 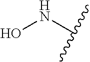 [SMILES: *NO] | 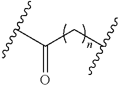 | 5 |
| 1005 | 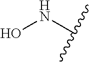 [SMILES: *NO] | 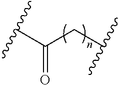 | 6 |
| 1006 | 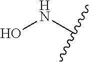 [SMILES: *NO] | 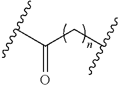 | 7 |
| 1007 | 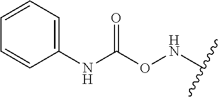 [SMILES: *NOC(=O)Nc1ccccc1] | 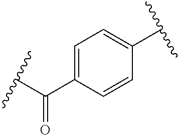 [SMILES: *C(=O)c1ccc(*)cc1] | — |
| 1008 | 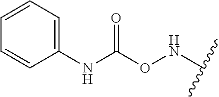 [SMILES: *NOC(=O)Nc1ccccc1] | 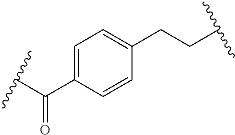 [SMILES: *CCc1ccc(C(*)=O)cc1] | |
| 1009 | 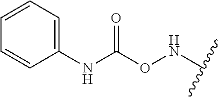 [SMILES: *NOC(=O)Nc1ccccc1] | 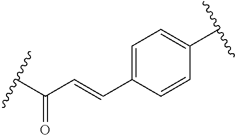 [SMILES: *C(=O)/C=C/c1ccc(*)cc1] | — |
| 1010 | 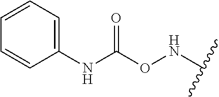 [SMILES: *NOC(=O)Nc1ccccc1] | 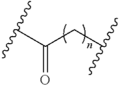 | 5 |
| 1011 | 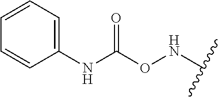 [SMILES: *NOC(=O)Nc1ccccc1] | 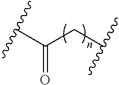 | 6 |
| 1012 | 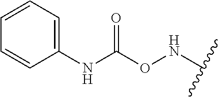 [SMILES: *NOC(=O)Nc1ccccc1] | 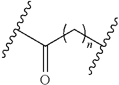 | 7 |
| 1013 | 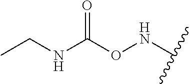 [SMILES: *NOC(=O)NCC] | 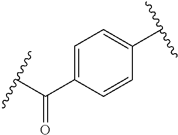 [SMILES: *C(=O)c1ccc(*)cc1] | — |
| 1014 | 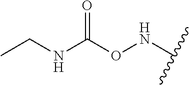 [SMILES: *NOC(=O)NCC] | 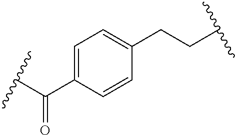 [SMILES: *CCc1ccc(C(*)=O)cc1] | |
| 1015 | 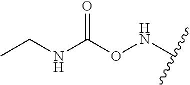 [SMILES: *NOC(=O)NCC] | 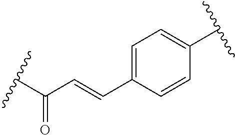 [SMILES: *C(=O)/C=C/c1ccc(*)cc1] | — |
| 1016 | 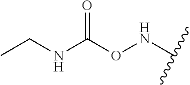 [SMILES: *NOC(=O)NCC] | 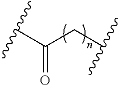 | 5 |
| 1017 | 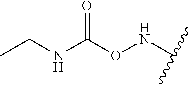 [SMILES: *NOC(=O)NCC] | 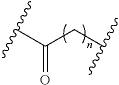 | 6 |
| 1018 | 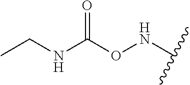 [SMILES: *NOC(=O)NCC] | 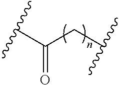 | 7 |
| 1019 | 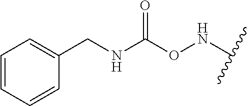 [SMILES: *NOC(=O)NCc1ccccc1] | 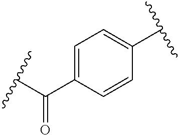 [SMILES: *C(=O)c1ccc(*)cc1] | — |
| 1020 | 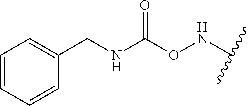 [SMILES: *NOC(=O)NCc1ccccc1] | 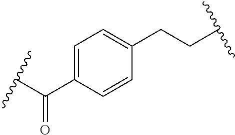 [SMILES: *CCc1ccc(C(*)=O)cc1] | |
| 1021 | 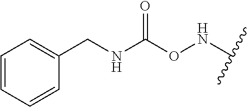 [SMILES: *NOC(=O)NCc1ccccc1] | 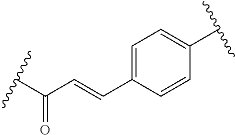 [SMILES: *C(=O)/C=C/c1ccc(*)cc1] | — |
| 1022 | 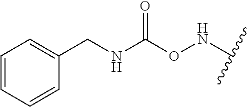 [SMILES: *NOC(=O)NCc1ccccc1] | 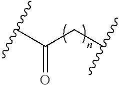 | 5 |
| 1023 | 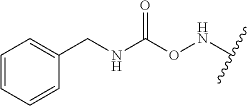 [SMILES: *NOC(=O)NCc1ccccc1] | 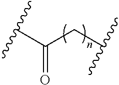 | 6 |
| 1024 | 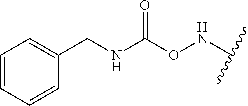 [SMILES: *NOC(=O)NCc1ccccc1] | 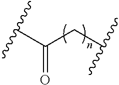 | 7 |
| 1025 | 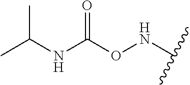 [SMILES: *NOC(=O)NC(C)C] | 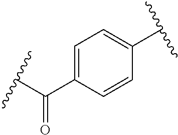 [SMILES: *C(=O)c1ccc(*)cc1] | — |
| 1026 | 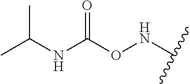 [SMILES: *NOC(=O)NC(C)C] | 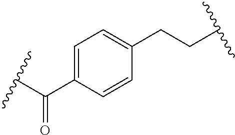 [SMILES: *CCc1ccc(C(*)=O)cc1] | |
| 1027 | 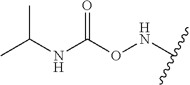 [SMILES: *NOC(=O)NC(C)C] | 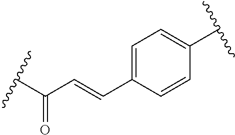 [SMILES: *C(=O)/C=C/c1ccc(*)cc1] | — |
| 1028 | 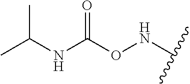 [SMILES: *NOC(=O)NC(C)C] | 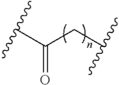 | 5 |
| 1029 | 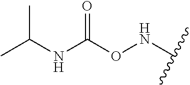 [SMILES: *NOC(=O)NC(C)C] | 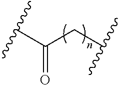 | 6 |
| 1030 | 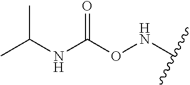 [SMILES: *NOC(=O)NC(C)C] | 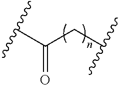 | 7 |
| 1031 | 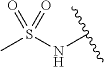 [SMILES: *NS(C)(=O)=O] | 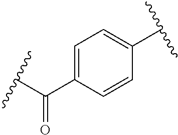 [SMILES: *C(=O)c1ccc(*)cc1] | — |
| 1032 | 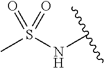 [SMILES: *NS(C)(=O)=O] | 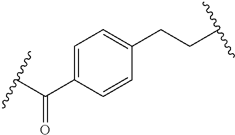 [SMILES: *CCc1ccc(C(*)=O)cc1] | |
| 1033 | 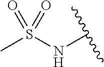 [SMILES: *NS(C)(=O)=O] | 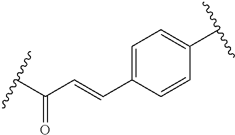 [SMILES: *C(=O)/C=C/c1ccc(*)cc1] | — |
| 1034 | 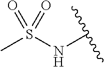 [SMILES: *NS(C)(=O)=O] | 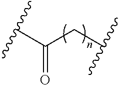 | 5 |
| 1035 | 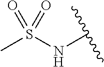 [SMILES: *NS(C)(=O)=O] | 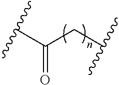 | 6 |
| 1036 | 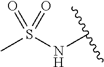 [SMILES: *NS(C)(=O)=O] | 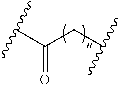 | 7 |
| 1037 | 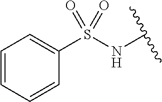 [SMILES: *NS(=O)(=O)c1ccccc1] | 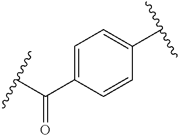 [SMILES: *C(=O)c1ccc(*)cc1] | — |
| 1038 | 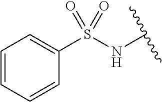 [SMILES: *NS(=O)(=O)c1ccccc1] | 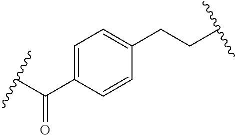 [SMILES: *CCc1ccc(C(*)=O)cc1] | |
| 1039 | 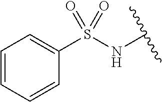 [SMILES: *NS(=O)(=O)c1ccccc1] | 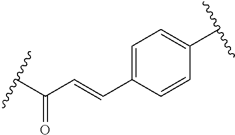 [SMILES: *C(=O)/C=C/c1ccc(*)cc1] | — |
| 1040 | 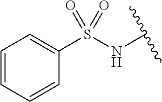 [SMILES: *NS(=O)(=O)c1ccccc1] | 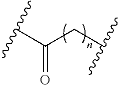 | 5 |
| 1041 | 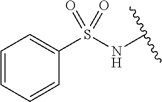 [SMILES: *NS(=O)(=O)c1ccccc1] | 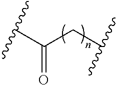 | 6 |
| 1042 | 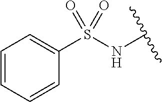 [SMILES: *NS(=O)(=O)c1ccccc1] | 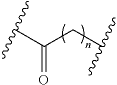 | 7 |
| 1043 | 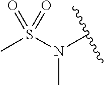 [SMILES: *N(C)S(C)(=O)=O] | 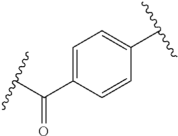 [SMILES: *C(=O)c1ccc(*)cc1] | — |
| 1044 | 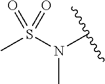 [SMILES: *N(C)S(C)(=O)=O] | 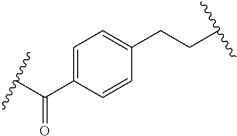 [SMILES: *CCc1ccc(C(*)=O)cc1] | |
| 1045 | 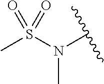 [SMILES: *N(C)S(C)(=O)=O] | 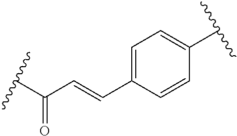 [SMILES: *C(=O)/C=C/c1ccc(*)cc1] | — |
| 1046 | 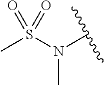 [SMILES: *N(C)S(C)(=O)=O] | 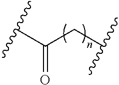 | 5 |
| 1047 | 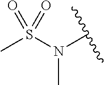 [SMILES: *N(C)S(C)(=O)=O] | 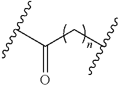 | 6 |
| 1048 | 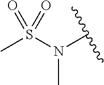 [SMILES: *N(C)S(C)(=O)=O] | 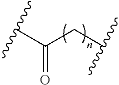 | 7 |
| 1049 | 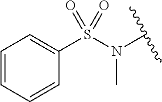 [SMILES: *N(C)S(=O)(=O)c1ccccc1] | 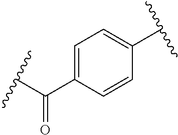 [SMILES: *C(=O)c1ccc(*)cc1] | — |
| 1050 | 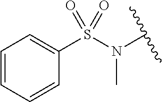 [SMILES: *N(C)S(=O)(=O)c1ccccc1] | 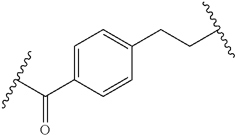 [SMILES: *CCc1ccc(C(*)=O)cc1] | |
| 1051 | 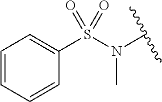 [SMILES: *N(C)S(=O)(=O)c1ccccc1] | 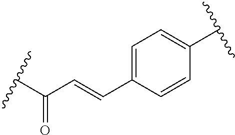 [SMILES: *C(=O)/C=C/c1ccc(*)cc1] | — |
| 1052 | 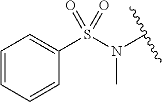 [SMILES: *N(C)S(=O)(=O)c1ccccc1] | 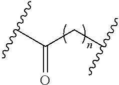 | 5 |
| 1053 | 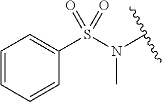 [SMILES: *N(C)S(=O)(=O)c1ccccc1] | 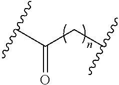 | 6 |
| 1054 | 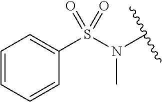 [SMILES: *N(C)S(=O)(=O)c1ccccc1] | 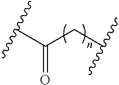 | 7 |
| 1055 | 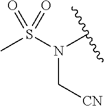 [SMILES: *N(CC#N)S(C)(=O)=O] | 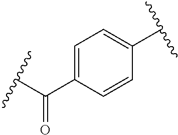 [SMILES: *C(=O)c1ccc(*)cc1] | — |
| 1056 | 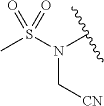 [SMILES: *N(CC#N)S(C)(=O)=O] | 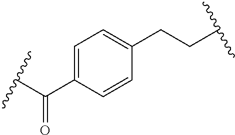 [SMILES: *CCc1ccc(C(*)=O)cc1] | |
| 1057 | 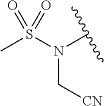 [SMILES: *N(CC#N)S(C)(=O)=O] | 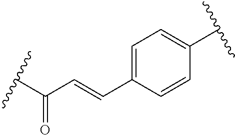 [SMILES: *C(=O)/C=C/c1ccc(*)cc1] | — |
| 1058 | 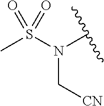 [SMILES: *N(CC#N)S(C)(=O)=O] | 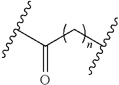 | 5 |
| 1059 | 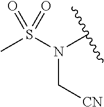 [SMILES: *N(CC#N)S(C)(=O)=O] | 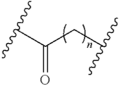 | 6 |
| 1060 | 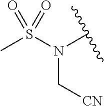 [SMILES: *N(CC#N)S(C)(=O)=O] | 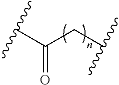 | 7 |
| 1061 | 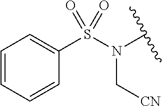 [SMILES: *N(CC#N)S(=O)(=O)c1ccccc1] | 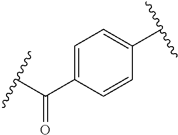 [SMILES: *C(=O)c1ccc(*)cc1] | — |
| 1062 | 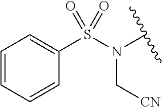 [SMILES: *N(CC#N)S(=O)(=O)c1ccccc1] | 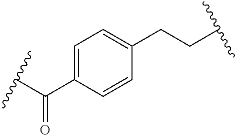 [SMILES: *CCc1ccc(C(*)=O)cc1] | |
| 1063 | 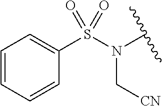 [SMILES: *N(CC#N)S(=O)(=O)c1ccccc1] | 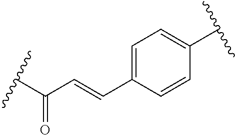 [SMILES: *C(=O)/C=C/c1ccc(*)cc1] | — |
| 1064 | 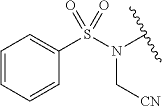 [SMILES: *N(CC#N)S(=O)(=O)c1ccccc1] | 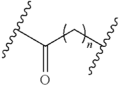 | 5 |
| 1065 | 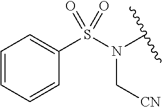 [SMILES: *N(CC#N)S(=O)(=O)c1ccccc1] | 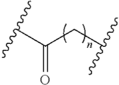 | 6 |
| 1066 | 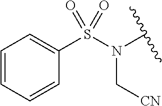 [SMILES: *N(CC#N)S(=O)(=O)c1ccccc1] | 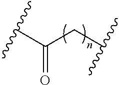 | 7 |
| 1067 | 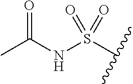 [SMILES: *S(=O)(=O)NC(C)=O] | 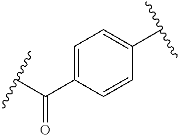 [SMILES: *C(=O)c1ccc(*)cc1] | — |
| 1068 | 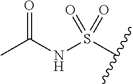 [SMILES: *S(=O)(=O)NC(C)=O] | 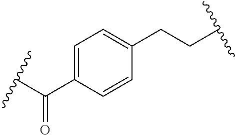 [SMILES: *CCc1ccc(C(*)=O)cc1] | |
| 1069 | 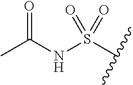 [SMILES: *S(=O)(=O)NC(C)=O] | 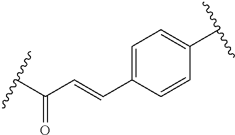 [SMILES: *C(=O)/C=C/c1ccc(*)cc1] | — |
| 1070 | 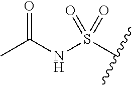 [SMILES: *S(=O)(=O)NC(C)=O] | 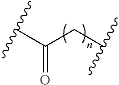 | 5 |
| 1071 | 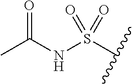 [SMILES: *S(=O)(=O)NC(C)=O] | 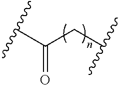 | 6 |
| 1072 | 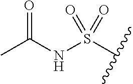 [SMILES: *S(=O)(=O)NC(C)=O] | 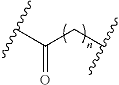 | 7 |
| 1073 | 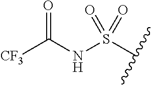 [SMILES: *S(=O)(=O)NC(=O)C(F)(F)F] | 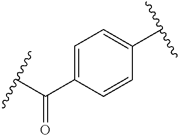 [SMILES: *C(=O)c1ccc(*)cc1] | — |
| 1074 | 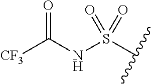 [SMILES: *S(=O)(=O)NC(=O)C(F)(F)F] | 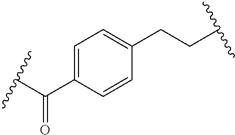 [SMILES: *CCc1ccc(C(*)=O)cc1] | |
| 1075 | 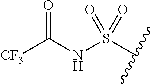 [SMILES: *S(=O)(=O)NC(=O)C(F)(F)F] | 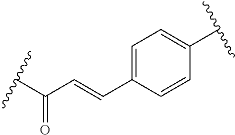 [SMILES: *C(=O)/C=C/c1ccc(*)cc1] | — |
| 1076 | 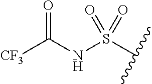 [SMILES: *S(=O)(=O)NC(=O)C(F)(F)F] | 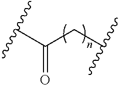 | 5 |
| 1077 | 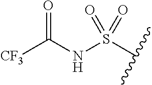 [SMILES: *S(=O)(=O)NC(=O)C(F)(F)F] | 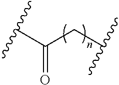 | 6 |
| 1078 | 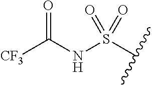 [SMILES: *S(=O)(=O)NC(=O)C(F)(F)F] | 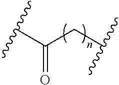 | 7 |
| 1079 | 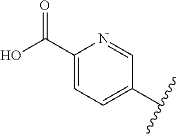 [SMILES: *c1ccc(C(=O)O)nc1] | 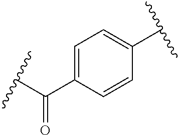 [SMILES: *C(=O)c1ccc(*)cc1] | — |
| 1080 | 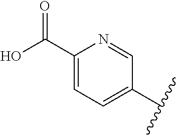 [SMILES: *c1ccc(C(=O)O)nc1] | 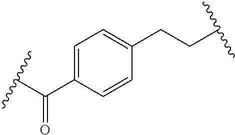 [SMILES: *CCc1ccc(C(*)=O)cc1] | |
| 1081 | 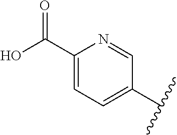 [SMILES: *c1ccc(C(=O)O)nc1] | 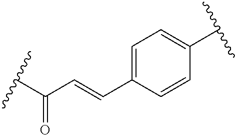 [SMILES: *C(=O)/C=C/c1ccc(*)cc1] | — |
| 1082 | 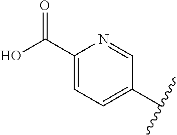 [SMILES: *c1ccc(C(=O)O)nc1] | 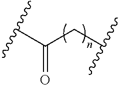 | 5 |
| 1083 | 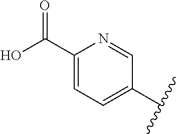 [SMILES: *c1ccc(C(=O)O)nc1] | 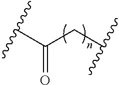 | 6 |
| 1084 | 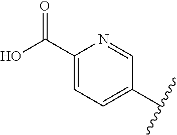 [SMILES: *c1ccc(C(=O)O)nc1] | 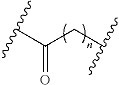 | 7 |
| 1085 | 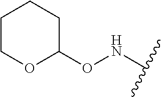 [SMILES: *NOC1CCCCO1] | 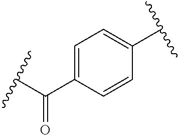 [SMILES: *C(=O)c1ccc(*)cc1] | — |
| 1086 | 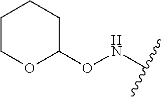 [SMILES: *NOC1CCCCO1] | 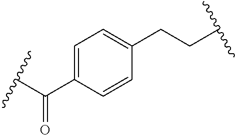 [SMILES: *CCc1ccc(C(*)=O)cc1] | |
| 1087 | 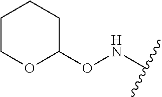 [SMILES: *NOC1CCCCO1] | 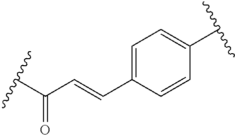 [SMILES: *C(=O)/C=C/c1ccc(*)cc1] | — |
| 1088 | 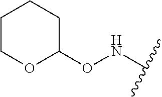 [SMILES: *NOC1CCCCO1] | 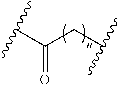 | 5 |
| 1089 | 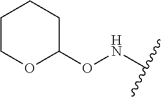 [SMILES: *NOC1CCCCO1] | 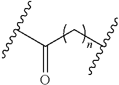 | 6 |
| 1090 | 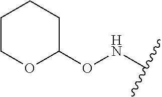 [SMILES: *NOC1CCCCO1] | 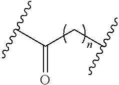 | 7 |
| 1091 | 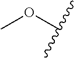 [SMILES: *OC] | 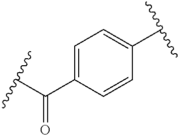 [SMILES: *C(=O)c1ccc(*)cc1] | — |
| 1092 | 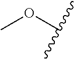 [SMILES: *OC] | 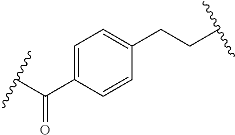 [SMILES: *CCc1ccc(C(*)=O)cc1] | |
| 1093 | 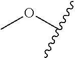 [SMILES: *OC] | 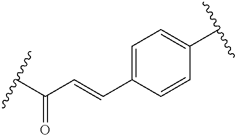 [SMILES: *C(=O)/C=C/c1ccc(*)cc1] | — |
| 1094 | 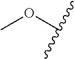 [SMILES: *OC] | 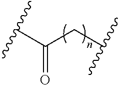 | 5 |
| 1095 | 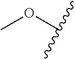 [SMILES: *OC] | 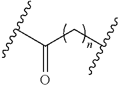 | 6 |
| 1096 | 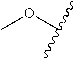 [SMILES: *OC] | 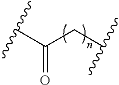 | 7 |
| 1097 | 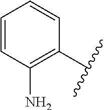 [SMILES: *c1ccccc1N] | 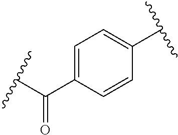 [SMILES: *C(=O)c1ccc(*)cc1] | — |
| 1098 | 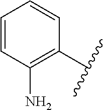 [SMILES: *c1ccccc1N] | 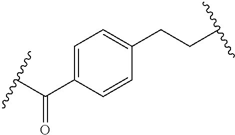 [SMILES: *CCc1ccc(C(*)=O)cc1] | |
| 1099 | 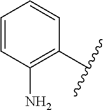 [SMILES: *c1ccccc1N] | 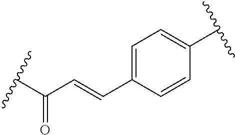 [SMILES: *C(=O)/C=C/c1ccc(*)cc1] | — |
| 1100 | 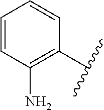 [SMILES: *c1ccccc1N] | 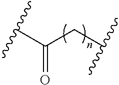 | 5 |
| 1101 | 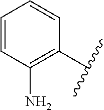 [SMILES: *c1ccccc1N] | 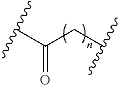 | 6 |
| 1102 | 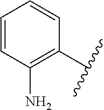 [SMILES: *c1ccccc1N] | 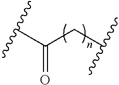 | 7 |
| 1103 | 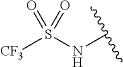 [SMILES: *NS(=O)(=O)C(F)(F)F] | 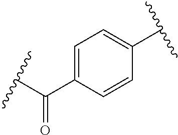 [SMILES: *C(=O)c1ccc(*)cc1] | — |
| 1104 | 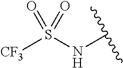 [SMILES: *NS(=O)(=O)C(F)(F)F] | 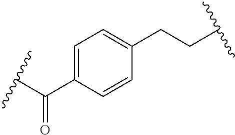 [SMILES: *CCc1ccc(C(*)=O)cc1] | |
| 1105 | 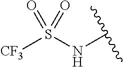 [SMILES: *NS(=O)(=O)C(F)(F)F] | 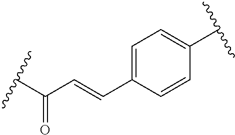 [SMILES: *C(=O)/C=C/c1ccc(*)cc1] | — |
| 1106 | 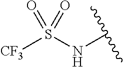 [SMILES: *NS(=O)(=O)C(F)(F)F] | 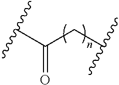 | 5 |
| 1107 | 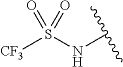 [SMILES: *NS(=O)(=O)C(F)(F)F] | 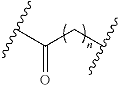 | 6 |
| 1108 | 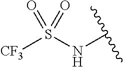 [SMILES: *NS(=O)(=O)C(F)(F)F] | 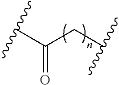 | 7 |
| 1109 | 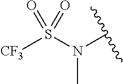 [SMILES: *N(C)S(=O)(=O)C(F)(F)F] | 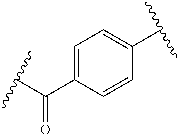 [SMILES: *C(=O)c1ccc(*)cc1] | — |
| 1110 | 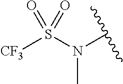 [SMILES: *N(C)S(=O)(=O)C(F)(F)F] | 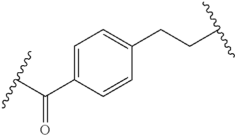 [SMILES: *CCc1ccc(C(*)=O)cc1] | |
| 1111 | 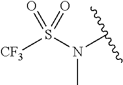 [SMILES: *N(C)S(=O)(=O)C(F)(F)F] | 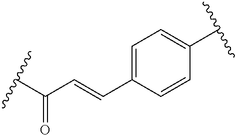 [SMILES: *C(=O)/C=C/c1ccc(*)cc1] | — |
| 1112 | 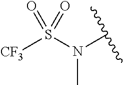 [SMILES: *N(C)S(=O)(=O)C(F)(F)F] | 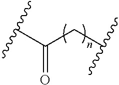 | 5 |
| 1113 | 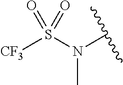 [SMILES: *N(C)S(=O)(=O)C(F)(F)F] | 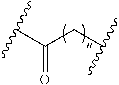 | 6 |
| 1114 | 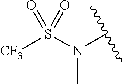 [SMILES: *N(C)S(=O)(=O)C(F)(F)F] | 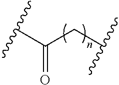 | 7 |

61. The compound of claim 51, wherein the compound of formula (I) is a compound of any one of formula 1115-1228, or a pharmaceutically acceptable salt, solvate, hydrate, cocrystal, or prodrug thereof, wherein in formula 1115-1228:
B is: 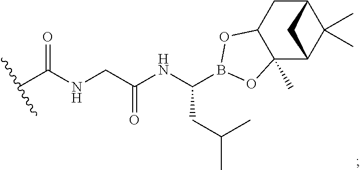 [SMILES: *C(=O)NCC(=O)N[C@@H](CC(C)C)B1OC2C[C@H]3C[C@H](C3(C)C)[C@@]2(C)O1]
and:

| Formula No. | A | L | n |
|---|---|---|---|
| 1115 | 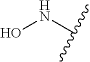 [SMILES: *NO] | 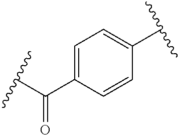 [SMILES: *C(=O)c1ccc(*)cc1] | — |
| 1116 | 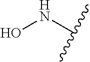 [SMILES: *NO] | 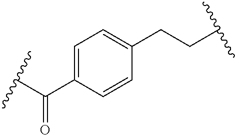 [SMILES: *CCc1ccc(C(*)=O)cc1] | |
| 1117 | 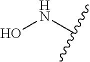 [SMILES: *NO] | 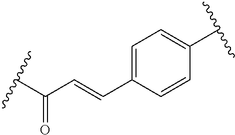 [SMILES: *C(=O)/C=C/c1ccc(*)cc1] | — |
| 1118 | 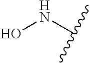 [SMILES: *NO] | 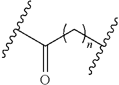 | 5 |
| 1119 | 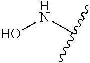 [SMILES: *NO] | 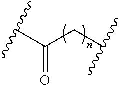 | 6 |
| 1120 | 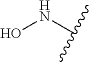 [SMILES: *NO] | 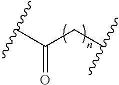 | 7 |
| 1121 | 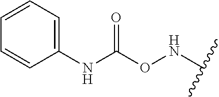 [SMILES: *NOC(=O)Nc1ccccc1] | 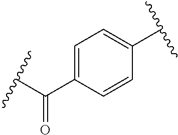 [SMILES: *C(=O)c1ccc(*)cc1] | — |
| 1122 | 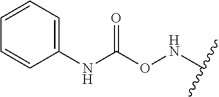 [SMILES: *NOC(=O)Nc1ccccc1] | 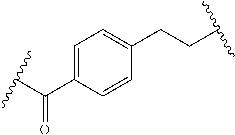 [SMILES: *CCc1ccc(C(*)=O)cc1] | |
| 1123 | 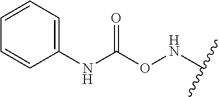 [SMILES: *NOC(=O)Nc1ccccc1] | 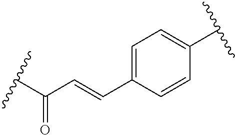 [SMILES: *C(=O)/C=C/c1ccc(*)cc1] | — |
| 1124 | 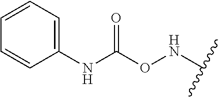 [SMILES: *NOC(=O)Nc1ccccc1] | 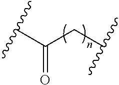 | 5 |
| 1125 | 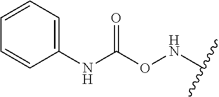 [SMILES: *NOC(=O)Nc1ccccc1] | 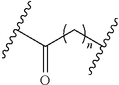 | 6 |
| 1126 | 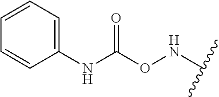 [SMILES: *NOC(=O)Nc1ccccc1] | 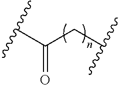 | 7 |
| 1127 | 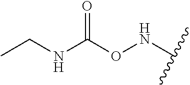 [SMILES: *NOC(=O)NCC] | 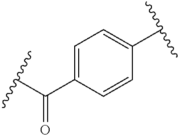 [SMILES: *C(=O)c1ccc(*)cc1] | — |
| 1128 | 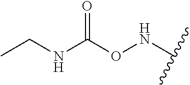 [SMILES: *NOC(=O)NCC] | 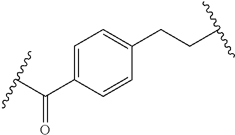 [SMILES: *CCc1ccc(C(*)=O)cc1] | |
| 1129 | 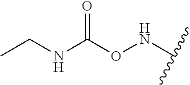 [SMILES: *NOC(=O)NCC] | 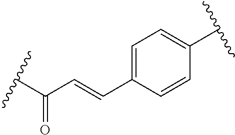 [SMILES: *C(=O)/C=C/c1ccc(*)cc1] | — |
| 1130 | 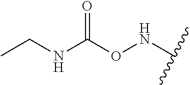 [SMILES: *NOC(=O)NCC] | 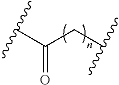 | 5 |
| 1131 | 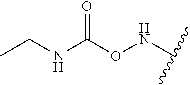 [SMILES: *NOC(=O)NCC] | 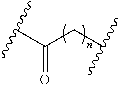 | 6 |
| 1132 | 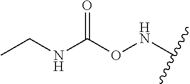 [SMILES: *NOC(=O)NCC] | 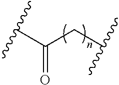 | 7 |
| 1133 | 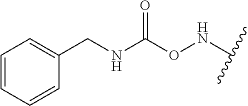 [SMILES: *NOC(=O)NCc1ccccc1] | 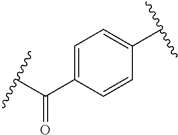 [SMILES: *C(=O)c1ccc(*)cc1] | — |
| 1134 | 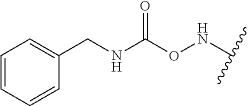 [SMILES: *NOC(=O)NCc1ccccc1] | 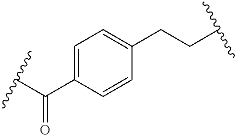 [SMILES: *CCc1ccc(C(*)=O)cc1] | |
| 1135 | 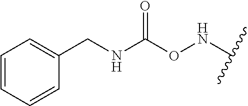 [SMILES: *NOC(=O)NCc1ccccc1] | 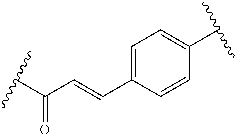 [SMILES: *C(=O)/C=C/c1ccc(*)cc1] | — |
| 1136 | 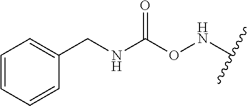 [SMILES: *NOC(=O)NCc1ccccc1] | 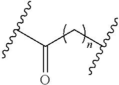 | 5 |
| 1137 | 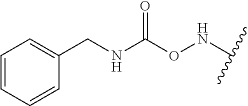 [SMILES: *NOC(=O)NCc1ccccc1] | 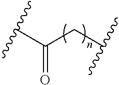 | 6 |
| 1138 | 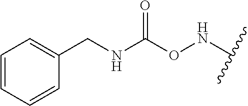 [SMILES: *NOC(=O)NCc1ccccc1] | 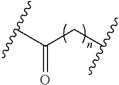 | 7 |
| 1139 | 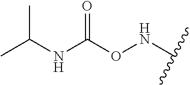 [SMILES: *NOC(=O)NC(C)C] | 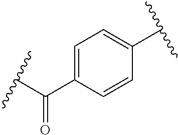 [SMILES: *C(=O)c1ccc(*)cc1] | — |
| 1140 | 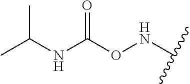 [SMILES: *NOC(=O)NC(C)C] | 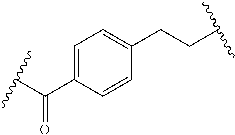 [SMILES: *CCc1ccc(C(*)=O)cc1] | |
| 1141 | 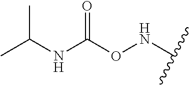 [SMILES: *NOC(=O)NC(C)C] | 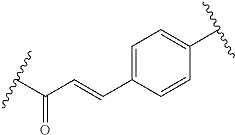 [SMILES: *C(=O)/C=C/c1ccc(*)cc1] | — |
| 1142 | 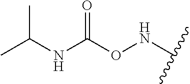 [SMILES: *NOC(=O)NC(C)C] | 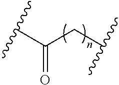 | 5 |
| 1143 | 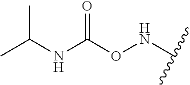 [SMILES: *NOC(=O)NC(C)C] | 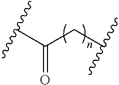 | 6 |
| 1144 | 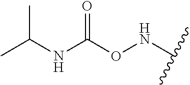 [SMILES: *NOC(=O)NC(C)C] | 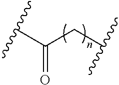 | 7 |
| 1145 | 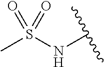 [SMILES: *NS(C)(=O)=O] | 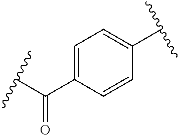 [SMILES: *C(=O)c1ccc(*)cc1] | — |
| 1146 | 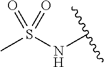 [SMILES: *NS(C)(=O)=O] | 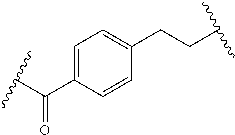 [SMILES: *CCc1ccc(C(*)=O)cc1] | |
| 1147 | 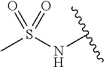 [SMILES: *NS(C)(=O)=O] | 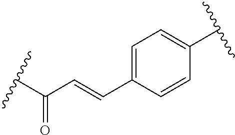 [SMILES: *C(=O)/C=C/c1ccc(*)cc1] | — |
| 1148 | 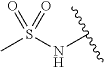 [SMILES: *NS(C)(=O)=O] | 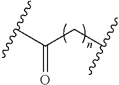 | 5 |
| 1149 | 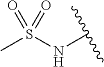 [SMILES: *NS(C)(=O)=O] | 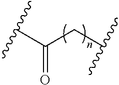 | 6 |
| 1150 | 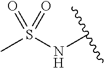 [SMILES: *NS(C)(=O)=O] | 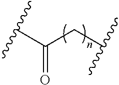 | 7 |
| 1151 | 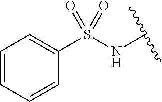 [SMILES: *NS(=O)(=O)c1ccccc1] | 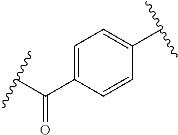 [SMILES: *C(=O)c1ccc(*)cc1] | — |
| 1152 | 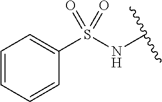 [SMILES: *NS(=O)(=O)c1ccccc1] | 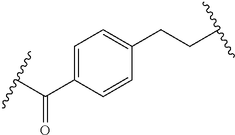 [SMILES: *CCc1ccc(C(*)=O)cc1] | |
| 1153 | 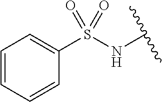 [SMILES: *NS(=O)(=O)c1ccccc1] | 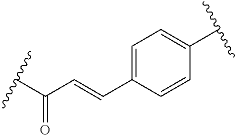 [SMILES: *C(=O)/C=C/c1ccc(*)cc1] | — |
| 1154 | 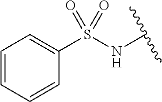 [SMILES: *NS(=O)(=O)c1ccccc1] | 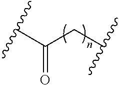 | 5 |
| 1155 | 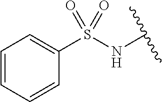 [SMILES: *NS(=O)(=O)c1ccccc1] | 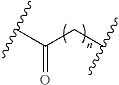 | 6 |
| 1156 | 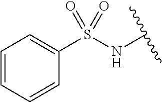 [SMILES: *NS(=O)(=O)c1ccccc1] | 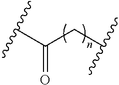 | 7 |
| 1157 | 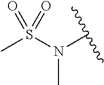 [SMILES: *N(C)S(C)(=O)=O] | 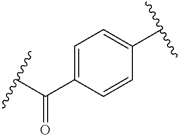 [SMILES: *C(=O)c1ccc(*)cc1] | — |
| 1158 | 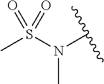 [SMILES: *N(C)S(C)(=O)=O] | 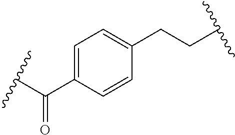 [SMILES: *CCc1ccc(C(*)=O)cc1] | |
| 1159 | 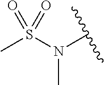 [SMILES: *N(C)S(C)(=O)=O] | 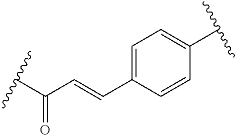 [SMILES: *C(=O)/C=C/c1ccc(*)cc1] | — |
| 1160 | 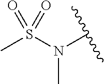 [SMILES: *N(C)S(C)(=O)=O] | 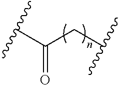 | 5 |
| 1161 | 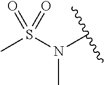 [SMILES: *N(C)S(C)(=O)=O] | 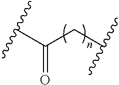 | 6 |
| 1162 | 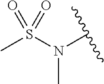 [SMILES: *N(C)S(C)(=O)=O] | 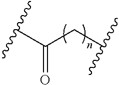 | 7 |
| 1163 | 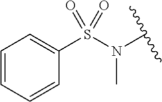 [SMILES: *N(C)S(=O)(=O)c1ccccc1] | 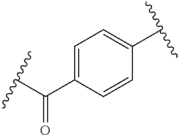 [SMILES: *C(=O)c1ccc(*)cc1] | — |
| 1164 | 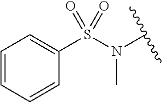 [SMILES: *N(C)S(=O)(=O)c1ccccc1] | 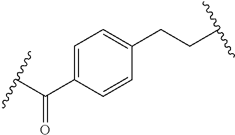 [SMILES: *CCc1ccc(C(*)=O)cc1] | |
| 1165 | 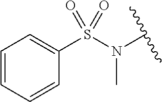 [SMILES: *N(C)S(=O)(=O)c1ccccc1] | 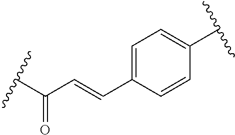 [SMILES: *C(=O)/C=C/c1ccc(*)cc1] | — |
| 1166 | 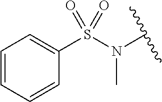 [SMILES: *N(C)S(=O)(=O)c1ccccc1] | 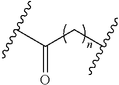 | 5 |
| 1167 | 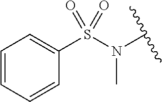 [SMILES: *N(C)S(=O)(=O)c1ccccc1] | 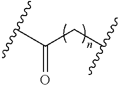 | 6 |
| 1168 | 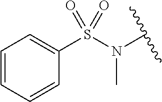 [SMILES: *N(C)S(=O)(=O)c1ccccc1] | 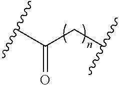 | 7 |
| 1169 | 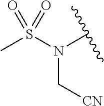 [SMILES: *N(CC#N)S(C)(=O)=O] | 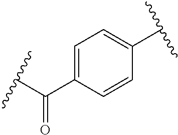 [SMILES: *C(=O)c1ccc(*)cc1] | — |
| 1170 | 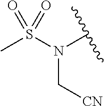 [SMILES: *N(CC#N)S(C)(=O)=O] | 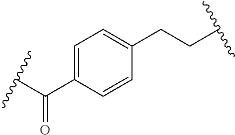 [SMILES: *CCc1ccc(C(*)=O)cc1] | |
| 1171 | 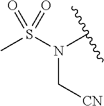 [SMILES: *N(CC#N)S(C)(=O)=O] | 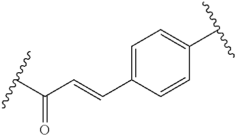 [SMILES: *C(=O)/C=C/c1ccc(*)cc1] | — |
| 1172 | 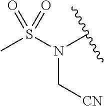 [SMILES: *N(CC#N)S(C)(=O)=O] | 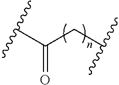 | 5 |
| 1173 | 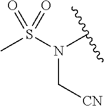 [SMILES: *N(CC#N)S(C)(=O)=O] | 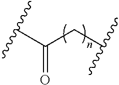 | 6 |
| 1174 | 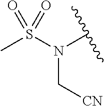 [SMILES: *N(CC#N)S(C)(=O)=O] | 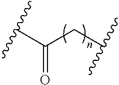 | 7 |
| 1175 | 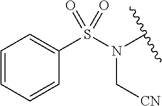 [SMILES: *N(CC#N)S(=O)(=O)c1ccccc1] | 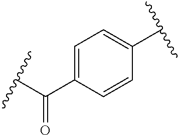 [SMILES: *C(=O)c1ccc(*)cc1] | — |
| 1176 | 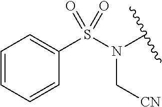 [SMILES: *N(CC#N)S(=O)(=O)c1ccccc1] | 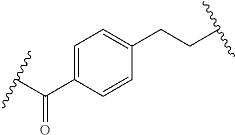 [SMILES: *CCc1ccc(C(*)=O)cc1] | |
| 1177 | 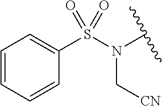 [SMILES: *N(CC#N)S(=O)(=O)c1ccccc1] | 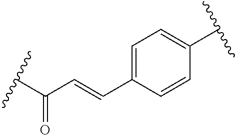 [SMILES: *C(=O)/C=C/c1ccc(*)cc1] | — |
| 1178 | 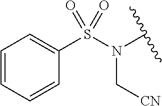 [SMILES: *N(CC#N)S(=O)(=O)c1ccccc1] | 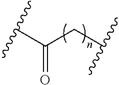 | 5 |
| 1179 | 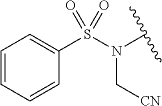 [SMILES: *N(CC#N)S(=O)(=O)c1ccccc1] | 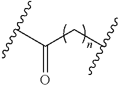 | 6 |
| 1180 | 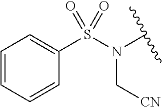 [SMILES: *N(CC#N)S(=O)(=O)c1ccccc1] | 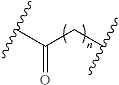 | 7 |
| 1181 | 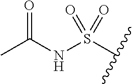 [SMILES: *S(=O)(=O)NC(C)=O] | 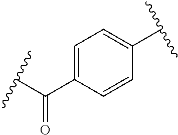 [SMILES: *C(=O)c1ccc(*)cc1] | — |
| 1182 | 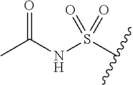 [SMILES: *S(=O)(=O)NC(C)=O] | 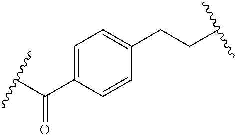 [SMILES: *CCc1ccc(C(*)=O)cc1] | |
| 1183 | 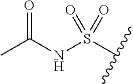 [SMILES: *S(=O)(=O)NC(C)=O] | 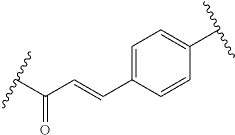 [SMILES: *C(=O)/C=C/c1ccc(*)cc1] | — |
| 1184 | 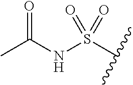 [SMILES: *S(=O)(=O)NC(C)=O] | 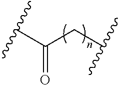 | 5 |
| 1185 | 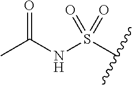 [SMILES: *S(=O)(=O)NC(C)=O] | 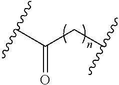 | 6 |
| 1186 | 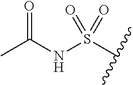 [SMILES: *S(=O)(=O)NC(C)=O] | 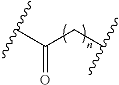 | 7 |
| 1187 | 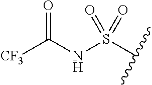 [SMILES: *S(=O)(=O)NC(=O)C(F)(F)F] | 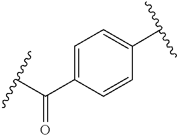 [SMILES: *C(=O)c1ccc(*)cc1] | — |
| 1188 | 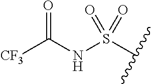 [SMILES: *S(=O)(=O)NC(=O)C(F)(F)F] | 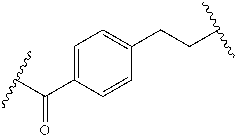 [SMILES: *CCc1ccc(C(*)=O)cc1] | |
| 1189 | 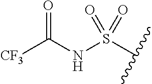 [SMILES: *S(=O)(=O)NC(=O)C(F)(F)F] | 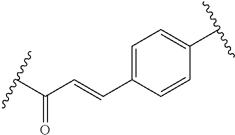 [SMILES: *C(=O)/C=C/c1ccc(*)cc1] | — |
| 1190 | 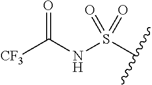 [SMILES: *S(=O)(=O)NC(=O)C(F)(F)F] | 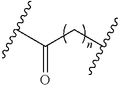 | 5 |
| 1191 | 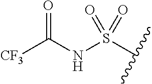 [SMILES: *S(=O)(=O)NC(=O)C(F)(F)F] | 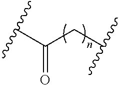 | 6 |
| 1192 | 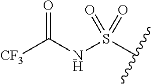 [SMILES: *S(=O)(=O)NC(=O)C(F)(F)F] | 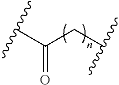 | 7 |
| 1193 | 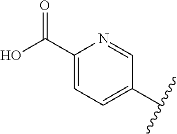 [SMILES: *c1ccc(C(=O)O)nc1] | 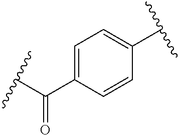 [SMILES: *C(=O)c1ccc(*)cc1] | — |
| 1194 | 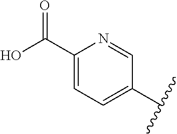 [SMILES: *c1ccc(C(=O)O)nc1] | 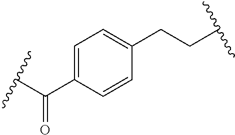 [SMILES: *CCc1ccc(C(*)=O)cc1] | |
| 1195 | 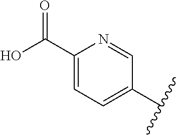 [SMILES: *c1ccc(C(=O)O)nc1] | 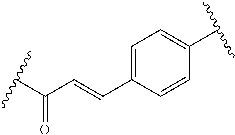 [SMILES: *C(=O)/C=C/c1ccc(*)cc1] | — |
| 1196 | 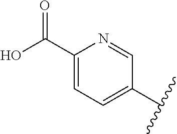 [SMILES: *c1ccc(C(=O)O)nc1] | 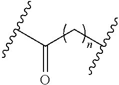 | 5 |
| 1197 | 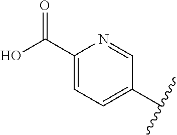 [SMILES: *c1ccc(C(=O)O)nc1] | 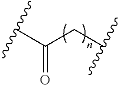 | 6 |
| 1198 | 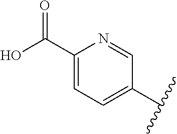 [SMILES: *c1ccc(C(=O)O)nc1] | 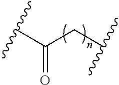 | 7 |
| 1199 | 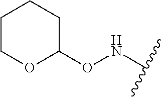 [SMILES: *NOC1CCCCO1] | 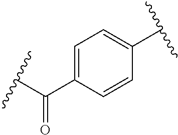 [SMILES: *C(=O)c1ccc(*)cc1] | — |
| 1200 | 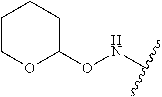 [SMILES: *NOC1CCCCO1] | 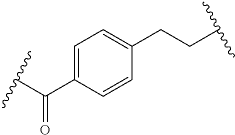 [SMILES: *CCc1ccc(C(*)=O)cc1] | |
| 1201 | 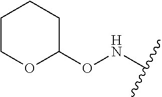 [SMILES: *NOC1CCCCO1] | 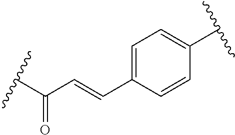 [SMILES: *C(=O)/C=C/c1ccc(*)cc1] | — |
| 1202 | 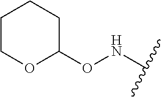 [SMILES: *NOC1CCCCO1] | 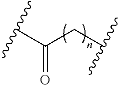 | 5 |
| 1203 | 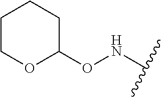 [SMILES: *NOC1CCCCO1] | 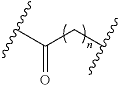 | 6 |
| 1204 | 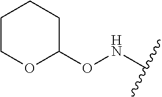 [SMILES: *NOC1CCCCO1] | 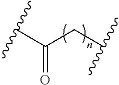 | 7 |
| 1205 | 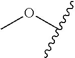 [SMILES: *OC] | 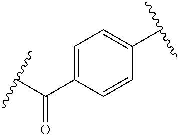 [SMILES: *C(=O)c1ccc(*)cc1] | — |
| 1206 | 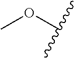 [SMILES: *OC] | 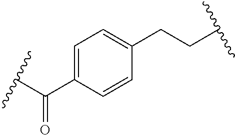 [SMILES: *CCc1ccc(C(*)=O)cc1] | |
| 1207 | 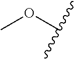 [SMILES: *OC] | 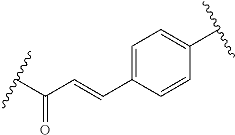 [SMILES: *C(=O)/C=C/c1ccc(*)cc1] | — |
| 1208 | 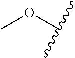 [SMILES: *OC] | 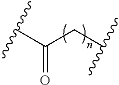 | 5 |
| 1209 | 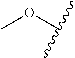 [SMILES: *OC] | 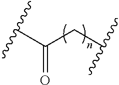 | 6 |
| 1210 | 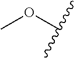 [SMILES: *OC] | 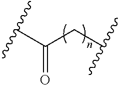 | 7 |
| 1211 | 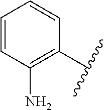 [SMILES: *c1ccccc1N] | 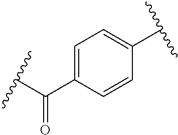 [SMILES: *C(=O)c1ccc(*)cc1] | — |
| 1212 | 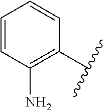 [SMILES: *c1ccccc1N] | 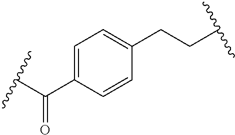 [SMILES: *CCc1ccc(C(*)=O)cc1] | |
| 1213 | 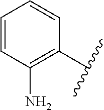 [SMILES: *c1ccccc1N] | 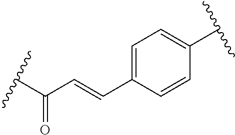 [SMILES: *C(=O)/C=C/c1ccc(*)cc1] | — |
| 1214 | 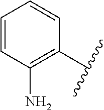 [SMILES: *c1ccccc1N] | 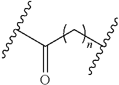 | 5 |
| 1215 | 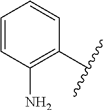 [SMILES: *c1ccccc1N] | 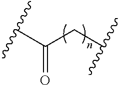 | 6 |
| 1216 | 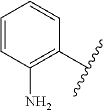 [SMILES: *c1ccccc1N] | 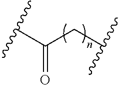 | 7 |
| 1217 | 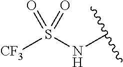 [SMILES: *NS(=O)(=O)C(F)(F)F] | 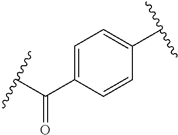 [SMILES: *C(=O)c1ccc(*)cc1] | — |
| 1218 | 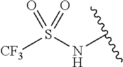 [SMILES: *NS(=O)(=O)C(F)(F)F] | 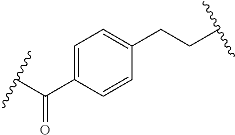 [SMILES: *CCc1ccc(C(*)=O)cc1] | |
| 1219 | 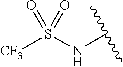 [SMILES: *NS(=O)(=O)C(F)(F)F] | 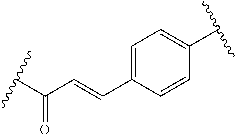 [SMILES: *C(=O)/C=C/c1ccc(*)cc1] | — |
| 1220 | 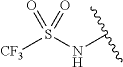 [SMILES: *NS(=O)(=O)C(F)(F)F] | 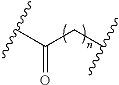 | 5 |
| 1221 | 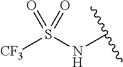 [SMILES: *NS(=O)(=O)C(F)(F)F] | 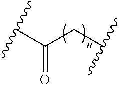 | 6 |
| 1222 | 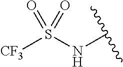 [SMILES: *NS(=O)(=O)C(F)(F)F] | 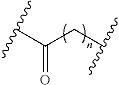 | 7 |
| 1223 | 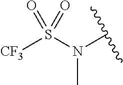 [SMILES: *N(C)S(=O)(=O)C(F)(F)F] | 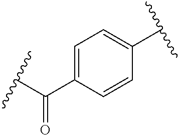 [SMILES: *C(=O)c1ccc(*)cc1] | — |
| 1224 | 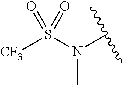 [SMILES: *N(C)S(=O)(=O)C(F)(F)F] | 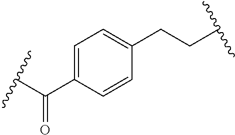 [SMILES: *CCc1ccc(C(*)=O)cc1] | |
| 1225 | 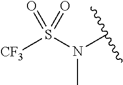 [SMILES: *N(C)S(=O)(=O)C(F)(F)F] | 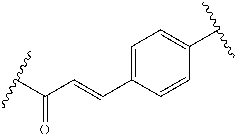 [SMILES: *C(=O)/C=C/c1ccc(*)cc1] | — |
| 1226 | 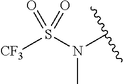 [SMILES: *N(C)S(=O)(=O)C(F)(F)F] | 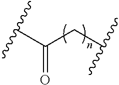 | 5 |
| 1227 | 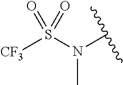 [SMILES: *N(C)S(=O)(=O)C(F)(F)F] | 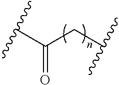 | 6 |
| 1228 | 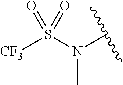 [SMILES: *N(C)S(=O)(=O)C(F)(F)F] | 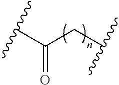 | 7 |

62. The compound of claim 51, wherein the compound of formula (I) is a compound ofany one of formula 1229-1342, or a pharmaceutically acceptable salt, solvate, hydrate, cocrystal, or prodrug thereof, wherein in formula 1229-1342:
B is: 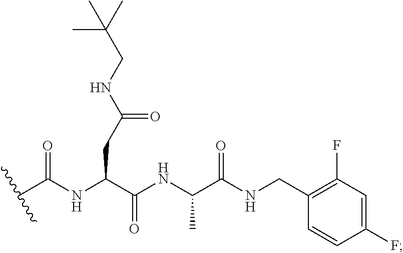 [SMILES: *C(=O)N[C@@H](CC(=O)NCC(C)(C)C)C(=O)N[C@@H](C)C(=O)NCc1ccc(F)cc1F]
and:

| Formula No. | A | L | n |
|---|---|---|---|
| 1229 | 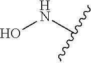 [SMILES: *NO] | 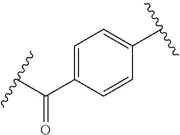 [SMILES: *C(=O)c1ccc(*)cc1] | — |
| 1230 | 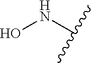 [SMILES: *NO] | 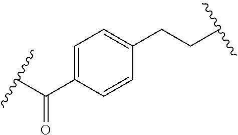 [SMILES: *CCc1ccc(C(*)=O)cc1] | |
| 1231 | 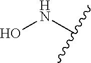 [SMILES: *NO] | 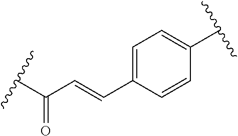 [SMILES: *C(=O)/C=C/c1ccc(*)cc1] | — |
| 1232 | 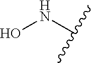 [SMILES: *NO] | 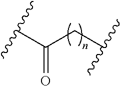 | 5 |
| 1233 | 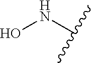 [SMILES: *NO] | 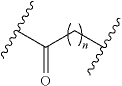 | 6 |
| 1234 | 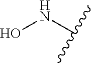 [SMILES: *NO] | 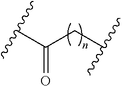 | 7 |
| 1235 | 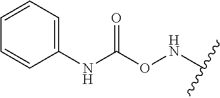 [SMILES: *NOC(=O)Nc1ccccc1] | 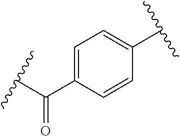 [SMILES: *C(=O)c1ccc(*)cc1] | — |
| 1236 | 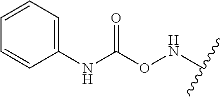 [SMILES: *NOC(=O)Nc1ccccc1] | 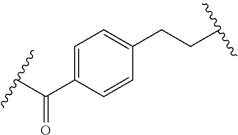 [SMILES: *CCc1ccc(C(*)=O)cc1] | |
| 1237 | 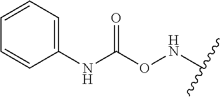 [SMILES: *NOC(=O)Nc1ccccc1] | 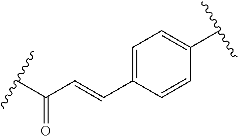 [SMILES: *C(=O)/C=C/c1ccc(*)cc1] | — |
| 1238 | 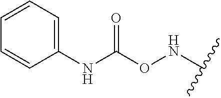 [SMILES: *NOC(=O)Nc1ccccc1] | 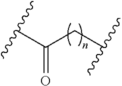 | 5 |
| 1239 | 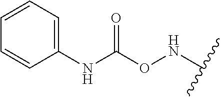 [SMILES: *NOC(=O)Nc1ccccc1] | 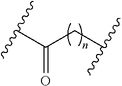 | 6 |
| 1240 | 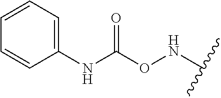 [SMILES: *NOC(=O)Nc1ccccc1] | 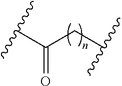 | 7 |
| 1241 | 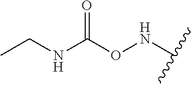 [SMILES: *NOC(=O)NCC] | 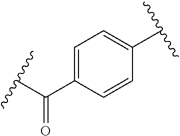 [SMILES: *C(=O)c1ccc(*)cc1] | — |
| 1242 | 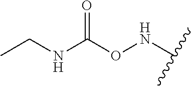 [SMILES: *NOC(=O)NCC] | 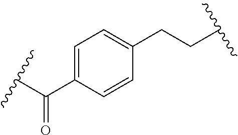 [SMILES: *CCc1ccc(C(*)=O)cc1] | |
| 1243 | 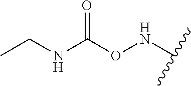 [SMILES: *NOC(=O)NCC] | 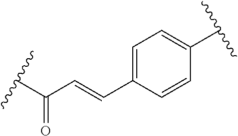 [SMILES: *C(=O)/C=C/c1ccc(*)cc1] | — |
| 1244 | 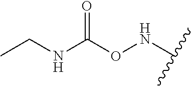 [SMILES: *NOC(=O)NCC] | 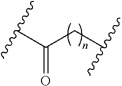 | 5 |
| 1245 | 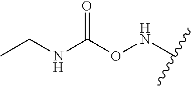 [SMILES: *NOC(=O)NCC] | 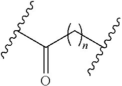 | 6 |
| 1246 | 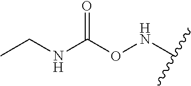 [SMILES: *NOC(=O)NCC] | 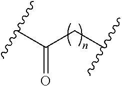 | 7 |
| 1247 | 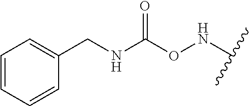 [SMILES: *NOC(=O)NCc1ccccc1] | 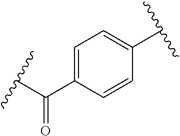 [SMILES: *C(=O)c1ccc(*)cc1] | — |
| 1248 | 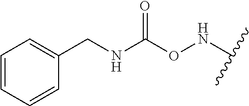 [SMILES: *NOC(=O)NCc1ccccc1] | 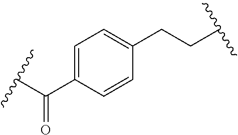 [SMILES: *CCc1ccc(C(*)=O)cc1] | |
| 1249 | 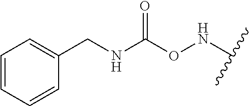 [SMILES: *NOC(=O)NCc1ccccc1] | 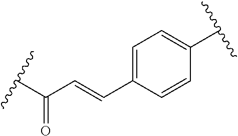 [SMILES: *C(=O)/C=C/c1ccc(*)cc1] | — |
| 1250 | 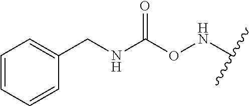 [SMILES: *NOC(=O)NCc1ccccc1] | 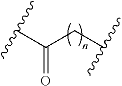 | 5 |
| 1251 | 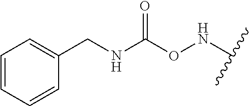 [SMILES: *NOC(=O)NCc1ccccc1] | 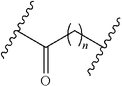 | 6 |
| 1252 | 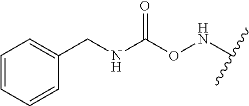 [SMILES: *NOC(=O)NCc1ccccc1] | 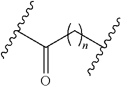 | 7 |
| 1253 | 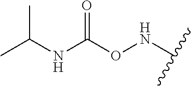 [SMILES: *NOC(=O)NC(C)C] | 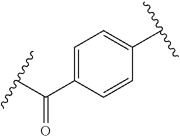 [SMILES: *C(=O)c1ccc(*)cc1] | — |
| 1254 | 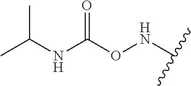 [SMILES: *NOC(=O)NC(C)C] | 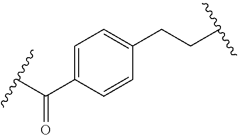 [SMILES: *CCc1ccc(C(*)=O)cc1] | |
| 1255 | 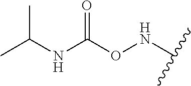 [SMILES: *NOC(=O)NC(C)C] | 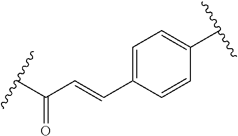 [SMILES: *C(=O)/C=C/c1ccc(*)cc1] | — |
| 1256 | 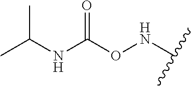 [SMILES: *NOC(=O)NC(C)C] | 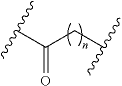 | 5 |
| 1257 | 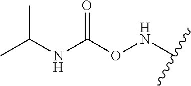 [SMILES: *NOC(=O)NC(C)C] | 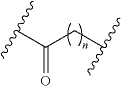 | 6 |
| 1258 | 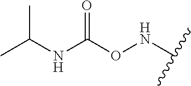 [SMILES: *NOC(=O)NC(C)C] | 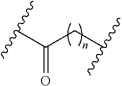 | 7 |
| 1259 | 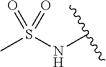 [SMILES: *NS(C)(=O)=O] | 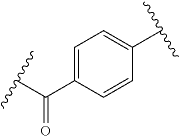 [SMILES: *C(=O)c1ccc(*)cc1] | — |
| 1260 | 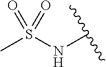 [SMILES: *NS(C)(=O)=O] | 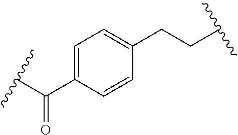 [SMILES: *CCc1ccc(C(*)=O)cc1] | |
| 1261 | 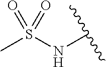 [SMILES: *NS(C)(=O)=O] | 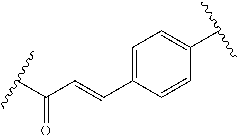 [SMILES: *C(=O)/C=C/c1ccc(*)cc1] | — |
| 1262 | 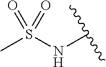 [SMILES: *NS(C)(=O)=O] | 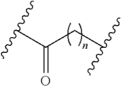 | 5 |
| 1263 | 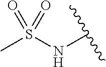 [SMILES: *NS(C)(=O)=O] | 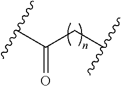 | 6 |
| 1264 | 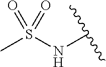 [SMILES: *NS(C)(=O)=O] | 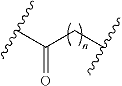 | 7 |
| 1265 | 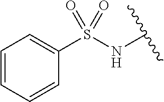 [SMILES: *NS(=O)(=O)c1ccccc1] | 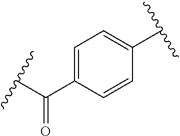 [SMILES: *C(=O)c1ccc(*)cc1] | — |
| 1266 | 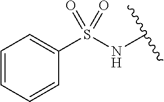 [SMILES: *NS(=O)(=O)c1ccccc1] | 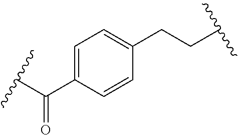 [SMILES: *CCc1ccc(C(*)=O)cc1] | |
| 1267 | 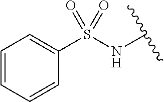 [SMILES: *NS(=O)(=O)c1ccccc1] | 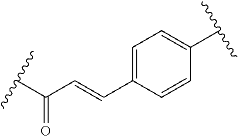 [SMILES: *C(=O)/C=C/c1ccc(*)cc1] | — |
| 1268 | 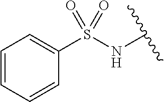 [SMILES: *NS(=O)(=O)c1ccccc1] | 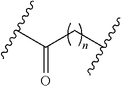 | 5 |
| 1269 | 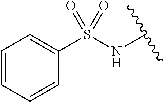 [SMILES: *NS(=O)(=O)c1ccccc1] | 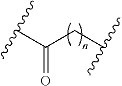 | 6 |
| 1270 | 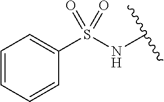 [SMILES: *NS(=O)(=O)c1ccccc1] | 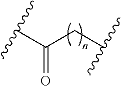 | 7 |
| 1271 | 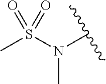 [SMILES: *N(C)S(C)(=O)=O] | 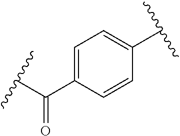 [SMILES: *C(=O)c1ccc(*)cc1] | — |
| 1272 | 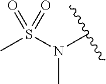 [SMILES: *N(C)S(C)(=O)=O] | 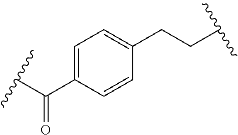 [SMILES: *CCc1ccc(C(*)=O)cc1] | |
| 1273 | 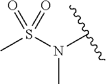 [SMILES: *N(C)S(C)(=O)=O] | 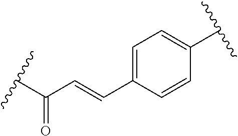 [SMILES: *C(=O)/C=C/c1ccc(*)cc1] | — |
| 1274 | 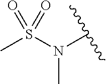 [SMILES: *N(C)S(C)(=O)=O] | 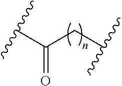 | 5 |
| 1275 | 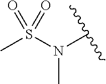 [SMILES: *N(C)S(C)(=O)=O] | 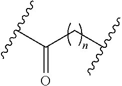 | 6 |
| 1276 | 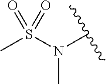 [SMILES: *N(C)S(C)(=O)=O] | 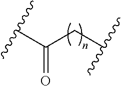 | 7 |
| 1277 | 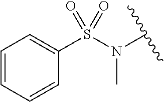 [SMILES: *N(C)S(=O)(=O)c1ccccc1] | 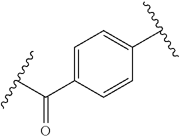 [SMILES: *C(=O)c1ccc(*)cc1] | — |
| 1278 | 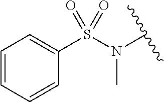 [SMILES: *N(C)S(=O)(=O)c1ccccc1] | 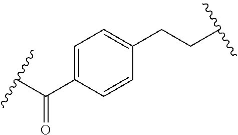 [SMILES: *CCc1ccc(C(*)=O)cc1] | |
| 1279 | 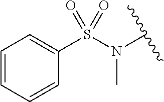 [SMILES: *N(C)S(=O)(=O)c1ccccc1] | 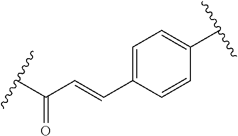 [SMILES: *C(=O)/C=C/c1ccc(*)cc1] | — |
| 1280 | 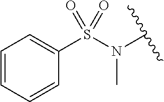 [SMILES: *N(C)S(=O)(=O)c1ccccc1] | 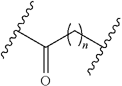 | 5 |
| 1281 | 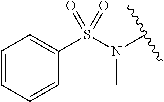 [SMILES: *N(C)S(=O)(=O)c1ccccc1] | 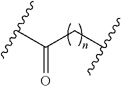 | 6 |
| 1282 | 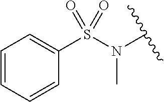 [SMILES: *N(C)S(=O)(=O)c1ccccc1] | 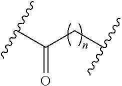 | 7 |
| 1283 | 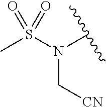 [SMILES: *N(CC#N)S(C)(=O)=O] | 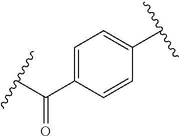 [SMILES: *C(=O)c1ccc(*)cc1] | — |
| 1284 | 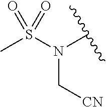 [SMILES: *N(CC#N)S(C)(=O)=O] | 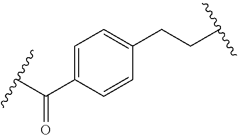 [SMILES: *CCc1ccc(C(*)=O)cc1] | |
| 1285 | 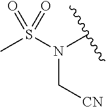 [SMILES: *N(CC#N)S(C)(=O)=O] | 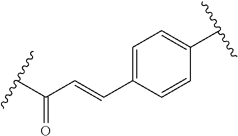 [SMILES: *C(=O)/C=C/c1ccc(*)cc1] | — |
| 1286 | 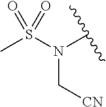 [SMILES: *N(CC#N)S(C)(=O)=O] | 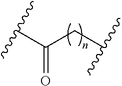 | 5 |
| 1287 | 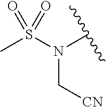 [SMILES: *N(CC#N)S(C)(=O)=O] | 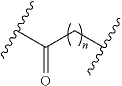 | 6 |
| 1288 | 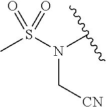 [SMILES: *N(CC#N)S(C)(=O)=O] | 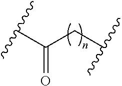 | 7 |
| 1289 | 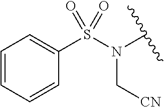 [SMILES: *N(CC#N)S(=O)(=O)c1ccccc1] | 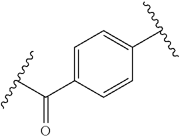 [SMILES: *C(=O)c1ccc(*)cc1] | — |
| 1290 | 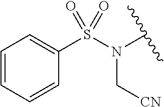 [SMILES: *N(CC#N)S(=O)(=O)c1ccccc1] | 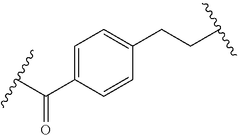 [SMILES: *CCc1ccc(C(*)=O)cc1] | |
| 1291 | 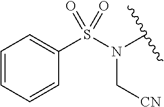 [SMILES: *N(CC#N)S(=O)(=O)c1ccccc1] | 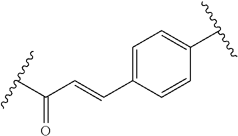 [SMILES: *C(=O)/C=C/c1ccc(*)cc1] | — |
| 1292 | 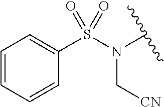 [SMILES: *N(CC#N)S(=O)(=O)c1ccccc1] | 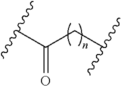 | 5 |
| 1293 | 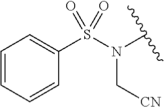 [SMILES: *N(CC#N)S(=O)(=O)c1ccccc1] | 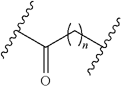 | 6 |
| 1294 | 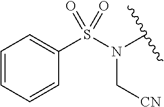 [SMILES: *N(CC#N)S(=O)(=O)c1ccccc1] | 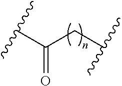 | 7 |
| 1295 | 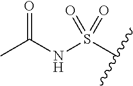 [SMILES: *S(=O)(=O)NC(C)=O] | 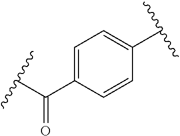 [SMILES: *C(=O)c1ccc(*)cc1] | — |
| 1296 | 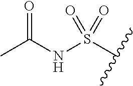 [SMILES: *S(=O)(=O)NC(C)=O] | 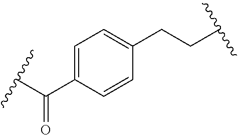 [SMILES: *CCc1ccc(C(*)=O)cc1] | |
| 1297 | 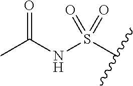 [SMILES: *S(=O)(=O)NC(C)=O] | 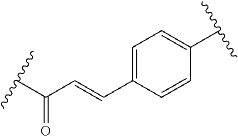 [SMILES: *C(=O)/C=C/c1ccc(*)cc1] | — |
| 1298 | 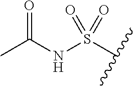 [SMILES: *S(=O)(=O)NC(C)=O] | 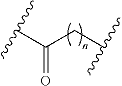 | 5 |
| 1299 | 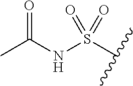 [SMILES: *S(=O)(=O)NC(C)=O] | 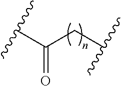 | 6 |
| 1300 | 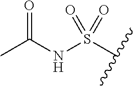 [SMILES: *S(=O)(=O)NC(C)=O] | 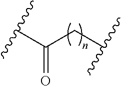 | 7 |
| 1301 | 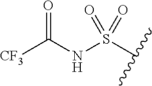 [SMILES: *S(=O)(=O)NC(=O)C(F)(F)F] | 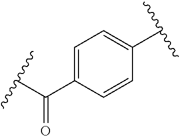 [SMILES: *C(=O)c1ccc(*)cc1] | — |
| 1302 | 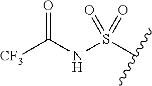 [SMILES: *S(=O)(=O)NC(=O)C(F)(F)F] | 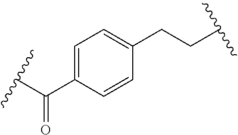 [SMILES: *CCc1ccc(C(*)=O)cc1] | |
| 1303 | 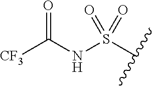 [SMILES: *S(=O)(=O)NC(=O)C(F)(F)F] | 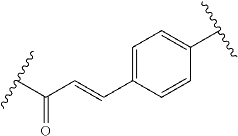 [SMILES: *C(=O)/C=C/c1ccc(*)cc1] | — |
| 1304 | 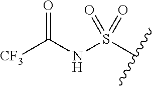 [SMILES: *S(=O)(=O)NC(=O)C(F)(F)F] | 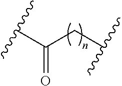 | 5 |
| 1305 | 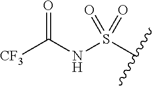 [SMILES: *S(=O)(=O)NC(=O)C(F)(F)F] | 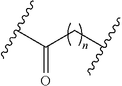 | 6 |
| 1306 | 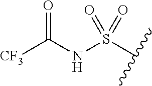 [SMILES: *S(=O)(=O)NC(=O)C(F)(F)F] | 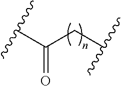 | 7 |
| 1307 | 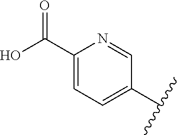 [SMILES: *c1ccc(C(=O)O)nc1] | 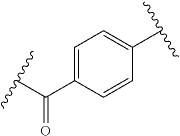 [SMILES: *C(=O)c1ccc(*)cc1] | — |
| 1308 | 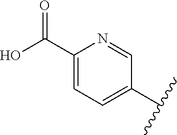 [SMILES: *c1ccc(C(=O)O)nc1] | 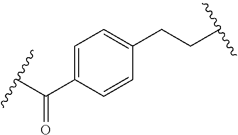 [SMILES: *CCc1ccc(C(*)=O)cc1] | |
| 1309 | 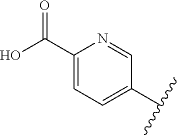 [SMILES: *c1ccc(C(=O)O)nc1] | 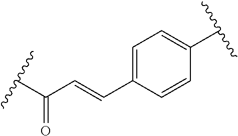 [SMILES: *C(=O)/C=C/c1ccc(*)cc1] | — |
| 1310 | 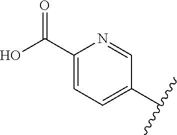 [SMILES: *c1ccc(C(=O)O)nc1] | 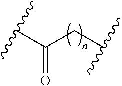 | 5 |
| 1311 | 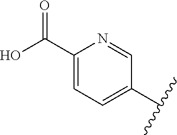 [SMILES: *c1ccc(C(=O)O)nc1] | 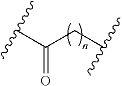 | 6 |
| 1312 | 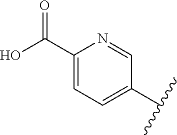 [SMILES: *c1ccc(C(=O)O)nc1] | 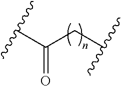 | 7 |
| 1313 | 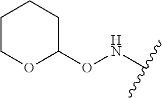 [SMILES: *NOC1CCCCO1] | 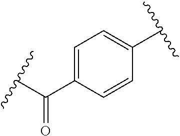 [SMILES: *C(=O)c1ccc(*)cc1] | — |
| 1314 | 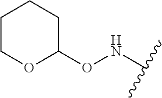 [SMILES: *NOC1CCCCO1] | 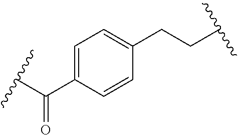 [SMILES: *CCc1ccc(C(*)=O)cc1] | |
| 1315 | 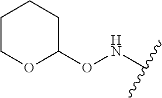 [SMILES: *NOC1CCCCO1] | 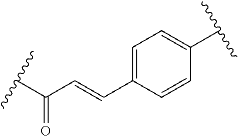 [SMILES: *C(=O)/C=C/c1ccc(*)cc1] | — |
| 1316 | 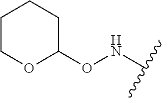 [SMILES: *NOC1CCCCO1] | 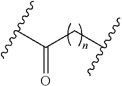 | 5 |
| 1317 | 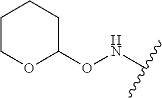 [SMILES: *NOC1CCCCO1] | 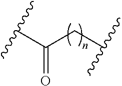 | 6 |
| 1318 | 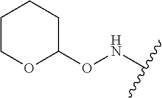 [SMILES: *NOC1CCCCO1] | 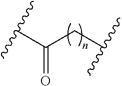 | 7 |
| 1319 | 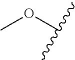 [SMILES: *OC] | 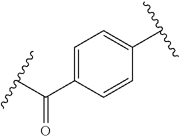 [SMILES: *C(=O)c1ccc(*)cc1] | — |
| 1320 | 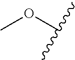 [SMILES: *OC] | 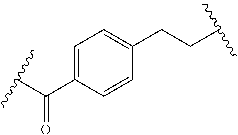 [SMILES: *CCc1ccc(C(*)=O)cc1] | |
| 1321 | 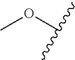 [SMILES: *OC] | 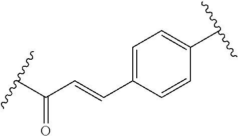 [SMILES: *C(=O)/C=C/c1ccc(*)cc1] | — |
| 1322 | 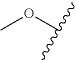 [SMILES: *OC] | 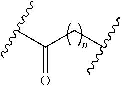 | 5 |
| 1323 | 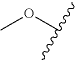 [SMILES: *OC] | 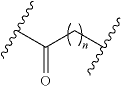 | 6 |
| 1324 | 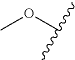 [SMILES: *OC] | 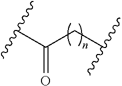 | 7 |
| 1325 | 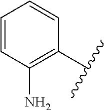 [SMILES: *c1ccccc1N] | 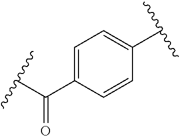 [SMILES: *C(=O)c1ccc(*)cc1] | — |
| 1326 | 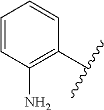 [SMILES: *c1ccccc1N] | 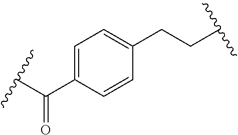 [SMILES: *CCc1ccc(C(*)=O)cc1] | |
| 1327 | 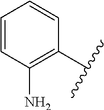 [SMILES: *c1ccccc1N] | 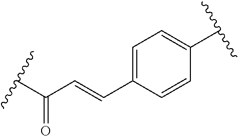 [SMILES: *C(=O)/C=C/c1ccc(*)cc1] | — |
| 1328 | 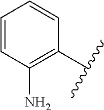 [SMILES: *c1ccccc1N] | 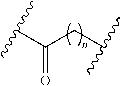 | 5 |
| 1329 | 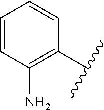 [SMILES: *c1ccccc1N] | 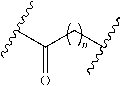 | 6 |
| 1330 | 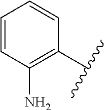 [SMILES: *c1ccccc1N] | 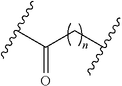 | 7 |
| 1331 | 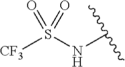 [SMILES: *NS(=O)(=O)C(F)(F)F] | 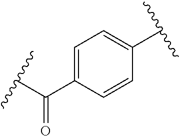 [SMILES: *C(=O)c1ccc(*)cc1] | — |
| 1332 | 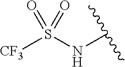 [SMILES: *NS(=O)(=O)C(F)(F)F] | 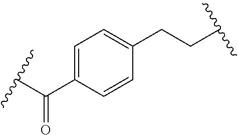 [SMILES: *CCc1ccc(C(*)=O)cc1] | |
| 1333 | 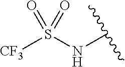 [SMILES: *NS(=O)(=O)C(F)(F)F] | 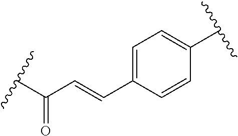 [SMILES: *C(=O)/C=C/c1ccc(*)cc1] | — |
| 1334 | 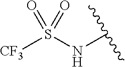 [SMILES: *NS(=O)(=O)C(F)(F)F] | 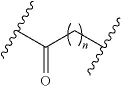 | 5 |
| 1335 | 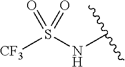 [SMILES: *NS(=O)(=O)C(F)(F)F] | 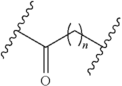 | 6 |
| 1336 | 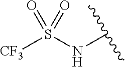 [SMILES: *NS(=O)(=O)C(F)(F)F] | 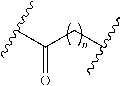 | 7 |
| 1337 | 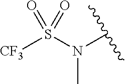 [SMILES: *N(C)S(=O)(=O)C(F)(F)F] | 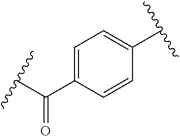 [SMILES: *C(=O)c1ccc(*)cc1] | — |
| 1338 | 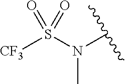 [SMILES: *N(C)S(=O)(=O)C(F)(F)F] | 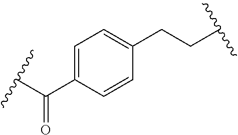 [SMILES: *CCc1ccc(C(*)=O)cc1] | |
| 1339 | 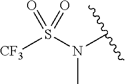 [SMILES: *N(C)S(=O)(=O)C(F)(F)F] | 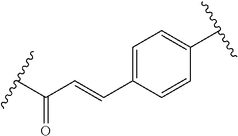 [SMILES: *C(=O)/C=C/c1ccc(*)cc1] | — |
| 1340 | 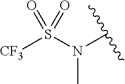 [SMILES: *N(C)S(=O)(=O)C(F)(F)F] | 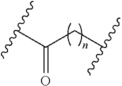 | 5 |
| 1341 | 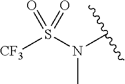 [SMILES: *N(C)S(=O)(=O)C(F)(F)F] | 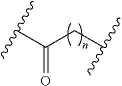 | 6 |
| 1342 | 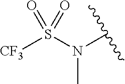 [SMILES: *N(C)S(=O)(=O)C(F)(F)F] | 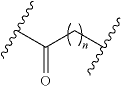 | 7 |

63. The compound of claim 51, wherein the compound of formula (I) is a compound ofany one of formula AMC-3-017, formula AMC-3-041, formula AMC-2-248, formula AMC-3-033, formula AMC-3-019, formula AMC-3-031, formula AMC-3-047, formula AMC-3-030, formula AMC-3-048, formula AMC-3-055, formula AMC-2-052, formula AMC-2-036, formula AMC-2-072, formula AMC-2-067, formula AMC-2-215, formula AMC-2-091, formula AMC-2-152, formula AMC-2-198, formula AMC-2-204, or formula AMC-2-225, or a pharmaceutically acceptable salt, solvate, hydrate, cocrystal, or prodrug thereof:

| Formula No. | Structure |
|---|---|
| AMC-3-017 | 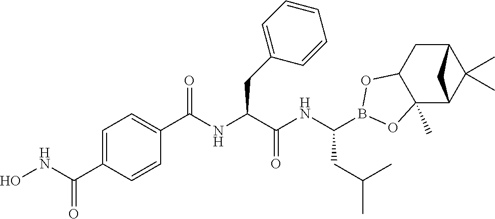 [SMILES: CC(C)C[C@H](NC(=O)[C@H](Cc1ccccc1)NC(=O)c1ccc(C(=O)NO)cc1)B1OC2C[C@H]3C[C@H](C3(C)C)[C@@]2(C)O1] |
| AMC-3-041 | 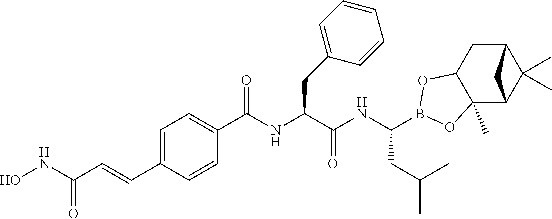 [SMILES: CC(C)C[C@H](NC(=O)[C@H](Cc1ccccc1)NC(=O)c1ccc(/C=C/C(=O)NO)cc1)B1OC2C[C@H]3C[C@H](C3(C)C)[C@@]2(C)O1] |
| AMC-2-248 | 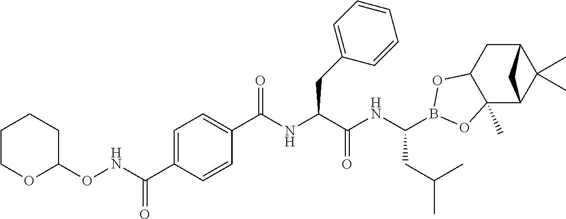 [SMILES: CC(C)C[C@H](NC(=O)[C@H](Cc1ccccc1)NC(=O)c1ccc(C(=O)NOC2CCCCO2)cc1)B1OC2C[C@H]3C[C@H](C3(C)C)[C@@]2(C)O1] |
| AMC-3-033 | 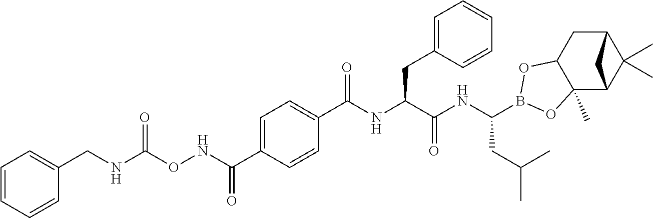 [SMILES: CC(C)C[C@H](NC(=O)[C@H](Cc1ccccc1)NC(=O)c1ccc(C(=O)NOC(=O)NCc2ccccc2)cc1)B1OC2C[C@H]3C[C@H](C3(C)C)[C@@]2(C)O1] |
| AMC-3-019 | 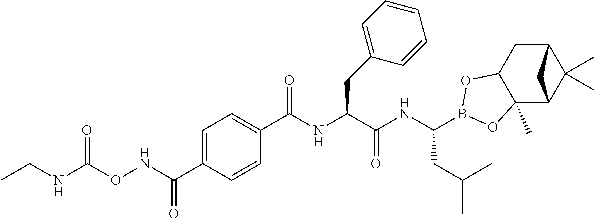 [SMILES: CCNC(=O)ONC(=O)c1ccc(C(=O)N[C@@H](Cc2ccccc2)C(=O)N[C@@H](CC(C)C)B2OC3C[C@H]4C[C@H](C4(C)C)[C@@]3(C)O2)cc1] |
| AMC-3-031 | 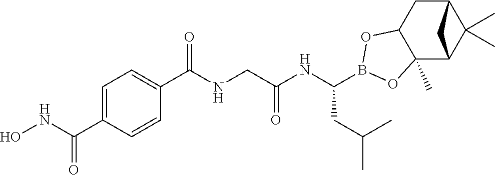 [SMILES: CC(C)C[C@H](NC(=O)CNC(=O)c1ccc(C(=O)NO)cc1)B1OC2C[C@H]3C[C@H](C3(C)C)[C@@]2(C)O1] |
| AMC-3-047 | 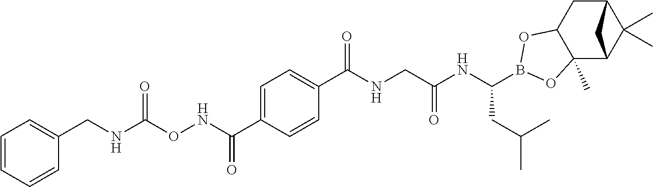 [SMILES: CC(C)C[C@H](NC(=O)CNC(=O)c1ccc(C(=O)NOC(=O)NCc2ccccc2)cc1)B1OC2C[C@H]3C[C@H](C3(C)C)[C@@]2(C)O1] |
| AMC-3-030 | 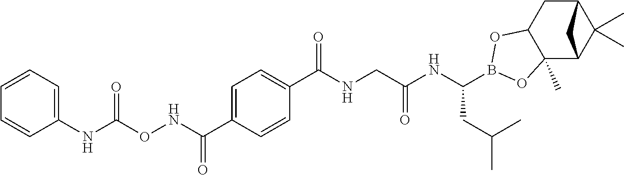 [SMILES: CC(C)C[C@H](NC(=O)CNC(=O)c1ccc(C(=O)NOC(=O)Nc2ccccc2)cc1)B1OC2C[C@H]3C[C@H](C3(C)C)[C@@]2(C)O1] |
| AMC-3-048 | 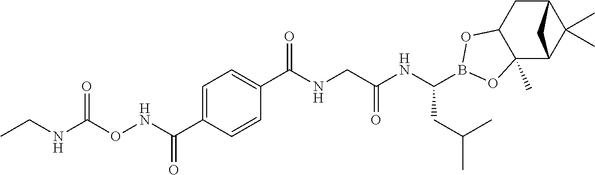 [SMILES: CCNC(=O)ONC(=O)c1ccc(C(=O)NCC(=O)N[C@@H](CC(C)C)B2OC3C[C@H]4C[C@H](C4(C)C)[C@@]3(C)O2)cc1] |
| AMC-3-055 | 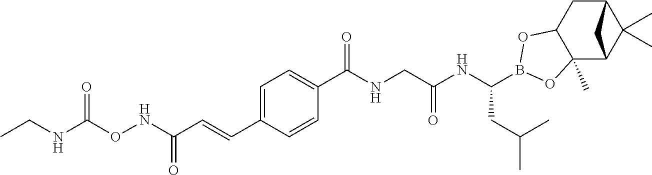 [SMILES: CCNC(=O)ONC(=O)/C=C/c1ccc(C(=O)NCC(=O)N[C@@H](CC(C)C)B2OC3C[C@H]4C[C@H](C4(C)C)[C@@]3(C)O2)cc1] |
| AMC-2-052 | 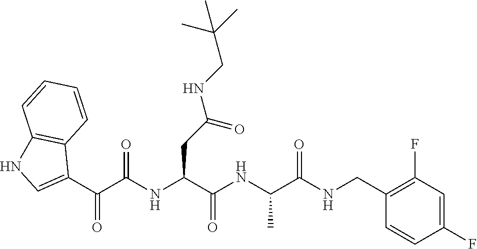 [SMILES: C[C@H](NC(=O)[C@H](CC(=O)NCC(C)(C)C)NC(=O)C(=O)c1c[nH]c2ccccc12)C(=O)NCc1ccc(F)cc1F] |
| AMC-2-036 | 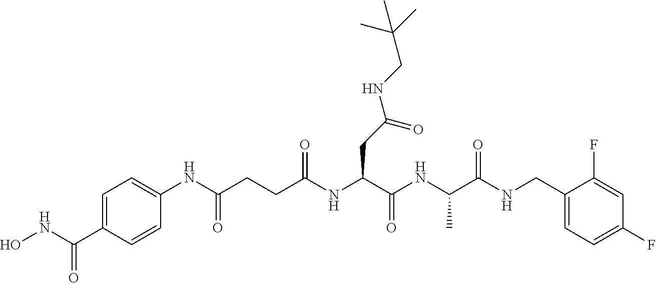 [SMILES: C[C@H](NC(=O)[C@H](CC(=O)NCC(C)(C)C)NC(=O)CCC(=O)Nc1ccc(C(=O)NO)cc1)C(=O)NCc1ccc(F)cc1F] |
| AMC-2-072 | 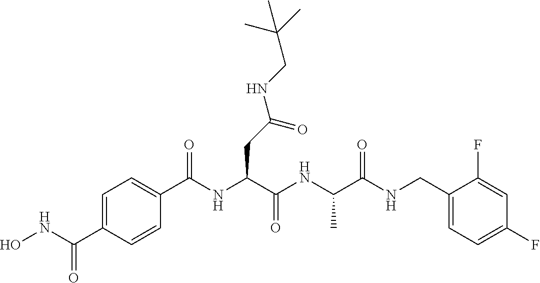 [SMILES: C[C@H](NC(=O)[C@H](CC(=O)NCC(C)(C)C)NC(=O)c1ccc(C(=O)NO)cc1)C(=O)NCc1ccc(F)cc1F] |
| AMC-2-067 | 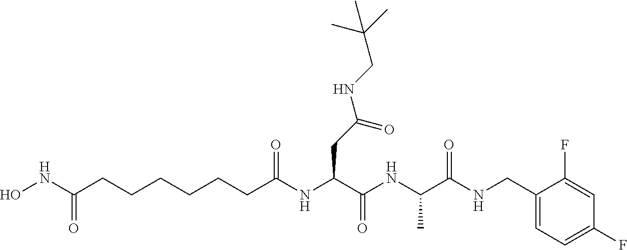 [SMILES: C[C@H](NC(=O)[C@H](CC(=O)NCC(C)(C)C)NC(=O)CCCCCCC(=O)NO)C(=O)NCc1ccc(F)cc1F] |
| AMC-2-215 | 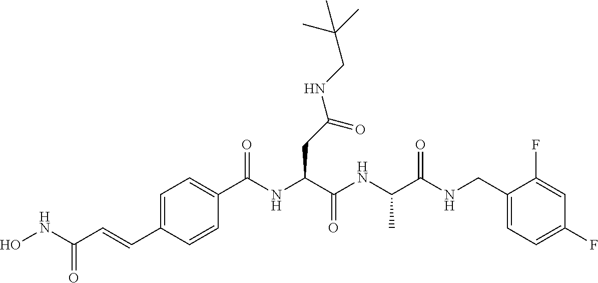 [SMILES: C[C@H](NC(=O)[C@H](CC(=O)NCC(C)(C)C)NC(=O)c1ccc(/C=C/C(=O)NO)cc1)C(=O)NCc1ccc(F)cc1F] |
| AMC-2-091 | 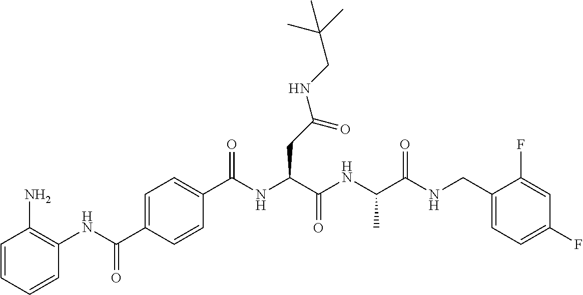 [SMILES: C[C@H](NC(=O)[C@H](CC(=O)NCC(C)(C)C)NC(=O)c1ccc(C(=O)Nc2ccccc2N)cc1)C(=O)NCc1ccc(F)cc1F] |
| AMC-2-152 | 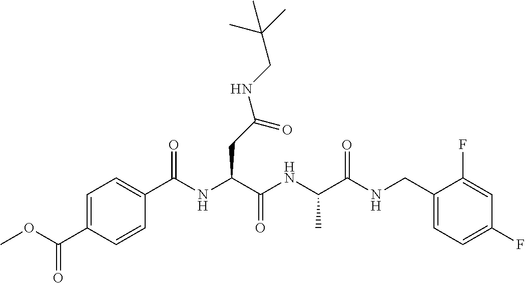 [SMILES: COC(=O)c1ccc(C(=O)N[C@@H](CC(=O)NCC(C)(C)C)C(=O)N[C@@H](C)C(=O)NCc2ccc(F)cc2F)cc1] |
| AMC-2-198 | 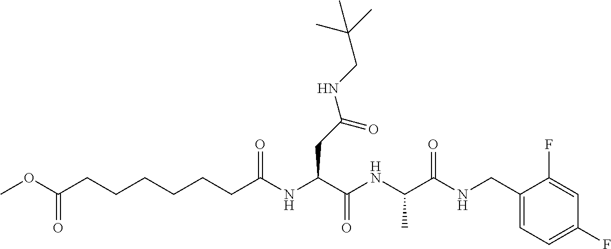 [SMILES: COC(=O)CCCCCCC(=O)N[C@@H](CC(=O)NCC(C)(C)C)C(=O)N[C@@H](C)C(=O)NCc1ccc(F)cc1F] |
| AMC-2-204 | 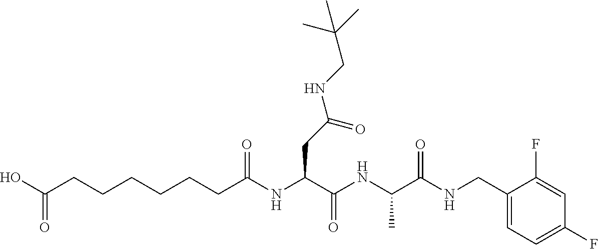 [SMILES: C[C@H](NC(=O)[C@H](CC(=O)NCC(C)(C)C)NC(=O)CCCCCCC(=O)O)C(=O)NCc1ccc(F)cc1F] |
| AMC-2-225 | 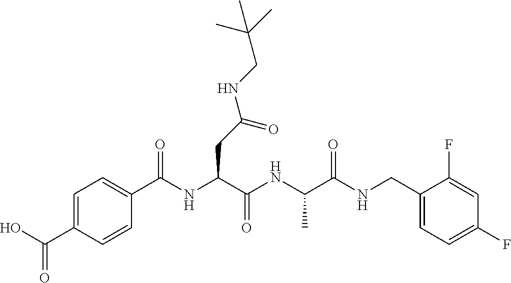 [SMILES: C[C@H](NC(=O)[C@H](CC(=O)NCC(C)(C)C)NC(=O)c1ccc(C(=O)O)cc1)C(=O)NCc1ccc(F)cc1F] |

64. A pharmaceutical composition comprising a compound of claim 51 or a pharmaceutically acceptable salt thereof, and a physiologically compatible carrier medium.

65. A method of treating or preventing a disease or disorder in a patient in need thereof, the method comprising administering a therapeutically effective amount of a compound of claim 51, or a pharmaceutically acceptable salt thereof.

66. The method of claim 65, wherein the disease or disorder is alleviated by:
(a) inhibiting HDAC6 protein activity in the patient;
(b) inhibiting proteasome activity in the patient; or
(c) inhibiting HDAC6 protein activity and inhibiting proteasome activity in the patient.

67. The method of claim 65, wherein the disease or disorder is cancer, an inflammatory disease or disorder, or an autoimmune disease or disorder.

68. The method of claim 67, wherein the disease or disorder is cancer.

69. The method of claim 68, wherein the cancer is a blood cancer.

70. The method of claim 69, wherein the cancer is acute multiple myeloma (MM).
